SEQUENCE LISTING (70 sequences)

SEQ ID NO: 1 - protein, 353 aa, Pyrococcus horikoshii
MEVRNMVDYELLKKVVEAPGVSGYEFLGIRDVVIEEIKDYVDEVKVDKLGNVIAHKKGEGPKVMIAAHMDQIGLMVTHIEKNGFLRVAPIGGVDPKTLIAQRFKVWIDKGKFIYGVGASVPPHIQKPEDRKKAPDWDQIFIDIGAESKEEAEDMGVKIGTVITWDGRLERLGKHRFVSIAFDDRIAVYTILEVAKQLKDAKADVYFVATVQEEVGLRGARTSAFGIEPDYGFAIDVTIAADIPGTPEHKQVTHLGKGTAIKIMDRSVICHPTIVRWLEELAKKHEIPYQLEILLGGGTDAGAIHLTKAGVPTGALSVPARYIHSNTEVVDERDVDATVELMTKALENIHELKI

SEQ ID NO: 2 - protein, 420 aa, unidentified
  source 1..420: note = AP30
MEVRNMVDYELLKKVVEAPGVSGYEFLGIRDVVIEEIKDYVDEVKVDKLGNVIAHKKGEGPKVMIAAHMDQIGLMVTHIEKNGFLRVAPIGGVDPKTLIAQRFKVWIDKGKFIYGVGASVPPHIQKPEDRKKAPDWDQIFIDIGAESKEEAEDMGVKIGTVITWDGRLERLGKHRFVSIAFDDRIAVYTILEVAKQLKDAKADVYFVATVQEEVGLRGARTSAFGIEPDYGFAIDVTIAADIPGTPEHKQVTHLGKGTAIKIMDRSVICHPTIVRWLEELAKKHEIPYQLEILLGGGTDAGAIHLTKAGVPTGALSVPARYIHSNTEVVDERDVDATVELMTKALENIHELKIGGSHHHHHHHHHHGGGSGGGSGGGSGLNDFFEAQKIEWHEGGGSGGGSGGGSGLNDFFEAQKIEWHE

SEQ ID NO: 3 - protein, 353 aa, Pyrococcus horikoshii
MDLKGGESMVDWKLMQEIIEAPGVSGYEHLGIRDIVVDVLKEVADEVKVDKLGNVIAHFKGSSPRIMVAAHMDKIGVMVNHIDKDGYLHIVPIGGVLPETLVAQRIRFFTEKGERYGVVGVLPPHLRRGQEDKGSKIDWDQIVVDVGASSKEEAEEMGFRVGTVGEFAPNFTRLNEHRFATPYLDDRICLYAMIEAARQLGDHEADIYIVGSVQEEVGLRGARVASYAINPEVGIAMDVTFAKQPHDKGKIVPELGKGPVMDVGPNINPKLRAFADEVAKKYEIPLQVEPSPRPTGTDANMQINREGVATAVLSIPIRYMHSQVELADARDVDNTIKLAKALLEELKPMDFTP

SEQ ID NO: 4 - protein, 364 aa, unidentified
  source 1..364: note = AP37
MDLKGGESMVDWKLMQEIIEAPGVSGYEHLGIRDIVVDVLKEVADEVKVDKLGNVIAHFKGSSPRIMVAAHMDKIGVMVNHIDKDGYLHIVPIGGVLPETLVAQRIRFFTEKGERYGVVGVLPPHLRRGQEDKGSKIDWDQIVVDVGASSKEEAEEMGFRVGTVGEFAPNFTRLNEHRFATPYLDDRICLYAMIEAARQLGDHEADIYIVGSVQEEVGLRGARVASYAINPEVGIAMDVTFAKQPHDKGKIVPELGKGPVMDVGPNINPKLRAFADEVAKKYEIPLQVEPSPRPTGTDANMQINREGVATAVLSIPIRYMHSQVELADARDVDNTIKLAKALLEELKPMDFTPGHHHHHHHHHH

SEQ ID NO: 5 - protein, 447 aa, Yersinia pestis
MTQQEYQNRRQALLAKMAPGSAAIIFAAPEATRSADSEYPYRQNSDFSYLTGFNEPEAVLILVKSDETHNHSVLFNRIRDLTAEIWFGRRLGQEAAPTKLAVDRALPFDEINEQLYLLLNRLDVIYHAQGQYAYADNIVFAALEKLRHGFRKNLRAPATLTDWRPWLHEMRLFKSAEEIAVLRRAGEISALAHTRAMEKCRPGMFEYQLEGEILHEFTRHGARYPAYNTIVGGGENGCILHYTENECELRDGDLVLIDAGCEYRGYAGDITRTFPVNGKFTPAQRAVYDIVLAAINKSLTLFRPGTSIREVTEEVVRIMVVGLVELGILKGDIEQLIAEQAHRPFFMHGLSHWLGMDVHDVGDYGSSDRGRILEPGMVLTVEPGLYIAPDADVPPQYRGIGIRIEDDIVITATGNENLTASVVKDPDDIEALMALNHAGENLYFQLE

SEQ ID NO: 6 - protein, 453 aa, unidentified
  source 1..453: note = yPIP-6x His
MTQQEYQNRRQALLAKMAPGSAAIIFAAPEATRSADSEYPYRQNSDFSYLTGFNEPEAVLILVKSDETHNHSVLFNRIRDLTAEIWFGRRLGQEAAPTKLAVDRALPFDEINEQLYLLLNRLDVIYHAQGQYAYADNIVFAALEKLRHGFRKNLRAPATLTDWRPWLHEMRLFKSAEEIAVLRRAGEISALAHTRAMEKCRPGMFEYQLEGEILHEFTRHGARYPAYNTIVGGGENGCILHYTENECELRDGDLVLIDAGCEYRGYAGDITRTFPVNGKFTPAQRAVYDIVLAAINKSLTLFRPGTSIREVTEEVVRIMVVGLVELGILKGDIEQLIAEQAHRPFFMHGLSHWLGMDVHDVGDYGSSDRGRILEPGMVLTVEPGLYIAPDADVPPQYRGIGIRIEDDIVITATGNENLTASVVKDPDDIEALMALNHAGENLYFQLEHHHHHH

SEQ ID NO: 7 - protein, 445 aa, unidentified
  source 1..445: note = yPIP
MTQQEYQNRRQALLAKMAPGSAAIIFAAPEATRSADSEYPYRQNSDFSYLTGFNEPEAVLILVKSDETHNHSVLFNRIRDLTAEIWFGRRLGQEAAPTKLAVDRALPFDEINEQLYLLLNRLDVIYHAQGQYAYADNIVFAALEKLRHGFRKNLRAPATLTDWRPWLHEMRLFKSAEEIAVLRRAGEISALAHTRAMEKCRPGMFEYQLEGEILHEFTRHGARYPAYNTIVGGGENGCILHYTENECELRDGDLVLIDAGCEYRGYAGDITRTFPVNGKFTPAQRAVYDIVLAAINKSLTLFRPGTSIREVTEEVVRIMVVGLVELGILKGDIEQLIAEQAHRPFFMHGLSHWLGMDVHDVGDYGSSDRGRILEPGMVLTVEPGLYIAPDADVPPQYRGIGIRIEDDIVITATGNENLTASVVKDPDDIEALMALNHAGENLYFQ

SEQ ID NO: 8 - protein, 454 aa, unidentified
  source 1..454: note = AP70
MTQQEYQNRRQALLAKMAPGSAAIIFAAPEATRSADSEYPYRQNSDFSYLTGFNEPEAVLILVKSDETHNHSVLFNRIRDLTAEIWFGRRLGQEAAPTKLAVDRALPFDEINEQLYLLLNRLDVIYHAQGQYAYADNIVFAALEKLRHGFRKNLRAPATLTDWRPWLHEMRLFKSAEEIAVLRRAGEISALAHTRAMEKCRPGMFEYQLEGEILHEFTRHGARYPAYNTIVGGGENGCILHYTENECELRDGDLVLIDAGCEYRGYAGDITRTFPVNGKFTPAQRAVYDIVLAAINKSLTLFRPGTSIREVTEEVVRIMVVGLVELGILKGDIEQLIAEQAHRPFFMHGLSHWLGMDVHDVGDYGSSDRGRILEPGMVLTVEPGLYIAPDADVPPQYRGIGIRIEDDIVITATGNENLTASVVKDPDDIEALMALNHAGENLYFQGGSHHHHHH

SEQ ID NO: 9 - protein, 901 aa, Legionella pneumophila
MMVKQGVFMKTDQSKVKKLSDYKSLDYFVIHVDLQIDLSKKPVESKARLTVVPNLNVDSHSNDLVLDGENMTLVSLQMNDNLLKENEYELTKDSLIIKNIPQNTPFTIEMTSLLGENTDLFGLYETEGVALVKAESEGLRRVFYLPDRPDNLATYKTTIIANQEDYPVLLSNGVLIEKKELPLGLHSVTWLDDVPKPSYLFALVAGNLQRSVTYYQTKSGRELPIEFYVPPSATSKCDFAKEVLKEAMAWDERTFNLECALRQHMVAGVDKYASGASEPTGLNLFNTENLFASPETKTDLGILRVLEVVAHEFFHYWSGDRVTIRDWFNLPLKEGLTTFRAAMFREELFGTDLIRLLDGKNLDERAPRQSAYTAVRSLYTAAAYEKSADIFRMMMLFIGKEPFIEAVAKFFKDNDGGAVTLEDFIESISNSSGKDLRSFLSWFTESGIPELIVTDELNPDTKQYFLKIKTVNGRNRPIPILMGLLDSSGAEIVADKLLIVDQEEIEFQFENIQTRPIPSLLRSFSAPVHMKYEYSYQDLLLLMQFDTNLYNRCEAAKQLISALINDFCIGKKIELSPQFFAVYKALLSDNSLNEWMLAELITLPSLEELIENQDKPDFEKLNEGRQLIQNALANELKTDFYNLLFRIQISGDDDKQKLKGFDLKQAGLRRLKSVCFSYLLNVDFEKTKEKLILQFEDALGKNMTETALALSMLCEINCEEADVALEDYYHYWKNDPGAVNNWFSIQALAHSPDVIERVKKLMRHGDFDLSNPNKVYALLGSFIKNPFGFHSVTGEGYQLVADAIFDLDKINPTLAANLTEKFTYWDKYDVNRQAMMISTLKIIYSNATSSDVRTMAKKGLDKVKEDLPLPIHLTFHGGSTMQDRTAQLIADGNKENAYQLH

SEQ ID NO: 10 - protein, 265 aa, Escherichia coli
MGTAISIKTPEDIEKMRVAGRLAAEVLEMIEPYVKPGVSTGELDRICNDYIVNEQHAVSACLGYHGYPKSVCISINEVVCHGIPDDAKLLKDGDIVNIDVTVIKDGFHGDTSKMFIVGKPTIMGERLCRITQESLYLALRMVKPGINLREIGAAIQKFVEAEGFSVVREYCGHGIGRGFHEEPQVLHYDSRETNVVLKPGMTFTIEPMVNAGKKEIRTMKDGWTVKTKDRSLSAQYEHTIVVTDNGCEILTLRKDDTIPAIISHD

SEQ ID NO: 11 - protein, 322 aa, Mycobacterium smegmatis
MGTLEANTNGPGSMLSRMPVSSRTVPFGDHETWVQVTTPENAQPHALPLIVLHGGPGMAHNYVANIAALADETGRTVIHYDQVGCGNSTHLPDAPADFWTPQLFVDEFHAVCTALGIERYHVLGQSWGGMLGAEIAVRQPSGLVSLAICNSPASMRLWSEAAGDLRAQLPAETRAALDRHEAAGTITHPDYLQAAAEFYRRHVCRVVPTPQDFADSVAQMEAEPTVYHTMNGPNEFHVVGTLGDWSVIDRLPDVTAPVLVIAGEHDEATPKTWQPFVDHIPDVRSHVFPGTSHCTHLEKPEEFRAVVAQFLHQHDLAADARV

SEQ ID NO: 12 - protein, 303 aa, Pyrococcus furiosus
MDTEKLMKAGEIAKKVREKAIKLARPGMLLLELAESIEKMIMELGGKPAFPVNLSINEIAAHYTPYKGDTTVLKEGDYLKIDVGVHIDGFIADTAVTVRVGMEEDELMEAAKEALNAAISVARAGVEIKELGKAIENEIRKRGFKPIVNLSGHKIERYKLHAGISIPNIYRPHDNYVLKEGDVFAIEPFATIGAGQVIEVPPTLIYMYVRDVPVRVAQARFLLAKIKREYGTLPFAYRWLQNDMPEGQLKLALKTLEKAGAIYGYPVLKEIRNGIVAQFEHTIIVEKDSVIVTQDMINKSTLE

SEQ ID NO: 13 - protein, 428 aa, Aeromonas sobria
HMSSPLHYVLDGIHCEPHFFTVPLDHQQPDDEETITLFGRTLCRKDRLDDELPWLLYLQGGPGFGAPRPSANGGWIKRALQEFRVLLLDQRGTGHSTPIHAELLAHLNPRQQADYLSHFRADSIVRDAELIREQLSPDHPWSLLGQSFGGFCSLTYLSLFPDSLHEVYLTGGVAPIGRSADEVYRATYQRVADKNRAFFARFPHAQAIANRLATHLQRHDVRLPNGQRLTVEQLQQQGLDLGASGAFEELYYLLEDAFIGEKLNPAFLYQVQAMQPFNTNPVFAILHELIYCEGAASHWAAERVRGEFPALAWAQGKDFAFTGEMIFPWMFEQFRELIPLKEAAHLLAEKADWGPLYDPVQLARNKVPVACAVYAEDMYVEFDYSRETLKGLSNSRAWITNEYEHNGLRVDGEQILDRLIRLNRDCLE

SEQ ID NO: 14 - protein, 348 aa, Pyrococcus furiosus
MKERLEKLVKFMDENSIDRVFIAKPVNVYYFSGTSPLGGGYIIVDGDEATLYVPELEYEMAKEESKLPVVKFKKFDEIYEILKNTETLGIEGTLSYSMVENFKEKSNVKEFKKIDDVIKDLRIIKTKEEIEIIEKACEIADKAVMAAIEEITEGKREREVAAKVEYLMKMNGAEKPAFDTIIASGHRSALPHGVASDKRIERGDLVVIDLGALYNHYNSDITRTIVVGSPNEKQREIYEIVLEAQKRAVEAAKPGMTAKELDSIAREIIKEYGYGDYFIHSLGHGVGLEIHEWPRISQYDETVLKEGMVITIEPGIYIPKLGGVRIEDTVLITENGAKRLTKTERELL

SEQ ID NO: 15 - protein, 298 aa, unidentified
  source 1..298: note = Elizabethkingia meningoseptica
MIPITTPVGNFKVWTKRFGTNPKIKVLLLHGGPAMTHEYMECFETFFQREGFEFYEYDQLGSYYSDQPTDEKLWNIDRFVDEVEQVRKAIHADKENFYVLGNSWGGILAMEYALKYQQNLKGLIVANMMASAPEYVKYAEVLSKQMKPEVLAEVRAIEAKKDYANPRYTELLFPNYYAQHICRLKEWPDALNRSLKHVNSTVYTLMQGPSELGMSSDARLAKWDIKNRLHEIATPTLMIGARYDTMDPKAMEEQSKLVQKGRYLYCPNGSHLAMWDDQKVFMDGVIKFIKDVDTKSFN

SEQ ID NO: 16 - protein, 310 aa, Neisseria gonorrhoeae
MYEIKQPFHSGYLQVSEIHQIYWEESGNPDGVPVIFLHGGPGAGASPECRGFFNPDVFRIVIIDQRGCGRSHPYACAEDNTTWDLVADIEKVREMLGIGKWLVFGGSWGSTLSLAYAQTHPERVKGLVLRGIFLCRPSETAWLNEAGGVSRIYPEQWQKFVAPIAENRRNRLIEAYHGLLFHQDEEVCLSAAKAWADWESYLIRFEPEGVDEDAYASLAIARLENHYFVNGGWLQGDKAILNNIGKIRHIPTVIVQGRYDLCTPMQSAWELSKAFPEAELRVVQAGHCAFDPPLADALVQAVEDILPRLL

SEQ ID NO: 17 - protein, 870 aa, Escherichia coli
MTQQPQAKYRHDYRAPDYQITDIDLTFDLDAQKTVVTAVSQAVRHGASDAPLRLNGEDLKLVSVHINDEPWTAWKEEEGALVISNLPERFTLKIINEISPAANTALEGLYQSGDALCTQCEAEGFRHITYYLDRPDVLARFTTKIIADKIKYPFLLSNGNRVAQGELENGRHWVQWQDPFPKPCYLFALVAGDFDVLRDTFTTRSGREVALELYVDRGNLDRAPWAMTSLKNSMKWDEERFGLEYDLDIYMIVAVDFFNMGAMENKGLNIFNSKYVLARTDTATDKDYLDIERVIGHEYFHNWTGNRVTCRDWFQLSLKEGLTVFRDQEFSSDLGSRAVNRINNVRTMRGLQFAEDASPMAHPIRPDMVIEMNNFYTLTVYEKGAEVIRMIHTLLGEENFQKGMQLYFERHDGSAATCDDFVQAMEDASNVDLSHFRRWYSQSGTPIVTVKDDYNPETEQYTLTISQRTPATPDQAEKQPLHIPFAIELYDNEGKVIPLQKGGHPVNSVLNVTQAEQTFVFDNVYFQPVPALLCEFSAPVKLEYKWSDQQLTFLMRHARNDFSRWDAAQSLLATYIKLNVARHQQGQPLSLPVHVADAFRAVLLDEKIDPALAAEILTLPSVNEMAELFDIIDPIAIAEVREALTRTLATELADELLAIYNANYQSEYRVEHEDIAKRTLRNACLRFLAFGETHLADVLVSKQFHEANNMTDALAALSAAVAAQLPCRDALMQEYDDKWHQNGLVMDKWFILQATSPAANVLETVRGLLQHRSFTMSNPNRIRSLIGAFAGSNPAAFHAEDGSGYLFLVEMLTDLNSRNPQVASRLIEPLIRLKRYDAKRQEKMRAALEQLKGLENLSGDLYEKITKALA

SEQ ID NO: 18 - protein, 889 aa, Plasmodium falciparum
PKIHYRKDYKPSGFIINQVTLNINIHDQETIVRSVLDMDISKHNVGEDLVFDGVGLKINEISINNKKLVEGEEYTYDNEFLTIFSKFVPKSKFAFSSEVIIHPETNYALTGLYKSKNIIVSQCEATGFRRITFFIDRPDMMAKYDVTVTADKEKYPVLLSNGDKVNEFEIPGGRHGARFNDPPLKPCYLFAVVAGDLKHLSATYITKYTKKKVELYVFSEEKYVSKLQWALECLKKSMAFDEDYFGLEYDLSRLNLVAVSDFNVGAMENKGLNIFNANSLLASKKNSIDFSYARILTVVGHEYFHQYTGNRVTLRDWFQLTLKEGLTVHRENLFSEEMTKTVTTRLSHVDLLRSVQFLEDSSPLSHPIRPESYVSMENFYTTTVYDKGSEVMRMYLTILGEEYYKKGFDIYIKKNDGNTATCEDFNYAMEQAYKMKKADNSANLNQYLLWFSQSGTPHVSFKYNYDAEKKQYSIHVNQYTKPDENQKEKKPLFIPISVGLINPENGKEMISQTTLELTKESDTFVFNNIAVKPIPSLFRGFSAPVYIEDQLTDEERILLLKYDSDAFVRYNSCTNIYMKQILMNYNEFLKAKNEKLESFQLTPVNAQFIDAIKYLLEDPHADAGFKSYIVSLPQDRYIINFVSNLDTDVLADTKEYIYKQIGDKLNDVYYKMFKSLEAKADDLTYFNDESHVDFDQMNMRTLRNTLLSLLSKAQYPNILNEIIEHSKSPYPSNWLTSLSVSAYFDKYFELYDKTYKLSKDDELLLQEWLKTVSRSDRKDIYEILKKLENEVLKDSKNPNDIRAVYLPFTNNLRRFHDISGKGYKLIAEVITKTDKFNPMVATQLCEPFKLWNKLDTKRQELMLNEMNTMLQEPQISNNLKEYLLRLTNK

SEQ ID NO: 19 - protein, 919 aa, unidentified
  source 1..919: note = Puromycin-sensitive aminopeptidase
MWLAAAAPSLARRLLFLGPPPPPLLLLVFSRSSRRRLHSLGLAAMPEKRPFERLPADVSPINYSLCLKPDLLDFTFEGKLEAAAQVRQATNQIVMNCADIDIITASYAPEGDEEIHATGFNYQNEDEKVTLSFPSTLQTGTGTLKIDFVGELNDKMKGFYRSKYTTPSGEVRYAAVTQFEATDARRAFPCWDEPAIKATFDISLVVPKDRVALSNMNVIDRKPYPDDENLVEVKFARTPVMSTYLVAFVVGEYDFVETRSKDGVCVRVYTPVGKAEQGKFALEVAAKTLPFYKDYFNVPYPLPKIDLIAIADFAAGAMENWGLVTYRETALLIDPKNSCSSSRQWVALVVGHELAHQWFGNLVTMEWWTHLWLNEGFASWIEYLCVDHCFPEYDIWTQFVSADYTRAQELDALDNSHPIEVSVGHPSEVDEIFDAISYSKGASVIRMLHDYIGDKDFKKGMNMYLTKFQQKNAATEDLWESLENASGKPIAAVMNTWTKQMGFPLIYVEAEQVEDDRLLRLSQKKFCAGGSYVGEDCPQWMVPITISTSEDPNQAKLKILMDKPEMNVVLKNVKPDQWVKLNLGTVGFYRTQYSSAMLESLLPGIRDLSLPPVDRLGLQNDLFSLARAGIISTVEVLKVMEAFVNEPNYTVWSDLSCNLGILSTLLSHTDFYEEIQEFVKDVFSPIGERLGWDPKPGEGHLDALLRGLVLGKLGKAGHKATLEEARRRFKDHVEGKQILSADLRSPVYLTVLKHGDGTTLDIMLKLHKQADMQEEKNRIERVLGATLLPDLIQKVLTFALSEEVRPQDTVSVIGGVAGGSKHGRKAAWKFIKDNWEELYNRYQGGFLISRLIKLSVEGFAVDKMAGEVKAFFESHPAPSAERTIQQCCENILLNAAWLKRDAESIHQYLLQRKASPPTV

SEQ ID NO: 20 - protein, 919 aa, unidentified
  source 1..919: note = NPEPPS E366V
MWLAAAAPSLARRLLFLGPPPPPLLLLVFSRSSRRRLHSLGLAAMPEKRPFERLPADVSPINYSLCLKPDLLDFTFEGKLEAAAQVRQATNQIVMNCADIDIITASYAPEGDEEIHATGFNYQNEDEKVTLSFPSTLQTGTGTLKIDFVGELNDKMKGFYRSKYTTPSGEVRYAAVTQFEATDARRAFPCWDEPAIKATFDISLVVPKDRVALSNMNVIDRKPYPDDENLVEVKFARTPVMSTYLVAFVVGEYDFVETRSKDGVCVRVYTPVGKAEQGKFALEVAAKTLPFYKDYFNVPYPLPKIDLIAIADFAAGAMENWGLVTYRETALLIDPKNSCSSSRQWVALVVGHVLAHQWFGNLVTMEWWTHLWLNEGFASWIEYLCVDHCFPEYDIWTQFVSADYTRAQELDALDNSHPIEVSVGHPSEVDEIFDAISYSKGASVIRMLHDYIGDKDFKKGMNMYLTKFQQKNAATEDLWESLENASGKPIAAVMNTWTKQMGFPLIYVEAEQVEDDRLLRLSQKKFCAGGSYVGEDCPQWMVPITISTSEDPNQAKLKILMDKPEMNVVLKNVKPDQWVKLNLGTVGFYRTQYSSAMLESLLPGIRDLSLPPVDRLGLQNDLFSLARAGIISTVEVLKVMEAFVNEPNYTVWSDLSCNLGILSTLLSHTDFYEEIQEFVKDVFSPIGERLGWDPKPGEGHLDALLRGLVLGKLGKAGHKATLEEARRRFKDHVEGKQILSADLRSPVYLTVLKHGDGTTLDIMLKLHKQADMQEEKNRIERVLGATLLPDLIQKVLTFALSEEVRPQDTVSVIGGVAGGSKHGRKAAWKFIKDNWEELYNRYQGGFLISRLIKLSVEGFAVDKMAGEVKAFFESHPAPSAERTIQQCCENILLNAAWLKRDAESIHQYLLQRKASPPTV

SEQ ID NO: 21 - protein, 864 aa, Francisella tularensis
MIYEFVMTDPKIKYLKDYKPSNYLIDETHLIFELDESKTRVTANLYIVANRENRENNTLVLDGVELKLLSIKLNNKHLSPAEFAVNENQLIINNVPEKFVLQTVVEINPSANTSLEGLYKSGDVFSTQCEATGFRKITYYLDRPDVMAAFTVKIIADKKKYPIILSNGDKIDSGDISDNQHFAVWKDPFKKPCYLFALVAGDLASIKDTYITKSQRKVSLEIYAFKQDIDKCHYAMQAVKDSMKWDEDRFGLEYDLDTFMIVAVPDFNAGAMENKGLNIFNTKYIMASNKTATDKDFELVQSVVGHEYFHNWTGDRVTCRDWFQLSLKEGLTVFRDQEFTSDLNSRDVKRIDDVRIIRSAQFAEDASPMSHPIRPESYIEMNNFYTVTVYNKGAEIIRMIHTLLGEEGFQKGMKLYFERHDGQAVTCDDFVNAMADANNRDFSLFKRWYAQSGTPNIKVSENYDASSQTYSLTLEQTTLPTADQKEKQALHIPVKMGLINPEGKNIAEQVIELKEQKQTYTFENIAAKPVASLFRDFSAPVKVEHKRSEKDLLHIVKYDNNAFNRWDSLQQIATNIILNNADLNDEFLNAFKSILHDKDLDKALISNALLIPIESTIAEAMRVIMVDDIVLSRKNVVNQLADKLKDDWLAVYQQCNDNKPYSLSAEQIAKRKLKGVCLSYLMNASDQKVGTDLAQQLFDNADNMTDQQTAFTELLKSNDKQVRDNAINEFYNRWRHEDLVVNKWLLSQAQISHESALDIVKGLVNHPAYNPKNPNKVYSLIGGFGANFLQYHCKDGLGYAFMADTVLALDKFNHQVAARMARNLMSWKRYDSDRQAMMKNALEKIKASNPSKNVFEIVSKSLES

SEQ ID NO: 22 - protein, 408 aa, Thermus aquaticus
MDAFTENLNKLAELAIRVGLNLEEGQEIVATAPIEAVDFVRLLAEKAYENGASLFTVLYGDNLIARKRLALVPEAHLDRAPAWLYEGMAKAFHEGAARLAVSGNDPKALEGLPPERVGRAQQAQSRAYRPTLSAITEFVTNWTIVPFAHPGWAKAVFPGLPEEEAVQRLWQAIFQATRVDQEDPVAAWEAHNRVLHAKVAFLNEKRFHALHFQGPGTDLTVGLAEGHLWQGGATPTKKGRLCNPNLPTEEVFTAPHRERVEGVVRASRPLALSGQLVEGLWARFEGGVAVEVGAEKGEEVLKKLLDTDEGARRLGEVALVPADNPIAKTGLVFFDTLFDENAASHIAFGQAYAENLEGRPSGEEFRRRGGNESMVHVDWMIGSEEVDVDGLLEDGTRVPLMRRGRWVI

SEQ ID NO: 23 - protein, 362 aa, Bacillus stearothermophilus
MAKLDETLTMLKALTDAKGVPGNEREARDVMKTYIAPYADEVTTDGLGSLIAKKEGKSGGPKVMIAGHLDEVGFMVTQIDDKGFIRFQTLGGWWSQVMLAQRVTIVTKKGDITGVIGSKPPHILPSEARKKPVEIKDMFIDIGATSREEAMEWGVRPGDMIVPYFEFTVLNNEKMLLAKAWDNRIGCAVAIDVLKQLKGVDHPNTVYGVGTVQEEVGLRGARTAAQFIQPDIAFAVDVGIAGDTPGVSEKEAMGKLGAGPHIVLYDATMVSHRGLREFVIEVAEELNIPHHFDAMPGVGTDAGAIHLTGIGVPSLTIAIPTRYIHSHAAILHRDDYENTVKLLVEVIKRLDADKVKQLTFDE

SEQ ID NO: 24 - protein, 484 aa, Vibrio cholera
MEDKVWISMGADAVGSLNPALSESLLPHSFASGSQVWIGEVAIDELAELSHTMHEQHNRCGGYMVHTSAQGAMAALMMPESIANFTIPAPSQQDLVNAWLPQVSADQITNTIRALSSFNNRFYTTTSGAQASDWLANEWRSLISSLPGSRIEQIKHSGYNQKSVVLTIQGSEKPDEWVIVGGHLDSTLGSHTNEQSIAPGADDDASGIASLSEIIRVLRDNNFRPKRSVALMAYAAEEVGLRGSQDLANQYKAQGKKVVSVLQLDMTNYRGSAEDIVFITDYTDSNLTQFLTTLIDEYLPELTYGYDRCGYACSDHASWHKAGFSAAMPFESKFKDYNPKIHTSQDTLANSDPTGNHAVKFTKLGLAYVIEMANAGSSQVPDDSVLQDGTAKINLSGARGTQKRFTFELSQSKPLTIQTYGGSGDVDLYVKYGSAPSKSNWDCRPYQNGNRETCSFNNAQPGIYHVMLDGYTNYNDVALKASTQ

SEQ ID NO: 25 - protein, 488 aa, Photobacterium halotolerans
MEDKVWISIGSDASQTVKSVMQSNARSLLPESLASNGPVWVGQVDYSQLAELSHHMHEDHQRCGGYMVHSSPESAIAASNMPQSLVAFSIPEISQQDTVNAWLPQVNSQAITGTITSLTSFINRFYTTTSGAQASDWLANEWRSLSASLPNASVRQVSHFGYNQKSVVLTITGSEKPDEWIVLGGHLDSTIGSHTNEQSVAPGADDDASGIASVTEIIRVLSENNFQPKRSIAFMAYAAEEVGLRGSQDLANQYKAEGKQVISALQLDMTNYKGSVEDIVFITDYTDSNLTTFLSQLVDEYLPSLTYGFDTCGYACSDHASWHKAGFSAAMPFEAKFNDYNPMIHTPNDTLQNSDPTASHAVKFTKLGLAYAIEMASTTGGTPPPTGNVLKDGVPVNGLSGATGSQVHYSFELPAQKNLQISTAGGSGDVDLYVSFGSEATKQNWDCRPYRNGNNEVCTFAGATPGTYSIMLDGYRQFSGVTLKASTQ

SEQ ID NO: 26 - protein, 871 aa, Yersinia pestis
MTQQPQAKYRHDYRAPDYTITDIDLDFALDAQKTTVTAVSKVKRQGTDVTPLILNGEDLTLISVSVDGQAWPHYRQQDNTLVIEQLPADFTLTIVNDIHPATNSALEGLYLSGEALCTQCEAEGFRHITYYLDRPDVLARFTTRIVADKSRYPYLLSNGNRVGQGELDDGRHWVKWEDPFPKPSYLFALVAGDFDVLQDKFITRSGREVALEIFVDRGNLDRADWAMTSLKNSMKWDETRFGLEYDLDIYMIVAVDFFNMGAMENKGLNVFNSKYVLAKAETATDKDYLNIEAVIGHEYFHNWTGNRVTCRDWFQLSLKEGLTVFRDQEFSSDLGSRSVNRIENVRVMRAAQFAEDASPMAHAIRPDKVIEMNNFYTLTVYEKGSEVIRMMHTLLGEQQFQAGMRLYFERHDGSAATCDDFVQAMEDVSNVDLSLFRRWYSQSGTPLLTVHDDYDVEKQQYHLFVSQKTLPTADQPEKLPLHIPLDIELYDSKGNVIPLQHNGLPVHHVLNVTEAEQTFTFDNVAQKPIPSLLREFSAPVKLDYPYSDQQLTFLMQHARNEFSRWDAAQSLLATYIKLNVAKYQQQQPLSLPAHVADAFRAILLDEHLDPALAAQILTLPSENEMAELFTTIDPQAISTVHEAITRCLAQELSDELLAVYVANMTPVYRIEHGDIAKRALRNTCLNYLAFGDEEFANKLVSLQYHQADNMTDSLAALAAAVAAQLPCRDELLAAFDVRWNHDGLVMDKWFALQATSPAANVLVQVRTLLKHPAFSLSNPNRTRSLIGSFASGNPAAFHAADGSGYQFLVEILSDLNTRNPQVAARLIEPLIRLKRYDAGRQALMRKALEQLKTLDNLSGDLYEKITKALAA

SEQ ID NO: 27 - protein, 483 aa, Vibrio anguillarum
MEEKVWISIGGDATQTALRSGAQSLLPENLINQTSVWVGQVPVSELATLSHEMHENHQRCGGYMVHPSAQSAMSVSAMPLNLNAFSAPEITQQTTVNAWLPSVSAQQITSTITTLTQFKNRFYTTSTGAQASNWIADHWRSLSASLPASKVEQITHSGYNQKSVMLTITGSEKPDEWVVIGGHLDSTLGSRTNESSIAPGADDDASGIAGVTEIIRLLSEQNFRPKRSIAFMAYAAEEVGLRGSQDLANRFKAEGKKVMSVMQLDMTNYQGSREDIVFITDYTDSNFTQYLTQLLDEYLPSLTYGFDTCGYACSDHASWHAVGYPAAMPFESKFNDYNPNIHSPQDTLQNSDPTGFHAVKFTKLGLAYVVEMGNASTPPTPSNQLKNGVPVNGLSASRNSKTWYQFELQEAGNLSIVLSGGSGDADLYVKYQTDADLQQYDCRPYRSGNNETCQFSNAQPGRYSILLHGYNNYSNASLVANAQ

SEQ ID NO: 28 - protein, 482 aa, unidentified
  source 1..482: note = Salinivibrio spYCSC6
MEDKKVWISIGADAQQTALSSGAQPLLAQSVAHNGQAWIGEVSESELAALSHEMHENHHRCGGYIVHSSAQSAMAASNMPLSRASFIAPAISQQALVTPWISQIDSALIVNTIDRLTDFPNRFYTTTSGAQASDWIKQRWQSLSAGLAGASVTQISHSGYNQASVMLTIEGSESPDEWVVVGGHLDSTIGSRTNEQSIAPGADDDASGIAAVTEVIRVLAQNNFQPKRSIAFVAYAAEEVGLRGSQDVANQFKQAGKDVRGVLQLDMTNYQGSAEDIVFITDYTDNQLTQYLTQLLDEYLPTLNYGFDTCGYACSDHASWHQVGYPAAMPFEAKFNDYNPNIHTPQDTLANSDSEGAHAAKFTKLGLAYTVELANADSSPNPGNELKLGEPINGLSGARGNEKYFNYRLDQSGELVIRTYGGSGDVDLYVKANGDVSTGNWDCRPYRSGNDEVCRFDNATPGNYAVMLRGYRTYDNVSLIVE

SEQ ID NO: 29 - protein, 301 aa, Vibrio proteolyticus
MPPITQQATVTAWLPQVDASQITGTISSLESFTNRFYTTTSGAQASDWIASEWQALSASLPNASVKQVSHSGYNQKSVVMTITGSEAPDEWIVIGGHLDSTIGSHTNEQSVAPGADDDASGIAAVTEVIRVLSENNFQPKRSIAFMAYAAEEVGLRGSQDLANQYKSEGKNVVSALQLDMTNYKGSAQDVVFITDYTDSNFTQYLTQLMDEYLPSLTYGFDTCGYACSDHASWHNAGYPAAMPFESKFNDYNPRIHTTQDTLANSDPTGSHAKKFTQLGLAYAIEMGSATGDTPTPGNQLE

SEQ ID NO: 30 - protein, 301 aa, Vibrio proteolyticus
MPPITQQATVTAWLPQVDASQITGTISSLESFTNRFYTTTSGAQASDWIASEWQFLSASLPNASVKQVSHSGYNQKSVVMTITGSEAPDEWIVIGGHLDSTIGSHTNEQSVAPGADDDASGIAAVTEVIRVLSENNFQPKRSIAFMAYAAEEVGLRGSQDLANQYKSEGKNVVSALQLDMTNYKGSAQDVVFITDYTDSNFTQYLTQLMDEYLPSLTYGFDTCGYACSDHASWHNAGYPAAMPFESKFNDYNPRIHTTQDTLANSDPTGSHAKKFTQLGLAYAIEMGSATGDTPTPGNQLE

SEQ ID NO: 31 - protein, 348 aa, Pyrococcus furiosus
MVDWELMKKIIESPGVSGYEHLGIRDLVVDILKDVADEVKIDKLGNVIAHFKGSAPKVMVAAHMDKIGLMVNHIDKDGYLRVVPIGGVLPETLIAQKIRFFTEKGERYGVVGVLPPHLRREAKDQGGKIDWDSIIVDVGASSREEAEEMGFRIGTIGEFAPNFTRLSEHRFATPYLDDRICLYAMIEAARQLGEHEADIYIVASVQEEIGLRGARVASFAIDPEVGIAMDVTFAKQPNDKGKIVPELGKGPVMDVGPNINPKLRQFADEVAKKYEIPLQVEPSPRPTGTDANVMQINREGVATAVLSIPIRYMHSQVELADARDVDNTIKLAKALLEELKPMDFTPLE

SEQ ID NO: 32 - protein, 10 aa, synthetic construct
DDGGGDDDFF

SEQ ID NO: 33 - protein, 19 aa, synthetic construct
  MOD_RES 19: note = azidolysine
LASSIAEANRFADIADYPX

SEQ ID NO: 34 - protein, 11 aa, synthetic construct
  MOD_RES 11: note = azidolysine
RLIFAYPDDDX

SEQ ID NO: 35 - protein, 9 aa, synthetic construct
  MOD_RES 9: note = azidolysine
RLIFAYPGX

SEQ ID NO: 36 - protein, 13 aa, synthetic construct
  MOD_RES 13: note = azidolysine
LAQFASIAAYASX

SEQ ID NO: 37 - protein, 7 aa, synthetic construct
RRLIFSK

SEQ ID NO: 38 - protein, 11 aa, synthetic construct
LQYRSRLQYMK

SEQ ID NO: 39 - protein, 9 aa, synthetic construct
FRLNLYELK

SEQ ID NO: 40 - protein, 10 aa, synthetic construct
DQQRLIFAGK

SEQ ID NO: 41 - protein, 24 aa, synthetic construct
CCACGCGTGGAACCCTTGGGATCC

SEQ ID NO: 42 - protein, 25 aa, synthetic construct
CCACGCGTGGAACCCTTGGGATCCA

SEQ ID NO: 43 - protein, 9 aa, synthetic construct
DDGGDDDFF

SEQ ID NO: 44 - protein, 11 aa, synthetic construct
DDGGGDDDFFK

SEQ ID NO: 45 - protein, 10 aa, synthetic construct
  MOD_RES 6..8: note = cysteic acid
DDGGGXXXFF

SEQ ID NO: 46 - protein, 11 aa, synthetic construct
  MOD_RES 6..8: note = cysteic acid
DDGGGXXXFFK

SEQ ID NO: 47 - protein, 7 aa, synthetic construct
EFLNRFY

SEQ ID NO: 48 - protein, 5 aa, synthetic construct
EGWRW

SEQ ID NO: 49 - protein, 10 aa, synthetic construct
ELISFCLDTK

SEQ ID NO: 50 - protein, 9 aa, synthetic construct
FFDDDGGDD

SEQ ID NO: 51 - protein, 11 aa, synthetic construct
GDDGDGDGDFF

SEQ ID NO: 52 - protein, 12 aa, synthetic construct
GDDGDGDGDFFK

SEQ ID NO: 53 - protein, 12 aa, synthetic construct
GDGDGDGDGDFF

SEQ ID NO: 54 - protein, 10 aa, synthetic construct
GGGGGDPDPD

SEQ ID NO: 55 - protein, 12 aa, synthetic construct
GGGGGDPDPDFF

SEQ ID NO: 56 - protein, 13 aa, synthetic construct
GGGGGDPDPDFFK

SEQ ID NO: 57 - protein, 11 aa, synthetic construct
GGGGGGDPDPD

SEQ ID NO: 58 - protein, 12 aa, synthetic construct
GGGGGGDPDPDK

SEQ ID NO: 59 - protein, 14 aa, synthetic construct
GGSSSGSGNDEEFQ

SEQ ID NO: 60 - protein, 15 aa, synthetic construct
GGSSSGSGNDEEFQK

SEQ ID NO: 61 - protein, 10 aa, synthetic construct
GPPPPPPPPG

SEQ ID NO: 62 - protein, 5 aa, synthetic construct
  SITE 1: note = isopeptide bond
EGWRW

SEQ ID NO: 63 - protein, 4 aa, synthetic construct
LIFA

SEQ ID NO: 64 - protein, 11 aa, synthetic construct
NNGGGDDDFFK

SEQ ID NO: 65 - protein, 10 aa, synthetic construct
NNGGGNNNFF

SEQ ID NO: 66 - protein, 11 aa, synthetic construct
NNGGGNNNFFK

SEQ ID NO: 67 - protein, 5 aa, synthetic construct
RLIFA

SEQ ID NO: 68 - protein, 16 aa, synthetic construct
SLIDRGVNFSLDTIEK

SEQ ID NO: 69 - protein, 13 aa, synthetic construct
TDYVNLIQELAPK

SEQ ID NO: 70 - DNA, 25 bp, synthetic construct
  source 1..25: mol_type = other DNA
tggagtcaaggtcctctgatgccat

(19) United States
(12) Patent Application Publication
(10) Pub. No.: US 2024/0228671 A1
(43) Pub. Date: Jul. 11, 2024

(54) POLYPEPTIDYL LINKERS

(71) Applicant: Quantum-Si Incorporated, Branford, CT (US)

(72) Inventors: Brian Reed, Madison, CT (US); Haidong Huang, Madison, CT (US); Manjula Pandey, Guilford, CT (US); Andrzej Wilczynski, East Haven, CT (US)

(73) Assignee: Quantum-Si Incorporated, Branford, CT (US)

(21) Appl. No.: 18/491,727

(22) Filed: Oct. 20, 2023

Related U.S. Application Data
(60) Provisional application No. 63/418,265, filed on Oct. 21, 2022.

(51) Int. Cl. C07K 19/00; C07K 7/04; C12Q 1/37; G01N 33/68

(52) U.S. Cl. CPC C07K 19/00; C07K 7/04; C12Q 1/37; G01N 33/6818

(57) ABSTRACT

Provided herein are compounds of Formulae (I) and (II), which comprise polypeptidyl groups. Also provided herein are methods of preparing compounds of Formulae (I) and (II). Further provided herein are methods of sequencing a polypeptide by reaction of compounds of Formula (II) with peptidases.

### CROSS-REFERENCE TO RELATED APPLICATIONS

The present application claims the benefit of priority of U.S. Provisional Application No. 63/418,265 filed Oct. 21, 2022, the entire content of which is incorporated herein by reference.

### REFERENCE TO AN ELECTRONIC SEQUENCE LISTING

The contents of the electronic sequence listing (R070870158US01-SEQ-DFC.xml; Size: 81,056 bytes; and Date of Creation: Oct. 20, 2023) is herein incorporated by reference in its entirety.

### BACKGROUND

Proteomics has emerged as an important and necessary complement to genomics and transcriptomics in the study of biological systems. The proteomic analysis of an individual organism can provide insights into cellular processes and response patterns, which can lead to improved diagnostic and therapeutic strategies. The complexity surrounding protein structure, composition, and modification present challenges in determining large-scale protein sequencing information for a biological sample.

Previous work has led to the development of methods of polypeptide sequencing that involve using a degradation process of a polypeptide with peptidases to produce an amino acid sequence representative of the polypeptide. See, e.g., PCT International Publication No. WO2020/102741A1, filed Nov. 15, 2019, and PCT International Publication No. WO2021/236983A2, filed May 20, 2021, each of which is incorporated by reference in its entirety. As the degradation process progresses during such sequencing, the polypeptide becomes shorter in length. Accordingly, the ability of the polypeptide to access the active sites of peptidases becomes increasingly less efficient, resulting in decreases in cutting efficiency (e.g., cut rate), cut depth, and information content of reads. There is a need for the development of strategies to overcome these challenges in polypeptide sequencing.

### SUMMARY

In such methods of polypeptide sequencing, the polypeptide is linked via a linker to an oligonucleotide, which together increase solubility and may be used to enable surface immobilization. One strategy to overcome the challenges associated with these methods is to modify the structure of the linker, which affects numerous parameters relevant for polypeptide sequencing, including conjugation rate, conjugation bias, aggregation of the conjugate, cutting kinetics, and pulse width. On the molecular level, the structure of the linker may affect the solvation of the polypeptide, the distance between the polypeptide and the oligonucleotide, and the potential secondary structures adopted by the polypeptide. The secondary structures adopted by the polypeptide may be influenced by the non-covalent interactions within the polypeptide, between the polypeptide and the linker, and/or between the polypeptide and the oligonucleotide. Relevant factors for the secondary structures include length, polarity, size, bulkiness, charge, and rigidity or flexibility of the linker, as well as terminal base pair stability.

The present application describes new linkers, as well as methods of preparation thereof. These new linkers can be coupled to polypeptides, including through click chemistry reactions, to form linker-polypeptide conjugates, which are useful for the sequencing of the polypeptide. The new linkers offer several benefits, including improvements in cutting efficiency, cut depth, and information content of reads.

Accordingly, in one aspect, provided herein is a compound of Formula (I):

L-Y (I),

or a salt thereof, wherein L and Y are defined herein.

In another aspect, provided herein is a compound of Formula (II):

Z-L-Y (II),

or a salt thereof, wherein L, Y, and Z are defined herein.

In another aspect, provided herein is a method of preparing a compound of Formula (II):

Z-L-Y (II),

or a salt thereof, comprising reacting a compound of Formula (I):

L-Y (I),

or a salt thereof, with a compound of formula Z—N₃, or a salt thereof, wherein L, Y, and Z are defined herein.

In another aspect, provided herein is a method of sequencing a polypeptide Z, the method comprising reacting a compound of Formula (II):

Z-L-Y (II),

or a salt thereof, with a peptidase, wherein L and Y are defined herein;
reacting the compound of Formula (II), or salt thereof, with a peptidase, in a degradation process;
obtaining data during the degradation process;
analyzing the data to determine portions of the data corresponding to amino acids that are sequentially exposed at a terminus of the polypeptide during the degradation process; and
outputting an amino acid sequence representative of the polypeptide.

The details of certain embodiments of the disclosure are set forth in the Detailed Description of Certain Embodiments, as described below. Other features, objects, and advantages of the disclosure will be apparent from the Definitions, Examples, and Claims.

### BRIEF DESCRIPTION OF THE DRAWINGS

FIG. 1A shows the structure of the C6 linker. FIG. 1B shows the structure of the aspartate-rich Q24D linker (SEQ ID NO: 43). Based on the TET aminopeptidase structural model, the minimum distance requirement for the linker is 33 Å. FIG. 1C shows improved access to aminopeptidase active site for the Q24D linker compared to the C6 linker.

FIG. 2 shows the predicted structure of Q24-sulfo-PEG3-DBCO, which indicates that DBCO is wrapped in PEG spacer and may become inaccessible to solvent, and that long and flexible spacers, polar or not, may reduce conjugation rate between DBCO and the polypeptide through click reactions.

FIG. 3A shows the predicted structure of Q24-EGWRW-DBCO (SEQ ID NO: 48), which indicates that EGWRW (SEQ ID NO: 48) forms a sacrificial spacer to lift DBCO away from DNA terminus, one tryptophan side chain stacks to terminal base pair, and the other tryptophan side chain stacks to DBCO, and arginine intercalates into the major groove of the duplex. FIG. 3B shows the arginine-base distance for Q24-EGWRW-DBCO (SEQ ID NO: 48).

FIGS. 4A-4B show the predicted starting structure (FIG. 4A) and relaxed structure (FIG. 4B) of Q24-QP423, which contains the C6 linker.

FIGS. 5A-5B show the predicted starting structure (FIG. 5A) and relaxed structure (FIG. 5B) of Q24D-QP423, which contains the DBCO-DDGGGDDDFFK(N₃) (SEQ ID NO: 44) polypeptidyl linker. There is no arginine-DNA interaction.

FIG. 6 shows the arginine-base distance for Q24-QP423 (blue, with C6 linker) and Q24D-QP423 (orange, with DBCO-DDGGGDDDFFK(N₃) (SEQ ID NO: 44) polypeptidyl linker).

FIGS. 7A-7B show protein-structure based design with a TET aminopeptidase and either linker DBCO-GGSSSGSGNDEEFQK(N3)-Q24 (SEQ ID NO: 60) (FIG. 7A) or linker DBCO-GGGGGGDPDPDK(N3)-Q24 (Q24GDP) (SEQ ID NO: 58) (FIG. 7B).

FIGS. 8A-8B show the cutting speed of QP423 with different linkers, using hTet/pfuTet as the cutters. FIG. 8A shows relative cutting rate normalized against the C6 linker. N linker sequence: NNGGGDDDFFK (SEQ ID NO: 64); GDP linker sequence: GGGGGGDPDPDK (SEQ ID NO: 58); GDPF linker sequence: GGGGGDPDPDFFK (SEQ ID NO: 56); D linker sequence: DDGGGDDDFFK (SEQ ID NO: 44). FIG. 8B shows relative cutting rate normalized against the D linker. Cutting of the first residue R is too fast with AP30/AP37 that the relative rate cannot be evaluated for the Cy spacer.

FIG. 9 shows that the Q24D linker improves cut depth. The average cut depth improved 76%, and 3+RS reads increased 3-fold.

FIG. 10 shows that the sample-prep compatible Q24D linker greatly facilitates cutting (SEQ ID NO: 50).

FIGS. 11A-11AE show improved sequencing performance with longer cut depth and more amino acids recognized in traces on average for the Q24D linker compared to the C6 linker. FIGS. 11A-11D show traces corresponding to four peptides resulting from the digestion of recombinant human protein CDNF (Cerebral dopamine neurotrophic factor, 161 amino acids): EFLNRFYK (SEQ ID NO: 47) (FIG. 11A), ELISFCLDTK (SEQ ID NO: 49) (FIG. 11B), TDYVNLIQELAPK (SEQ ID NO: 69) (FIG. 11C), and SLIDRGVNFSLDTIEK (SEQ ID NO: 68) (FIG. 11D). FIG. 11E shows that software analysis successfully identified substantially more reads corresponding to each peptide with QL581 (containing the Q24D linker) compared to QL580 (containing the C6 linker).

FIG. 12 shows an example overview of real-time dynamic protein sequencing. Protein samples are digested into peptide fragments, immobilized in nanoscale reaction chambers, and incubated with a mixture of freely-diffusing N-terminal amino acid (NAA) recognizers and aminopeptidases that carry out the sequencing process (SEQ ID NOs: 67 and 63). The labeled recognizers bind on and off to the peptide when one of their cognate NAAs is exposed at the N-terminus, thereby producing characteristic pulsing patterns. The NAA is cleaved by an aminopeptidase, exposing the next amino acid for recognition. The temporal order of NAA recognition and the kinetics of binding enable peptide identification and are sensitive to features that modulate binding kinetics, such as post-translational modifications (PTMs).

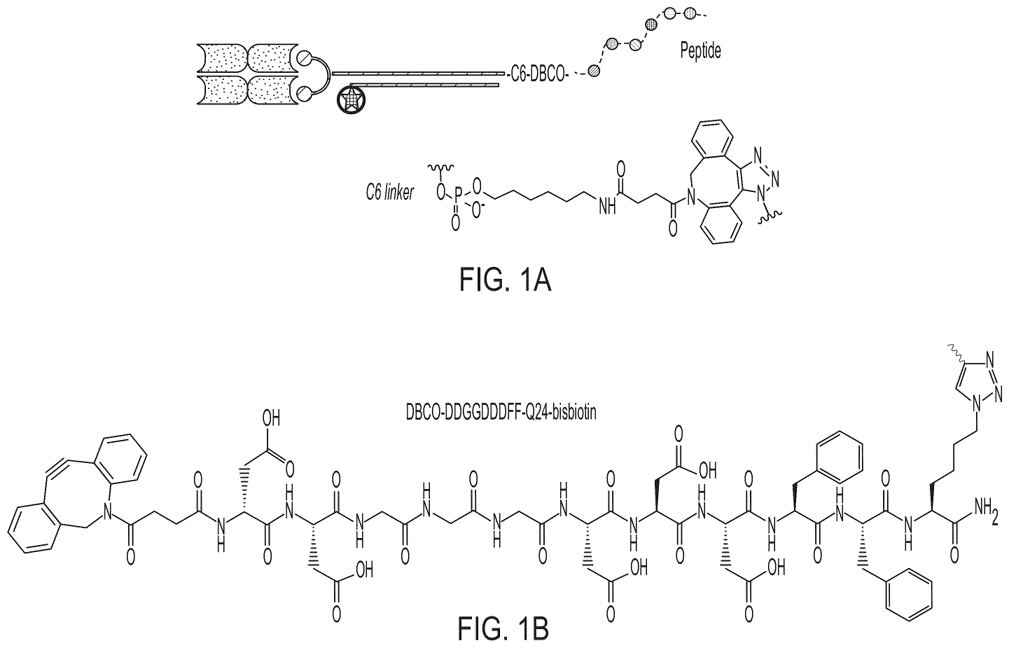

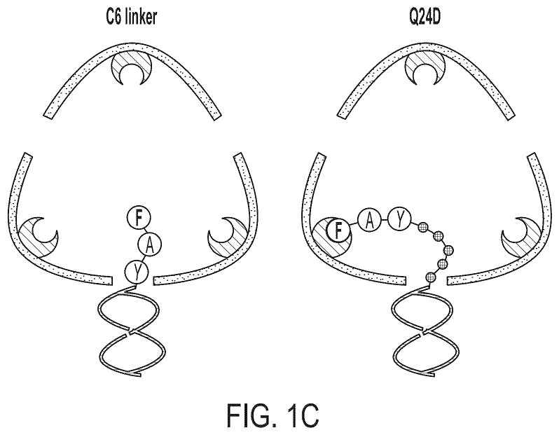

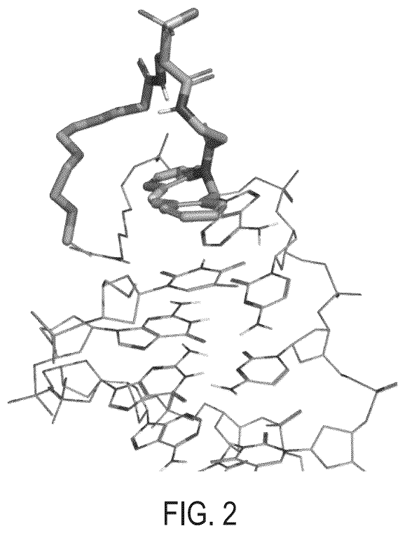

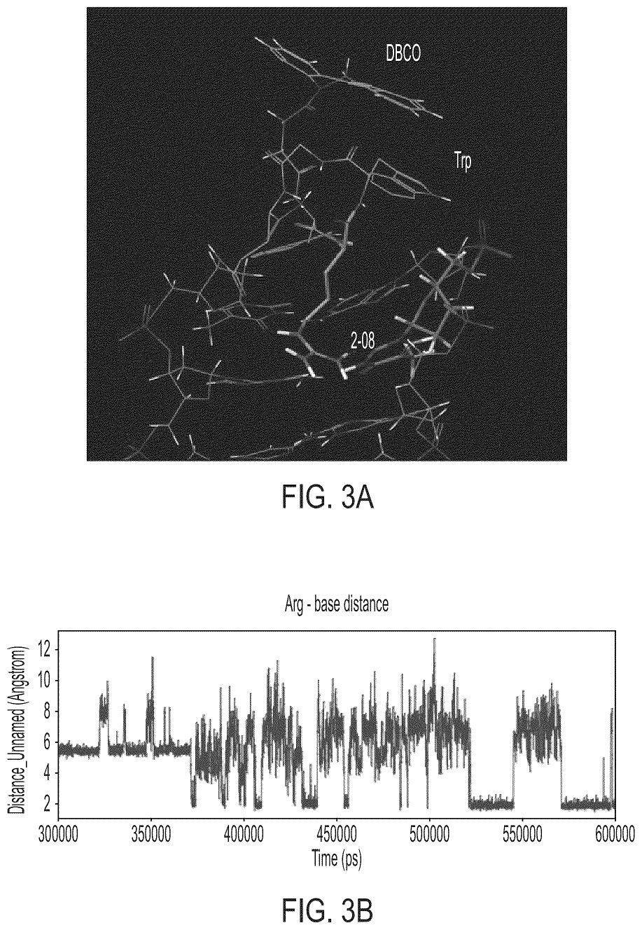

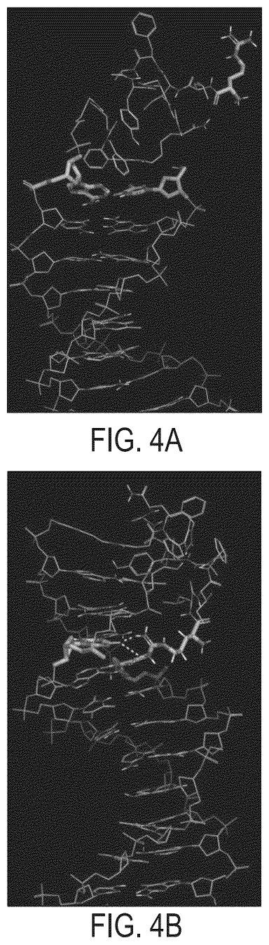

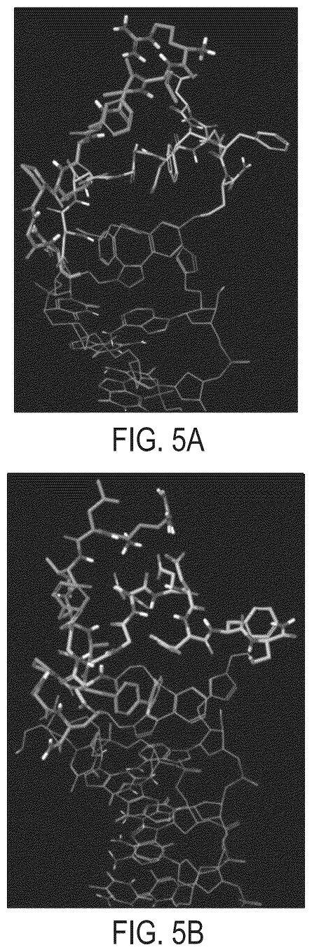

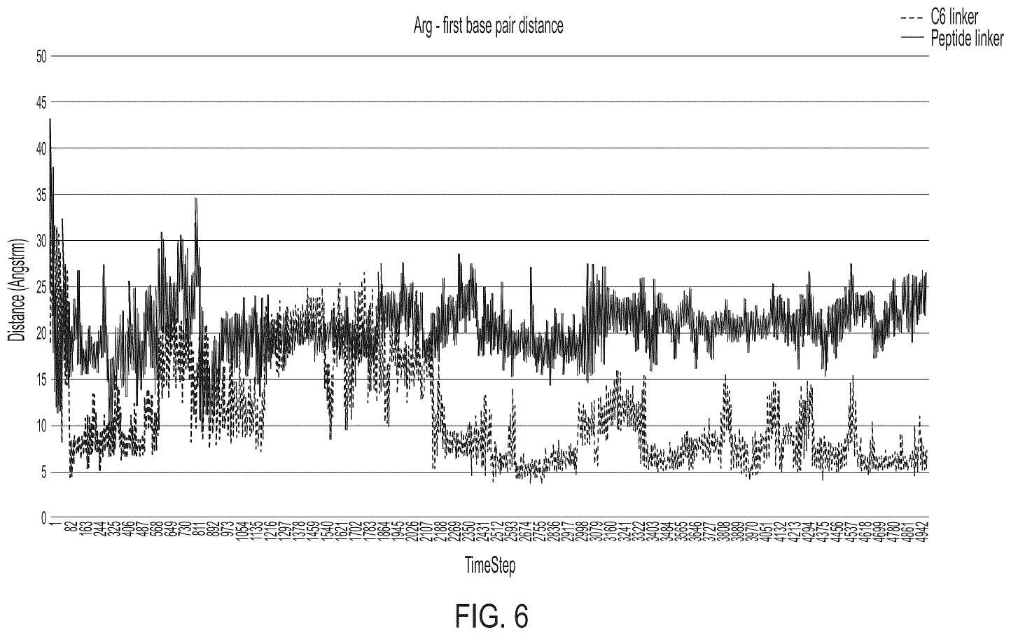

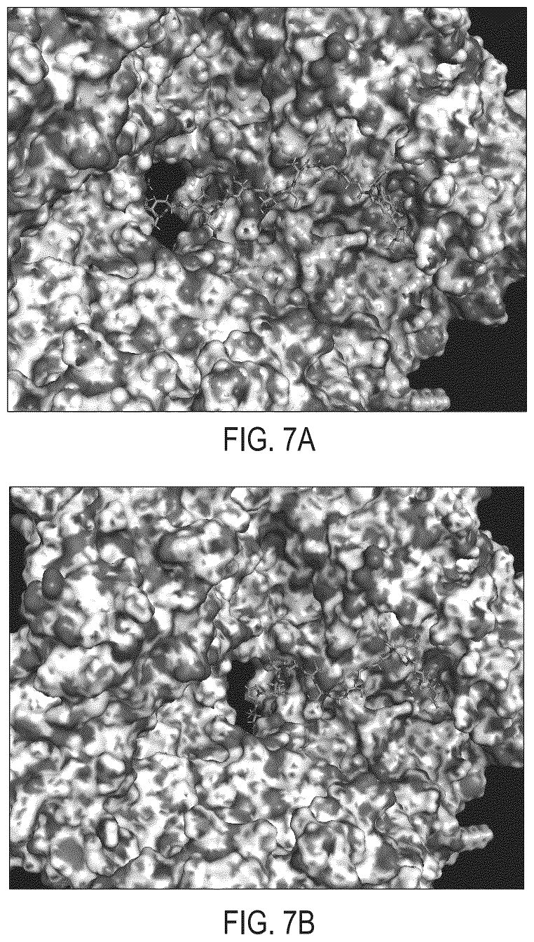

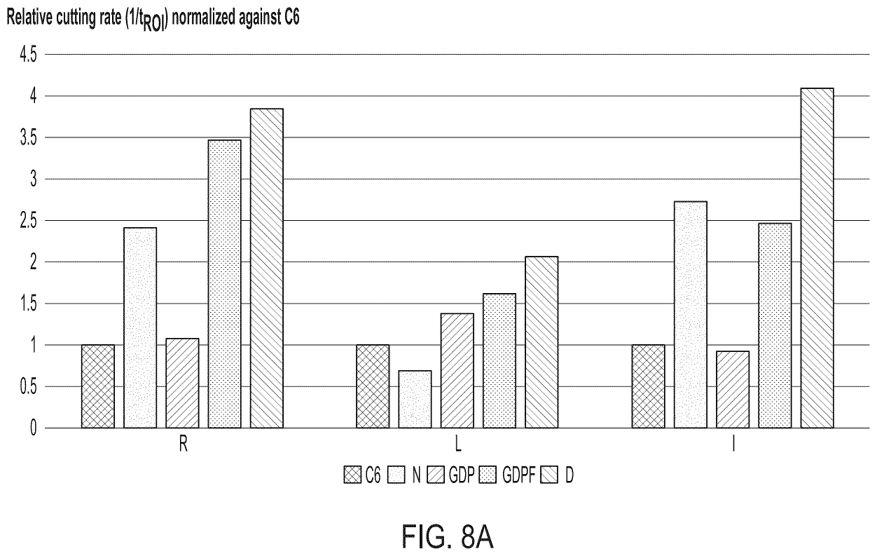

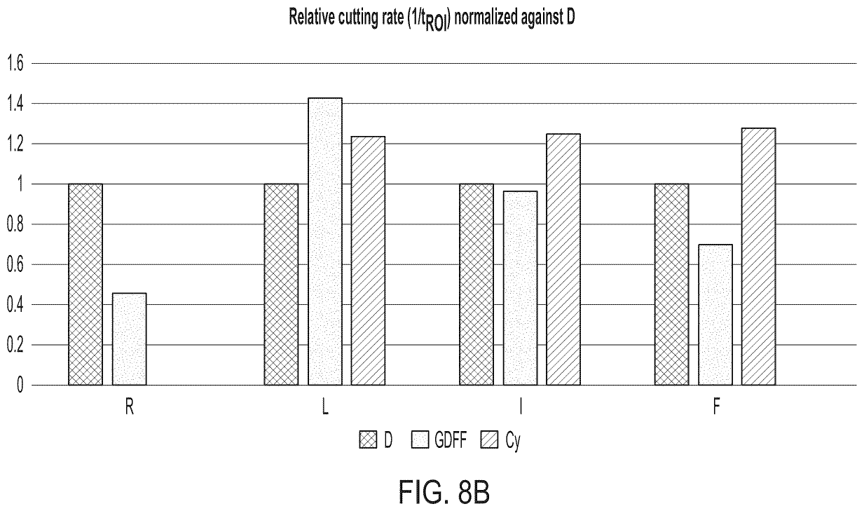

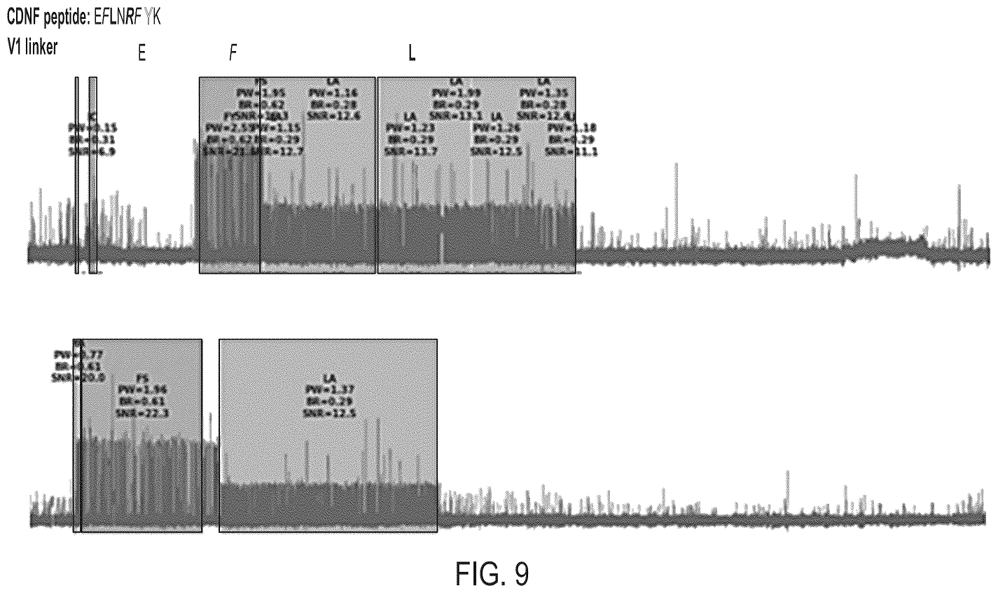

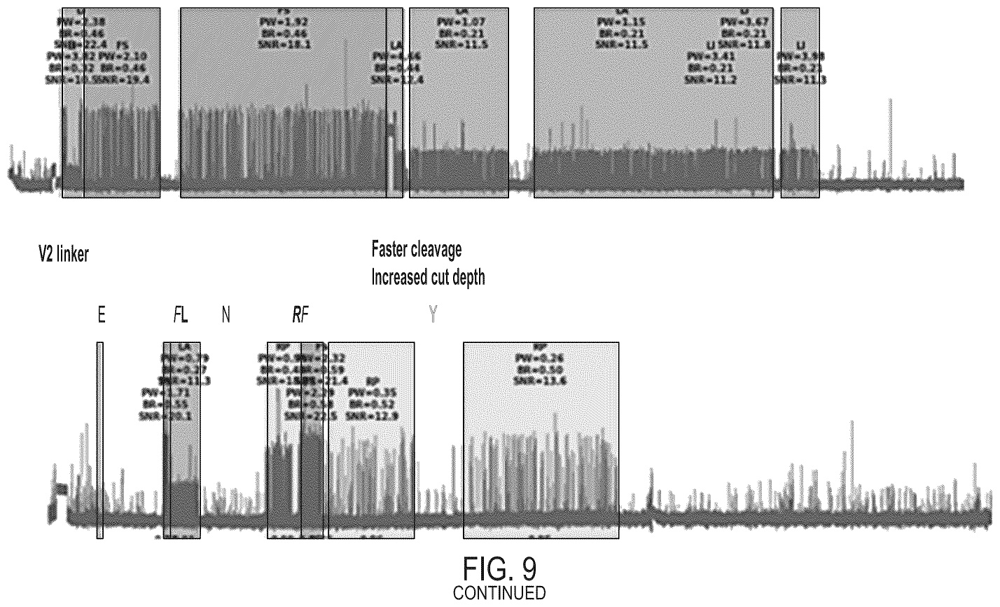

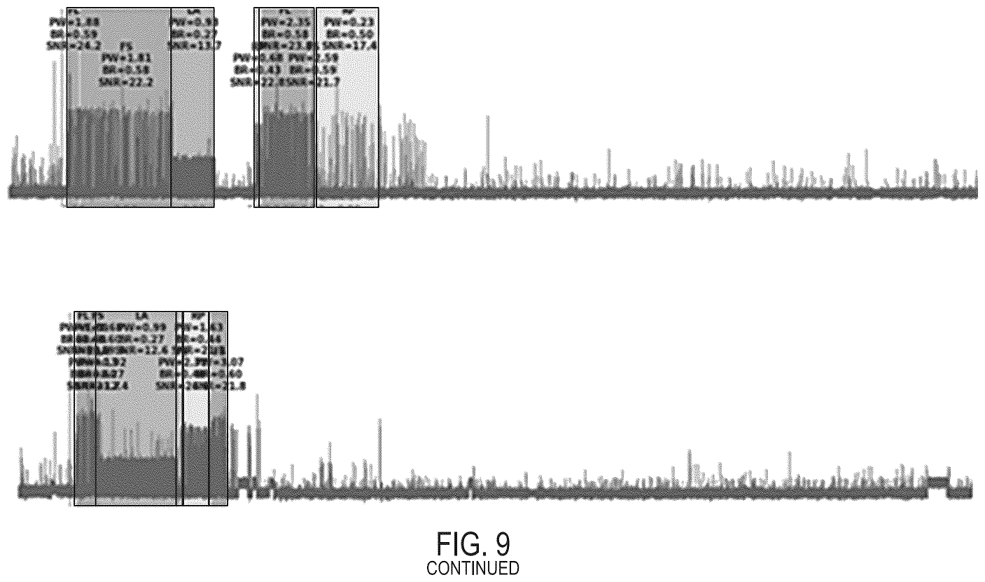

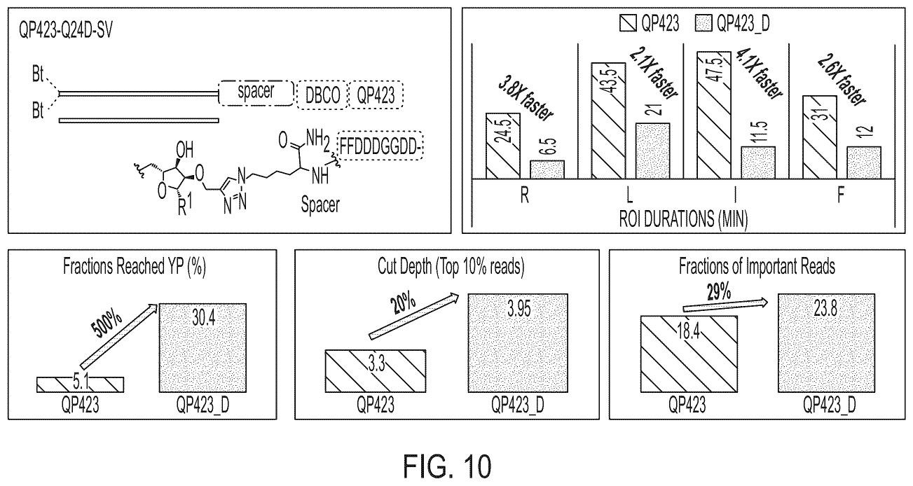

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values (“range”) is listed, it encompasses each value and sub-range within the range. A range is inclusive of the values at the two ends of the range unless otherwise provided. For example, “C₁₋₆ alkyl” encompasses C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

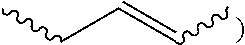

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁_₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{cc})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{aa}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{f})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “boronyl” refers to boranes, boronic acids, boronic esters, borinic acids, and borinic esters, e.g., boronyl groups of the formula —B(R^{aa})₂, —B(OR^{cc})₂, and —BR^{aa}(OR^{cc}), wherein R^{aa} and R^{cc} are as defined herein.

The term “phosphino” refers to the group —P(R^{cc})₂, wherein R^{cc} is as defined herein.

The term “phosphono” refers to the group —(P═O)(OR^{cc})₂, wherein R^{aa} and R^{cc} are as defined herein.

The term “phosphoramido” refers to the group —O(P═O)(N(R^{bb})₂)₂, wherein each R^{bb} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{aa}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{a}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, 4,4′-dimethoxytrityl (4,4′-dimethoxytriphenylmethyl, DMTr, or DMT), a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and Claims. The invention is not limited in any manner by the above exemplary listing of substituents.

As used herein, the term “salt” refers to any and all salts and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

As used herein, the term “work up” refers to any single step or series of multiple steps relating to isolating and/or purifying one or more products of a chemical reaction (e.g., from any remaining starting material, other reagents, solvents, or byproducts of the chemical reaction). Working up a reaction may include removing solvents by, for example, evaporation or lyophilization. Working up a reaction may also include performing liquid-liquid extraction, for example, by separating the reaction mixture into organic and aqueous layers. In some embodiments, working up a reaction includes quenching the reaction to deactivate any unreacted reagents. Working up a reaction may also include cooling a reaction mixture to induce precipitation of solids from the mixture, which may be collected or removed by, for example, filtration, decantation, or centrifugation. Working up a reaction can also include purifying one or more products of the reaction by chromatography. Other methods may also be used to purify one or more reaction products, including, but not limited to, distillation and recrystallization. Other processes for working up a reaction are known in the art, and a person of ordinary skill in the art would readily be capable of determining other appropriate methods that could be employed in working up a particular reaction.

As used herein, the term “about X,” or “approximately X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonuculeotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be introduced directly into the tissue site.

The polynucleotides may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, isotopes (e.g., radioactive isotopes), biotin, and the like.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

Amino acid residues may be indicated by their corresponding single letter codes, e.g., R (arginine), H (histidine), K (lysine), D (aspartic acid), E (glutamic acid), S (serine), T (threonine), N (asparagine), Q (glutamine), C (cysteine), G (glycine), P (proline), A (alanine), V (valine), I (isoleucine), L (leucine), M (methionine), F (phenylalanine), Y (tyrosine), W (tryptophan).

A “peptidase,” “protease,” or “proteinase” is an enzyme that catalyzes the hydrolysis of a peptide bond. Peptidases digest polypeptides into shorter fragments and may be generally classified into endopeptidases and exopeptidases, which cleave a polypeptide chain internally and terminally, respectively. An exopeptidase in accordance with the application may be an “aminopeptidase” or a “carboxypeptidase,” which cleaves a single amino acid from an amino- or a carboxy-terminus, respectively. A peptidase (e.g., an aminopeptidase) may also be referred to as a “cutter” or a “cleaving reagent.”

A “TET aminopeptidase” is composed of 12 monomers that assemble into a tetrahedral structure with 3 active sites in each corner. To access the active sites for digestion, a polypeptide may pass through a pore that leads into the central chamber of the tetrahedron. Each of the 4 faces of the tetrahedron contain one pore in the center of the face. The pore is narrow and does not permit larger compounds (e.g., double-stranded DNA) to pass through.

The term “avidin protein” refers to a biotin-binding protein, generally having a biotin binding site at each of four subunits of the avidin protein. Avidin proteins include, for example, avidin, streptavidin, traptavidin, tamavidin, bradavidin, xenavidin, and homologs and variants thereof. In some cases, the monomeric, dimeric, or tetrameric form of the avidin protein can be used. In some embodiments, the avidin protein of an avidin protein complex is streptavidin in a tetrameric form (e.g., a homotetramer).

The terms “cut depth” or “cutting depth” refer to the degree to which amino acids are sequentially exposed at a terminus of a polypeptide during a degradation process occurring during sequencing of the polypeptide. An increased cut depth indicates that more amino acids are sequentially exposed, and so more of the polypeptide is sequenced. A decreased cut depth indicates that fewer amino acids are sequentially exposed, and so less of the polypeptide is sequenced.

The term “percentage of reads that terminate at a specific residue” refers to the percentage of reads that terminate at the last recognizable position during sequencing of the polypeptide, or at a favorable position preceding the last recognizable position during sequencing of the polypeptide. An increase in the percentage of reads that terminate at a specific residue indicates that a greater portion of the total number of reads reach the specific residue. A decrease in the percentage of reads that terminate at a specific residue indicates that a lesser portion of the total number of reads reach the specific residue.

The terms “cut rate,” “cutting rate,” “cut speed,” or “cutting speed” refer to the rate at which amino acids are sequentially exposed at a terminus of a polypeptide during a degradation process occurring during sequencing of the polypeptide. The cutting rate may be calculated as 1/t_{ROI}, wherein t_{ROI} is the duration that a recognizable amino acid (i.e., a recognition segment, or a region of interest) is reversibly bound by a fluorescent labeled recognizer. An increased cut rate indicates that amino acids are more quickly sequentially exposed, and so sequencing of the polypeptide occurs more quickly. A decreased cut rate indicates that amino acids are more slowly sequentially exposed, and so sequencing of the polypeptide occurs more slowly. The cutting rate of compounds may be normalized against the cutting rate of a control compound.

The term “click chemistry” refers to a chemical synthesis technique introduced by K. Barry Sharpless of The Scripps Research Institute, describing chemistry tailored to generate covalent bonds quickly and reliably by joining small units comprising reactive groups together. See, e.g., Kolb, Finn and Sharpless Angewandte Chemie International Edition (2001) 40: 2004-2021; Evans, Australian Journal of Chemistry (2007) 60: 384-395). Exemplary coupling reactions (some of which may be classified as “click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2]cycloaddition). Exemplary click chemistry reactions include, but are not limited to, azide-alkyne Huisgen cycloaddition; and Diels-Alder reactions (e.g., tetrazine [4+2] cycloaddition). In some embodiments, click chemistry reactions are modular, wide in scope, give high chemical yields, generate inoffensive byproducts, are stereospecific, exhibit a large thermodynamic driving force >84 kJ/mol to favor a reaction with a single reaction product, and/or can be carried out under physiological conditions. In some embodiments, a click chemistry reaction exhibits high atom economy, can be carried out under simple reaction conditions, use readily available starting materials and reagents, uses no toxic solvents or use a solvent that is benign or easily removed (preferably water), and/or provides simple product isolation by non-chromatographic methods (crystallization or distillation).

The term “click chemistry handle,” as used herein, refers to a reactant, or a reactive group, that can partake in a click chemistry reaction. For example, a strained alkyne, e.g., a cyclooctyne, is a click chemistry handle, since it can partake in a strain-promoted cycloaddition (see, e.g., Table 1). In general, click chemistry reactions require at least two molecules comprising click chemistry handles that can react with each other. Such click chemistry handle pairs that are reactive with each other are sometimes referred to herein as partner click chemistry handles. For example, an azide is a partner click chemistry handle to a cyclooctyne or any other alkyne. Exemplary click chemistry handles suitable for use according to some aspects of this invention are described herein, for example, in Tables 1 and 2. In some embodiments, click chemistry handles are used that can react to form covalent bonds in the presence of a metal catalyst, e.g., copper (II). In some embodiments, click chemistry handles are used that can react to form covalent bonds in the absence of a metal catalyst. Additional suitable click chemistry handles are well known to those of skill in the art, and such click chemistry handles include, but are not limited to, the click chemistry reaction partners, groups, and handles described in Becer, Hoogenboom, and Schubert, Click Chemistry beyond Metal-Catalyzed Cycloaddition, Angewandte Chemie International Edition (2009) 48: 4900-4908 and PCT/US2012/044584 and references therein, which references are incorporated herein by reference for click chemistry handles and methodology.

TABLE 1
Exemplary click chemistry handles and reactions.

| | |
|---|---|
| 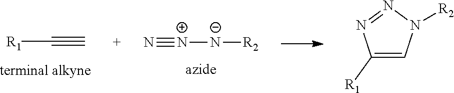 | 1,3-dipolar cycloaddition |
| 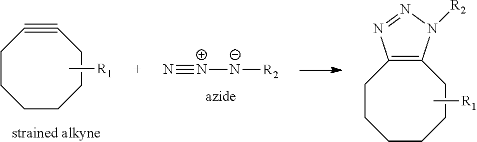 | Strain-promoted cycloaddition |
| 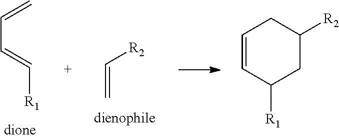 | Diels-Alder reaction |
| 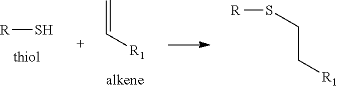 | Thiol-ene reaction |

TABLE 2
Exemplary click chemistry handles and reactions (from Becer, Hoogenboom, and Schubert,
Click Chemistry Beyond Metal-Catalyzed Cycloaddition, Angewandte Chemie International Edition (2009) 48: 4900-4908.).

| | Reagent A | Reagent B | Mechanism | Notes on reaction^{[a]} | Reference |
|---|---|---|---|---|---|
| 0 | azide | alkyne | Cu-catalyzed [3 + 2] | 2 h at 60° C. in H₂O | [9] |
| | | | azide-alkyne cycloaddition | | |
| | | | (CuAAC) | | |
| 1 | azide | cyclooctyne | strain-promoted [3 + 2] azide-alkyne cycloaddition | 1 h at RT | [6- |
| | | | (SPAAC) | | 8, 10, 11] |
| 2 | azide | activated | [3 + 2] Huisgen cycloaddition | 4 h at 50° C. | [12] |
| | | alkyne | | | |
| 3 | azide | electron-deficient | [3 + 2] cycloaddition | 12 h at RT in H₂O | [13] |
| | | alkyne | | | |
| 4 | azide | aryne | [3 + 2] cycloaddition | 4 h at RT in THF with crown ether or | [14, 15] |
| | | | | 24 h at RT in CH₃CN | |
| 5 | tetrazine | alkene | Diels-Alder retro-[4 + 2] cycloaddition | 40 min at 25° C. (100% yield) | [36-38] |
| | | | | N₂ is the only by-product | |
| 6 | tetrazole | alkene | 1,3-dipolar cycloaddition | few min UV irradiation and then overnight | [39, 40] |
| | | | (photoclick) | at 4° C. | |
| 7 | dithioester | diene | hetero-Diels-Alder cycloaddition | 10 min at RT | [43] |
| 8 | anthracene | maleimide | [4 + 2] Diels-Alder reaction | 2 days at reflux in toluene | [41] |
| 9 | thiol | alkene | radical addition | 30 min UV (quantitative conv.) or | [19-23] |
| | | | (thio click) | 24 h UV irradiation (>96%) | |
| 10 | thiol | enone | Michael addition | 24 h at RT in CH₃CN | [27] |
| 11 | thiol | maleimide | Michael addition | 1 h at 40° C. in THF or | [24-26] |
| | | | | 16 h at RT in dioxane | |
| 12 | thiol | para-fluoro | nucleophilic substitution | overnight at RT in DMF or | [32] |
| | | | | 60 min 40° C. in DMF | |
| 13 | amine | para-fluoro | nucleophilic substitution | 20 min MW at 95° C. in NMP as solvent | [30] |

^{[a]}RT = room temperature, DMF = N,N-dimethylformamide, NMP = N-methylpyrolidone, THF = tetrahydrofuran, CH₃CN = acetonitrile.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Compounds

In one aspect, provided herein is a compound of Formula (I):

L-Y (I),

or a salt thereof, wherein:
L comprises a polypeptidyl group; and
Y comprises an oligonucleotide.

In another aspect, provided herein is a compound of Formula (II):

Z-L-Y (II),

or a salt thereof, wherein:
L comprises a polypeptidyl group;
Y comprises an oligonucleotide; and
Z is a polypeptide.

In certain embodiments, the polypeptidyl group comprises at least 5 amino acid residues. In certain embodiments, the polypeptidyl group comprises at least 6 amino acid residues. In certain embodiments, the polypeptidyl group comprises at least 7 amino acid residues. In certain embodiments, the polypeptidyl group comprises at least 8 amino acid residues. In certain embodiments, the polypeptidyl group comprises at least 9 amino acid residues. In certain embodiments, the polypeptidyl group comprises at least 10 amino acid residues. In certain embodiments, the polypeptidyl group comprises at least 11 amino acid residues. In certain embodiments, the polypeptidyl group comprises at least 12 amino acid residues. In certain embodiments, the polypeptidyl group comprises at least 13 amino acid residues. In certain embodiments, the polypeptidyl group comprises at least 14 amino acid residues. In certain embodiments, the polypeptidyl group comprises at least 15 amino acid residues. In certain embodiments, the polypeptidyl group comprises at least 16 amino acid residues. In certain embodiments, the polypeptidyl group comprises at least 17 amino acid residues. In certain embodiments, the polypeptidyl group comprises at least 18 amino acid residues. In certain embodiments, the polypeptidyl group comprises at least 19 amino acid residues. In certain embodiments, the polypeptidyl group comprises at least 20 amino acid residues. In certain embodiments, the polypeptidyl group comprises between 5 and 20 amino acid residues, inclusive. In certain embodiments, the polypeptidyl group comprises between 5 and 18 amino acid residues, inclusive. In certain embodiments, the polypeptidyl group comprises between 5 and 15 amino acid residues, inclusive. In certain embodiments, the polypeptidyl group comprises between 7 and 13 amino acid residues, inclusive. In certain embodiments, the polypeptidyl group comprises between 9 and 11 amino acid residues, inclusive. In certain embodiments, the polypeptidyl group comprises between 7 and 20 amino acid residues, inclusive. In certain embodiments, the polypeptidyl group comprises between 9 and 20 amino acid residues, inclusive. In certain embodiments, the polypeptidyl group comprises between 11 and 20 amino acid residues, inclusive. In certain embodiments, the polypeptidyl group comprises between 7 and 18 amino acid residues, inclusive. In certain embodiments, the polypeptidyl group comprises between 9 and 18 amino acid residues, inclusive. In certain embodiments, the polypeptidyl group comprises between 11 and 18 amino acid residues, inclusive. In certain embodiments, the polypeptidyl group comprises between 7 and 15 amino acid residues, inclusive. In certain embodiments, the polypeptidyl group comprises between 8 and 15 amino acid residues, inclusive. In certain embodiments, the polypeptidyl group comprises between 9 and 15 amino acid residues, inclusive. In certain embodiments, the polypeptidyl group comprises between 9 and 14 amino acid residues, inclusive. In certain embodiments, the polypeptidyl group comprises between 9 and 13 amino acid residues, inclusive. In certain embodiments, the polypeptidyl group comprises between 9 and 12 amino acid residues, inclusive. In certain embodiments, the polypeptidyl group comprises between 10 and 15 amino acid residues, inclusive. In certain embodiments, the polypeptidyl group comprises between 10 and 14 amino acid residues, inclusive. In certain embodiments, the polypeptidyl group comprises between 10 and 13 amino acid residues, inclusive. In certain embodiments, the polypeptidyl group comprises between 10 and 12 amino acid residues, inclusive. In certain embodiments, the polypeptidyl group comprises between 11 and 15 amino acid residues, inclusive. In certain embodiments, the polypeptidyl group comprises between 11 and 14 amino acid residues, inclusive. In certain embodiments, the polypeptidyl group comprises between 11 and 13 amino acid residues, inclusive. In certain embodiments, the polypeptidyl group comprises between 11 and 12 amino acid residues, inclusive. In certain embodiments, the polypeptidyl group comprises 5 amino acid residues. In certain embodiments, the polypeptidyl group comprises 6 amino acid residues. In certain embodiments, the polypeptidyl group comprises 7 amino acid residues. In certain embodiments, the polypeptidyl group comprises 8 amino acid residues. In certain embodiments, the polypeptidyl group comprises 9 amino acid residues. In certain embodiments, the polypeptidyl group comprises 10 amino acid residues. In certain embodiments, the polypeptidyl group comprises 11 amino acid residues. In certain embodiments, the polypeptidyl group comprises 12 amino acid residues. In certain embodiments, the polypeptidyl group comprises 13 amino acid residues. In certain embodiments, the polypeptidyl group 14 amino acid residues. In certain embodiments, the polypeptidyl group comprises 15 amino acid residues. In certain embodiments, the polypeptidyl group comprises 16 amino acid residues. In certain embodiments, the polypeptidyl group comprises 17 amino acid residues. In certain embodiments, the polypeptidyl group comprises 18 amino acid residues. In certain embodiments, the polypeptidyl group comprises 19 amino acid residues. In certain embodiments, the polypeptidyl group comprises 20 amino acid residues.

In certain embodiments, the polypeptidyl group is at least about 20 Å in length. In certain embodiments, the polypeptidyl group is at least about 25 Å in length. In certain embodiments, the polypeptidyl group is at least about 30 Å in length. In certain embodiments, the polypeptidyl group is at least about 33 Å in length. In certain embodiments, the polypeptidyl group is at least about 35 Å in length. In certain embodiments, the polypeptidyl group is at least about 40 Å in length. In certain embodiments, the polypeptidyl group is at least about 45 Å in length. In certain embodiments, the polypeptidyl group is at least about 50 Å in length. In certain embodiments, the polypeptidyl group is at least about 55 Å in length. In certain embodiments, the polypeptidyl group is at least about 60 Å in length. In certain embodiments, the polypeptidyl group is at least about 65 Å in length. In certain embodiments, the polypeptidyl group is at least about 70 Å in length. In certain embodiments, the polypeptidyl group is at least about 75 Å in length. In certain embodiments, the polypeptidyl group is between about 20 Å and about 75 Å in length, inclusive. In certain embodiments, the polypeptidyl group is between about 20 Å and about 70 Å in length, inclusive. In certain embodiments, the polypeptidyl group is between about 20 Å and about 65 Å in length, inclusive. In certain embodiments, the polypeptidyl group is between about 20 Å and about 60 Å in length, inclusive. In certain embodiments, the polypeptidyl group is between about 20 Å and about 55 Å in length, inclusive. In certain embodiments, the polypeptidyl group is between about 20 Å and about 50 Å in length, inclusive. In certain embodiments, the polypeptidyl group is between about 20 Å and about 45 Å in length, inclusive. In certain embodiments, the polypeptidyl group is between about 20 Å and about 40 Å in length, inclusive. In certain embodiments, the polypeptidyl group is between about 20 Å and about 35 Å in length, inclusive. In certain embodiments, the polypeptidyl group is between about 25 Å and about 75 Å in length, inclusive. In certain embodiments, the polypeptidyl group is between about 25 Å and about 70 Å in length, inclusive. In certain embodiments, the polypeptidyl group is between about 25 Å and about 65 Å in length, inclusive. In certain embodiments, the polypeptidyl group is between about 25 Å and about 60 Å in length, inclusive. In certain embodiments, the polypeptidyl group is between about 25 Å and about 55 Å in length, inclusive. In certain embodiments, the polypeptidyl group is between about 25 Å and about 50 Å in length, inclusive. In certain embodiments, the polypeptidyl group is between about 25 Å and about 45 Å in length, inclusive. In certain embodiments, the polypeptidyl group is between about 25 Å and about 40 Å in length, inclusive. In certain embodiments, the polypeptidyl group is between about 25 Å and about 35 Å in length, inclusive. In certain embodiments, the polypeptidyl group is between about 30 Å and about 75 Å in length, inclusive. In certain embodiments, the polypeptidyl group is between about 30 Å and about 70 Å in length, inclusive. In certain embodiments, the polypeptidyl group is between about 30 Å and about 65 Å in length, inclusive. In certain embodiments, the polypeptidyl group is between about 30 Å and about 60 Å in length, inclusive. In certain embodiments, the polypeptidyl group is between about 30 Å and about 55 Å in length, inclusive. In certain embodiments, the polypeptidyl group is between about 30 Å and about 50 Å in length, inclusive. In certain embodiments, the polypeptidyl group is between about 30 Å and about 45 Å in length, inclusive. In certain embodiments, the polypeptidyl group is between about 30 Å and about 40 Å in length, inclusive. In certain embodiments, the polypeptidyl group is between about 30 Å and about 35 Å in length, inclusive. In certain embodiments, the polypeptidyl group is about 20 Å in length. In certain embodiments, the polypeptidyl group is about 25 Å in length. In certain embodiments, the polypeptidyl group is about 30 Å in length. In certain embodiments, the polypeptidyl group is about 33 Å in length. In certain embodiments, the polypeptidyl group is about 35 Å in length. In certain embodiments, the polypeptidyl group is about 40 Å in length. In certain embodiments, the polypeptidyl group is about 45 Å in length. In certain embodiments, the polypeptidyl group is about 50 Å in length. In certain embodiments, the polypeptidyl group is about 55 Å in length. In certain embodiments, the polypeptidyl group is about 60 Å in length. In certain embodiments, the polypeptidyl group is about 65 Å in length. In certain embodiments, the polypeptidyl group is about 70 Å in length. In certain embodiments, the polypeptidyl group is about 75 Å in length.

In certain embodiments, the polypeptidyl group comprises between 10 and 15 amino acid residues, inclusive, and the polypeptidyl group is between about 25 Å and about 50 Å in length, inclusive. In certain embodiments, the polypeptidyl group comprises between 10 and 14 amino acid residues, inclusive, and the polypeptidyl group is between about 25 Å and about 50 Å in length, inclusive. In certain embodiments, the polypeptidyl group comprises between 10 and 13 amino acid residues, inclusive, and the polypeptidyl group is between about 25 Å and about 50 Å in length, inclusive. In certain embodiments, the polypeptidyl group comprises between 10 and 12 amino acid residues, inclusive, and the polypeptidyl group is between about 25 Å and about 50 Å in length, inclusive. In certain embodiments, the polypeptidyl group comprises 10 amino acid residues, and the polypeptidyl group is between about 25 Å and about 50 Å in length, inclusive. In certain embodiments, the polypeptidyl group comprises 11 amino acid residues, and the polypeptidyl group is between about 25 Å and about 50 Å in length, inclusive. In certain embodiments, the polypeptidyl group comprises 12 amino acid residues, and the polypeptidyl group is between about 25 Å and about 50 Å in length, inclusive. In certain embodiments, the polypeptidyl group comprises between 10 and 15 amino acid residues, inclusive, and the polypeptidyl group is between about 25 Å and about 45 Å in length, inclusive. In certain embodiments, the polypeptidyl group comprises between 10 and 14 amino acid residues, inclusive, and the polypeptidyl group is between about 25 Å and about 45 Å in length, inclusive. In certain embodiments, the polypeptidyl group comprises between 10 and 13 amino acid residues, inclusive, and the polypeptidyl group is between about 25 Å and about 45 Å in length, inclusive. In certain embodiments, the polypeptidyl group comprises between 10 and 12 amino acid residues, inclusive, and the polypeptidyl group is between about 25 Å and about 45 Å in length, inclusive. In certain embodiments, the polypeptidyl group comprises 10 amino acid residues, and the polypeptidyl group is between about 25 Å and about 45 Å in length, inclusive. In certain embodiments, the polypeptidyl group comprises 11 amino acid residues, and the polypeptidyl group is between about 25 Å and about 45 Å in length, inclusive. In certain embodiments, the polypeptidyl group comprises 12 amino acid residues, and the polypeptidyl group is between about 25 Å and about 45 Å in length, inclusive. In certain embodiments, the polypeptidyl group comprises between 10 and 15 amino acid residues, inclusive, and the polypeptidyl group is between about 25 Å and about 40 Å in length, inclusive. In certain embodiments, the polypeptidyl group comprises between 10 and 14 amino acid residues, inclusive, and the polypeptidyl group is between about 25 Å and about 40 Å in length, inclusive. In certain embodiments, the polypeptidyl group comprises between 10 and 13 amino acid residues, inclusive, and the polypeptidyl group is between about 25 Å and about 40 Å in length, inclusive. In certain embodiments, the polypeptidyl group comprises between 10 and 12 amino acid residues, inclusive, and the polypeptidyl group is between about 25 Å and about 40 Å in length, inclusive. In certain embodiments, the polypeptidyl group comprises 10 amino acid residues, and the polypeptidyl group is between about 25 Å and about 40 Å in length, inclusive. In certain embodiments, the polypeptidyl group comprises 11 amino acid residues, and the polypeptidyl group is between about 25 Å and about 40 Å in length, inclusive. In certain embodiments, the polypeptidyl group comprises 12 amino acid residues, and the polypeptidyl group is between about 25 Å and about 40 Å in length, inclusive. In certain embodiments, the polypeptidyl group comprises between 10 and 15 amino acid residues, inclusive, and the polypeptidyl group is between about 25 Å and about 35 Å in length, inclusive. In certain embodiments, the polypeptidyl group comprises between 10 and 14 amino acid residues, inclusive, and the polypeptidyl group is between about 25 Å and about 35 Å in length, inclusive. In certain embodiments, the polypeptidyl group comprises between 10 and 13 amino acid residues, inclusive, and the polypeptidyl group is between about 25 Å and about 35 Å in length, inclusive. In certain embodiments, the polypeptidyl group comprises between 10 and 12 amino acid residues, inclusive, and the polypeptidyl group is between about 25 Å and about 35 Å in length, inclusive. In certain embodiments, the polypeptidyl group comprises 10 amino acid residues, and the polypeptidyl group is between about 25 Å and about 35 Å in length, inclusive. In certain embodiments, the polypeptidyl group comprises 11 amino acid residues, and the polypeptidyl group is between about 25 Å and about 35 Å in length, inclusive. In certain embodiments, the polypeptidyl group comprises 12 amino acid residues, and the polypeptidyl group is between about 25 Å and about 35 Å in length, inclusive. In certain embodiments, the polypeptidyl group comprises between 10 and 15 amino acid residues, inclusive, and the polypeptidyl group is between about 30 Å and about 35 Å in length, inclusive. In certain embodiments, the polypeptidyl group comprises between 10 and 14 amino acid residues, inclusive, and the polypeptidyl group is between about 30 Å and about 35 Å in length, inclusive. In certain embodiments, the polypeptidyl group comprises between 10 and 13 amino acid residues, inclusive, and the polypeptidyl group is between about 30 Å and about 35 Å in length, inclusive. In certain embodiments, the polypeptidyl group comprises between 10 and 12 amino acid residues, inclusive, and the polypeptidyl group is between about 30 Å and about 35 Å in length, inclusive. In certain embodiments, the polypeptidyl group comprises 10 amino acid residues, and the polypeptidyl group is between about 30 Å and about 35 Å in length, inclusive. In certain embodiments, the polypeptidyl group comprises 11 amino acid residues, and the polypeptidyl group is between about 30 Å and about 35 Å in length, inclusive. In certain embodiments, the polypeptidyl group comprises 12 amino acid residues, and the polypeptidyl group is between about 30 Å and about 35 Å in length, inclusive. In certain embodiments, the polypeptidyl group comprises between 10 and 15 amino acid residues, inclusive, and the polypeptidyl group is about 33 Å in length. In certain embodiments, the polypeptidyl group comprises between 10 and 14 amino acid residues, inclusive, and the polypeptidyl group is about 33 Å in length. In certain embodiments, the polypeptidyl group comprises between 10 and 13 amino acid residues, inclusive, and the polypeptidyl group is about 33 Å in length. In certain embodiments, the polypeptidyl group comprises between 10 and 12 amino acid residues, inclusive, and the polypeptidyl group is about 33 Å in length. In certain embodiments, the polypeptidyl group comprises 10 amino acid residues, and the polypeptidyl group is about 33 Å in length. In certain embodiments, the polypeptidyl group comprises 11 amino acid residues, and the polypeptidyl group is about 33 Å in length. In certain embodiments, the polypeptidyl group comprises 12 amino acid residues, and the polypeptidyl group is about 33 Å in length.

In certain embodiments, the polypeptidyl group comprises at least 1 negatively charged moiety at physiological pH. In certain embodiments, the polypeptidyl group comprises at least 2 negatively charged moieties at physiological pH. In certain embodiments, the polypeptidyl group comprises at least 3 negatively charged moieties at physiological pH. In certain embodiments, the polypeptidyl group comprises at least 4 negatively charged moieties at physiological pH. In certain embodiments, the polypeptidyl group comprises at least 5 negatively charged moieties at physiological pH. In certain embodiments, the polypeptidyl group comprises at least 6 negatively charged moieties at physiological pH. In certain embodiments, the polypeptidyl group comprises at least 7 negatively charged moieties at physiological pH. In certain embodiments, the polypeptidyl group comprises at least 8 negatively charged moieties at physiological pH. In certain embodiments, the polypeptidyl group comprises at least 9 negatively charged moieties at physiological pH. In certain embodiments, the polypeptidyl group comprises at least 10 negatively charged moieties at physiological pH. In certain embodiments, the polypeptidyl group comprises between 1 and 10 negatively charged moieties at physiological pH, inclusive. in certain embodiments, the polypeptidyl group comprises between 2 and 10 negatively charged moieties at physiological pH, inclusive. In certain embodiments, the polypeptidyl group comprises between 3 and 10 negatively charged moieties at physiological pH, inclusive. In certain embodiments, the polypeptidyl group comprises between 4 and 10 negatively charged moieties at physiological pH, inclusive. In certain embodiments, the polypeptidyl group comprises between 5 and 10 negatively charged moieties at physiological pH, inclusive. In certain embodiments, the polypeptidyl group comprises between 1 and 9 negatively charged moieties at physiological pH, inclusive. In certain embodiments, the polypeptidyl group comprises between 2 and 9 negatively charged moieties at physiological pH, inclusive. In certain embodiments, the polypeptidyl group comprises between 3 and 9 negatively charged moieties at physiological pH, inclusive. In certain embodiments, the polypeptidyl group comprises between 4 and 9 negatively charged moieties at physiological pH, inclusive. In certain embodiments, the polypeptidyl group comprises between 5 and 9 negatively charged moieties at physiological pH, inclusive. In certain embodiments, the polypeptidyl group comprises between 1 and 8 negatively charged moieties at physiological pH, inclusive. In certain embodiments, the polypeptidyl group comprises between 2 and 8 negatively charged moieties at physiological pH, inclusive. In certain embodiments, the polypeptidyl group comprises between 3 and 8 negatively charged moieties at physiological pH, inclusive. In certain embodiments, the polypeptidyl group comprises between 4 and 8 negatively charged moieties at physiological pH, inclusive. In certain embodiments, the polypeptidyl group comprises between 5 and 8 negatively charged moieties at physiological pH, inclusive. In certain embodiments, the polypeptidyl group comprises between 1 and 7 negatively charged moieties at physiological pH, inclusive. In certain embodiments, the polypeptidyl group comprises between 2 and 7 negatively charged moieties at physiological pH, inclusive. In certain embodiments, the polypeptidyl group comprises between 3 and 7 negatively charged moieties at physiological pH, inclusive. In certain embodiments, the polypeptidyl group comprises between 4 and 7 negatively charged moieties at physiological pH, inclusive. In certain embodiments, the polypeptidyl group comprises between 5 and 7 negatively charged moieties at physiological pH, inclusive. In certain embodiments, the polypeptidyl group comprises between 1 and 6 negatively charged moieties at physiological pH, inclusive. In certain embodiments, the polypeptidyl group comprises between 2 and 6 negatively charged moieties at physiological pH, inclusive. In certain embodiments, the polypeptidyl group comprises between 3 and 6 negatively charged moieties at physiological pH, inclusive. In certain embodiments, the polypeptidyl group comprises between 4 and 6 negatively charged moieties at physiological pH, inclusive. In certain embodiments, the polypeptidyl group comprises between 5 and 6 negatively charged moieties at physiological pH, inclusive. In certain embodiments, the polypeptidyl group comprises between 1 and 5 negatively charged moieties at physiological pH, inclusive. In certain embodiments, the polypeptidyl group comprises between 2 and 5 negatively charged moieties at physiological pH, inclusive. In certain embodiments, the polypeptidyl group comprises between 3 and 5 negatively charged moieties at physiological pH, inclusive. In certain embodiments, the polypeptidyl group comprises between 4 and 5 negatively charged moieties at physiological pH, inclusive. In certain embodiments, the polypeptidyl group comprises 1 negatively charged moiety at physiological pH. In certain embodiments, the polypeptidyl group comprises 2 negatively charged moieties at physiological pH. In certain embodiments, the polypeptidyl group comprises 3 negatively charged moieties at physiological pH. In certain embodiments, the polypeptidyl group comprises 4 negatively charged moieties at physiological pH. In certain embodiments, the polypeptidyl group comprises 5 negatively charged moieties at physiological pH. In certain embodiments, the polypeptidyl group comprises 6 negatively charged moieties at physiological pH. In certain embodiments, the polypeptidyl group comprises 7 negatively charged moieties at physiological pH. In certain embodiments, the polypeptidyl group comprises 8 negatively charged moieties at physiological pH. In certain embodiments, the polypeptidyl group comprises 9 negatively charged moieties at physiological pH. In certain embodiments, the polypeptidyl group comprises 10 negatively charged moieties at physiological pH.

In certain embodiments, the polypeptidyl group comprises between 3 and 6 negatively charged moieties at physiological pH, inclusive, and the polypeptidyl group is between about 25 Å and about 50 Å in length, inclusive. In certain embodiments, the polypeptidyl group comprises between 3 and 6 negatively charged moieties at physiological pH, inclusive, and the polypeptidyl group is between about 25 Å and about 45 Å in length, inclusive. In certain embodiments, the polypeptidyl group comprises between 3 and 6 negatively charged moieties at physiological pH, inclusive, and the polypeptidyl group is between about 25 Å and about 40 Å in length, inclusive. In certain embodiments, the polypeptidyl group comprises between 3 and 6 negatively charged moieties at physiological pH, inclusive, and the polypeptidyl group is between about 25 Å and about 35 Å in length, inclusive. In certain embodiments, the polypeptidyl group comprises between 3 and 6 negatively charged moieties at physiological pH, inclusive, and the polypeptidyl group is between about 30 Å and about 35 Å in length, inclusive. In certain embodiments, the polypeptidyl group comprises between 3 and 6 negatively charged moieties at physiological pH, inclusive, and the polypeptidyl group is about 33 Å in length. In certain embodiments, the polypeptidyl group comprises between 4 and 6 negatively charged moieties at physiological pH, inclusive, and the polypeptidyl group is between about 25 Å and about 50 Å in length, inclusive. In certain embodiments, the polypeptidyl group comprises between 4 and 6 negatively charged moieties at physiological pH, inclusive, and the polypeptidyl group is between about 25 Å and about 45 Å in length, inclusive. In certain embodiments, the polypeptidyl group comprises between 4 and 6 negatively charged moieties at physiological pH, inclusive, and the polypeptidyl group is between about 25 Å and about 40 Å in length, inclusive. In certain embodiments, the polypeptidyl group comprises between 4 and 6 negatively charged moieties at physiological pH, inclusive, and the polypeptidyl group is between about 25 Å and about 35 Å in length, inclusive. In certain embodiments, the polypeptidyl group comprises between 4 and 6 negatively charged moieties at physiological pH, inclusive, and the polypeptidyl group is between about 30 Å and about 35 Å in length, inclusive. In certain embodiments, the polypeptidyl group comprises between 4 and 6 negatively charged moieties at physiological pH, inclusive, and the polypeptidyl group is about 33 Å in length. In certain embodiments, the polypeptidyl group comprises between 5 and 6 negatively charged moieties at physiological pH, inclusive, and the polypeptidyl group is between about 25 Å and about 50 Å in length, inclusive. In certain embodiments, the polypeptidyl group comprises between 5 and 6 negatively charged moieties at physiological pH, inclusive, and the polypeptidyl group is between about 25 Å and about 45 Å in length, inclusive. In certain embodiments, the polypeptidyl group comprises between 5 and 6 negatively charged moieties at physiological pH, inclusive, and the polypeptidyl group is between about 25 Å and about 40 Å in length, inclusive. In certain embodiments, the polypeptidyl group comprises between 5 and 6 negatively charged moieties at physiological pH, inclusive, and the polypeptidyl group is between about 25 Å and about 35 Å in length, inclusive. In certain embodiments, the polypeptidyl group comprises between 5 and 6 negatively charged moieties at physiological pH, inclusive, and the polypeptidyl group is between about 30 Å and about 35 Å in length, inclusive. In certain embodiments, the polypeptidyl group comprises between 5 and 6 negatively charged moieties at physiological pH, inclusive, and the polypeptidyl group is about 33 Å in length.

In certain embodiments, the polypeptidyl group comprises at least 1 aspartate residue. In certain embodiments, the polypeptidyl group comprises at least 2 aspartate residues. In certain embodiments, the polypeptidyl group comprises at least 3 aspartate residues. In certain embodiments, the polypeptidyl group comprises at least 4 aspartate residues. In certain embodiments, the polypeptidyl group comprises at least 5 aspartate residues. In certain embodiments, the polypeptidyl group comprises at least 6 aspartate residues. In certain embodiments, the polypeptidyl group comprises at least 7 aspartate residues. In certain embodiments, the polypeptidyl group comprises at least 8 aspartate residues. In certain embodiments, the polypeptidyl group comprises at least 9 aspartate residues. In certain embodiments, the polypeptidyl group comprises at least 10 aspartate residues. In certain embodiments, the polypeptidyl group comprises at least 11 aspartate residues. In certain embodiments, the polypeptidyl group comprises at least 12 aspartate residues. In certain embodiments, the polypeptidyl group comprises at least 13 aspartate residues. In certain embodiments, the polypeptidyl group comprises at least 14 aspartate residues. In certain embodiments, the polypeptidyl group comprises at least 15 aspartate residues. In certain embodiments, the polypeptidyl group comprises between 1 and 15 aspartate residues, inclusive. In certain embodiments, the polypeptidyl group comprises between 1 and 14 aspartate residues, inclusive. In certain embodiments, the polypeptidyl group comprises between 1 and 13 aspartate residues, inclusive. In certain embodiments, the polypeptidyl group comprises between 1 and 12 aspartate residues, inclusive. In certain embodiments, the polypeptidyl group comprises between 1 and 11 aspartate residues, inclusive. In certain embodiments, the polypeptidyl group comprises between 1 and 10 aspartate residues, inclusive. In certain embodiments, the polypeptidyl group comprises between 2 and 10 aspartate residues, inclusive. In certain embodiments, the polypeptidyl group comprises between 3 and 10 aspartate residues, inclusive. In certain embodiments, the polypeptidyl group comprises between 4 and 10 aspartate residues, inclusive. In certain embodiments, the polypeptidyl group comprises between 5 and 10 aspartate residues, inclusive. In certain embodiments, the polypeptidyl group comprises between 1 and 9 aspartate residues, inclusive. In certain embodiments, the polypeptidyl group comprises between 2 and 9 aspartate residues, inclusive. In certain embodiments, the polypeptidyl group comprises between 3 and 9 aspartate residues, inclusive. In certain embodiments, the polypeptidyl group comprises between 4 and 9 aspartate residues, inclusive. In certain embodiments, the polypeptidyl group comprises between 5 and 9 aspartate residues, inclusive. In certain embodiments, the polypeptidyl group comprises between 1 and 8 aspartate residues, inclusive. In certain embodiments, the polypeptidyl group comprises between 2 and 8 aspartate residues, inclusive. In certain embodiments, the polypeptidyl group comprises between 3 and 8 aspartate residues, inclusive. In certain embodiments, the polypeptidyl group comprises between 4 and 8 aspartate residues, inclusive. In certain embodiments, the polypeptidyl group comprises between 5 and 8 aspartate residues, inclusive. In certain embodiments, the polypeptidyl group comprises between 1 and 7 aspartate residues, inclusive. In certain embodiments, the polypeptidyl group comprises between 2 and 7 aspartate residues, inclusive. In certain embodiments, the polypeptidyl group comprises between 3 and 7 aspartate residues, inclusive. In certain embodiments, the polypeptidyl group comprises between 4 and 7 aspartate residues, inclusive. In certain embodiments, the polypeptidyl group comprises between 5 and 7 aspartate residues, inclusive. In certain embodiments, the polypeptidyl group comprises between 1 and 6 aspartate residues, inclusive. In certain embodiments, the polypeptidyl group comprises between 2 and 6 aspartate residues, inclusive. In certain embodiments, the polypeptidyl group comprises between 3 and 6 aspartate residues, inclusive. In certain embodiments, the polypeptidyl group comprises between 4 and 6 aspartate residues, inclusive. In certain embodiments, the polypeptidyl group comprises between 5 and 6 aspartate residues, inclusive. In certain embodiments, the polypeptidyl group comprises between 1 and 5 aspartate residues, inclusive. In certain embodiments, the polypeptidyl group comprises between 2 and 5 aspartate residues, inclusive. In certain embodiments, the polypeptidyl group comprises between 3 and 5 aspartate residues, inclusive. In certain embodiments, the polypeptidyl group comprises between 4 and 5 aspartate residues, inclusive. In certain embodiments, the polypeptidyl group comprises 1 aspartate residue. In certain embodiments, the polypeptidyl group comprises 2 aspartate residues. In certain embodiments, the polypeptidyl group comprises 3 aspartate residues. In certain embodiments, the polypeptidyl group comprises 4 aspartate residues. In certain embodiments, the polypeptidyl group comprises 5 aspartate residues. In certain embodiments, the polypeptidyl group comprises 6 aspartate residues. In certain embodiments, the polypeptidyl group comprises 7 aspartate residues. In certain embodiments, the polypeptidyl group comprises 8 aspartate residues. In certain embodiments, the polypeptidyl group comprises 9 aspartate residues. In certain embodiments, the polypeptidyl group comprises 10 aspartate residues. In certain embodiments, the polypeptidyl group comprises 11 aspartate residues. In certain embodiments, the polypeptidyl group comprises 12 aspartate residues. In certain embodiments, the polypeptidyl group comprises 13 aspartate residues. In certain embodiments, the polypeptidyl group comprises 14 aspartate residues. In certain embodiments, the polypeptidyl group comprises 15 aspartate residues.

In certain embodiments, the polypeptidyl group comprises at least 1 phenylalanine residue. In certain embodiments, the polypeptidyl group comprises at least 2 phenylalanine residues. In certain embodiments, the polypeptidyl group comprises at least 3 phenylalanine residues. In certain embodiments, the polypeptidyl group comprises at least 4 phenylalanine residues. In certain embodiments, the polypeptidyl group comprises at least 5 phenylalanine residues. In certain embodiments, the polypeptidyl group comprises at least 6 phenylalanine residues. In certain embodiments, the polypeptidyl group comprises at least 7 phenylalanine residues. In certain embodiments, the polypeptidyl group comprises at least 8 phenylalanine residues. In certain embodiments, the polypeptidyl group comprises at least 9 phenylalanine residues. In certain embodiments, the polypeptidyl group comprises at least 10 phenylalanine residues. In certain embodiments, the polypeptidyl group comprises between 1 and 10 phenylalanine residues, inclusive. In certain embodiments, the polypeptidyl group comprises between 1 and 9 phenylalanine residues, inclusive. In certain embodiments, the polypeptidyl group comprises between 1 and 8 phenylalanine residues, inclusive. In certain embodiments, the polypeptidyl group comprises between 1 and 7 phenylalanine residues, inclusive. In certain embodiments, the polypeptidyl group comprises between 1 and 6 phenylalanine residues, inclusive. In certain embodiments, the polypeptidyl group comprises between 1 and 5 phenylalanine residues, inclusive. In certain embodiments, the polypeptidyl group comprises between 1 and 4 phenylalanine residues, inclusive. In certain embodiments, the polypeptidyl group comprises between 1 and 3 phenylalanine residues, inclusive. In certain embodiments, the polypeptidyl group comprises between 1 and 2 phenylalanine residues, inclusive. In certain embodiments, the polypeptidyl group comprises between 2 and 10 phenylalanine residues, inclusive. In certain embodiments, the polypeptidyl group comprises between 2 and 9 phenylalanine residues, inclusive. In certain embodiments, the polypeptidyl group comprises between 2 and 8 phenylalanine residues, inclusive. In certain embodiments, the polypeptidyl group comprises between 2 and 7 phenylalanine residues, inclusive. In certain embodiments, the polypeptidyl group comprises between 2 and 6 phenylalanine residues, inclusive. In certain embodiments, the polypeptidyl group comprises between 2 and 5 phenylalanine residues, inclusive. In certain embodiments, the polypeptidyl group comprises between 2 and 4 phenylalanine residues, inclusive. In certain embodiments, the polypeptidyl group comprises between 2 and 3 phenylalanine residues, inclusive. In certain embodiments, the polypeptidyl group comprises 1 phenylalanine residue. In certain embodiments, the polypeptidyl group comprises 2 phenylalanine residues. In certain embodiments, the polypeptidyl group comprises 3 phenylalanine residues. In certain embodiments, the polypeptidyl group comprises 4 phenylalanine residues. In certain embodiments, the polypeptidyl group comprises 5 phenylalanine residues. In certain embodiments, the polypeptidyl group comprises 6 phenylalanine residues. In certain embodiments, the polypeptidyl group comprises 7 phenylalanine residues. In certain embodiments, the polypeptidyl group comprises 8 phenylalanine residues. In certain embodiments, the polypeptidyl group comprises 9 phenylalanine residues. In certain embodiments, the polypeptidyl group comprises 10 phenylalanine residues.

In certain embodiments, the polypeptidyl group comprises between 3 and 7 aspartate residues, inclusive, and the polypeptidyl group comprises between 1 and 4 phenylalanine residues, inclusive. In certain embodiments, the polypeptidyl group comprises between 4 and 7 aspartate residues, inclusive, and the polypeptidyl group comprises between 1 and 4 phenylalanine residues, inclusive. In certain embodiments, the polypeptidyl group comprises between 5 and 7 aspartate residues, inclusive, and the polypeptidyl group comprises between 1 and 4 phenylalanine residues, inclusive. In certain embodiments, the polypeptidyl group comprises between 4 and 6 aspartate residues, inclusive, and the polypeptidyl group comprises between 1 and 4 phenylalanine residues, inclusive. In certain embodiments, the polypeptidyl group comprises between 5 and 6 aspartate residues, inclusive, and the polypeptidyl group comprises between 1 and 4 phenylalanine residues, inclusive. In certain embodiments, the polypeptidyl group comprises 5 aspartate residues, and the polypeptidyl group comprises between 1 and 4 phenylalanine residues, inclusive. In certain embodiments, the polypeptidyl group comprises 6 aspartate residues, and the polypeptidyl group comprises between 1 and 4 phenylalanine residues, inclusive. In certain embodiments, the polypeptidyl group comprises between 3 and 7 aspartate residues, inclusive, and the polypeptidyl group comprises between 1 and 3 phenylalanine residues, inclusive. In certain embodiments, the polypeptidyl group comprises between 4 and 7 aspartate residues, inclusive, and the polypeptidyl group comprises between 1 and 3 phenylalanine residues, inclusive. In certain embodiments, the polypeptidyl group comprises between 5 and 7 aspartate residues, inclusive, and the polypeptidyl group comprises between 1 and 3 phenylalanine residues, inclusive. In certain embodiments, the polypeptidyl group comprises between 4 and 6 aspartate residues, inclusive, and the polypeptidyl group comprises between 1 and 3 phenylalanine residues, inclusive. In certain embodiments, the polypeptidyl group comprises between 5 and 6 aspartate residues, inclusive, and the polypeptidyl group comprises between 1 and 3 phenylalanine residues, inclusive. In certain embodiments, the polypeptidyl group comprises 5 aspartate residues, and the polypeptidyl group comprises between 1 and 3 phenylalanine residues, inclusive. In certain embodiments, the polypeptidyl group comprises 6 aspartate residues, and the polypeptidyl group comprises between 1 and 3 phenylalanine residues, inclusive. In certain embodiments, the polypeptidyl group comprises between 3 and 7 aspartate residues, inclusive, and the polypeptidyl group comprises between 1 and 2 phenylalanine residues, inclusive. In certain embodiments, the polypeptidyl group comprises between 4 and 7 aspartate residues, inclusive, and the polypeptidyl group comprises between 1 and 2 phenylalanine residues, inclusive. In certain embodiments, the polypeptidyl group comprises between 5 and 7 aspartate residues, inclusive, and the polypeptidyl group comprises between 1 and 2 phenylalanine residues, inclusive. In certain embodiments, the polypeptidyl group comprises between 4 and 6 aspartate residues, inclusive, and the polypeptidyl group comprises between 1 and 2 phenylalanine residues, inclusive. In certain embodiments, the polypeptidyl group comprises between 5 and 6 aspartate residues, inclusive, and the polypeptidyl group comprises between 1 and 2 phenylalanine residues, inclusive. In certain embodiments, the polypeptidyl group comprises 5 aspartate residues, and the polypeptidyl group comprises between 1 and 2 phenylalanine residues, inclusive. In certain embodiments, the polypeptidyl group comprises 6 aspartate residues, and the polypeptidyl group comprises between 1 and 2 phenylalanine residues, inclusive. In certain embodiments, the polypeptidyl group comprises between 3 and 7 aspartate residues, inclusive, and the polypeptidyl group comprises 1 phenylalanine residue. In certain embodiments, the polypeptidyl group comprises between 4 and 7 aspartate residues, inclusive, and the polypeptidyl group comprises 1 phenylalanine residue. In certain embodiments, the polypeptidyl group comprises between 5 and 7 aspartate residues, inclusive, and the polypeptidyl group comprises 1 phenylalanine residue. In certain embodiments, the polypeptidyl group comprises between 4 and 6 aspartate residues, inclusive, and the polypeptidyl group comprises 1 phenylalanine residue. In certain embodiments, the polypeptidyl group comprises between 5 and 6 aspartate residues, inclusive, and the polypeptidyl group comprises 1 phenylalanine residue. In certain embodiments, the polypeptidyl group comprises 5 aspartate residues, and the polypeptidyl group comprises 1 phenylalanine residue. In certain embodiments, the polypeptidyl group comprises 6 aspartate residues, and the polypeptidyl group comprises 1 phenylalanine residue. In certain embodiments, the polypeptidyl group comprises between 3 and 7 aspartate residues, inclusive, and the polypeptidyl group comprises 2 phenylalanine residues. In certain embodiments, the polypeptidyl group comprises between 4 and 7 aspartate residues, inclusive, and the polypeptidyl group comprises 2 phenylalanine residues. In certain embodiments, the polypeptidyl group comprises between 5 and 7 aspartate residues, inclusive, and the polypeptidyl group comprises 2 phenylalanine residues. In certain embodiments, the polypeptidyl group comprises between 4 and 6 aspartate residues, inclusive, and the polypeptidyl group comprises 2 phenylalanine residues. In certain embodiments, the polypeptidyl group comprises between 5 and 6 aspartate residues, inclusive, and the polypeptidyl group comprises 2 phenylalanine residues. In certain embodiments, the polypeptidyl group comprises 5 aspartate residues, and the polypeptidyl group comprises 2 phenylalanine residues. In certain embodiments, the polypeptidyl group comprises 6 aspartate residues, and the polypeptidyl group comprises 2 phenylalanine residues.

In certain embodiments, the polypeptidyl group comprises at least 1 glycine residue. In certain embodiments, the polypeptidyl group comprises at least 2 glycine residues. In certain embodiments, the polypeptidyl group comprises at least 3 glycine residues. In certain embodiments, the polypeptidyl group comprises at least 4 glycine residues. In certain embodiments, the polypeptidyl group comprises at least 5 glycine residues. In certain embodiments, the polypeptidyl group comprises at least 6 glycine residues. In certain embodiments, the polypeptidyl group comprises at least 7 glycine residues. In certain embodiments, the polypeptidyl group comprises at least 8 glycine residues. In certain embodiments, the polypeptidyl group comprises at least 9 glycine residues. In certain embodiments, the polypeptidyl group comprises at least 10 glycine residues. In certain embodiments, the polypeptidyl group comprises between 1 and 10 glycine residues, inclusive. In certain embodiments, the polypeptidyl group comprises between 1 and 9 glycine residues, inclusive. In certain embodiments, the polypeptidyl group comprises between 1 and 8 glycine residues, inclusive. In certain embodiments, the polypeptidyl group comprises between 1 and 7 glycine residues, inclusive. In certain embodiments, the polypeptidyl group comprises between 1 and 6 glycine residues, inclusive. In certain embodiments, the polypeptidyl group comprises between 1 and 5 glycine residues, inclusive. In certain embodiments, the polypeptidyl group comprises between 1 and 4 glycine residues, inclusive. In certain embodiments, the polypeptidyl group comprises between 1 and 3 glycine residues, inclusive. In certain embodiments, the polypeptidyl group comprises between 1 and 2 glycine residues, inclusive. In certain embodiments, the polypeptidyl group comprises between 2 and 10 glycine residues, inclusive. In certain embodiments, the polypeptidyl group comprises between 2 and 9 glycine residues, inclusive. In certain embodiments, the polypeptidyl group comprises between 2 and 8 glycine residues, inclusive. In certain embodiments, the polypeptidyl group comprises between 2 and 7 glycine residues, inclusive. In certain embodiments, the polypeptidyl group comprises between 2 and 6 glycine residues, inclusive. In certain embodiments, the polypeptidyl group comprises between 2 and 5 glycine residues, inclusive. In certain embodiments, the polypeptidyl group comprises between 2 and 4 glycine residues, inclusive. In certain embodiments, the polypeptidyl group comprises between 2 and 3 glycine residues, inclusive. In certain embodiments, the polypeptidyl group comprises between 3 and 10 glycine residues, inclusive. In certain embodiments, the polypeptidyl group comprises between 3 and 9 glycine residues, inclusive. In certain embodiments, the polypeptidyl group comprises between 3 and 8 glycine residues, inclusive. In certain embodiments, the polypeptidyl group comprises between 3 and 7 glycine residues, inclusive. In certain embodiments, the polypeptidyl group comprises between 3 and 6 glycine residues, inclusive. In certain embodiments, the polypeptidyl group comprises between 3 and 5 glycine residues, inclusive. In certain embodiments, the polypeptidyl group comprises between 3 and 4 glycine residues, inclusive. In certain embodiments, the polypeptidyl group comprises 1 glycine residue. In certain embodiments, the polypeptidyl group comprises 2 glycine residues. In certain embodiments, the polypeptidyl group comprises 3 glycine residues. In certain embodiments, the polypeptidyl group comprises 4 glycine residues. In certain embodiments, the polypeptidyl group comprises 5 glycine residues. In certain embodiments, the polypeptidyl group comprises 6 glycine residues. In certain embodiments, the polypeptidyl group comprises 7 glycine residues. In certain embodiments, the polypeptidyl group comprises 8 glycine residues. In certain embodiments, the polypeptidyl group comprises 9 glycine residues. In certain embodiments, the polypeptidyl group comprises 10 glycine residues.

In certain embodiments, the polypeptidyl group comprises between 3 and 7 aspartate residues, inclusive, and the polypeptidyl group comprises between 1 and 4 glycine residues, inclusive. In certain embodiments, the polypeptidyl group comprises between 4 and 7 aspartate residues, inclusive, and the polypeptidyl group comprises between 1 and 4 glycine residues, inclusive. In certain embodiments, the polypeptidyl group comprises between 5 and 7 aspartate residues, inclusive, and the polypeptidyl group comprises between 1 and 4 glycine residues, inclusive. In certain embodiments, the polypeptidyl group comprises between 4 and 6 aspartate residues, inclusive, and the polypeptidyl group comprises between 1 and 4 glycine residues, inclusive. In certain embodiments, the polypeptidyl group comprises between 5 and 6 aspartate residues, inclusive, and the polypeptidyl group comprises between 1 and 4 glycine residues, inclusive. In certain embodiments, the polypeptidyl group comprises 5 aspartate residues, and the polypeptidyl group comprises between 1 and 4 glycine residues, inclusive. In certain embodiments, the polypeptidyl group comprises 6 aspartate residues, and the polypeptidyl group comprises between 1 and 4 glycine residues, inclusive. In certain embodiments, the polypeptidyl group comprises between 3 and 7 aspartate residues, inclusive, and the polypeptidyl group comprises between 1 and 3 glycine residues, inclusive. In certain embodiments, the polypeptidyl group comprises between 4 and 7 aspartate residues, inclusive, and the polypeptidyl group comprises between 1 and 3 glycine residues, inclusive. In certain embodiments, the polypeptidyl group comprises between 5 and 7 aspartate residues, inclusive, and the polypeptidyl group comprises between 1 and 3 glycine residues, inclusive. In certain embodiments, the polypeptidyl group comprises between 4 and 6 aspartate residues, inclusive, and the polypeptidyl group comprises between 1 and 3 glycine residues, inclusive. In certain embodiments, the polypeptidyl group comprises between 5 and 6 aspartate residues, inclusive, and the polypeptidyl group comprises between 1 and 3 glycine residues, inclusive. In certain embodiments, the polypeptidyl group comprises 5 aspartate residues, and the polypeptidyl group comprises between 1 and 3 glycine residues, inclusive. In certain embodiments, the polypeptidyl group comprises 6 aspartate residues, and the polypeptidyl group comprises between 1 and 3 glycine residues, inclusive. In certain embodiments, the polypeptidyl group comprises between 3 and 7 aspartate residues, inclusive, and the polypeptidyl group comprises between 2 and 3 glycine residues, inclusive. In certain embodiments, the polypeptidyl group comprises between 4 and 7 aspartate residues, inclusive, and the polypeptidyl group comprises between 2 and 3 glycine residues, inclusive. In certain embodiments, the polypeptidyl group comprises between 5 and 7 aspartate residues, inclusive, and the polypeptidyl group comprises between 2 and 3 glycine residues, inclusive. In certain embodiments, the polypeptidyl group comprises between 4 and 6 aspartate residues, inclusive, and the polypeptidyl group comprises between 2 and 3 glycine residues, inclusive. In certain embodiments, the polypeptidyl group comprises between 5 and 6 aspartate residues, inclusive, and the polypeptidyl group comprises between 2 and 3 glycine residues, inclusive. In certain embodiments, the polypeptidyl group comprises 5 aspartate residues, and the polypeptidyl group comprises between 2 and 3 glycine residues, inclusive. In certain embodiments, the polypeptidyl group comprises 6 aspartate residues, and the polypeptidyl group comprises between 2 and 3 glycine residues, inclusive. In certain embodiments, the polypeptidyl group comprises between 3 and 7 aspartate residues, inclusive, and the polypeptidyl group comprises 2 glycine residues. In certain embodiments, the polypeptidyl group comprises between 4 and 7 aspartate residues, inclusive, and the polypeptidyl group comprises 2 glycine residues. In certain embodiments, the polypeptidyl group comprises between 5 and 7 aspartate residues, inclusive, and the polypeptidyl group comprises 2 glycine residues. In certain embodiments, the polypeptidyl group comprises between 4 and 6 aspartate residues, inclusive, and the polypeptidyl group comprises 2 glycine residues. In certain embodiments, the polypeptidyl group comprises between 5 and 6 aspartate residues, inclusive, and the polypeptidyl group comprises 2 glycine residues. In certain embodiments, the polypeptidyl group comprises 5 aspartate residues, and the polypeptidyl group comprises 2 glycine residues. In certain embodiments, the polypeptidyl group comprises 6 aspartate residues, and the polypeptidyl group comprises 2 glycine residues. In certain embodiments, the polypeptidyl group comprises between 3 and 7 aspartate residues, inclusive, and the polypeptidyl group comprises 3 glycine residues. In certain embodiments, the polypeptidyl group comprises between 4 and 7 aspartate residues, inclusive, and the polypeptidyl group comprises 3 glycine residues. In certain embodiments, the polypeptidyl group comprises between 5 and 7 aspartate residues, inclusive, and the polypeptidyl group comprises 3 glycine residues. In certain embodiments, the polypeptidyl group comprises between 4 and 6 aspartate residues, inclusive, and the polypeptidyl group comprises 3 glycine residues. In certain embodiments, the polypeptidyl group comprises between 5 and 6 aspartate residues, inclusive, and the polypeptidyl group comprises 3 glycine residues. In certain embodiments, the polypeptidyl group comprises 5 aspartate residues, and the polypeptidyl group comprises 3 glycine residues. In certain embodiments, the polypeptidyl group comprises 6 aspartate residues, and the polypeptidyl group comprises 3 glycine residues.

In certain embodiments, the polypeptidyl group comprises at least 1 proline residue. In certain embodiments, the polypeptidyl group comprises at least 2 proline residues. In certain embodiments, the polypeptidyl group comprises at least 3 proline residues. In certain embodiments, the polypeptidyl group comprises at least 4 proline residues. In certain embodiments, the polypeptidyl group comprises at least 5 proline residues. In certain embodiments, the polypeptidyl group comprises at least 6 proline residues. In certain embodiments, the polypeptidyl group comprises at least 7 proline residues. In certain embodiments, the polypeptidyl group comprises at least 8 proline residues. In certain embodiments, the polypeptidyl group comprises at least 9 proline residues. In certain embodiments, the polypeptidyl group comprises at least 10 proline residues. In certain embodiments, the polypeptidyl group comprises between 1 and 10 proline residues, inclusive. In certain embodiments, the polypeptidyl group comprises between 1 and 9 proline residues, inclusive. In certain embodiments, the polypeptidyl group comprises between 1 and 8 proline residues, inclusive. In certain embodiments, the polypeptidyl group comprises between 1 and 7 proline residues, inclusive. In certain embodiments, the polypeptidyl group comprises between 1 and 6 proline residues, inclusive. In certain embodiments, the polypeptidyl group comprises between 1 and 5 proline residues, inclusive. In certain embodiments, the polypeptidyl group comprises between 1 and 4 proline residues, inclusive. In certain embodiments, the polypeptidyl group comprises between 1 and 3 proline residues, inclusive. In certain embodiments, the polypeptidyl group comprises between 1 and 2 proline residues, inclusive. In certain embodiments, the polypeptidyl group comprises between 2 and 10 proline residues, inclusive. In certain embodiments, the polypeptidyl group comprises between 2 and 9 proline residues, inclusive. In certain embodiments, the polypeptidyl group comprises between 2 and 8 proline residues, inclusive. In certain embodiments, the polypeptidyl group comprises between 2 and 7 proline residues, inclusive. In certain embodiments, the polypeptidyl group comprises between 2 and 6 proline residues, inclusive. In certain embodiments, the polypeptidyl group comprises between 2 and 5 proline residues, inclusive. In certain embodiments, the polypeptidyl group comprises between 2 and 4 proline residues, inclusive. In certain embodiments, the polypeptidyl group comprises between 2 and 3 proline residues, inclusive. In certain embodiments, the polypeptidyl group comprises 1 proline residue. In certain embodiments, the polypeptidyl group comprises 2 proline residues. In certain embodiments, the polypeptidyl group comprises 3 proline residues. In certain embodiments, the polypeptidyl group comprises 4 proline residues. In certain embodiments, the polypeptidyl group comprises 5 proline residues. In certain embodiments, the polypeptidyl group comprises 6 proline residues. In certain embodiments, the polypeptidyl group comprises 7 proline residues. In certain embodiments, the polypeptidyl group comprises 8 proline residues. In certain embodiments, the polypeptidyl group comprises 9 proline residues. In certain embodiments, the polypeptidyl group comprises 10 proline residues.

In certain embodiments, the polypeptidyl group comprises between 3 and 7 aspartate residues, inclusive, and the polypeptidyl group comprises between 1 and 4 proline residues, inclusive. In certain embodiments, the polypeptidyl group comprises between 4 and 7 aspartate residues, inclusive, and the polypeptidyl group comprises between 1 and 4 proline residues, inclusive. In certain embodiments, the polypeptidyl group comprises between 5 and 7 aspartate residues, inclusive, and the polypeptidyl group comprises between 1 and 4 proline residues, inclusive. In certain embodiments, the polypeptidyl group comprises between 4 and 6 aspartate residues, inclusive, and the polypeptidyl group comprises between 1 and 4 proline residues, inclusive. In certain embodiments, the polypeptidyl group comprises between 5 and 6 aspartate residues, inclusive, and the polypeptidyl group comprises between 1 and 4 proline residues, inclusive. In certain embodiments, the polypeptidyl group comprises 5 aspartate residues, and the polypeptidyl group comprises between 1 and 4 proline residues, inclusive. In certain embodiments, the polypeptidyl group comprises 6 aspartate residues, and the polypeptidyl group comprises between 1 and 4 proline residues, inclusive. In certain embodiments, the polypeptidyl group comprises between 3 and 7 aspartate residues, inclusive, and the polypeptidyl group comprises between 1 and 3 proline residues, inclusive. In certain embodiments, the polypeptidyl group comprises between 4 and 7 aspartate residues, inclusive, and the polypeptidyl group comprises between 1 and 3 proline residues, inclusive. In certain embodiments, the polypeptidyl group comprises between 5 and 7 aspartate residues, inclusive, and the polypeptidyl group comprises between 1 and 3 proline residues, inclusive. In certain embodiments, the polypeptidyl group comprises between 4 and 6 aspartate residues, inclusive, and the polypeptidyl group comprises between 1 and 3 proline residues, inclusive. In certain embodiments, the polypeptidyl group comprises between 5 and 6 aspartate residues, inclusive, and the polypeptidyl group comprises between 1 and 3 proline residues, inclusive. In certain embodiments, the polypeptidyl group comprises 5 aspartate residues, and the polypeptidyl group comprises between 1 and 3 proline residues, inclusive. In certain embodiments, the polypeptidyl group comprises 6 aspartate residues, and the polypeptidyl group comprises between 1 and 3 proline residues, inclusive. In certain embodiments, the polypeptidyl group comprises between 3 and 7 aspartate residues, inclusive, and the polypeptidyl group comprises between 1 and 2 proline residues, inclusive. In certain embodiments, the polypeptidyl group comprises between 4 and 7 aspartate residues, inclusive, and the polypeptidyl group comprises between 1 and 2 proline residues, inclusive. In certain embodiments, the polypeptidyl group comprises between 5 and 7 aspartate residues, inclusive, and the polypeptidyl group comprises between 1 and 2 proline residues, inclusive. In certain embodiments, the polypeptidyl group comprises between 4 and 6 aspartate residues, inclusive, and the polypeptidyl group comprises between 1 and 2 proline residues, inclusive. In certain embodiments, the polypeptidyl group comprises between 5 and 6 aspartate residues, inclusive, and the polypeptidyl group comprises between 1 and 2 proline residues, inclusive. In certain embodiments, the polypeptidyl group comprises 5 aspartate residues, and the polypeptidyl group comprises between 1 and 2 proline residues, inclusive. In certain embodiments, the polypeptidyl group comprises 6 aspartate residues, and the polypeptidyl group comprises between 1 and 2 proline residues, inclusive. In certain embodiments, the polypeptidyl group comprises between 3 and 7 aspartate residues, inclusive, and the polypeptidyl group comprises 1 proline residue. In certain embodiments, the polypeptidyl group comprises between 4 and 7 aspartate residues, inclusive, and the polypeptidyl group comprises 1 proline residue. In certain embodiments, the polypeptidyl group comprises between 5 and 7 aspartate residues, inclusive, and the polypeptidyl group comprises 1 proline residue. In certain embodiments, the polypeptidyl group comprises between 4 and 6 aspartate residues, inclusive, and the polypeptidyl group comprises 1 proline residue. In certain embodiments, the polypeptidyl group comprises between 5 and 6 aspartate residues, inclusive, and the polypeptidyl group comprises 1 proline residue. In certain embodiments, the polypeptidyl group comprises 5 aspartate residues, and the polypeptidyl group comprises 1 proline residue. In certain embodiments, the polypeptidyl group comprises 6 aspartate residues, and the polypeptidyl group comprises 1 proline residue. In certain embodiments, the polypeptidyl group comprises between 3 and 7 aspartate residues, inclusive, and the polypeptidyl group comprises 2 proline residues. In certain embodiments, the polypeptidyl group comprises between 4 and 7 aspartate residues, inclusive, and the polypeptidyl group comprises 2 proline residues. In certain embodiments, the polypeptidyl group comprises between 5 and 7 aspartate residues, inclusive, and the polypeptidyl group comprises 2 proline residues. In certain embodiments, the polypeptidyl group comprises between 4 and 6 aspartate residues, inclusive, and the polypeptidyl group comprises 2 proline residues. In certain embodiments, the polypeptidyl group comprises between 5 and 6 aspartate residues, inclusive, and the polypeptidyl group comprises 2 proline residues. In certain embodiments, the polypeptidyl group comprises 5 aspartate residues, and the polypeptidyl group comprises 2 proline residues. In certain embodiments, the polypeptidyl group comprises 6 aspartate residues, and the polypeptidyl group comprises 2 proline residues.

In certain embodiments, the polypeptidyl group comprises at least 1 GP repeat. In certain embodiments, the polypeptidyl group comprises at least 2 GP repeats. In certain embodiments, the polypeptidyl group comprises at least 3 GP repeats. In certain embodiments, the polypeptidyl group comprises at least 4 GP repeats. In certain embodiments, the polypeptidyl group comprises at least 5 GP repeats. In certain embodiments, the polypeptidyl group comprises between 1 and 5 GP repeats, inclusive. In certain embodiments, the polypeptidyl group comprises between 1 and 4 GP repeats, inclusive. In certain embodiments, the polypeptidyl group comprises between 1 and 3 GP repeats, inclusive. In certain embodiments, the polypeptidyl group comprises between 1 and 2 GP repeats, inclusive. In certain embodiments, the polypeptidyl group comprises 1 GP repeat. In certain embodiments, the polypeptidyl group comprises 2 GP repeats. In certain embodiments, the polypeptidyl group comprises 3 GP repeats. In certain embodiments, the polypeptidyl group comprises 4 GP repeats. In certain embodiments, the polypeptidyl group comprises 5 GP repeats.

In certain embodiments, the polypeptidyl group comprises at least 1 GG repeat. In certain embodiments, the polypeptidyl group comprises at least 2 GG repeats. In certain embodiments, the polypeptidyl group comprises at least 3 GG repeats. In certain embodiments, the polypeptidyl group comprises at least 4 GG repeats. In certain embodiments, the polypeptidyl group comprises at least 5 GG repeats. In certain embodiments, the polypeptidyl group comprises between 1 and 5 GG repeats, inclusive. In certain embodiments, the polypeptidyl group comprises between 1 and 4 GG repeats, inclusive. In certain embodiments, the polypeptidyl group comprises between 1 and 3 GG repeats, inclusive. In certain embodiments, the polypeptidyl group comprises between 1 and 2 GG repeats, inclusive. In certain embodiments, the polypeptidyl group comprises 1 GG repeat. In certain embodiments, the polypeptidyl group comprises 2 GG repeats. In certain embodiments, the polypeptidyl group comprises 3 GG repeats. In certain embodiments, the polypeptidyl group comprises 4 GG repeats. In certain embodiments, the polypeptidyl group comprises 5 GG repeats.

In certain embodiments, the polypeptidyl group comprises at least 1 GGG repeat. In certain embodiments, the polypeptidyl group comprises at least 2 GGG repeats. In certain embodiments, the polypeptidyl group comprises at least 3 GGG repeats. In certain embodiments, the polypeptidyl group comprises at least 4 GGG repeats. In certain embodiments, the polypeptidyl group comprises at least 5 GGG repeats. In certain embodiments, the polypeptidyl group comprises between 1 and 5 GGG repeats, inclusive. In certain embodiments, the polypeptidyl group comprises between 1 and 4 GGG repeats, inclusive. In certain embodiments, the polypeptidyl group comprises between 1 and 3 GGG repeats, inclusive. In certain embodiments, the polypeptidyl group comprises between 1 and 2 GGG repeats, inclusive. In certain embodiments, the polypeptidyl group comprises 1 GGG repeat. In certain embodiments, the polypeptidyl group comprises 2 GGG repeats. In certain embodiments, the polypeptidyl group comprises 3 GGG repeats. In certain embodiments, the polypeptidyl group comprises 4 GGG repeats. In certain embodiments, the polypeptidyl group comprises 5 GGG repeats.

In certain embodiments, the polypeptidyl group comprises at least 1 DD repeat. In certain embodiments, the polypeptidyl group comprises at least 2 DD repeats. In certain embodiments, the polypeptidyl group comprises at least 3 DD repeats. In certain embodiments, the polypeptidyl group comprises at least 4 DD repeats. In certain embodiments, the polypeptidyl group comprises at least 5 DD repeats. In certain embodiments, the polypeptidyl group comprises between 1 and 5 DD repeats, inclusive. In certain embodiments, the polypeptidyl group comprises between 1 and 4 DD repeats, inclusive. In certain embodiments, the polypeptidyl group comprises between 1 and 3 DD repeats, inclusive. In certain embodiments, the polypeptidyl group comprises between 1 and 2 DD repeats, inclusive. In certain embodiments, the polypeptidyl group comprises 1 DD repeat. In certain embodiments, the polypeptidyl group comprises 2 DD repeats. In certain embodiments, the polypeptidyl group comprises 3 DD repeats. In certain embodiments, the polypeptidyl group comprises 4 DD repeats. In certain embodiments, the polypeptidyl group comprises 5 DD repeats.

In certain embodiments, the polypeptidyl group comprises between 1 and 3 GG repeats, inclusive, and the polypeptidyl group comprises between 1 and 3 DD repeats, inclusive. In certain embodiments, the polypeptidyl group comprises between 1 and 2 GG repeats, inclusive, and the polypeptidyl group comprises between 1 and 3 DD repeats, inclusive. In certain embodiments, the polypeptidyl group comprises 1 GG repeat, and the polypeptidyl group comprises between 1 and 3 DD repeats, inclusive. In certain embodiments, the polypeptidyl group comprises 2 GG repeats, and the polypeptidyl group comprises between 1 and 3 DD repeats, inclusive. In certain embodiments, the polypeptidyl group comprises between 1 and 3 GG repeats, inclusive, and the polypeptidyl group comprises between 1 and 2 DD repeats, inclusive. In certain embodiments, the polypeptidyl group comprises between 1 and 2 GG repeats, inclusive, and the polypeptidyl group comprises between 1 and 2 DD repeats, inclusive. In certain embodiments, the polypeptidyl group comprises 1 GG repeat, and the polypeptidyl group comprises between 1 and 2 DD repeats, inclusive. In certain embodiments, the polypeptidyl group comprises 2 GG repeats, and the polypeptidyl group comprises between 1 and 2 DD repeats, inclusive. In certain embodiments, the polypeptidyl group comprises between 1 and 3 GG repeats, inclusive, and the polypeptidyl group comprises 1 DD repeat. In certain embodiments, the polypeptidyl group comprises between 1 and 2 GG repeats, inclusive, and the polypeptidyl group comprises 1 DD repeat. In certain embodiments, the polypeptidyl group comprises 1 GG repeat, and the polypeptidyl group comprises 1 DD repeat. In certain embodiments, the polypeptidyl group comprises 2 GG repeats, and the polypeptidyl group comprises 1 DD repeat. In certain embodiments, the polypeptidyl group comprises between 1 and 3 GG repeats, inclusive, and the polypeptidyl group comprises 2 DD repeats. In certain embodiments, the polypeptidyl group comprises between 1 and 2 GG repeats, inclusive, and the polypeptidyl group comprises 2 DD repeats. In certain embodiments, the polypeptidyl group comprises 1 GG repeat, and the polypeptidyl group comprises 2 DD repeats. In certain embodiments, the polypeptidyl group comprises 2 GG repeats, and the polypeptidyl group comprises 2 DD repeats. In certain embodiments, the polypeptidyl group comprises between 1 and 3 GG repeats, inclusive, and the polypeptidyl group comprises 3 DD repeats. In certain embodiments, the polypeptidyl group comprises between 1 and 2 GG repeats, inclusive, and the polypeptidyl group comprises 3 DD repeats. In certain embodiments, the polypeptidyl group comprises 1 GG repeat, and the polypeptidyl group comprises 3 DD repeats. In certain embodiments, the polypeptidyl group comprises 2 GG repeats, and the polypeptidyl group comprises 3 DD repeats.

In certain embodiments, the polypeptidyl group comprises between 1 and 3 GGG repeats, inclusive, and the polypeptidyl group comprises between 1 and 3 DD repeats, inclusive. In certain embodiments, the polypeptidyl group comprises between 1 and 2 GGG repeats, inclusive, and the polypeptidyl group comprises between 1 and 3 DD repeats, inclusive. In certain embodiments, the polypeptidyl group comprises 1 GGG repeat, and the polypeptidyl group comprises between 1 and 3 DD repeats, inclusive. In certain embodiments, the polypeptidyl group comprises 2 GGG repeats, and the polypeptidyl group comprises between 1 and 3 DD repeats, inclusive. In certain embodiments, the polypeptidyl group comprises between 1 and 3 GGG repeats, inclusive, and the polypeptidyl group comprises between 1 and 2 DD repeats, inclusive. In certain embodiments, the polypeptidyl group comprises between 1 and 2 GGG repeats, inclusive, and the polypeptidyl group comprises between 1 and 2 DD repeats, inclusive. In certain embodiments, the polypeptidyl group comprises 1 GGG repeat, and the polypeptidyl group comprises between 1 and 2 DD repeats, inclusive. In certain embodiments, the polypeptidyl group comprises 2 GGG repeats, and the polypeptidyl group comprises between 1 and 2 DD repeats, inclusive. In certain embodiments, the polypeptidyl group comprises between 1 and 3 GGG repeats, inclusive, and the polypeptidyl group comprises 1 DD repeat. In certain embodiments, the polypeptidyl group comprises between 1 and 2 GGG repeats, inclusive, and the polypeptidyl group comprises 1 DD repeat. In certain embodiments, the polypeptidyl group comprises 1 GGG repeat, and the polypeptidyl group comprises 1 DD repeat. In certain embodiments, the polypeptidyl group comprises 2 GGG repeats, and the polypeptidyl group comprises 1 DD repeat. In certain embodiments, the polypeptidyl group comprises between 1 and 3 GGG repeats, inclusive, and the polypeptidyl group comprises 2 DD repeats. In certain embodiments, the polypeptidyl group comprises between 1 and 2 GGG repeats, inclusive, and the polypeptidyl group comprises 2 DD repeats. In certain embodiments, the polypeptidyl group comprises 1 GGG repeat, and the polypeptidyl group comprises 2 DD repeats. In certain embodiments, the polypeptidyl group comprises 2 GGG repeats, and the polypeptidyl group comprises 2 DD repeats. In certain embodiments, the polypeptidyl group comprises between 1 and 3 GGG repeats, inclusive, and the polypeptidyl group comprises 3 DD repeats. In certain embodiments, the polypeptidyl group comprises between 1 and 2 GGG repeats, inclusive, and the polypeptidyl group comprises 3 DD repeats. In certain embodiments, the polypeptidyl group comprises 1 GGG repeat, and the polypeptidyl group comprises 3 DD repeats. In certain embodiments, the polypeptidyl group comprises 2 GGG repeats, and the polypeptidyl group comprises 3 DD repeats.

In certain embodiments, the polypeptidyl group comprises at least 1 DDD repeat. In certain embodiments, the polypeptidyl group comprises at least 2 DDD repeats. In certain embodiments, the polypeptidyl group comprises at least 3 DDD repeats. In certain embodiments, the polypeptidyl group comprises at least 4 DDD repeats. In certain embodiments, the polypeptidyl group comprises at least 5 DDD repeats. In certain embodiments, the polypeptidyl group comprises between 1 and 5 DDD repeats, inclusive. In certain embodiments, the polypeptidyl group comprises between 1 and 4 DDD repeats, inclusive. In certain embodiments, the polypeptidyl group comprises between 1 and 3 DDD repeats, inclusive. In certain embodiments, the polypeptidyl group comprises between 1 and 2 DDD repeats, inclusive. In certain embodiments, the polypeptidyl group comprises 1 DDD repeat. In certain embodiments, the polypeptidyl group comprises 2 DDD repeats. In certain embodiments, the polypeptidyl group comprises 3 DDD repeats. In certain embodiments, the polypeptidyl group comprises 4 DDD repeats. In certain embodiments, the polypeptidyl group comprises 5 DDD repeats.

In certain embodiments, the polypeptidyl group comprises between 1 and 3 GG repeats, inclusive, and the polypeptidyl group comprises between 1 and 3 DDD repeats, inclusive. In certain embodiments, the polypeptidyl group comprises between 1 and 2 GG repeats, inclusive, and the polypeptidyl group comprises between 1 and 3 DDD repeats, inclusive. In certain embodiments, the polypeptidyl group comprises 1 GG repeat, and the polypeptidyl group comprises between 1 and 3 DDD repeats, inclusive. In certain embodiments, the polypeptidyl group comprises 2 GG repeats, and the polypeptidyl group comprises between 1 and 3 DDD repeats, inclusive. In certain embodiments, the polypeptidyl group comprises between 1 and 3 GG repeats, inclusive, and the polypeptidyl group comprises between 1 and 2 DDD repeats, inclusive. In certain embodiments, the polypeptidyl group comprises between 1 and 2 GG repeats, inclusive, and the polypeptidyl group comprises between 1 and 2 DDD repeats, inclusive. In certain embodiments, the polypeptidyl group comprises 1 GG repeat, and the polypeptidyl group comprises between 1 and 2 DDD repeats, inclusive. In certain embodiments, the polypeptidyl group comprises 2 GG repeats, and the polypeptidyl group comprises between 1 and 2 DDD repeats, inclusive. In certain embodiments, the polypeptidyl group comprises between 1 and 3 GG repeats, inclusive, and the polypeptidyl group comprises 1 DDD repeat. In certain embodiments, the polypeptidyl group comprises between 1 and 2 GG repeats, inclusive, and the polypeptidyl group comprises 1 DDD repeat. In certain embodiments, the polypeptidyl group comprises 1 GG repeat, and the polypeptidyl group comprises 1 DDD repeat. In certain embodiments, the polypeptidyl group comprises 2 GG repeats, and the polypeptidyl group comprises 1 DDD repeat. In certain embodiments, the polypeptidyl group comprises between 1 and 3 GG repeats, inclusive, and the polypeptidyl group comprises 2 DDD repeats. In certain embodiments, the polypeptidyl group comprises between 1 and 2 GG repeats, inclusive, and the polypeptidyl group comprises 2 DDD repeats. In certain embodiments, the polypeptidyl group comprises 1 GG repeat, and the polypeptidyl group comprises 2 DDD repeats. In certain embodiments, the polypeptidyl group comprises 2 GG repeats, and the polypeptidyl group comprises 2 DDD repeats. In certain embodiments, the polypeptidyl group comprises between 1 and 3 GG repeats, inclusive, and the polypeptidyl group comprises 3 DDD repeats. In certain embodiments, the polypeptidyl group comprises between 1 and 2 GG repeats, inclusive, and the polypeptidyl group comprises 3 DDD repeats. In certain embodiments, the polypeptidyl group comprises 1 GG repeat, and the polypeptidyl group comprises 3 DDD repeats. In certain embodiments, the polypeptidyl group comprises 2 GG repeats, and the polypeptidyl group comprises 3 DDD repeats.

In certain embodiments, the polypeptidyl group comprises between 1 and 3 GGG repeats, inclusive, and the polypeptidyl group comprises between 1 and 3 DDD repeats, inclusive. In certain embodiments, the polypeptidyl group comprises between 1 and 2 GGG repeats, inclusive, and the polypeptidyl group comprises between 1 and 3 DDD repeats, inclusive. In certain embodiments, the polypeptidyl group comprises 1 GGG repeat, and the polypeptidyl group comprises between 1 and 3 DDD repeats, inclusive. In certain embodiments, the polypeptidyl group comprises 2 GGG repeats, and the polypeptidyl group comprises between 1 and 3 DDD repeats, inclusive. In certain embodiments, the polypeptidyl group comprises between 1 and 3 GGG repeats, inclusive, and the polypeptidyl group comprises between 1 and 2 DDD repeats, inclusive. In certain embodiments, the polypeptidyl group comprises between 1 and 2 GGG repeats, inclusive, and the polypeptidyl group comprises between 1 and 2 DDD repeats, inclusive. In certain embodiments, the polypeptidyl group comprises 1 GGG repeat, and the polypeptidyl group comprises between 1 and 2 DDD repeats, inclusive. In certain embodiments, the polypeptidyl group comprises 2 GGG repeats, and the polypeptidyl group comprises between 1 and 2 DDD repeats, inclusive. In certain embodiments, the polypeptidyl group comprises between 1 and 3 GGG repeats, inclusive, and the polypeptidyl group comprises 1 DDD repeat. In certain embodiments, the polypeptidyl group comprises between 1 and 2 GGG repeats, inclusive, and the polypeptidyl group comprises 1 DDD repeat. In certain embodiments, the polypeptidyl group comprises 1 GGG repeat, and the polypeptidyl group comprises 1 DDD repeat. In certain embodiments, the polypeptidyl group comprises 2 GGG repeats, and the polypeptidyl group comprises 1 DDD repeat. In certain embodiments, the polypeptidyl group comprises between 1 and 3 GGG repeats, inclusive, and the polypeptidyl group comprises 2 DDD repeats. In certain embodiments, the polypeptidyl group comprises between 1 and 2 GGG repeats, inclusive, and the polypeptidyl group comprises 2 DDD repeats. In certain embodiments, the polypeptidyl group comprises 1 GGG repeat, and the polypeptidyl group comprises 2 DDD repeats. In certain embodiments, the polypeptidyl group comprises 2 GGG repeats, and the polypeptidyl group comprises 2 DDD repeats. In certain embodiments, the polypeptidyl group comprises between 1 and 3 GGG repeats, inclusive, and the polypeptidyl group comprises 3 DDD repeats. In certain embodiments, the polypeptidyl group comprises between 1 and 2 GGG repeats, inclusive, and the polypeptidyl group comprises 3 DDD repeats. In certain embodiments, the polypeptidyl group comprises 1 GGG repeat, and the polypeptidyl group comprises 3 DDD repeats. In certain embodiments, the polypeptidyl group comprises 2 GGG repeats, and the polypeptidyl group comprises 3 DDD repeats.

In certain embodiments, the polypeptidyl group comprises at least 1 FF repeat. In certain embodiments, the polypeptidyl group comprises at least 2 FF repeats. In certain embodiments, the polypeptidyl group comprises at least 3 FF repeats. In certain embodiments, the polypeptidyl group comprises at least 4 FF repeats. In certain embodiments, the polypeptidyl group comprises at least 5 FF repeats. In certain embodiments, the polypeptidyl group comprises between 1 and 5 FF repeats, inclusive. In certain embodiments, the polypeptidyl group comprises between 1 and 4 FF repeats, inclusive. In certain embodiments, the polypeptidyl group comprises between 1 and 3 FF repeats, inclusive. In certain embodiments, the polypeptidyl group comprises between 1 and 2 FF repeats, inclusive. In certain embodiments, the polypeptidyl group comprises 1 FF repeat. In certain embodiments, the polypeptidyl group comprises 2 FF repeats. In certain embodiments, the polypeptidyl group comprises 3 FF repeats. In certain embodiments, the polypeptidyl group comprises 4 FF repeats. In certain embodiments, the polypeptidyl group comprises 5 FF repeats.

In certain embodiments, the polypeptidyl group comprises between 1 and 3 GG repeats, inclusive, and the polypeptidyl group comprises between 1 and 3 FF repeats, inclusive. In certain embodiments, the polypeptidyl group comprises between 1 and 2 GG repeats, inclusive, and the polypeptidyl group comprises between 1 and 3 FF repeats, inclusive. In certain embodiments, the polypeptidyl group comprises 1 GG repeat, and the polypeptidyl group comprises between 1 and 3 FF repeats, inclusive. In certain embodiments, the polypeptidyl group comprises 2 GG repeats, and the polypeptidyl group comprises between 1 and 3 FF repeats, inclusive. In certain embodiments, the polypeptidyl group comprises between 1 and 3 GG repeats, inclusive, and the polypeptidyl group comprises between 1 and 2 FF repeats, inclusive. In certain embodiments, the polypeptidyl group comprises between 1 and 2 GG repeats, inclusive, and the polypeptidyl group comprises between 1 and 2 FF repeats, inclusive. In certain embodiments, the polypeptidyl group comprises 1 GG repeat, and the polypeptidyl group comprises between 1 and 2 FF repeats, inclusive. In certain embodiments, the polypeptidyl group comprises 2 GG repeats, and the polypeptidyl group comprises between 1 and 2 FF repeats, inclusive. In certain embodiments, the polypeptidyl group comprises between 1 and 3 GG repeats, inclusive, and the polypeptidyl group comprises 1 FF repeat. In certain embodiments, the polypeptidyl group comprises between 1 and 2 GG repeats, inclusive, and the polypeptidyl group comprises 1 FF repeat. In certain embodiments, the polypeptidyl group comprises 1 GG repeat, and the polypeptidyl group comprises 1 FF repeat. In certain embodiments, the polypeptidyl group comprises 2 GG repeats, and the polypeptidyl group comprises 1 FF repeat. In certain embodiments, the polypeptidyl group comprises between 1 and 3 GG repeats, inclusive, and the polypeptidyl group comprises 2 FF repeats. In certain embodiments, the polypeptidyl group comprises between 1 and 2 GG repeats, inclusive, and the polypeptidyl group comprises 2 FF repeats. In certain embodiments, the polypeptidyl group comprises 1 GG repeat, and the polypeptidyl group comprises 2 FF repeats. In certain embodiments, the polypeptidyl group comprises 2 GG repeats, and the polypeptidyl group comprises 2 FF repeats.

In certain embodiments, the polypeptidyl group comprises between 1 and 3 GGG repeats, inclusive, and the polypeptidyl group comprises between 1 and 3 FF repeats, inclusive. In certain embodiments, the polypeptidyl group comprises between 1 and 2 GGG repeats, inclusive, and the polypeptidyl group comprises between 1 and 3 FF repeats, inclusive. In certain embodiments, the polypeptidyl group comprises 1 GGG repeat, and the polypeptidyl group comprises between 1 and 3 FF repeats, inclusive. In certain embodiments, the polypeptidyl group comprises 2 GGG repeats, and the polypeptidyl group comprises between 1 and 3 FF repeats, inclusive. In certain embodiments, the polypeptidyl group comprises between 1 and 3 GGG repeats, inclusive, and the polypeptidyl group comprises between 1 and 2 FF repeats, inclusive. In certain embodiments, the polypeptidyl group comprises between 1 and 2 GGG repeats, inclusive, and the polypeptidyl group comprises between 1 and 2 FF repeats, inclusive. In certain embodiments, the polypeptidyl group comprises 1 GGG repeat, and the polypeptidyl group comprises between 1 and 2 FF repeats, inclusive. In certain embodiments, the polypeptidyl group comprises 2 GGG repeats, and the polypeptidyl group comprises between 1 and 2 FF repeats, inclusive. In certain embodiments, the polypeptidyl group comprises between 1 and 3 GGG repeats, inclusive, and the polypeptidyl group comprises 1 FF repeat. In certain embodiments, the polypeptidyl group comprises between 1 and 2 GGG repeats, inclusive, and the polypeptidyl group comprises 1 FF repeat. In certain embodiments, the polypeptidyl group comprises 1 GGG repeat, and the polypeptidyl group comprises 1 FF repeat. In certain embodiments, the polypeptidyl group comprises 2 GGG repeats, and the polypeptidyl group comprises 1 FF repeat. In certain embodiments, the polypeptidyl group comprises between 1 and 3 GGG repeats, inclusive, and the polypeptidyl group comprises 2 FF repeats. In certain embodiments, the polypeptidyl group comprises between 1 and 2 GGG repeats, inclusive, and the polypeptidyl group comprises 2 FF repeats. In certain embodiments, the polypeptidyl group comprises 1 GGG repeat, and the polypeptidyl group comprises 2 FF repeats. In certain embodiments, the polypeptidyl group comprises 2 GGG repeats, and the polypeptidyl group comprises 2 FF repeats.

In certain embodiments, the polypeptidyl group comprises between 1 and 3 FF repeats, inclusive, and the polypeptidyl group comprises between 1 and 3 DD repeats, inclusive. In certain embodiments, the polypeptidyl group comprises between 1 and 2 FF repeats, inclusive, and the polypeptidyl group comprises between 1 and 3 DD repeats, inclusive. In certain embodiments, the polypeptidyl group comprises 1 FF repeat, and the polypeptidyl group comprises between 1 and 3 DD repeats, inclusive. In certain embodiments, the polypeptidyl group comprises 2 FF repeats, and the polypeptidyl group comprises between 1 and 3 DD repeats, inclusive. In certain embodiments, the polypeptidyl group comprises between 1 and 3 FF repeats, inclusive, and the polypeptidyl group comprises between 1 and 2 DD repeats, inclusive. In certain embodiments, the polypeptidyl group comprises between 1 and 2 FF repeats, inclusive, and the polypeptidyl group comprises between 1 and 2 DD repeats, inclusive. In certain embodiments, the polypeptidyl group comprises 1 FF repeat, and the polypeptidyl group comprises between 1 and 2 DD repeats, inclusive. In certain embodiments, the polypeptidyl group comprises 2 FF repeats, and the polypeptidyl group comprises between 1 and 2 DD repeats, inclusive. In certain embodiments, the polypeptidyl group comprises between 1 and 3 FF repeats, inclusive, and the polypeptidyl group comprises 1 DD repeat. In certain embodiments, the polypeptidyl group comprises between 1 and 2 FF repeats, inclusive, and the polypeptidyl group comprises 1 DD repeat. In certain embodiments, the polypeptidyl group comprises 1 FF repeat, and the polypeptidyl group comprises 1 DD repeat. In certain embodiments, the polypeptidyl group comprises 2 FF repeats, and the polypeptidyl group comprises 1 DD repeat. In certain embodiments, the polypeptidyl group comprises between 1 and 3 FF repeats, inclusive, and the polypeptidyl group comprises 2 DD repeats. In certain embodiments, the polypeptidyl group comprises between 1 and 2 FF repeats, inclusive, and the polypeptidyl group comprises 2 DD repeats. In certain embodiments, the polypeptidyl group comprises 1 FF repeat, and the polypeptidyl group comprises 2 DD repeats. In certain embodiments, the polypeptidyl group comprises 2 FF repeats, and the polypeptidyl group comprises 2 DD repeats. In certain embodiments, the polypeptidyl group comprises between 1 and 3 FF repeats, inclusive, and the polypeptidyl group comprises 3 DD repeats. In certain embodiments, the polypeptidyl group comprises between 1 and 2 FF repeats, inclusive, and the polypeptidyl group comprises 3 DD repeats. In certain embodiments, the polypeptidyl group comprises 1 FF repeat, and the polypeptidyl group comprises 3 DD repeats. In certain embodiments, the polypeptidyl group comprises 2 FF repeats, and the polypeptidyl group comprises 3 DD repeats.

In certain embodiments, the polypeptidyl group comprises between 1 and 3 FF repeats, inclusive, and the polypeptidyl group comprises between 1 and 3 DDD repeats, inclusive. In certain embodiments, the polypeptidyl group comprises between 1 and 2 FF repeats, inclusive, and the polypeptidyl group comprises between 1 and 3 DDD repeats, inclusive. In certain embodiments, the polypeptidyl group comprises 1 FF repeat, and the polypeptidyl group comprises between 1 and 3 DDD repeats, inclusive. In certain embodiments, the polypeptidyl group comprises 2 FF repeats, and the polypeptidyl group comprises between 1 and 3 DDD repeats, inclusive. In certain embodiments, the polypeptidyl group comprises between 1 and 3 FF repeats, inclusive, and the polypeptidyl group comprises between 1 and 2 DDD repeats, inclusive. In certain embodiments, the polypeptidyl group comprises between 1 and 2 FF repeats, inclusive, and the polypeptidyl group comprises between 1 and 2 DDD repeats, inclusive. In certain embodiments, the polypeptidyl group comprises 1 FF repeat, and the polypeptidyl group comprises between 1 and 2 DDD repeats, inclusive. In certain embodiments, the polypeptidyl group comprises 2 FF repeats, and the polypeptidyl group comprises between 1 and 2 DDD repeats, inclusive. In certain embodiments, the polypeptidyl group comprises between 1 and 3 FF repeats, inclusive, and the polypeptidyl group comprises 1 DDD repeat. In certain embodiments, the polypeptidyl group comprises between 1 and 2 FF repeats, inclusive, and the polypeptidyl group comprises 1 DDD repeat. In certain embodiments, the polypeptidyl group comprises 1 FF repeat, and the polypeptidyl group comprises 1 DDD repeat. In certain embodiments, the polypeptidyl group comprises 2 FF repeats, and the polypeptidyl group comprises 1 DDD repeat. In certain embodiments, the polypeptidyl group comprises between 1 and 3 FF repeats, inclusive, and the polypeptidyl group comprises 2 DDD repeats. In certain embodiments, the polypeptidyl group comprises between 1 and 2 FF repeats, inclusive, and the polypeptidyl group comprises 2 DDD repeats. In certain embodiments, the polypeptidyl group comprises 1 FF repeat, and the polypeptidyl group comprises 2 DDD repeats. In certain embodiments, the polypeptidyl group comprises 2 FF repeats, and the polypeptidyl group comprises 2 DDD repeats. In certain embodiments, the polypeptidyl group comprises between 1 and 3 FF repeats, inclusive, and the polypeptidyl group comprises 3 DDD repeats. In certain embodiments, the polypeptidyl group comprises between 1 and 2 FF repeats, inclusive, and the polypeptidyl group comprises 3 DDD repeats. In certain embodiments, the polypeptidyl group comprises 1 FF repeat, and the polypeptidyl group comprises 3 DDD repeats. In certain embodiments, the polypeptidyl group comprises 2 FF repeats, and the polypeptidyl group comprises 3 DDD repeats.

In certain embodiments, the oligonucleotide and the polypeptide are separated by at least 20 Å. In certain embodiments, the oligonucleotide and the polypeptide are separated by at least 25 Å. In certain embodiments, the oligonucleotide and the polypeptide are separated by at least 30 Å. In certain embodiments, the oligonucleotide and the polypeptide are separated by at least 33 Å. In certain embodiments, the oligonucleotide and the polypeptide are separated by at least 35 Å. In certain embodiments, the oligonucleotide and the polypeptide are separated by at least 40 Å. In certain embodiments, the oligonucleotide and the polypeptide are separated by at least 45 Å. In certain embodiments, the oligonucleotide and the polypeptide are separated by at least 50 Å. In certain embodiments, the oligonucleotide and the polypeptide are separated by at least 55 Å. In certain embodiments, the oligonucleotide and the polypeptide are separated by at least 60 Å. In certain embodiments, the oligonucleotide and the polypeptide are separated by at least 65 Å. In certain embodiments, the oligonucleotide and the polypeptide are separated by at least 70 Å. In certain embodiments, the oligonucleotide and the polypeptide are separated by at least 75 Å. In certain embodiments, the oligonucleotide and the polypeptide are separated by between about 20 Å and about 75 Å, inclusive. In certain embodiments, the oligonucleotide and the polypeptide are separated by between about 20 Å and about 70 Å, inclusive. In certain embodiments, the oligonucleotide and the polypeptide are separated by between about 20 Å and about 65 Å, inclusive. In certain embodiments, the oligonucleotide and the polypeptide are separated by between about 20 Å and about 60 Å, inclusive. In certain embodiments, the oligonucleotide and the polypeptide are separated by between about 20 Å and about 55 Å, inclusive. In certain embodiments, the oligonucleotide and the polypeptide are separated by between about 20 Å and about 50 Å, inclusive. In certain embodiments, the oligonucleotide and the polypeptide are separated by between about 20 Å and about 45 Å, inclusive. In certain embodiments, the oligonucleotide and the polypeptide are separated by between about 20 Å and about 40 Å, inclusive. In certain embodiments, the oligonucleotide and the polypeptide are separated by between about 20 Å and about 35 Å, inclusive. In certain embodiments, the oligonucleotide and the polypeptide are separated by between about 25 Å and about 75 Å, inclusive. In certain embodiments, the oligonucleotide and the polypeptide are separated by between about 25 Å and about 70 Å, inclusive. In certain embodiments, the oligonucleotide and the polypeptide are separated by between about 25 Å and about 65 Å, inclusive. In certain embodiments, the oligonucleotide and the polypeptide are separated by between about 25 Å and about 60 Å, inclusive. In certain embodiments, the oligonucleotide and the polypeptide are separated by between about 25 Å and about 55 Å, inclusive. In certain embodiments, the oligonucleotide and the polypeptide are separated by between about 25 Å and about 50 Å, inclusive. In certain embodiments, the oligonucleotide and the polypeptide are separated by between about 25 Å and about 45 Å, inclusive. In certain embodiments, the oligonucleotide and the polypeptide are separated by between about 25 Å and about 40 Å, inclusive. In certain embodiments, the oligonucleotide and the polypeptide are separated by between about 25 Å and about 35 Å, inclusive. In certain embodiments, the oligonucleotide and the polypeptide are separated by between about 30 Å and about 75 Å, inclusive. In certain embodiments, the oligonucleotide and the polypeptide are separated by between about 30 Å and about 70 Å, inclusive. In certain embodiments, the oligonucleotide and the polypeptide are separated by between about 30 Å and about 65 Å, inclusive. In certain embodiments, the oligonucleotide and the polypeptide are separated by between about 30 Å and about 60 Å, inclusive. In certain embodiments, the oligonucleotide and the polypeptide are separated by between about 30 Å and about 55 Å, inclusive. In certain embodiments, the oligonucleotide and the polypeptide are separated by between about 30 Å and about 50 Å, inclusive. In certain embodiments, the oligonucleotide and the polypeptide are separated by between about 30 Å and about 45 Å, inclusive. In certain embodiments, the oligonucleotide and the polypeptide are separated by between about 30 Å and about 40 Å, inclusive. In certain embodiments, the oligonucleotide and the polypeptide are separated by between about 30 Å and about 35 Å, inclusive. In certain embodiments, the oligonucleotide and the polypeptide are separated by about 20 Å. In certain embodiments, the oligonucleotide and the polypeptide are separated by about 25 Å. In certain embodiments, the oligonucleotide and the polypeptide are separated by about 30 Å. In certain embodiments, the oligonucleotide and the polypeptide are separated by about 33 Å. In certain embodiments, the oligonucleotide and the polypeptide are separated by about 35 Å. In certain embodiments, the oligonucleotide and the polypeptide are separated by about 40 Å. In certain embodiments, the oligonucleotide and the polypeptide are separated by about 45 Å. In certain embodiments, the oligonucleotide and the polypeptide are separated by about 50 Å. In certain embodiments, the oligonucleotide and the polypeptide are separated by about 55 Å. In certain embodiments, the oligonucleotide and the polypeptide are separated by about 60 Å. In certain embodiments, the oligonucleotide and the polypeptide are separated by about 65 Å. In certain embodiments, the oligonucleotide and the polypeptide are separated by about 70 Å. In certain embodiments, the oligonucleotide and the polypeptide are separated by about 75 Å.

In certain embodiments, the polypeptidyl group comprises a moiety selected from:

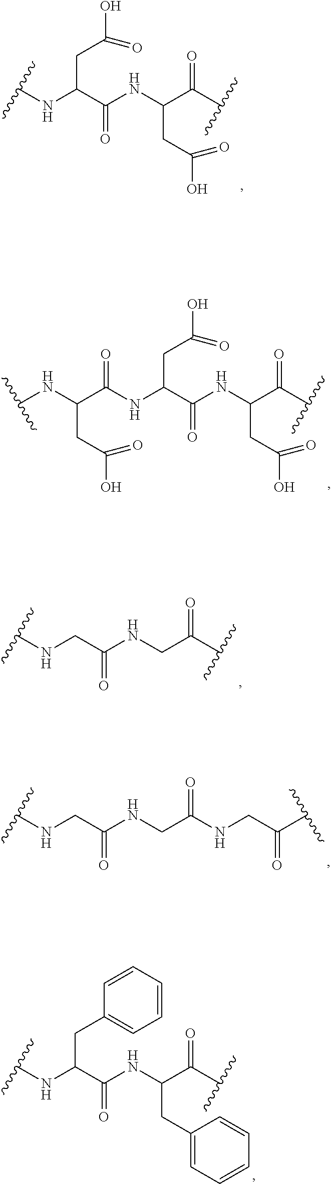 [SMILES: *NC(CC(=O)O)C(=O)NC(CC(=O)O)C(*)=O ; *NC(CC(=O)O)C(=O)NC(CC(=O)O)C(=O)NC(CC(=O)O)C(*)=O ; *NCC(=O)NCC(*)=O ; *NCC(=O)NCC(=O)NCC(*)=O ; *NC(Cc1ccccc1)C(=O)NC(Cc1ccccc1)C(*)=O]

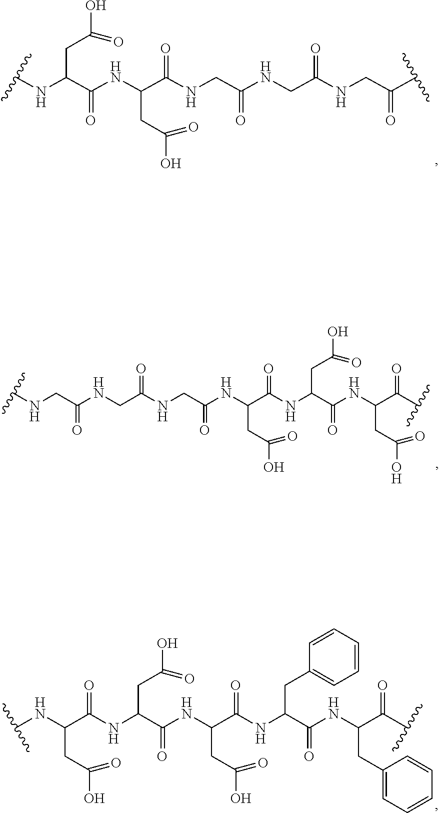 [SMILES: *NC(CC(=O)O)C(=O)NC(CC(=O)O)C(=O)NCC(=O)NCC(=O)NCC(*)=O ; *NCC(=O)NCC(=O)NCC(=O)NC(CC(=O)O)C(=O)NC(CC(=O)O)C(=O)NC(CC(=O)O)C(*)=O ; *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof. In certain embodiments, the polypeptidyl group comprise

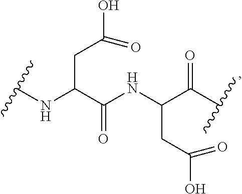 [SMILES: *NC(CC(=O)O)C(=O)NC(CC(=O)O)C(*)=O]

or a salt thereof. In certain embodiments, the polypeptidyl group comprises

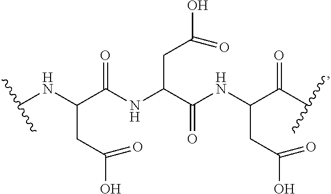 [SMILES: *NC(CC(=O)O)C(=O)NC(CC(=O)O)C(=O)NC(CC(=O)O)C(*)=O]

or a salt thereof. In certain embodiments, the polypeptidyl group comprises

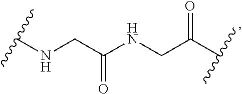 [SMILES: *NCC(=O)NCC(*)=O]

or a salt thereof. In certain embodiments, the polypeptidyl group comprises

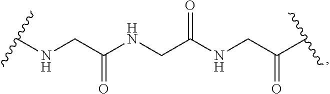 [SMILES: *NCC(=O)NCC(=O)NCC(*)=O]

or a salt thereof. In certain embodiments, the polypeptidyl group comprises

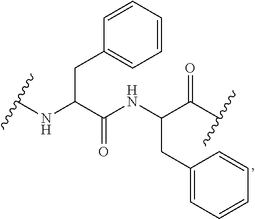 [SMILES: *NC(Cc1ccccc1)C(=O)NC(Cc1ccccc1)C(*)=O]

or a salt thereof. In certain embodiments, the polypeptidyl group comprises

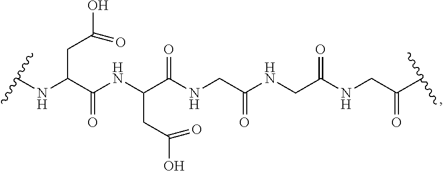 [SMILES: *NC(CC(=O)O)C(=O)NC(CC(=O)O)C(=O)NCC(=O)NCC(=O)NCC(*)=O]

or a salt thereof. In certain embodiments, the polypeptidyl group comprises

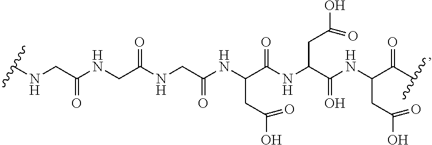 [SMILES: *NCC(=O)NCC(=O)NCC(=O)NC(CC(=O)O)C(=O)NC(CC(=O)O)C(=O)NC(CC(=O)O)C(*)=O]

or a salt thereof. In certain embodiments, the polypeptidyl group comprises

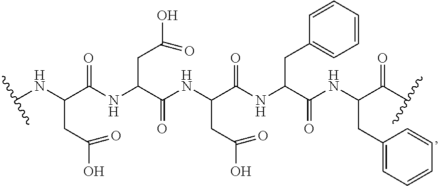 [SMILES: *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof.

In certain embodiments, the polypeptidyl group comprises

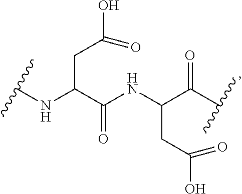 [SMILES: *NC(CC(=O)O)C(=O)NC(CC(=O)O)C(*)=O]

or a salt thereof, and

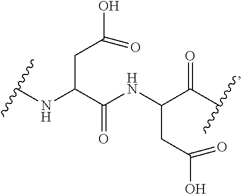 [SMILES: *NC(CC(=O)O)C(=O)NC(CC(=O)O)C(*)=O]

or a salt thereof. In certain embodiments, the polypeptidyl group comprises

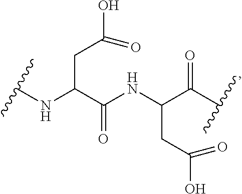 [SMILES: *NC(CC(=O)O)C(=O)NC(CC(=O)O)C(*)=O]

or a salt thereof, and

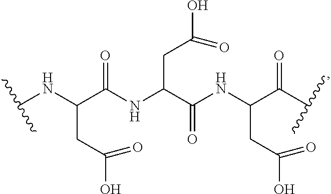 [SMILES: *NC(CC(=O)O)C(=O)NC(CC(=O)O)C(=O)NC(CC(=O)O)C(*)=O]

or a salt thereof. In certain embodiments, the polypeptidyl group comprises

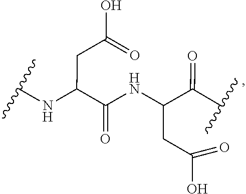 [SMILES: *NC(CC(=O)O)C(=O)NC(CC(=O)O)C(*)=O]

or a salt thereof, and

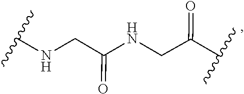 [SMILES: *NCC(=O)NCC(*)=O]

or a salt thereof. In certain embodiments, the polypeptidyl group comprises

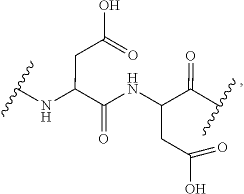 [SMILES: *NC(CC(=O)O)C(=O)NC(CC(=O)O)C(*)=O]

or a salt thereof, and

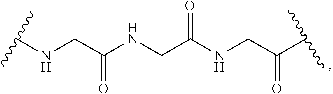 [SMILES: *NCC(=O)NCC(=O)NCC(*)=O]

or a salt thereof. In certain embodiments, the polypeptidyl group comprises

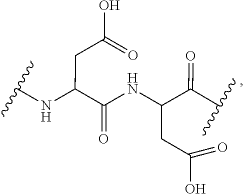 [SMILES: *NC(CC(=O)O)C(=O)NC(CC(=O)O)C(*)=O]

or a salt thereof, and

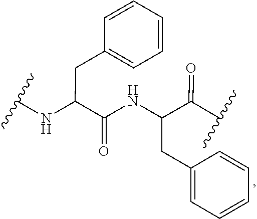 [SMILES: *NC(Cc1ccccc1)C(=O)NC(Cc1ccccc1)C(*)=O]

or a salt thereof. In certain embodiments, the polypeptidyl group comprises

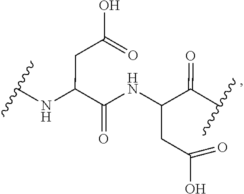 [SMILES: *NC(CC(=O)O)C(=O)NC(CC(=O)O)C(*)=O]

or a salt thereof, and

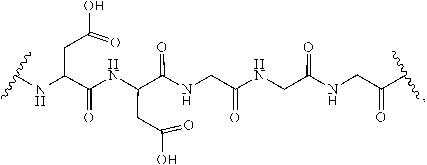 [SMILES: *NC(CC(=O)O)C(=O)NC(CC(=O)O)C(=O)NCC(=O)NCC(=O)NCC(*)=O]

or a salt thereof. In certain embodiments, the polypeptidyl group comprises

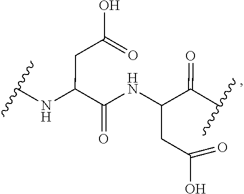 [SMILES: *NC(CC(=O)O)C(=O)NC(CC(=O)O)C(*)=O]

or a salt thereof, and

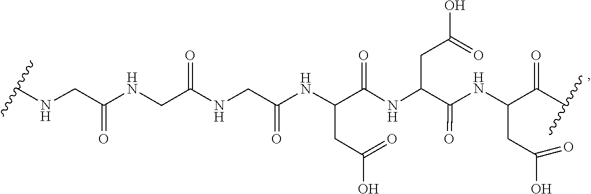 [SMILES: *NCC(=O)NCC(=O)NCC(=O)NC(CC(=O)O)C(=O)NC(CC(=O)O)C(=O)NC(CC(=O)O)C(*)=O]

or a salt thereof. In certain embodiments, the polypeptidyl group comprises

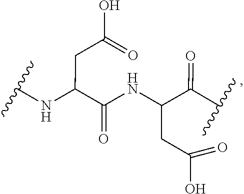 [SMILES: *NC(CC(=O)O)C(=O)NC(CC(=O)O)C(*)=O]

or a salt thereof, and

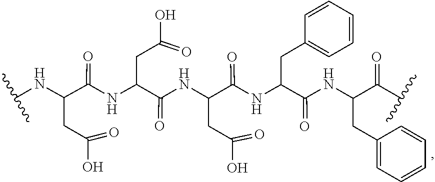 [SMILES: *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof. In certain embodiments, the polypeptidyl group comprises

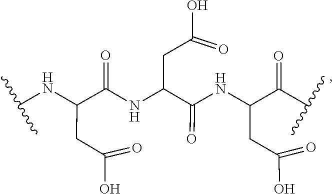 [SMILES: *NC(CC(=O)O)C(=O)NC(CC(=O)O)C(=O)NC(CC(=O)O)C(*)=O]

or a salt thereof, and

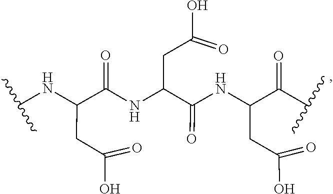 [SMILES: *NC(CC(=O)O)C(=O)NC(CC(=O)O)C(=O)NC(CC(=O)O)C(*)=O]

or a salt thereof. In certain embodiments, the polypeptidyl group comprises

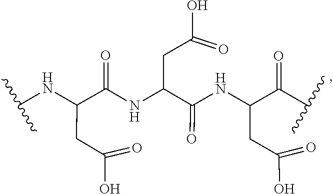 [SMILES: *NC(CC(=O)O)C(=O)NC(CC(=O)O)C(=O)NC(CC(=O)O)C(*)=O]

or a salt thereof, and

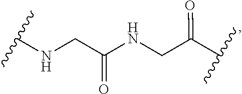 [SMILES: *NCC(=O)NCC(*)=O]

or a salt thereof. In certain embodiments, the polypeptidyl group comprises

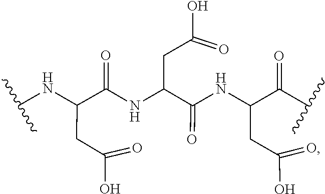 [SMILES: *NC(CC(=O)O)C(=O)NC(CC(=O)O)C(=O)NC(CC(=O)O)C(*)=O]

or a salt thereof, and

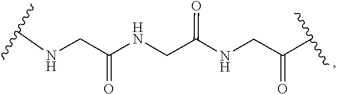 [SMILES: *NCC(=O)NCC(=O)NCC(*)=O]

or a salt thereof. In certain embodiments, the polypeptidyl group comprises

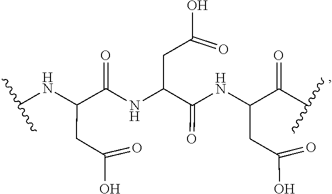 [SMILES: *NC(CC(=O)O)C(=O)NC(CC(=O)O)C(=O)NC(CC(=O)O)C(*)=O]

or a salt thereof, and

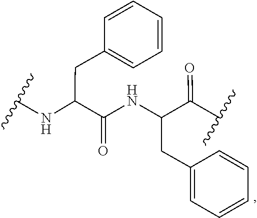 [SMILES: *NC(Cc1ccccc1)C(=O)NC(Cc1ccccc1)C(*)=O]

or a salt thereof. In certain embodiments, the polypeptidyl group comprises

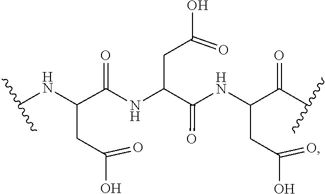 [SMILES: *NC(CC(=O)O)C(=O)NC(CC(=O)O)C(=O)NC(CC(=O)O)C(*)=O]

or a salt thereof, and

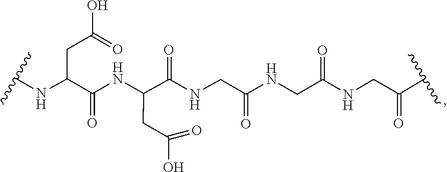 [SMILES: *NC(CC(=O)O)C(=O)NC(CC(=O)O)C(=O)NCC(=O)NCC(=O)NCC(*)=O]

or a salt thereof. In certain embodiments, the polypeptidyl group comprises

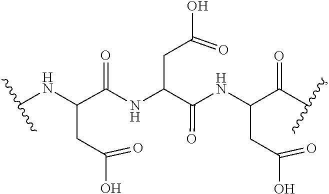 [SMILES: *NC(CC(=O)O)C(=O)NC(CC(=O)O)C(=O)NC(CC(=O)O)C(*)=O]

or a salt thereof, and

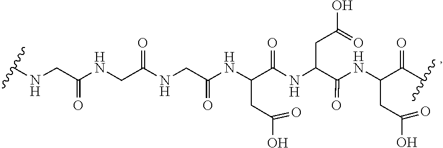 [SMILES: *NCC(=O)NCC(=O)NCC(=O)NC(CC(=O)O)C(=O)NC(CC(=O)O)C(=O)NC(CC(=O)O)C(*)=O]

or a salt thereof. In certain embodiments, the polypeptidyl group comprises

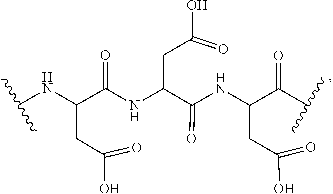 [SMILES: *NC(CC(=O)O)C(=O)NC(CC(=O)O)C(=O)NC(CC(=O)O)C(*)=O]

or a salt thereof, and

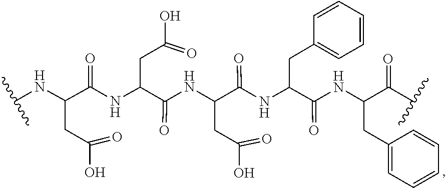 [SMILES: *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof. In certain embodiments, the polypeptidyl group comprises

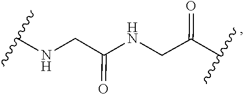 [SMILES: *NCC(=O)NCC(*)=O]

or a salt thereof, and

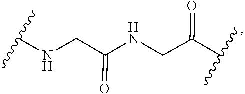 [SMILES: *NCC(=O)NCC(*)=O]

or a salt thereof. In certain embodiments, the polypeptidyl group comprises

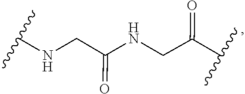 [SMILES: *NCC(=O)NCC(*)=O]

or a salt thereof, and

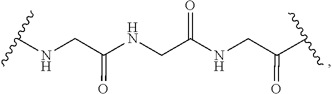 [SMILES: *NCC(=O)NCC(=O)NCC(*)=O]

or a salt thereof. In certain embodiments, the polypeptidyl group comprises

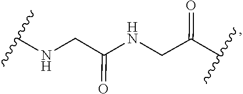 [SMILES: *NCC(=O)NCC(*)=O]

or a salt thereof, and

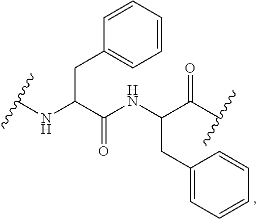 [SMILES: *NC(Cc1ccccc1)C(=O)NC(Cc1ccccc1)C(*)=O]

or a salt thereof. In certain embodiments, the polypeptidyl group comprises

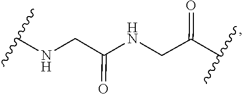 [SMILES: *NCC(=O)NCC(*)=O]

or a salt thereof, and

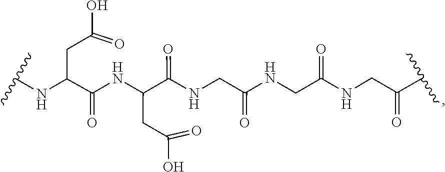 [SMILES: *NC(CC(=O)O)C(=O)NC(CC(=O)O)C(=O)NCC(=O)NCC(=O)NCC(*)=O]

or a salt thereof. In certain embodiments, the polypeptidyl group comprises

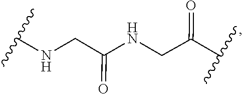 [SMILES: *NCC(=O)NCC(*)=O]

or a salt thereof, and

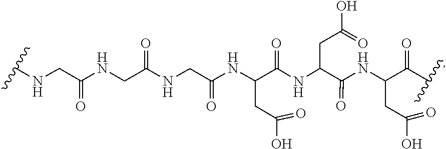 [SMILES: *NCC(=O)NCC(=O)NCC(=O)NC(CC(=O)O)C(=O)NC(CC(=O)O)C(=O)NC(CC(=O)O)C(*)=O]

or a salt thereof. In certain embodiments, the polypeptidyl group comprises

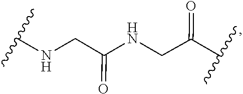 [SMILES: *NCC(=O)NCC(*)=O]

or a salt thereof, and

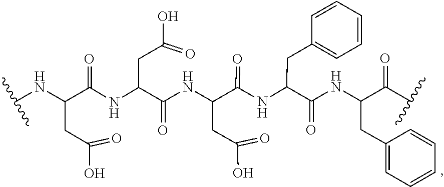 [SMILES: *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof. In certain embodiments, the polypeptidyl group comprises

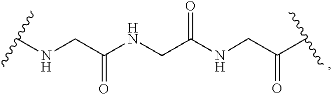 [SMILES: *NCC(=O)NCC(=O)NCC(*)=O]

or a salt thereof, and

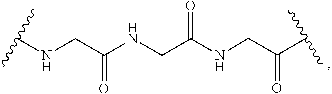 [SMILES: *NCC(=O)NCC(=O)NCC(*)=O]

or a salt thereof. In certain embodiments, the polypeptidyl group comprises

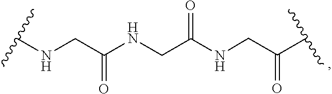 [SMILES: *NCC(=O)NCC(=O)NCC(*)=O]

or a salt thereof, and

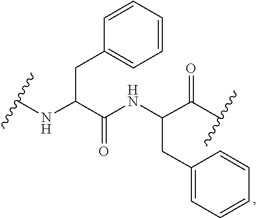 [SMILES: *NC(Cc1ccccc1)C(=O)NC(Cc1ccccc1)C(*)=O]

or a salt thereof. In certain embodiments, the polypeptidyl group comprises

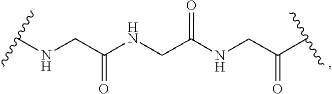 [SMILES: *NCC(=O)NCC(=O)NCC(*)=O]

or a salt thereof, and

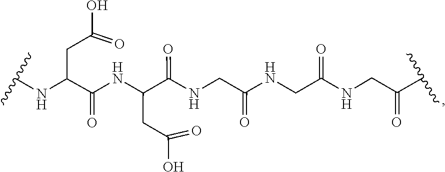 [SMILES: *NC(CC(=O)O)C(=O)NC(CC(=O)O)C(=O)NCC(=O)NCC(=O)NCC(*)=O]

or a salt thereof. In certain embodiments, the polypeptidyl group comprises

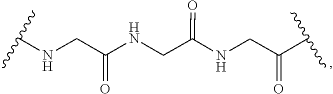 [SMILES: *NCC(=O)NCC(=O)NCC(*)=O]

or a salt thereof, and

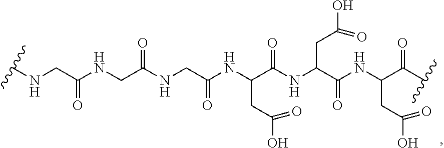 [SMILES: *NCC(=O)NCC(=O)NCC(=O)NC(CC(=O)O)C(=O)NC(CC(=O)O)C(=O)NC(CC(=O)O)C(*)=O]

or a salt thereof. In certain embodiments, the polypeptidyl group comprises

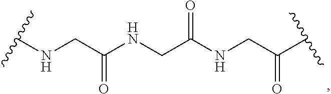 [SMILES: *NCC(=O)NCC(=O)NCC(*)=O]

or a salt thereof, and

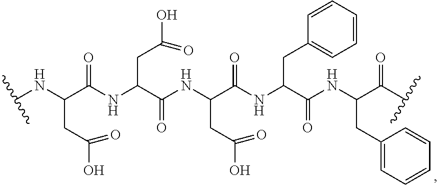 [SMILES: *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof. In certain embodiments, the polypeptidyl group comprises

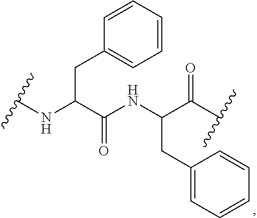 [SMILES: *NC(Cc1ccccc1)C(=O)NC(Cc1ccccc1)C(*)=O]

or a salt thereof, and

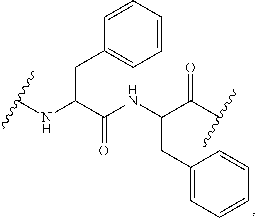 [SMILES: *NC(Cc1ccccc1)C(=O)NC(Cc1ccccc1)C(*)=O]

or a salt thereof. In certain embodiments, the polypeptidyl group comprises

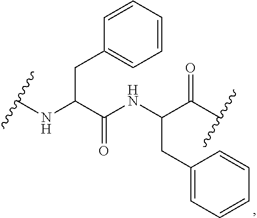 [SMILES: *NC(Cc1ccccc1)C(=O)NC(Cc1ccccc1)C(*)=O]

or a salt thereof, and

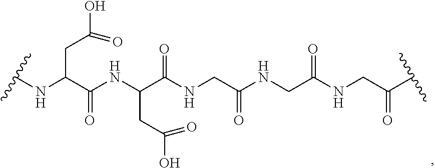 [SMILES: *NC(CC(=O)O)C(=O)NC(CC(=O)O)C(=O)NCC(=O)NCC(=O)NCC(*)=O]

or a salt thereof. In certain embodiments, the polypeptidyl group comprises

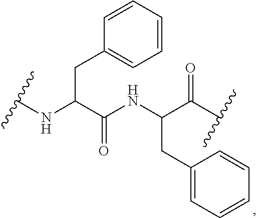 [SMILES: *NC(Cc1ccccc1)C(=O)NC(Cc1ccccc1)C(*)=O]

or a salt thereof, and

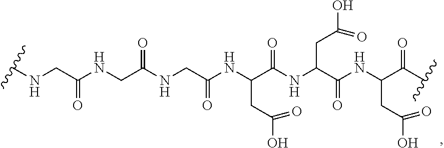 [SMILES: *NCC(=O)NCC(=O)NCC(=O)NC(CC(=O)O)C(=O)NC(CC(=O)O)C(=O)NC(CC(=O)O)C(*)=O]

or a salt thereof. In certain embodiments, the polypeptidyl group comprises

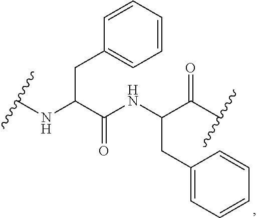 [SMILES: *NC(Cc1ccccc1)C(=O)NC(Cc1ccccc1)C(*)=O]

or a salt thereof, and

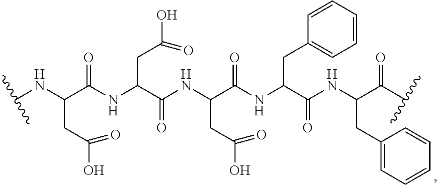 [SMILES: *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof. In certain embodiments, the polypeptidyl group comprises

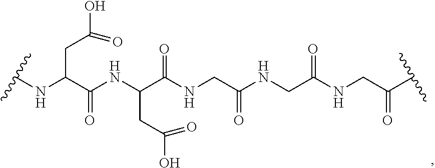 [SMILES: *NC(CC(=O)O)C(=O)NC(CC(=O)O)C(=O)NCC(=O)NCC(=O)NCC(*)=O]

or a salt thereof, and

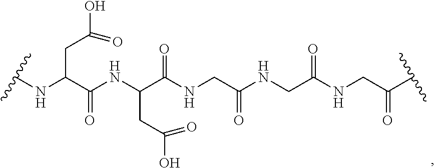 [SMILES: *NC(CC(=O)O)C(=O)NC(CC(=O)O)C(=O)NCC(=O)NCC(=O)NCC(*)=O]

or a salt thereof. In certain embodiments, the polypeptidyl group comprises

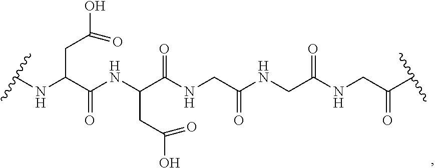 [SMILES: *NC(CC(=O)O)C(=O)NC(CC(=O)O)C(=O)NCC(=O)NCC(=O)NCC(*)=O]

or a salt thereof, and

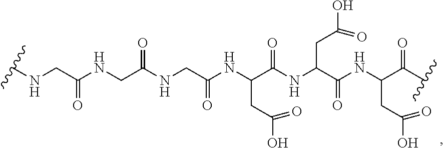 [SMILES: *NCC(=O)NCC(=O)NCC(=O)NC(CC(=O)O)C(=O)NC(CC(=O)O)C(=O)NC(CC(=O)O)C(*)=O]

or a salt thereof. In certain embodiments, the polypeptidyl group comprises

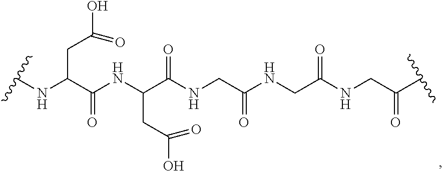 [SMILES: *NC(CC(=O)O)C(=O)NC(CC(=O)O)C(=O)NCC(=O)NCC(=O)NCC(*)=O]

or a salt thereof, and

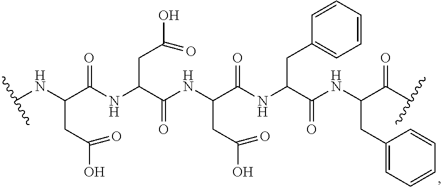 [SMILES: *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof. In certain embodiments, the polypeptidyl group comprises

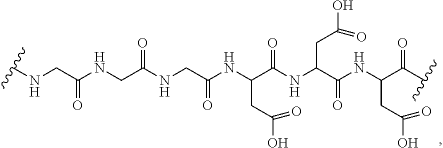 [SMILES: *NCC(=O)NCC(=O)NCC(=O)NC(CC(=O)O)C(=O)NC(CC(=O)O)C(=O)NC(CC(=O)O)C(*)=O]

or a salt thereof, and

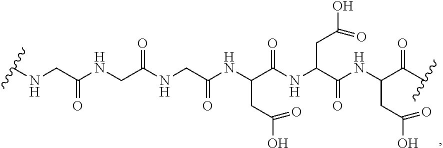 [SMILES: *NCC(=O)NCC(=O)NCC(=O)NC(CC(=O)O)C(=O)NC(CC(=O)O)C(=O)NC(CC(=O)O)C(*)=O]

or a salt thereof. In certain embodiments, the polypeptidyl group comprises

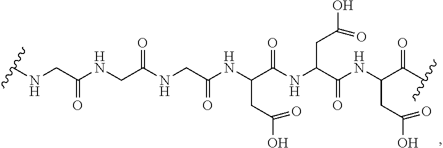 [SMILES: *NCC(=O)NCC(=O)NCC(=O)NC(CC(=O)O)C(=O)NC(CC(=O)O)C(=O)NC(CC(=O)O)C(*)=O]

or a salt thereof and

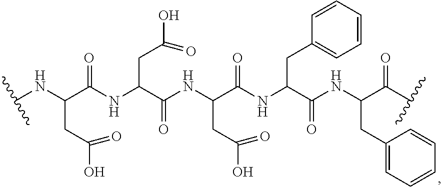 [SMILES: *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof. In certain embodiments, the polypeptidyl group comprises

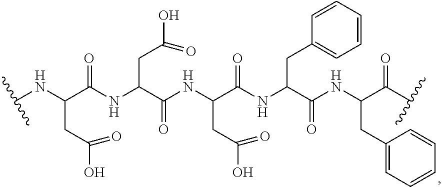 [SMILES: *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof, and

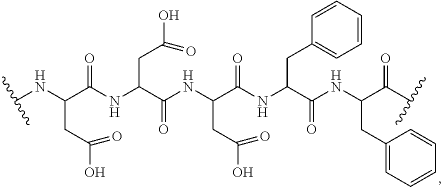 [SMILES: *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof.

In certain embodiments, the polypeptidyl group comprises a moiety selected from:

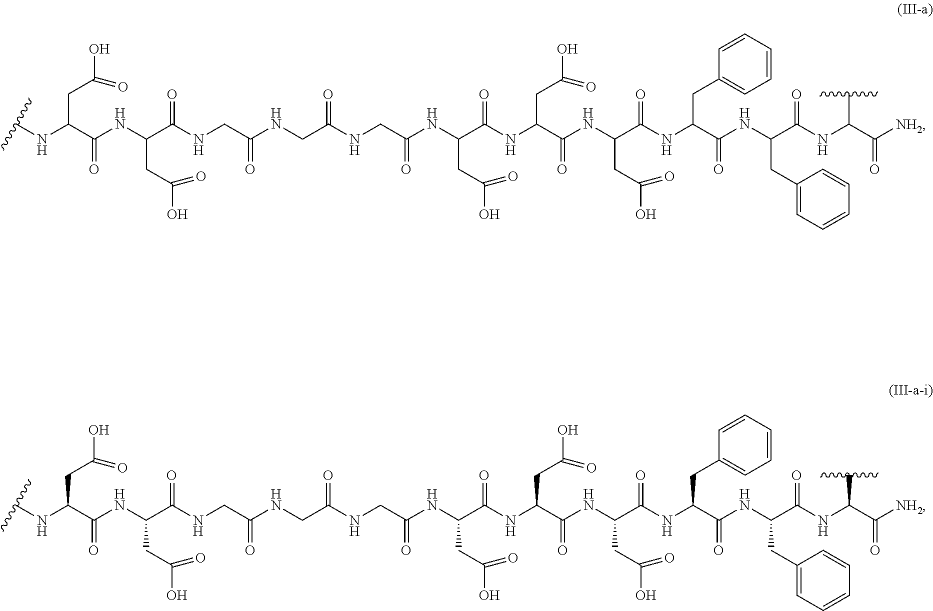 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O ; *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof. In certain embodiments, the polypeptidyl group comprises a moiety of formula

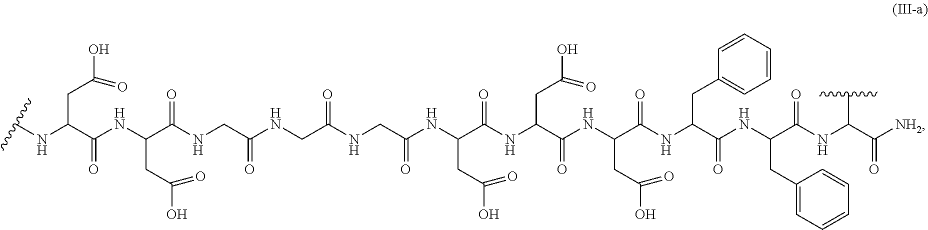 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, the polypeptidyl group comprises a moiety of formula

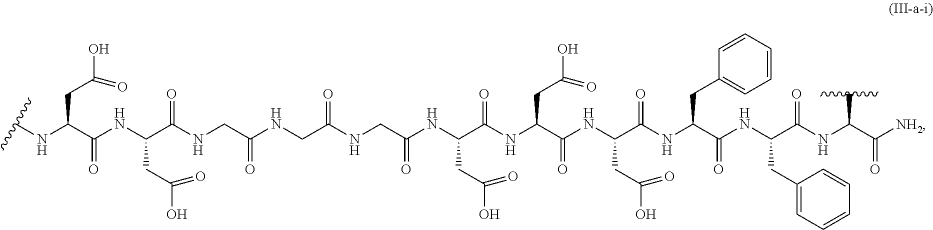 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof. In certain embodiments, the polypeptidyl group comprises a moiety of formula

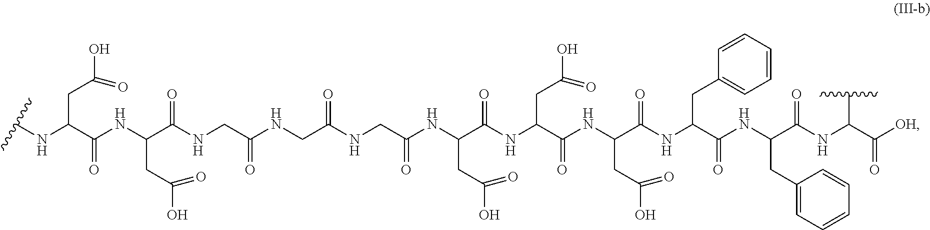 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(=O)O]

or a salt thereof. In certain embodiments, the polypeptidyl group comprises a moiety of formula

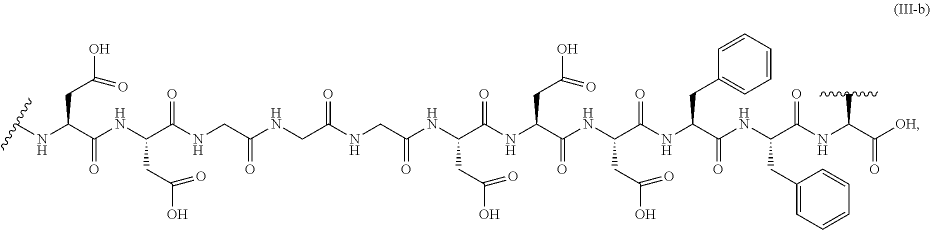 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(=O)O]

or a salt thereof.

In certain embodiments, the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), or a salt thereof, wherein Cy is cysteic acid. In certain embodiments, the polypeptidyl group comprises a sequence GPPPPPPPPG (SEQ ID NO: 61), or a salt thereof. In certain embodiments, the polypeptidyl group comprises a sequence isoEGWRW (SEQ ID NO: 62), or a salt thereof. In certain embodiments, the polypeptidyl group comprises a sequence DDGGGDDDFF (SEQ ID NO: 32), or a salt thereof. In certain embodiments, the polypeptidyl group comprises a sequence GGSSSGSGNDEEFQ (SEQ ID NO: 59), or a salt thereof. In certain embodiments, the polypeptidyl group comprises a sequence GGGGGDPDPD (SEQ ID NO: 54), or a salt thereof. In certain embodiments, the polypeptidyl group comprises a sequence GGGGGDPDPDFF (SEQ ID NO: 55), or a salt thereof. In certain embodiments, the polypeptidyl group comprises a sequence GGGGGGDPDPD (SEQ ID NO: 57), or a salt thereof. In certain embodiments, the polypeptidyl group comprises a sequence GDGDGDGDGDFF (SEQ ID NO: 53), or a salt thereof. In certain embodiments, the polypeptidyl group comprises a sequence GDDGDGDGDFF (SEQ ID NO: 51), or a salt thereof. In certain embodiments, the polypeptidyl group comprises a sequence NNGGGNNNFF (SEQ ID NO: 65), or a salt thereof. In certain embodiments, the polypeptidyl group comprises a sequence DDGGGCyCyCyFF (SEQ ID NO: 45), or a salt thereof, wherein Cy is cysteic acid. In certain embodiments, the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), wherein Cy is cysteic acid. In certain embodiments, the polypeptidyl group comprises a sequence GPPPPPPPPG (SEQ ID NO: 61). In certain embodiments, the polypeptidyl group comprises a sequence isoEGWRW (SEQ ID NO: 62). In certain embodiments, the polypeptidyl group comprises a sequence DDGGGDDDFF (SEQ ID NO: 32). In certain embodiments, the polypeptidyl group comprises a sequence GGSSSGSGNDEEFQ (SEQ ID NO: 59). In certain embodiments, the polypeptidyl group comprises a sequence GGGGGDPDPD (SEQ ID NO: 54). In certain embodiments, the polypeptidyl group comprises a sequence GGGGGDPDPDFF (SEQ ID NO: 55). In certain embodiments, the polypeptidyl group comprises a sequence GGGGGGDPDPD (SEQ ID NO: 57). In certain embodiments, the polypeptidyl group comprises a sequence GDGDGDGDGDFF (SEQ ID NO: 53). In certain embodiments, the polypeptidyl group comprises a sequence GDDGDGDGDFF (SEQ ID NO: 51). In certain embodiments, the polypeptidyl group comprises a sequence NNGGGNNNFF (SEQ ID NO: 65). In certain embodiments, the polypeptidyl group comprises a sequence DDGGGCyCyCyFF (SEQ ID NO: 45), wherein Cy is cysteic acid.

In certain embodiments, L further comprises at least one of optionally substituted alkylene, optionally substituted alkenylene, optionally substituted alkynylene, optionally substituted heteroalkylene, optionally substituted heteroalkenylene, optionally substituted heteroalkynylene, optionally substituted heterocyclylene, optionally substituted carbocyclylene, optionally substituted arylene, optionally substituted heteroarylene, a peptidyl group, a dipeptidyl group, a polypeptidyl group, a click chemistry handle, or a combination thereof.

In certain embodiments, L further comprises optionally substituted alkylene. In certain embodiments, L further comprises optionally substituted C₁₋₁₂ alkylene. In certain embodiments, L further comprises optionally substituted C₁₋₁₀ alkylene. In certain embodiments, L further comprises optionally substituted C₁₋₆ alkylene. In certain embodiments, L further comprises unsubstituted C₁₋₆ alkylene. In certain embodiments, L further comprises substituted C₁₋₆ alkylene. In certain embodiments, L further comprises substituted C₁₋₆ alkylene substituted with one or more oxo groups. In certain embodiments, L further comprises substituted C₁₋₆ alkylene substituted with one oxo group. In certain embodiments, L further comprises substituted C₁₋₆ alkylene substituted with two oxo groups. In certain embodiments, L further comprises substituted or unsubstituted methylene, substituted or unsubstituted ethylene, substituted or unsubstituted n-propylene, substituted or unsubstituted isopropylene, substituted or unsubstituted n-butylene, substituted or unsubstituted tert-butylene, substituted or unsubstituted sec-butylene, substituted or unsubstituted isobutylene, substituted or unsubstituted n-pentylene, substituted or unsubstituted 3-pentanylene, substituted or unsubstituted amylene, substituted or unsubstituted neopentylene, substituted or unsubstituted 3-methylene-2-butanylene, substituted or unsubstituted tert-amylene, or substituted or unsubstituted n-hexylene. In certain embodiments, L further comprises unsubstituted methylene. In certain embodiments, L further comprises substituted methylene. In certain embodiments, L further comprises unsubstituted n-butylene. In certain embodiments, L further comprises substituted n-butylene. In certain embodiments, L further comprises substituted n-butylene substituted with one or more oxo groups. In certain embodiments, L further comprises substituted n-butylene substituted with one oxo group. In certain embodiments, L further comprises substituted n-butylene substituted with two oxo groups. In certain embodiments, L further comprises

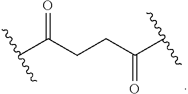 [SMILES: *C(=O)CCC(*)=O]

In certain embodiments, L further comprises optionally substituted alkenylene. In certain embodiments, L further comprises optionally substituted C₂₋₁₂ alkenylene. In certain embodiments, L further comprises optionally substituted C₂₋₆ alkenylene. In certain embodiments, L further comprises substituted or unsubstituted ethenylene, substituted or unsubstituted 1-propenylene, substituted or unsubstituted 2-propenylene, substituted or unsubstituted 1-butenylene, substituted or unsubstituted 2-butenylene, substituted or unsubstituted butadienylene, substituted or unsubstituted pentenylene, substituted or unsubstituted pentadienylene, or substituted or unsubstituted hexenylene. In certain embodiments, L further comprises optionally substituted alkynylene. In certain embodiments, L further comprises optionally substituted C₂₋₁₂ alkynylene. In certain embodiments, L further comprises optionally substituted C₂₋₆ alkynylene. In certain embodiments, L further comprises substituted or unsubstituted ethynylene, substituted or unsubstituted 1-propynylene, substituted or unsubstituted 2-propynylene, substituted or unsubstituted 1-butynylene, substituted or unsubstituted 2-butynylene, substituted or unsubstituted pentynylene, or substituted or unsubstituted hexynylene. In certain embodiments, L further comprises optionally substituted heteroalkylene. In certain embodiments, L further comprises optionally substituted heteroC₁₋₁₂ alkylene. In certain embodiments, L further comprises optionally substituted heteroC₁₋₆ alkylene. In certain embodiments, L further comprises optionally substituted heteroalkenylene. In certain embodiments, L further comprises optionally substituted heteroC₁₋₁₂ alkenylene. In certain embodiments, L further comprises optionally substituted heteroC₁₋₆ alkenylene. In certain embodiments, L further comprises optionally substituted heteroalkynylene. In certain embodiments, L further comprises optionally substituted heteroC₁₋₁₂ alkynylene. In certain embodiments, L further comprises optionally substituted heteroC₁₋₆ alkynylene. In certain embodiments, L further comprises optionally substituted carbocyclylene. In certain embodiments, L further comprises optionally substituted C₃₋₁₄ cycloalkylene. In certain embodiments, L further comprises optionally substituted heterocyclylene. In certain embodiments, L further comprises optionally substituted 5-10 membered heterocyclylene. In certain embodiments, L further comprises optionally substituted arylene. In certain embodiments, L further comprises optionally substituted 6-14 membered arylene. In certain embodiments, L further comprises optionally substituted phenylene. In certain embodiments, L further comprises substituted phenylene. In certain embodiments, L further comprises unsubstituted phenylene. In certain embodiments, L further comprises optionally substituted heteroarylene. In certain embodiments, L further comprises optionally substituted 5-14 membered heteroarylene. In certain embodiments, L further comprises optionally substituted monocyclic heteroarylene. In certain embodiments, L further comprises optionally substituted 5- to 6-membered, monocyclic heteroarylene. In certain embodiments, L further comprises optionally substituted pyrrolylene, optionally substituted furanylene, optionally substituted thiophenylene, optionally substituted imidazolylene, optionally substituted pyrazolylene, optionally substituted oxazolylene, optionally substituted isoxazolylene, optionally substituted thiazolylene, optionally substituted isothiazolylene, optionally substituted triazolylene, optionally substituted oxadiazolylene, optionally substituted thiadiazolylene, or optionally substituted tetrazolylene. In certain embodiments, L further comprises optionally substituted pyridinylene, optionally substituted pyridazinylene, optionally substituted pyrimidinylene, optionally substituted pyrazinylene, optionally substituted triazinylene, optionally substituted tetrazinylene, optionally substituted oxepinylene, or optionally substituted thiepinylene. In certain embodiments, L further comprises optionally substituted bicyclic heteroarylene (e.g. optionally substituted bicyclic, 9- or 10-membered heteroarylene, wherein 1, 2, 3, or 4 atoms in the heteroarylene ring system are independently oxygen, nitrogen, or sulfur). In certain embodiments, L further comprises optionally substituted triazolylene. In certain embodiments, L further comprises heteroarylene optionally substituted with one or more of halogen, optionally substituted alkylene, optionally substituted alkenylene, optionally substituted alkynylene, optionally substituted heteroalkylene, optionally substituted heteroalkenylene, optionally substituted heteroalkynylene, optionally substituted carbocyclylene, optionally substituted heterocyclylene, optionally substituted arylene, optionally substituted heteroarylene, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, and/or —B(OR^{A})₂ groups. In certain embodiments, L further comprises

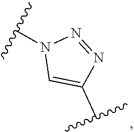 [SMILES: *c1cn(*)nn1]

or a salt thereof. In certain embodiments, L further comprises

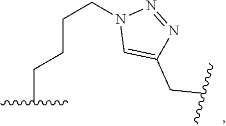 [SMILES: *CCCCn1cc(C*)nn1]

or a salt thereof. In certain embodiments, L further comprises a peptidyl group. In certain embodiments, L further comprises a dipeptidyl group. In certain embodiments, L further comprises a polypeptidyl group.

In certain embodiments, L further comprises a click chemistry handle. In certain embodiments, the click chemistry handle comprises an alkene. In certain embodiments, the click chemistry handle comprises a diene. In certain embodiments, the click chemistry handle comprises a dienophile. In certain embodiments, the click chemistry handle comprises a thiol. In certain embodiments, the click chemistry handle comprises a nitrile oxide. In certain embodiments, the click chemistry handle comprises a tetrazine. In certain embodiments, the click chemistry handle comprises an alkyne. In certain embodiments, the click chemistry handle comprises a terminal alkyne. In certain embodiments, the click chemistry handle comprises a strained alkyne. In certain embodiments, the click chemistry handle comprises an optionally substituted cyclooctyne. In certain embodiments, the click chemistry handle comprises a substituted cyclooctyne. In some embodiments, the click chemistry handle can react to form covalent bonds in the presence of a metal catalyst (e.g., copper (II)). In some embodiments, the click chemistry handle comprises a strained alkyne and can react to form covalent bonds in the presence of a metal catalyst (e.g., copper (II)). In some embodiments, the click chemistry handle comprises an optionally substituted cyclooctyne and can react to form covalent bonds in the presence of a metal catalyst (e.g., copper (II)). In some embodiments, the click chemistry handle comprises a substituted cyclooctyne and can react to form covalent bonds in the presence of a metal catalyst (e.g., copper (II)). In some embodiments, the click chemistry handle can react to form covalent bonds in the absence of a metal catalyst. In some embodiments, the click chemistry handle comprises a strained alkyne and can react to form covalent bonds in the absence of a metal catalyst. In some embodiments, the click chemistry handle comprises an optionally substituted cyclooctyne and can react to form covalent bonds in the absence of a metal catalyst. In some embodiments, the click chemistry handle comprises a substituted cyclooctyne and can react to form covalent bonds in the absence of a metal catalyst. In certain embodiments, the click chemistry handle comprises dibenzoazacyclooctyne (DIBAC or DBCO), biarylazacyclooctynone (BARAC), dibenzocyclooctyne (DIBO), difluorinated cyclooctyne (DIFO), bicyclononyne (BCN), dimethoxyazacyclooctyne (DIMAC), monofluorinated cyclooctyne (MOFO), cyclooctyne (OCT), and/or aryl-less cyclooctyne (ALO).

In certain embodiments, the click chemistry handle is of Formula (IV) or Formula (V):

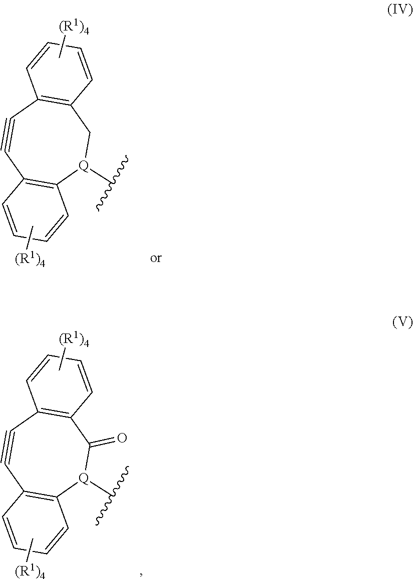

or a salt thereof, wherein:
each instance of R¹ is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
Q is CH or N.

In certain embodiments, each instance of R¹ is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂. In certain embodiments, at least one instance of R¹ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

In certain embodiments, at least one instance of R¹ is hydrogen. In certain embodiments, at least two instances of R¹ are hydrogen. In certain embodiments, at least three instances of R¹ are hydrogen. In certain embodiments, at least four instances of R¹ are hydrogen. In certain embodiments, at least five instances of R¹ are hydrogen. In certain embodiments, at least six instances of R¹ are hydrogen. In certain embodiments, at least seven instances of R¹ are hydrogen. In certain embodiments, at least eight instances of R¹ are hydrogen. In certain embodiments, all instances of R¹ are hydrogen.

In certain embodiments, each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring. In certain embodiments, at least one occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring. In certain embodiments, at least one occurrence of R^{A} is hydrogen.

In certain embodiments, Q is CH. In certain embodiments, Q is N. In certain embodiments, at least one instance of R¹ is hydrogen, and Q is CH. In certain embodiments, at least one instance of R¹ is hydrogen, Q is N. In certain embodiments, all instances of R¹ are hydrogen, and Q is CH. In certain embodiments, all instances of R¹ are hydrogen, and Q is N.

In certain embodiments, the click chemistry handle is of formula

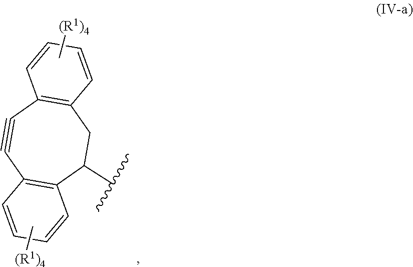

or a salt thereof. In certain embodiments, the click chemistry handle is of formula

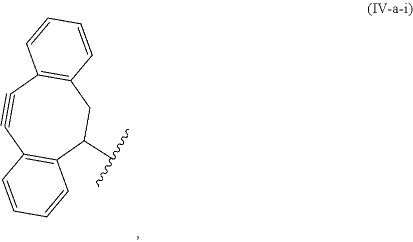 [SMILES: *C1Cc2ccccc2C#Cc2ccccc21]

or a salt thereof. In certain embodiments, the click chemistry handle is of formula

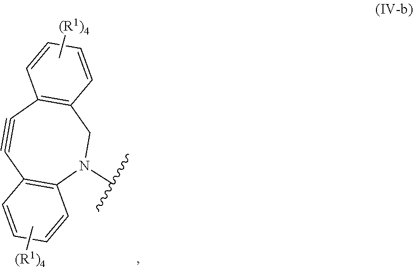

or a salt thereof. In certain embodiments, the click chemistry handle is of formula

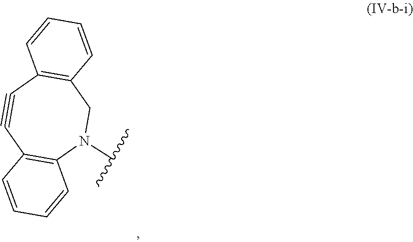 [SMILES: *N1Cc2ccccc2C#Cc2ccccc21]

or a salt thereof. In certain embodiments, the click chemistry handle is of formula

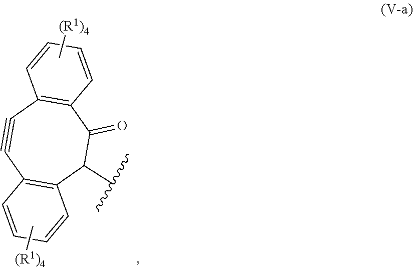

or a salt thereof. In certain embodiments, the click chemistry handle is of formula

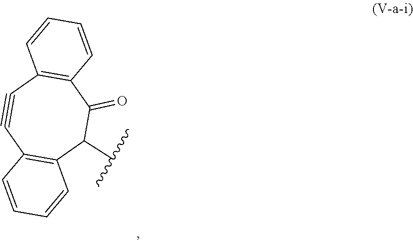 [SMILES: *C1C(=O)c2ccccc2C#Cc2ccccc21]

or a salt thereof. In certain embodiments, the click chemistry handle is of formula

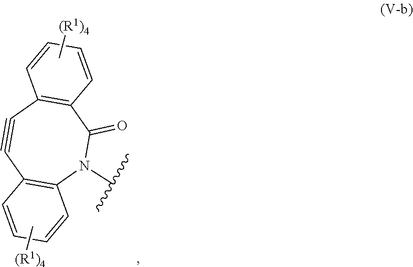

or a salt thereof. In certain embodiments, the click chemistry handle is of formula

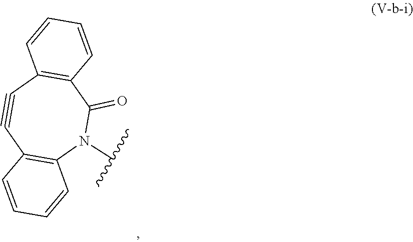 [SMILES: *N1C(=O)c2ccccc2C#Cc2ccccc21]

or a salt thereof.

In certain embodiments, L comprises a click chemistry handle of Formula (IV) or Formula (V) and optionally substituted alkylene. In certain embodiments, L comprises a click chemistry handle of Formula (IV) or Formula (V) and optionally substituted C₁₋₁₂ alkylene. In certain embodiments, L comprises a click chemistry handle of Formula (IV) or Formula (V) and optionally substituted C₁₋₁₀ alkylene. In certain embodiments, L comprises a click chemistry handle of Formula (IV) or Formula (V) and optionally substituted C₁₋₆ alkylene. In certain embodiments, L comprises a click chemistry handle of Formula (IV) or Formula (V) and unsubstituted C₁₋₆ alkylene. In certain embodiments, L comprises a click chemistry handle of Formula (IV) or Formula (V) and substituted C₁₋₆ alkylene. In certain embodiments, L comprises a click chemistry handle of Formula (IV) or Formula (V) and substituted C₁₋₆ alkylene substituted with one or more oxo groups. In certain embodiments, L comprises a click chemistry handle of Formula (IV) or Formula (V) and substituted C₁₋₆ alkylene substituted with one oxo group. In certain embodiments, L comprises a click chemistry handle of Formula (IV) or Formula (V) and substituted C₁₋₆ alkylene substituted with two oxo groups. In certain embodiments, L comprises a click chemistry handle of Formula (IV) or Formula (V) and substituted or unsubstituted methylene, substituted or unsubstituted ethylene, substituted or unsubstituted n-propylene, substituted or unsubstituted isopropylene, substituted or unsubstituted n-butylene, substituted or unsubstituted tert-butylene, substituted or unsubstituted sec-butylene, substituted or unsubstituted isobutylene, substituted or unsubstituted n-pentylene, substituted or unsubstituted 3-pentanylene, substituted or unsubstituted amylene, substituted or unsubstituted neopentylene, substituted or unsubstituted 3-methylene-2-butanylene, substituted or unsubstituted tert-amylene, or substituted or unsubstituted n-hexylene. In certain embodiments, L comprises a click chemistry handle of Formula (IV) or Formula (V) and unsubstituted methylene. In certain embodiments, L comprises a click chemistry handle of Formula (IV) or Formula (V) and substituted methylene. In certain embodiments, L comprises a click chemistry handle of Formula (IV) or Formula (V) and unsubstituted n-butylene. In certain embodiments, L comprises a click chemistry handle of Formula (IV) or Formula (V) and substituted n-butylene. In certain embodiments, L comprises a click chemistry handle of Formula (IV) or Formula (V) and substituted n-butylene substituted with one or more oxo groups. In certain embodiments, L comprises a click chemistry handle of Formula (IV) or Formula (V) and substituted n-butylene substituted with one oxo group. In certain embodiments, L comprises a click chemistry handle of Formula (IV) or Formula (V) and substituted n-butylene substituted with two oxo groups. In certain embodiments, L comprises

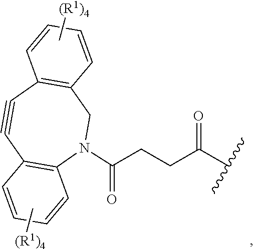

or a salt thereof. In certain embodiments, L comprises

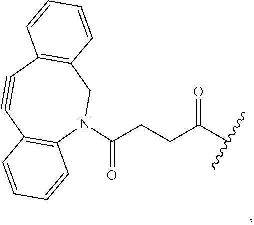 [SMILES: *C(=O)CCC(=O)N1Cc2ccccc2C#Cc2ccccc21]

or a salt thereof. In certain embodiments, L comprises

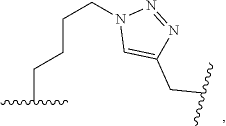 [SMILES: *CCCCn1cc(C*)nn1]

or a salt thereof. In certain embodiments, L comprises

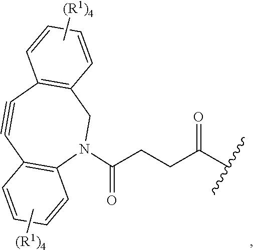

or a salt thereof, and

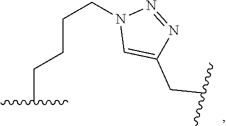 [SMILES: *CCCCn1cc(C*)nn1]

or a salt thereof. In certain embodiments, L comprises

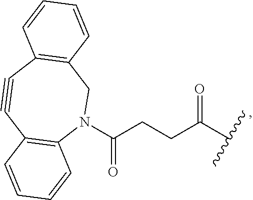 [SMILES: *C(=O)CCC(=O)N1Cc2ccccc2C#Cc2ccccc21]

or a salt thereof, and

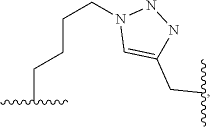 [SMILES: *CCCCN1C=C(C*)NN1]

or a salt thereof. In certain embodiments, L comprises

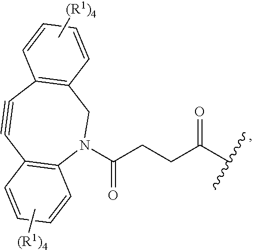

or a salt thereof, and

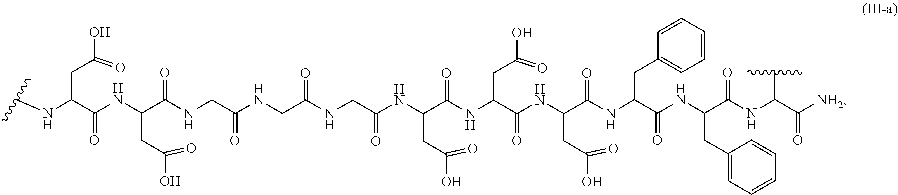 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

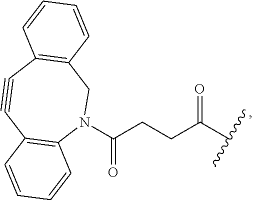 [SMILES: *C(=O)CCC(=O)N1Cc2ccccc2C#Cc2ccccc21]

or a salt thereof, and

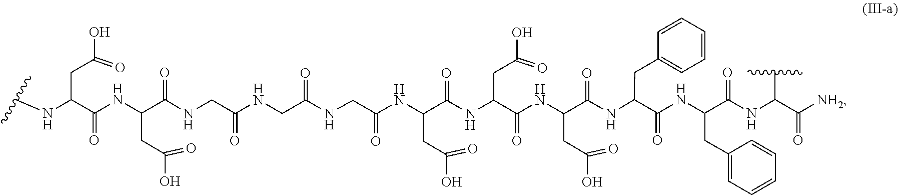 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

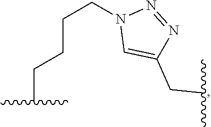 [SMILES: *CCCCn1cc(C*)nn1]

or a salt thereof, and

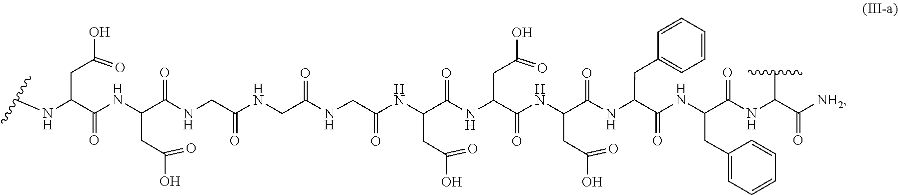 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

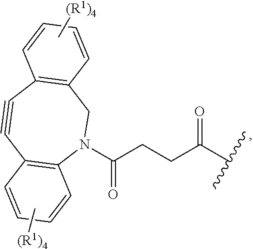

or a salt thereof,

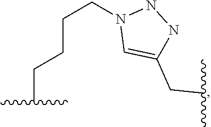 [SMILES: *CCCCN1C=C(C*)NN1]

or a salt thereof, and

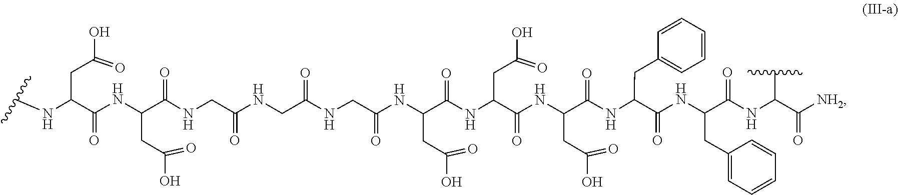 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

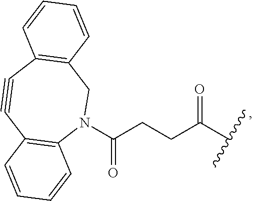 [SMILES: *C(=O)CCC(=O)N1Cc2ccccc2C#Cc2ccccc21]

or a salt thereof,

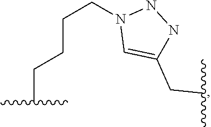 [SMILES: *CCCCN1C=C(C*)NN1]

or a salt thereof, and

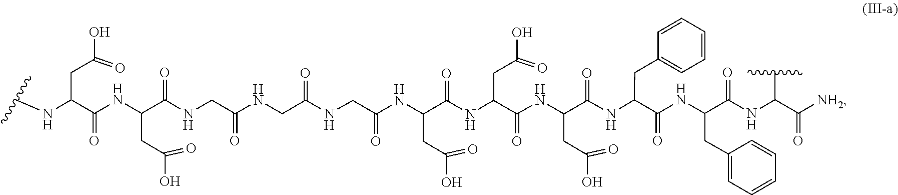 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

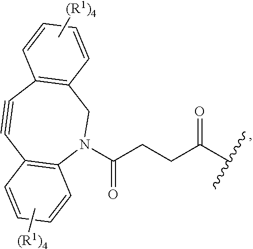

or a salt thereof, and

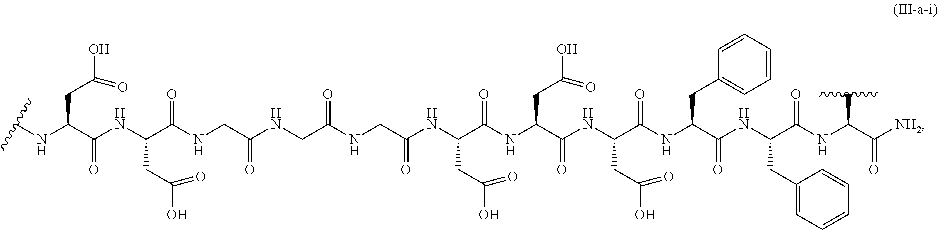 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof. In certain embodiments, L comprises

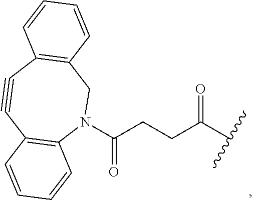 [SMILES: *C(=O)CCC(=O)N1Cc2ccccc2C#Cc2ccccc21]

or a salt thereof, and

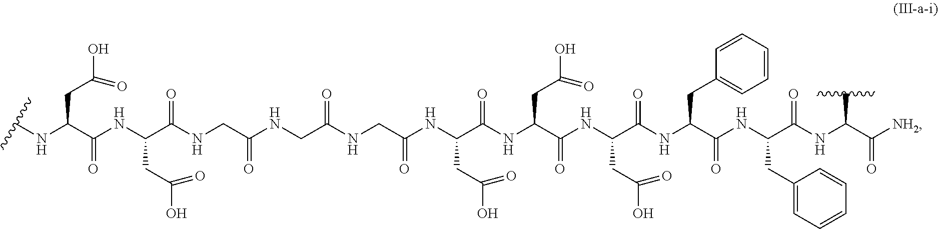 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof. In certain embodiments, L comprises

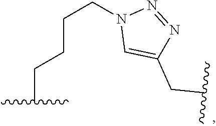 [SMILES: *CCCCn1cc(C*)nn1]

or a salt thereof, and

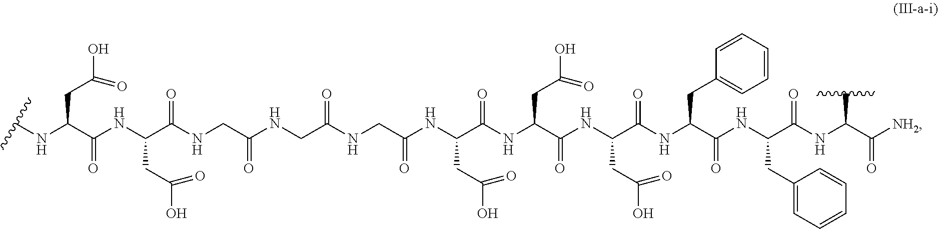 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof. In certain embodiments, L comprises

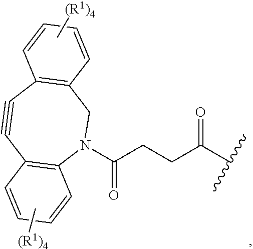

or a salt thereof,

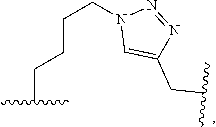 [SMILES: *CCCCn1cc(C*)nn1]

or a salt thereof, and

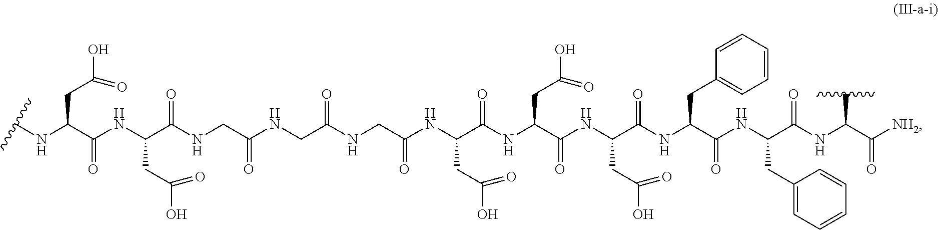 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof. In certain embodiments, L comprises

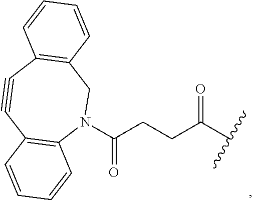 [SMILES: *C(=O)CCC(=O)N1Cc2ccccc2C#Cc2ccccc21]

or a salt thereof,

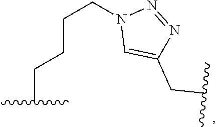 [SMILES: *CCCCn1cc(C*)nn1]

or a salt thereof, and

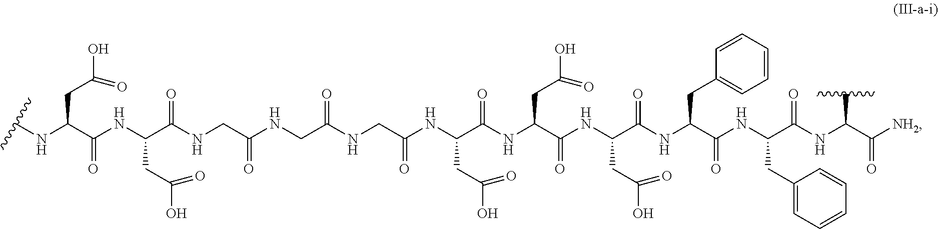 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof.

In certain embodiments, L comprises

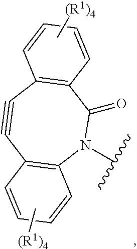

or a salt thereof. In certain embodiments, L comprises

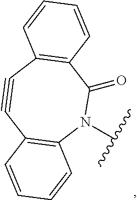 [SMILES: *N1C(=O)c2ccccc2C#Cc2ccccc21]

or a salt thereof. In certain embodiments, L comprises

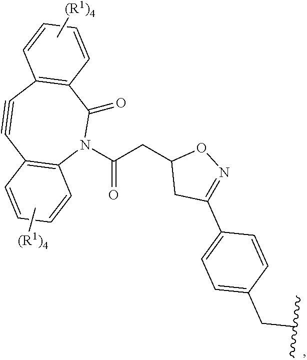

or a salt thereof. In certain embodiments, L comprises

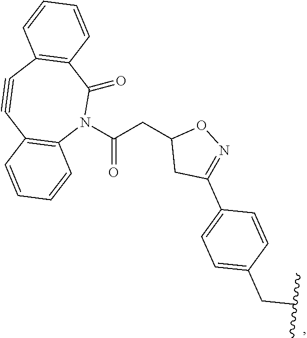 [SMILES: *Cc1ccc(C2=NOC(CC(=O)N3C(=O)c4ccccc4C#Cc4ccccc43)C2)cc1]

or a salt thereof. In certain embodiments, L comprises

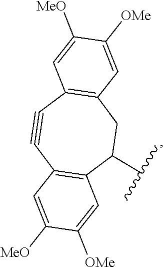 [SMILES: *C1Cc2cc(OC)c(OC)cc2C#Cc2cc(OC)c(OC)cc21]

or a salt thereof. In certain embodiments, L comprises

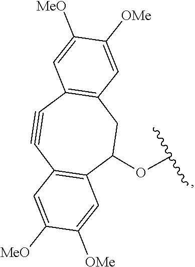 [SMILES: *OC1Cc2cc(OC)c(OC)cc2C#Cc2cc(OC)c(OC)cc21]

or a salt thereof. In certain embodiments, L comprises

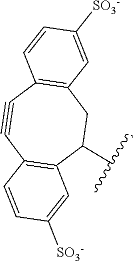

or a salt thereof. In certain embodiments, L comprises

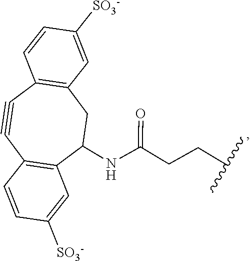

or a salt thereof. In certain embodiments, L comprises

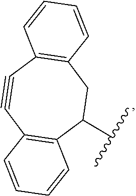 [SMILES: *C1Cc2ccccc2C#Cc2ccccc21]

or a salt thereof. In certain embodiments, L comprises

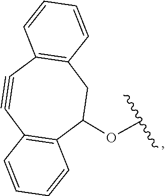 [SMILES: *OC1Cc2ccccc2C#Cc2ccccc21]

or a salt thereof.

In certain embodiments, the click chemistry handle is of formula (VI):

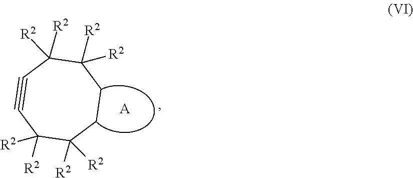

or a salt thereof, wherein:
each instance of R² is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂, or two instances of R² attached to the same carbon atom are taken together to form ═O or ═S;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
Ring A is optionally substituted carbocyclyl, optionally substituted heterocyclyl, optionally substituted aryl, or optionally substituted heteroaryl.

In certain embodiments, each instance of R² is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂. In certain embodiments, at least one instance of R² is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

In certain embodiments, at least one instance of R² is hydrogen. In certain embodiments, at least two instances of R² are hydrogen. In certain embodiments, at least three instances of R² are hydrogen. In certain embodiments, at least four instances of R² are hydrogen. In certain embodiments, at least five instances of R² are hydrogen. In certain embodiments, at least six instances of R² are hydrogen. In certain embodiments, at least seven instances of R² are hydrogen. In certain embodiments, at least eight instances of R² are hydrogen. In certain embodiments, all instances of R² are hydrogen.

In certain embodiments, Ring A is optionally substituted carbocyclyl. In certain embodiments, Ring A is optionally substituted heterocyclyl. In certain embodiments, Ring A is optionally substituted aryl. In certain embodiments, Ring A is optionally substituted heteroaryl.

In certain embodiments, the click chemistry handle is of Formula (VI-a):

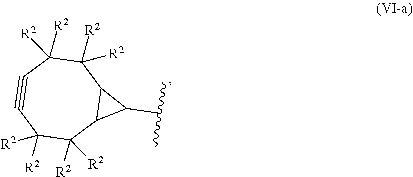

or a salt thereof. In certain embodiments, the click chemistry handle is of formula

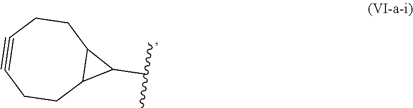 [SMILES: *C1C2CCC#CCCC12]

or a salt thereof. In certain embodiments, the click chemistry handle is of formula

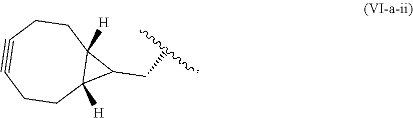

or a salt thereof. In certain embodiments, the click chemistry handle is of formula

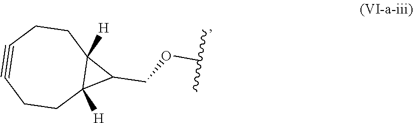

or a salt thereof.

In certain embodiments, the click chemistry handle is of Formula (VI-b):

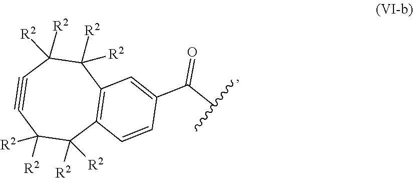

or a salt thereof. In certain embodiments, the click chemistry handle is of formula

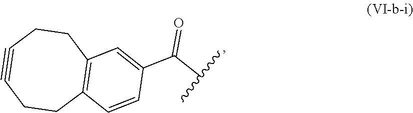 [SMILES: *C(=O)c1ccc2c(c1)CCC#CCC2]

or a salt thereof.

In certain embodiments, the click chemistry handle is of Formula (VI-c):

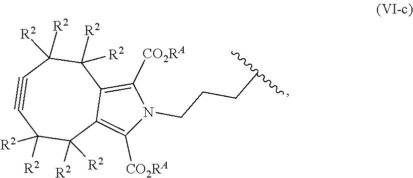

or a salt thereof. In certain embodiments, the click chemistry handle is of formula

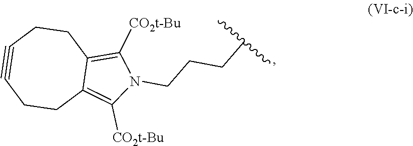 [SMILES: *CCCn1c(C(=O)OC(C)(C)C)c2c(c1C(=O)OC(C)(C)C)CCC#CCC2]

or a salt thereof. In certain embodiments, the click chemistry handle is of formula

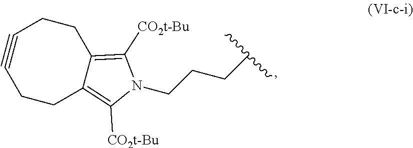 [SMILES: *CCCn1c(C(=O)OC(C)(C)C)c2c(c1C(=O)OC(C)(C)C)CCC#CCC2]

or a salt thereof.

In certain embodiments, L comprises a click chemistry handle of Formula (VI) and optionally substituted alkylene. In certain embodiments, L comprises a click chemistry handle of Formula (VI) and optionally substituted C₁₋₁₂ alkylene. In certain embodiments, L comprises a click chemistry handle of Formula (VI) and optionally substituted C₁₋₁₀ alkylene. In certain embodiments, L comprises a click chemistry handle of Formula (VI) and optionally substituted C₁₋₆ alkylene. In certain embodiments, L comprises a click chemistry handle of Formula (VI) and unsubstituted C₁₋₆ alkylene. In certain embodiments, L comprises a click chemistry handle of Formula (VI) and substituted C₁₋₆ alkylene. In certain embodiments, L comprises a click chemistry handle of Formula (VI) and substituted C₁₋₆ alkylene substituted with one or more oxo groups. In certain embodiments, L comprises a click chemistry handle of Formula (VI) and substituted C₁₋₆ alkylene substituted with one oxo group. In certain embodiments, L comprises a click chemistry handle of Formula (VI) and substituted C₁₋₆ alkylene substituted with two oxo groups. In certain embodiments, L comprises a click chemistry handle of Formula (VI) and substituted or unsubstituted methylene, substituted or unsubstituted ethylene, substituted or unsubstituted n-propylene, substituted or unsubstituted isopropylene, substituted or unsubstituted n-butylene, substituted or unsubstituted tert-butylene, substituted or unsubstituted sec-butylene, substituted or unsubstituted isobutylene, substituted or unsubstituted n-pentylene, substituted or unsubstituted 3-pentanylene, substituted or unsubstituted amylene, substituted or unsubstituted neopentylene, substituted or unsubstituted 3-methylene-2-butanylene, substituted or unsubstituted tert-amylene, or substituted or unsubstituted n-hexylene. In certain embodiments, L comprises a click chemistry handle of Formula (VI) and unsubstituted methylene. In certain embodiments, L comprises a click chemistry handle of Formula (VI) and substituted methylene. In certain embodiments, L comprises a click chemistry handle of Formula (VI) and unsubstituted n-butylene. In certain embodiments, L comprises a click chemistry handle of Formula (VI) and substituted n-butylene. In certain embodiments, L comprises a click chemistry handle of Formula (VI) and substituted n-butylene substituted with one or more oxo groups. In certain embodiments, L comprises a click chemistry handle of Formula (VI) and substituted n-butylene substituted with one oxo group. In certain embodiments, L comprises a click chemistry handle of Formula (VI) and substituted n-butylene substituted with two oxo groups. In certain embodiments, L comprises

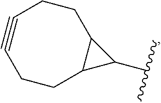 [SMILES: *C1C2CCC#CCCC12]

or a salt thereof. In certain embodiments, L comprises

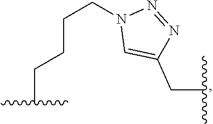 [SMILES: *CCCCn1cc(C*)nn1]

or a salt thereof. In certain embodiments, L comprises

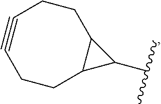 [SMILES: *C1C2CCC#CCCC12]

or a salt thereof, and

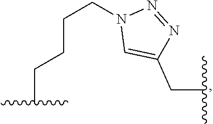 [SMILES: *CCCCn1cc(C*)nn1]

or a salt thereof. In certain embodiments, L comprises

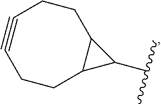 [SMILES: *C1C2CCC#CCCC12]

or a salt thereof, and

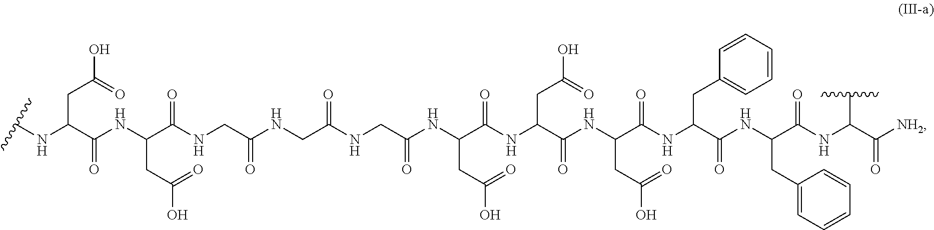 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

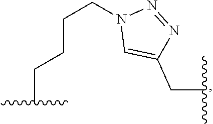 [SMILES: *CCCCn1cc(C*)nn1]

or a salt thereof, and

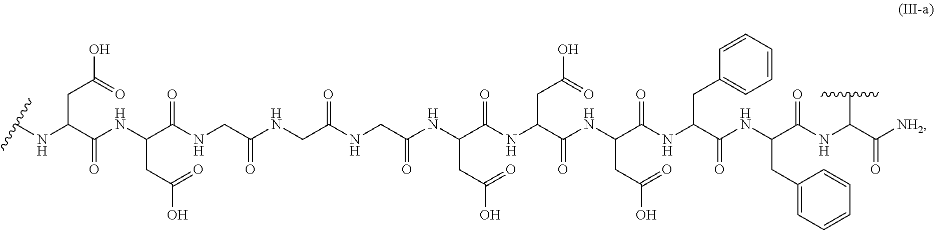 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

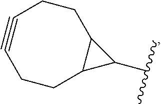 [SMILES: *C1C2CCC#CCCC12]

or a salt thereof,

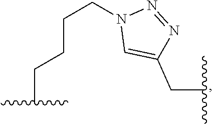 [SMILES: *CCCCn1cc(C*)nn1]

or a salt thereof, and

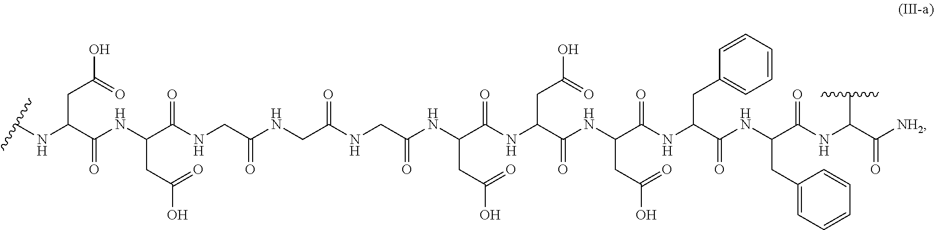 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

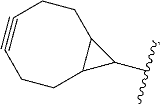 [SMILES: *C1C2CCC#CCCC12]

or a salt thereof, and

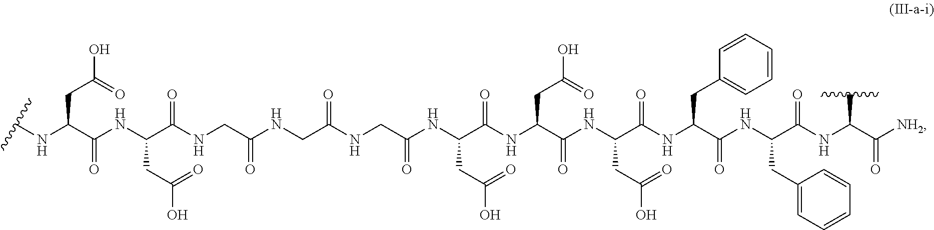 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof. In certain embodiments, L comprises

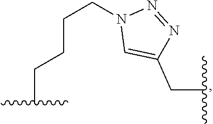 [SMILES: *CCCCn1cc(C*)nn1]

or a salt thereof, and

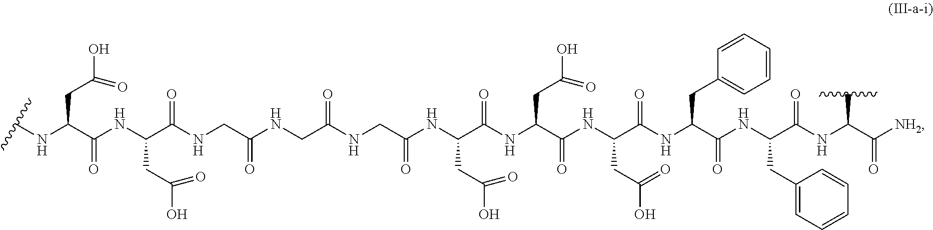 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof. In certain embodiments, L comprises

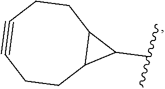 [SMILES: *C1C2CCC#CCCC12]

or a salt thereof,

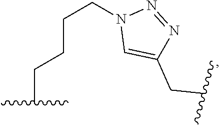 [SMILES: *CCCCn1cc(C*)nn1]

or a salt thereof, and

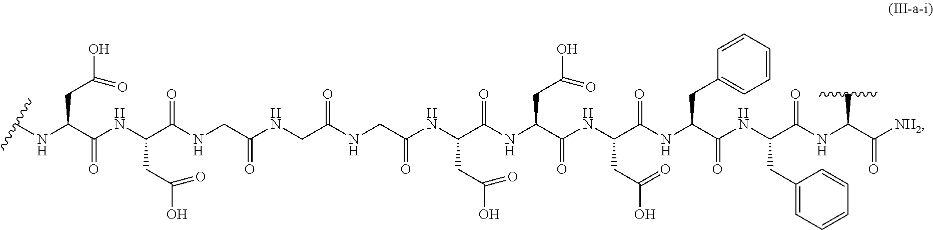 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof.

In certain embodiments, the click chemistry handle is of Formulae (VII-a), (VII-b), (VII-c), or (VII-d):

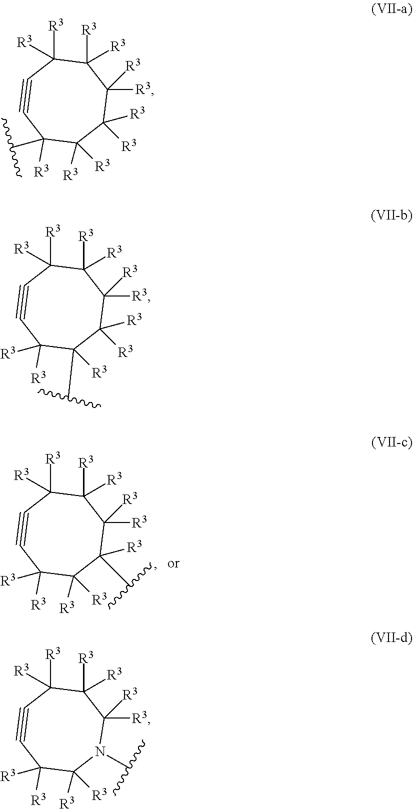

or a salt thereof, wherein:
each instance of R³ is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂, or two instances of R³ attached to the same carbon atom are taken together to form ═O or ═S; and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring.

In certain embodiments, each instance of R³ is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂. In certain embodiments, at least one instance of R³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

In certain embodiments, at least one instance of R³ is hydrogen. In certain embodiments, at least two instances of R³ are hydrogen. In certain embodiments, at least three instances of R³ are hydrogen. In certain embodiments, at least four instances of R³ are hydrogen. In certain embodiments, at least five instances of R³ are hydrogen. In certain embodiments, at least six instances of R³ are hydrogen. In certain embodiments, at least seven instances of R³ are hydrogen. In certain embodiments, at least eight instances of R³ are hydrogen. In certain embodiments, at least nine instances of R³ are hydrogen. In certain embodiments, all instances of R³ are hydrogen. In certain embodiments, at least one instance of R³ is halogen. In certain embodiments, at least two instances of R³ are halogen. In certain embodiments, at least three instances of R³ are halogen. In certain embodiments, at least four instances of R³ are halogen. In certain embodiments, at least five instances of R³ are halogen. In certain embodiments, at least six instances of R³ are halogen. In certain embodiments, at least seven instances of R³ are halogen. In certain embodiments, at least eight instances of R³ are halogen. In certain embodiments, all instances of R³ are halogen. In certain embodiments, at least one instance of R³ is fluorine. In certain embodiments, at least two instances of R³ are fluorine. In certain embodiments, at least three instances of R³ are fluorine. In certain embodiments, at least four instances of R³ are fluorine. In certain embodiments, at least five instances of R³ are fluorine. In certain embodiments, at least six instances of R³ are fluorine. In certain embodiments, at least seven instances of R³ are fluorine. In certain embodiments, at least eight instances of R³ are fluorine. In certain embodiments, all instances of R³ are fluorine. In certain embodiments, two instances of R³ are halogen, and nine instances of R³ are hydrogen. In certain embodiments, two instances of R³ are fluorine, and nine instances of R³ are hydrogen.

In certain embodiments, the click chemistry handle is of formula

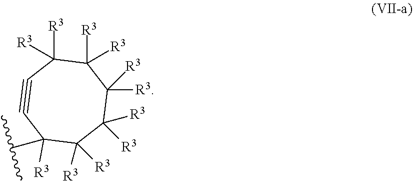

In certain embodiments, the click chemistry handle is of formula

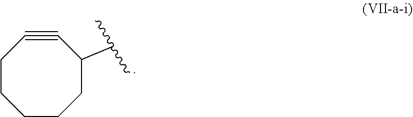 [SMILES: *C1C#CCCCCC1]

In certain embodiments, the click chemistry handle is of formula

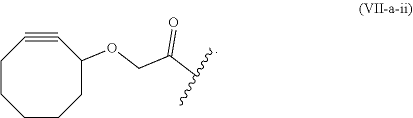 [SMILES: *C(=O)COC1C#CCCCCC1]

In certain embodiments, the click chemistry handle is of formula

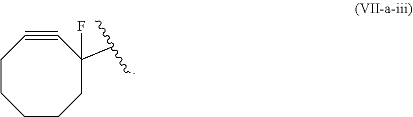 [SMILES: *C1(F)C#CCCCCC1]

In certain embodiments, the click chemistry handle is of formula

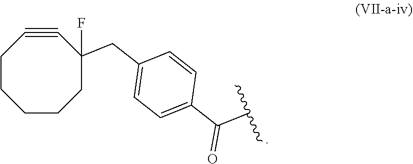 [SMILES: *C(=O)c1ccc(CC2(F)C#CCCCCC2)cc1]

In certain embodiments, the click chemistry handle is of formula

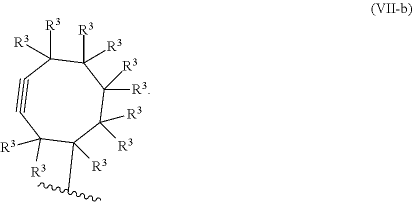

In certain embodiments, the click chemistry handle is of formula

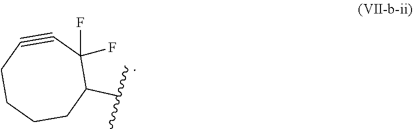 [SMILES: *C1CCCCC#CC1(F)F]

In certain embodiments, the click chemistry handle is of formula

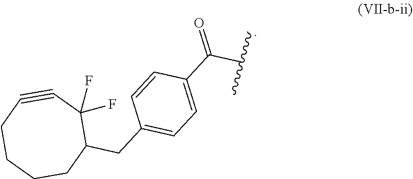 [SMILES: *C(=O)c1ccc(CC2CCCCC#CC2(F)F)cc1]

In certain embodiments, the click chemistry handle is of formula

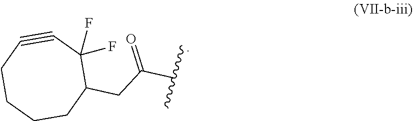 [SMILES: *C(=O)CC1CCCCC#CC1(F)F]

In certain embodiments, the click chemistry handle is of formula

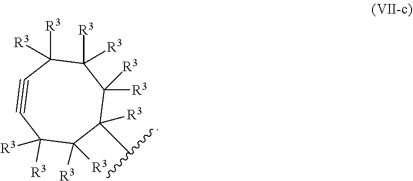

In certain embodiments, the click chemistry handle is of formula

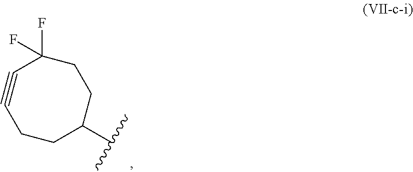 [SMILES: *C1CCC#CC(F)(F)CC1]

(or a salt thereof. In certain embodiments, the click chemistry handle is of formula

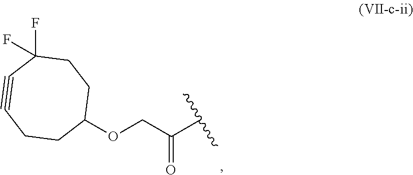 [SMILES: *C(=O)COC1CCC#CC(F)(F)CC1]

or a salt thereof.

In certain embodiments, the click chemistry handle is of formula

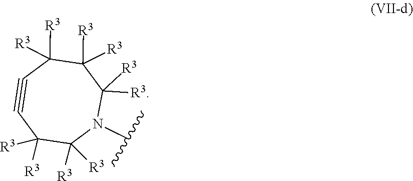

In certain embodiments, the click chemistry handle is of formula

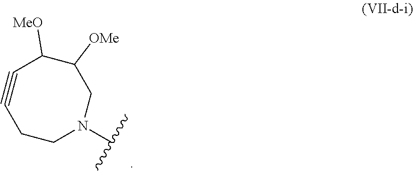 [SMILES: *N1CCC#CC(OC)C(OC)C1]

In certain embodiments, the click chemistry handle is of formula

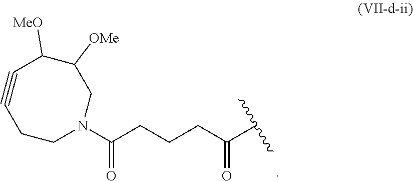 [SMILES: *C(=O)CCCC(=O)N1CCC#CC(OC)C(OC)C1]

In certain embodiments, the click chemistry handle is of formula

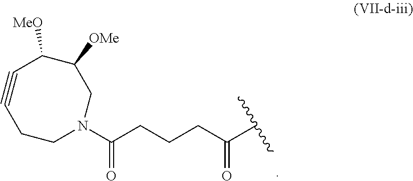 [SMILES: *C(=O)CCCC(=O)N1CCC#C[C@H](OC)[C@@H](OC)C1]

In certain embodiments, L comprises a click chemistry handle of Formulae (VII-a), (VII-b), (VII-c), or (VII-d) and optionally substituted alkylene. In certain embodiments, L comprises a click chemistry handle of Formulae (VII-a), (VII-b), (VII-c), or (VII-d) and optionally substituted C₁₋₁₂ alkylene. In certain embodiments, L comprises a click chemistry handle of Formulae (VII-a), (VII-b), (VII-c), or (VII-d) and optionally substituted C₁₋₁₀ alkylene. In certain embodiments, L comprises a click chemistry handle of Formulae (VII-a), (VII-b), (VII-c), or (VII-d) and optionally substituted C₁₋₆ alkylene. In certain embodiments, L comprises a click chemistry handle of Formulae (VII-a), (VII-b), (VII-c), or (VII-d) and unsubstituted C₁₋₆ alkylene. In certain embodiments, L comprises a click chemistry handle of Formulae (VII-a), (VII-b), (VII-c), or (VII-d) and substituted C₁₋₆ alkylene. In certain embodiments, L comprises a click chemistry handle of Formulae (VII-a), (VII-b), (VII-c), or (VII-d) and substituted C₁₋₆ alkylene substituted with one or more oxo groups. In certain embodiments, L comprises a click chemistry handle of Formulae (VII-a), (VII-b), (VII-c), or (VII-d) and substituted C₁₋₆ alkylene substituted with one oxo group. In certain embodiments, L comprises a click chemistry handle of Formulae (VII-a), (VII-b), (VII-c), or (VII-d) and substituted C₁₋₆ alkylene substituted with two oxo groups. In certain embodiments, L comprises a click chemistry handle of Formulae (VII-a), (VII-b), (VII-c), or (VII-d) and substituted or unsubstituted methylene, substituted or unsubstituted ethylene, substituted or unsubstituted n-propylene, substituted or unsubstituted isopropylene, substituted or unsubstituted n-butylene, substituted or unsubstituted tert-butylene, substituted or unsubstituted sec-butylene, substituted or unsubstituted isobutylene, substituted or unsubstituted n-pentylene, substituted or unsubstituted 3-pentanylene, substituted or unsubstituted amylene, substituted or unsubstituted neopentylene, substituted or unsubstituted 3-methylene-2-butanylene, substituted or unsubstituted tert-amylene, or substituted or unsubstituted n-hexylene. In certain embodiments, L comprises a click chemistry handle of Formulae (VII-a), (VII-b), (VII-c), or (VII-d) and unsubstituted methylene. In certain embodiments, L comprises a click chemistry handle of Formulae (VII-a), (VII-b), (VII-c), or (VII-d) and substituted methylene. In certain embodiments, L comprises a click chemistry handle of Formulae (VII-a), (VII-b), (VII-c), or (VII-d) and unsubstituted n-butylene. In certain embodiments, L comprises a click chemistry handle of Formulae (VII-a), (VII-b), (VII-c), or (VII-d) and substituted n-butylene. In certain embodiments, L comprises a click chemistry handle of Formulae (VII-a), (VII-b), (VII-c), or (VII-d) and substituted n-butylene substituted with one or more oxo groups. In certain embodiments, L comprises a click chemistry handle of Formulae (VII-a), (VII-b), (VII-c), or (VII-d) and substituted n-butylene substituted with one oxo group. In certain embodiments, L comprises a click chemistry handle of Formulae (VII-a), (VII-b), (VII-c), or (VII-d) and substituted n-butylene substituted with two oxo groups. In certain embodiments, L comprises

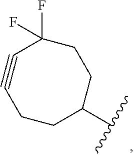 [SMILES: *C1CCC#CC(F)(F)CC1]

or a salt thereof. In certain embodiments, L comprises

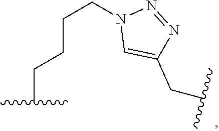 [SMILES: *CCCCn1cc(C*)nn1]

or a salt thereof. In certain embodiments, L comprises

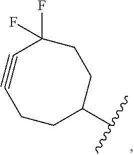 [SMILES: *C1CCC#CC(F)(F)CC1]

or a salt thereof, and

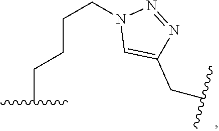 [SMILES: *CCCCn1cc(C*)nn1]

or a salt thereof. In certain embodiments, L comprises

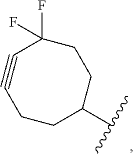 [SMILES: *C1CCC#CC(F)(F)CC1]

or a salt thereof, and

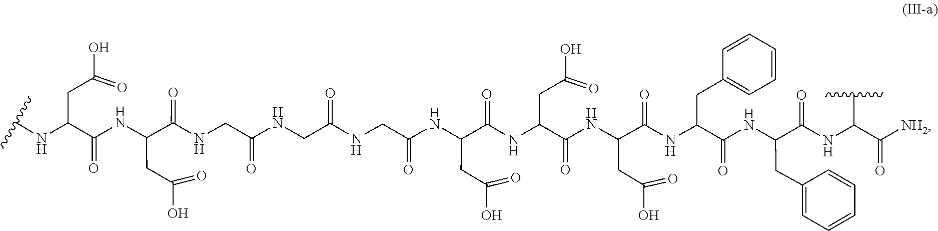 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

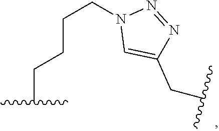 [SMILES: *CCCCn1cc(C*)nn1]

or a salt thereof, and

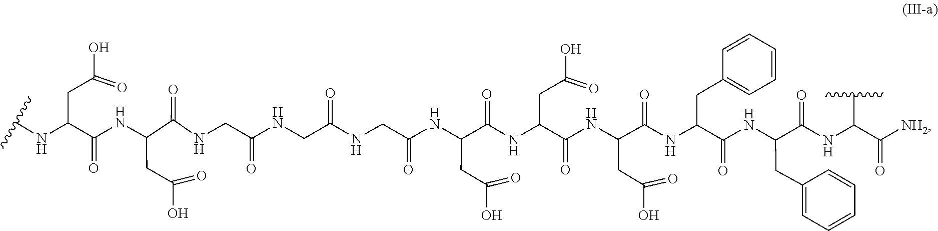 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

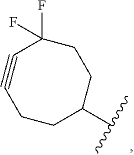 [SMILES: *C1CCC#CC(F)(F)CC1]

or a salt thereof,

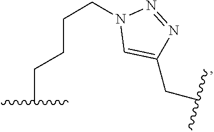 [SMILES: *CCCCn1cc(C*)nn1]

or a salt thereof, and

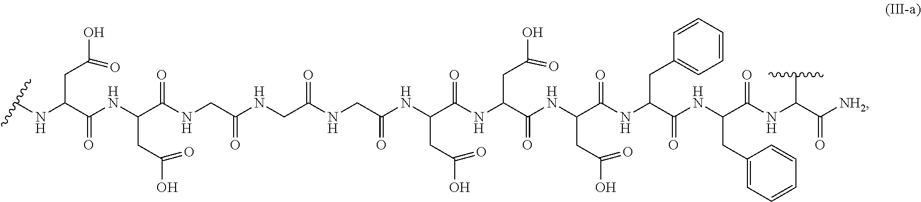 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

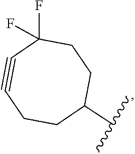 [SMILES: *C1CCC#CC(F)(F)CC1]

or a salt thereof, and

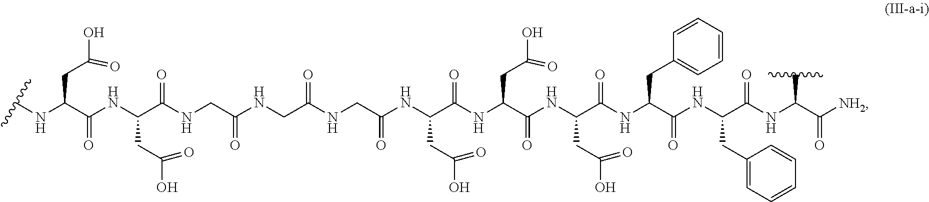 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof. In certain embodiments, L comprises

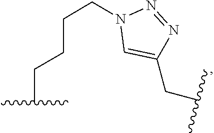 [SMILES: *CCCCn1cc(C*)nn1]

or a salt thereof, and

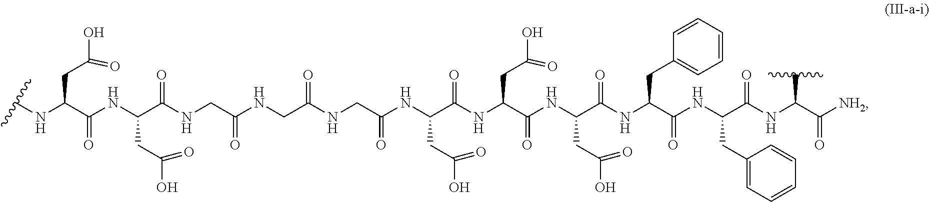 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof. In certain embodiments, L comprises

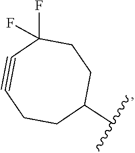 [SMILES: *C1CCC#CC(F)(F)CC1]

or a salt thereof,

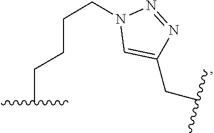 [SMILES: *CCCCn1cc(C*)nn1]

or a salt thereof, and

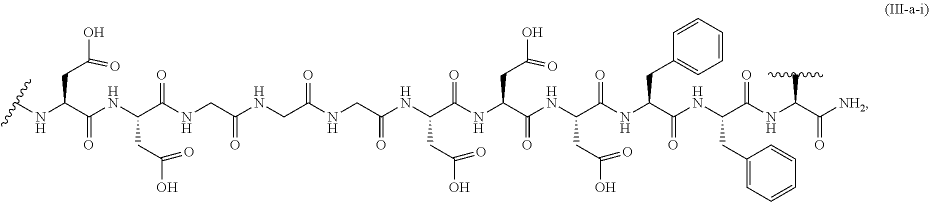 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof.

In certain embodiments, L comprises a moiety selected from:

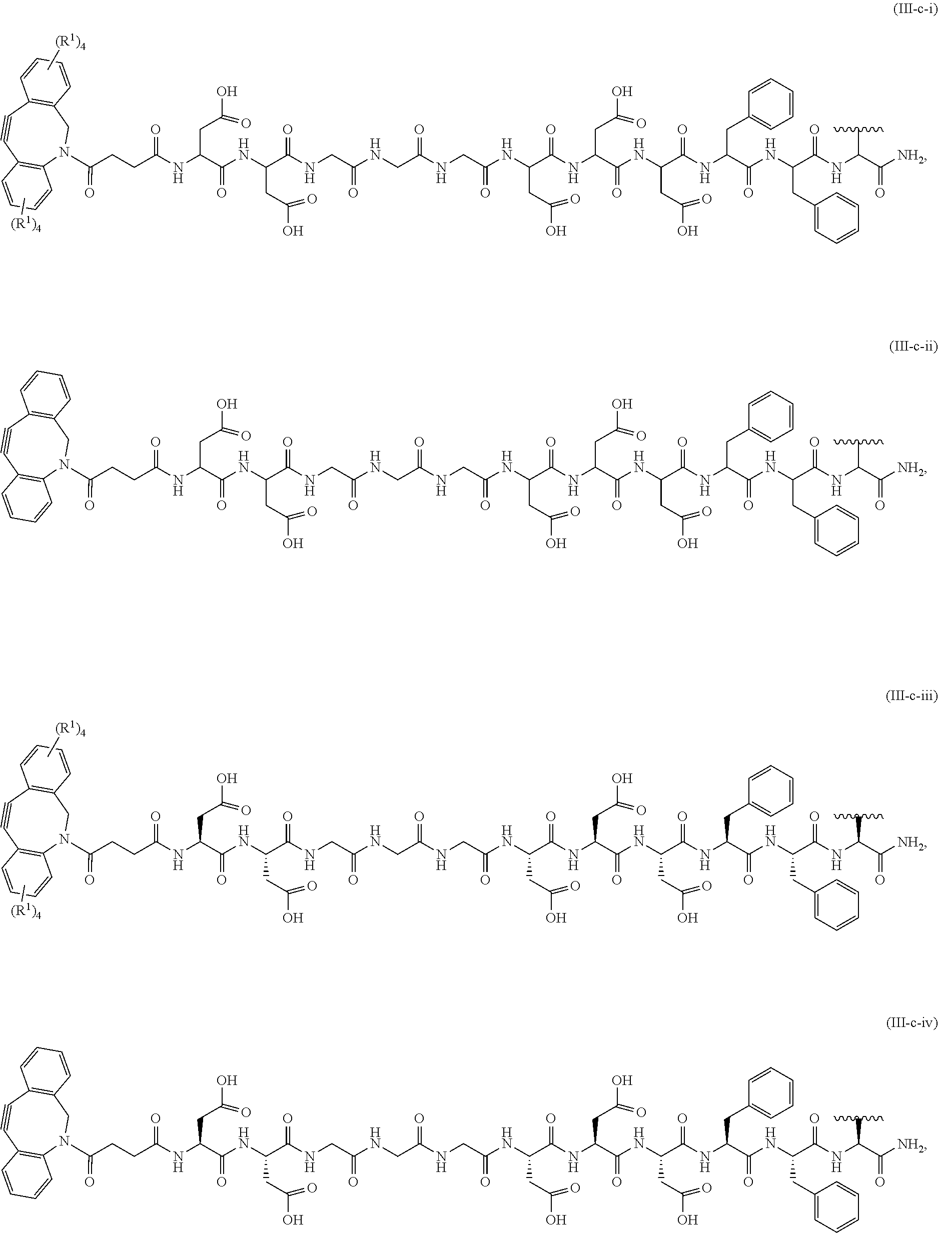

or a salt thereof. In certain embodiments, L comprises

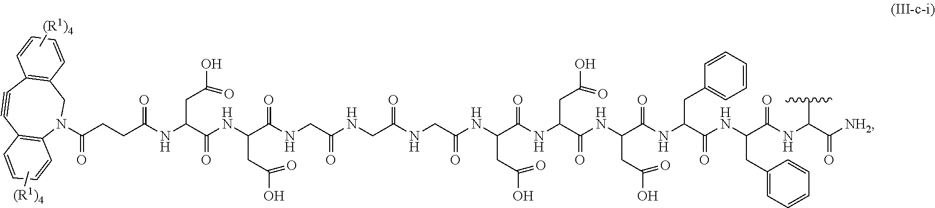

or a salt thereof. In certain embodiments, L comprises

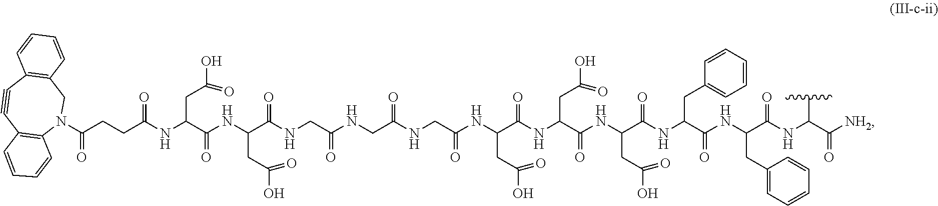 [SMILES: *C(NC(=O)C(Cc1ccccc1)NC(=O)C(Cc1ccccc1)NC(=O)C(CC(=O)O)NC(=O)C(CC(=O)O)NC(=O)C(CC(=O)O)NC(=O)CNC(=O)CNC(=O)CNC(=O)C(CC(=O)O)NC(=O)C(CC(=O)O)NC(=O)CCC(=O)N1Cc2ccccc2C#Cc2ccccc21)C(N)=O]

or a salt thereof. In certain embodiments, L comprises

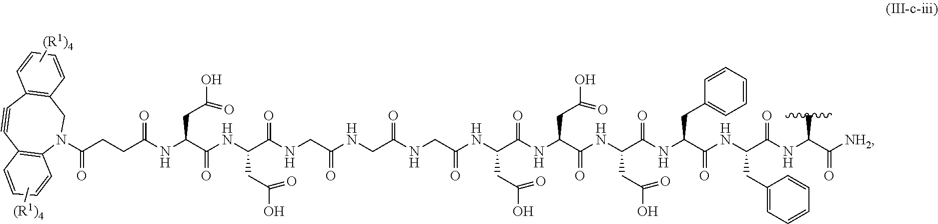

or a salt thereof. In certain embodiments, L comprises

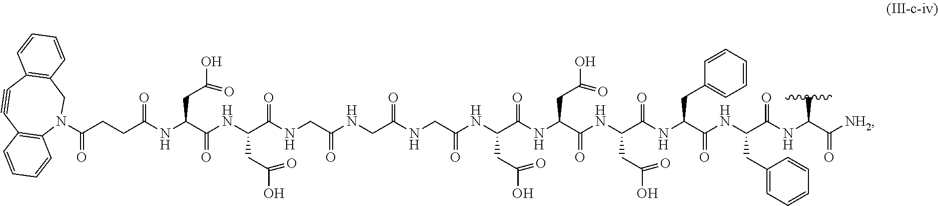 [SMILES: *[C@H](NC(=O)[C@H](Cc1ccccc1)NC(=O)[C@H](Cc1ccccc1)NC(=O)[C@H](CC(=O)O)NC(=O)[C@H](CC(=O)O)NC(=O)[C@H](CC(=O)O)NC(=O)CNC(=O)CNC(=O)CNC(=O)[C@H](CC(=O)O)NC(=O)[C@H](CC(=O)O)NC(=O)CCC(=O)N1Cc2ccccc2C#Cc2ccccc21)C(N)=O]

or a salt thereof. In certain embodiments, L comprises

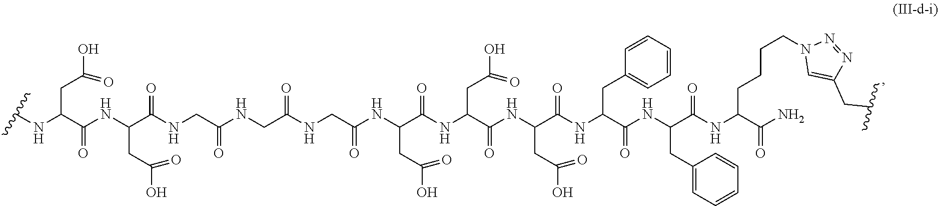 [SMILES: *Cc1cn(CCCCC(NC(=O)C(Cc2ccccc2)NC(=O)C(Cc2ccccc2)NC(=O)C(CC(=O)O)NC(=O)C(CC(=O)O)NC(=O)C(CC(=O)O)NC(=O)CNC(=O)CNC(=O)CNC(=O)C(CC(=O)O)NC(=O)C(CC(=O)O)N*)C(N)=O)nn1]

or a salt thereof. In certain embodiments, L comprises

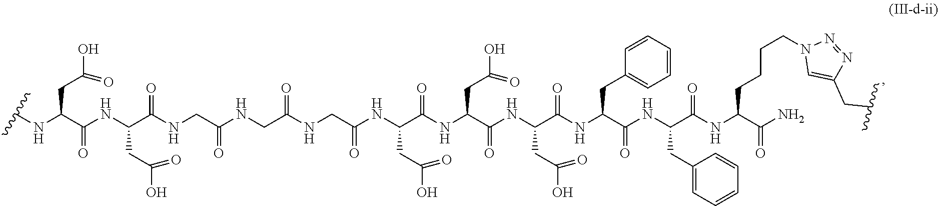 [SMILES: *Cc1cn(CCCC[C@H](NC(=O)[C@H](Cc2ccccc2)NC(=O)[C@H](Cc2ccccc2)NC(=O)[C@H](CC(=O)O)NC(=O)[C@H](CC(=O)O)NC(=O)[C@H](CC(=O)O)NC(=O)CNC(=O)CNC(=O)CNC(=O)[C@H](CC(=O)O)NC(=O)[C@H](CC(=O)O)N*)C(N)=O)nn1]

or a salt thereof. In certain embodiments, L comprises

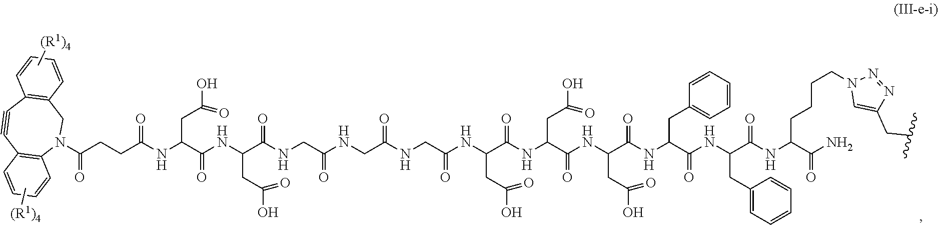

or a salt thereof. In certain embodiments, L comprises

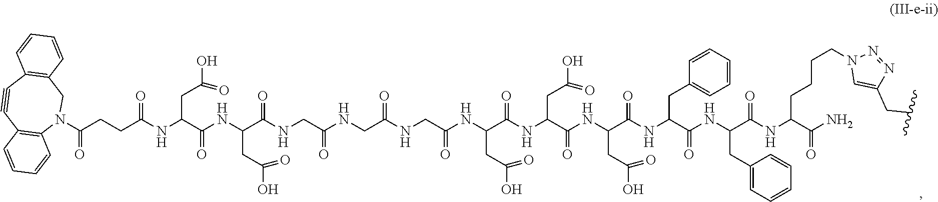 [SMILES: *Cc1cn(CCCCC(NC(=O)C(Cc2ccccc2)NC(=O)C(Cc2ccccc2)NC(=O)C(CC(=O)O)NC(=O)C(CC(=O)O)NC(=O)C(CC(=O)O)NC(=O)CNC(=O)CNC(=O)CNC(=O)C(CC(=O)O)NC(=O)C(CC(=O)O)NC(=O)CCC(=O)N2Cc3ccccc3C#Cc3ccccc32)C(N)=O)nn1]

or a salt thereof. In certain embodiments, L comprises

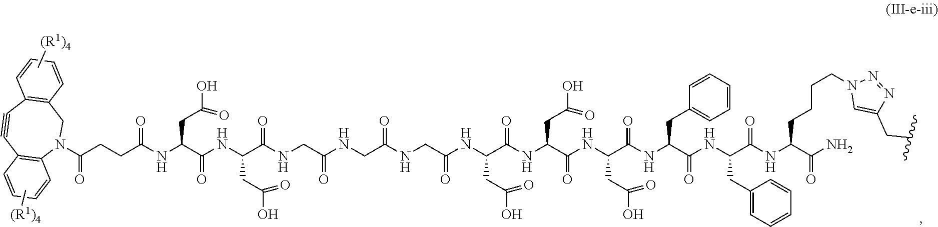

or a salt thereof. In certain embodiments, L comprises

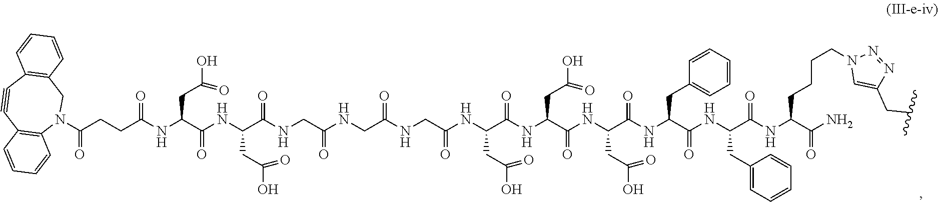 [SMILES: *Cc1cn(CCCC[C@H](NC(=O)[C@H](Cc2ccccc2)NC(=O)[C@H](Cc2ccccc2)NC(=O)[C@H](CC(=O)O)NC(=O)[C@H](CC(=O)O)NC(=O)[C@H](CC(=O)O)NC(=O)CNC(=O)CNC(=O)CNC(=O)[C@H](CC(=O)O)NC(=O)[C@H](CC(=O)O)NC(=O)CCC(=O)N2Cc3ccccc3C#Cc3ccccc32)C(N)=O)nn1]

or a salt thereof.

In certain embodiments, the compound is of Formulae (I-a-i), (I-a-ii), (I-b-i), or (I-b-ii):

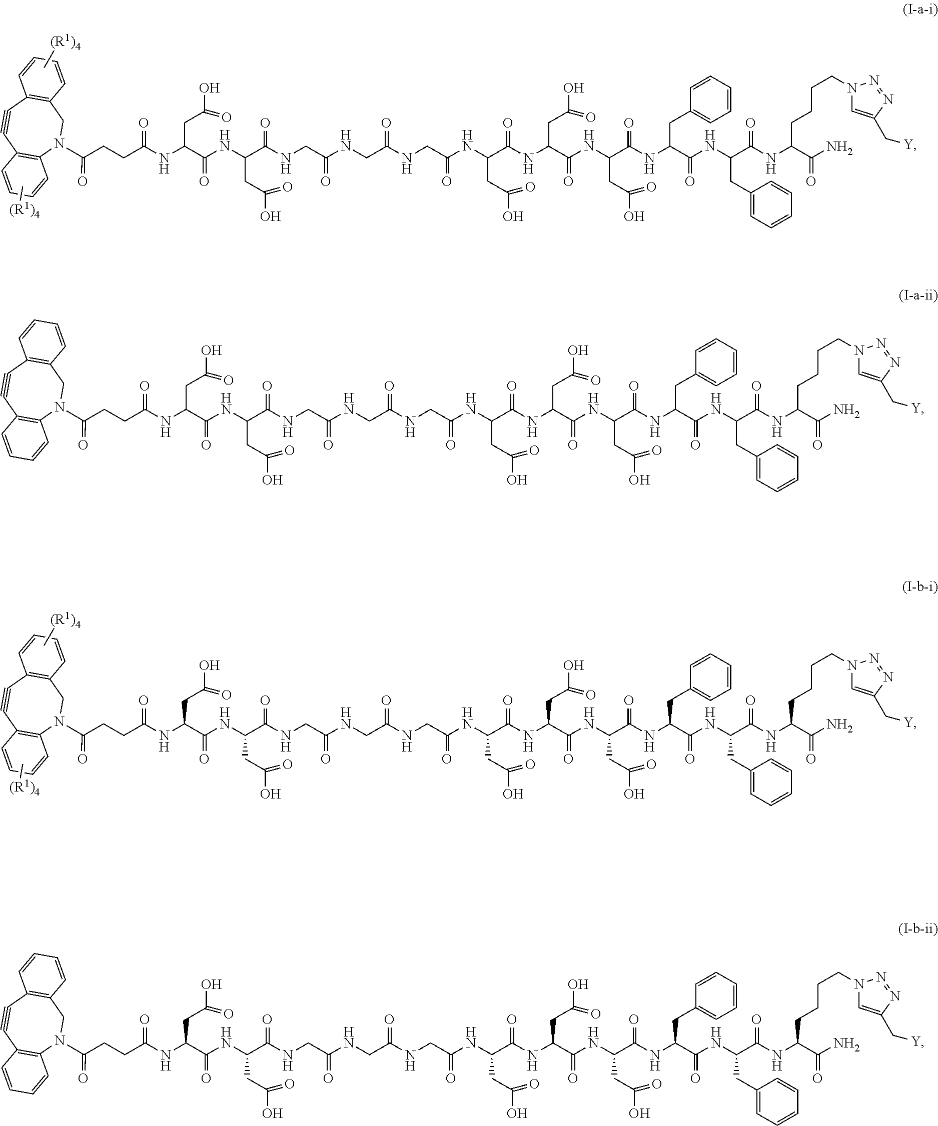

or a salt thereof. In certain embodiments the compound is of Formula (I-a-i):

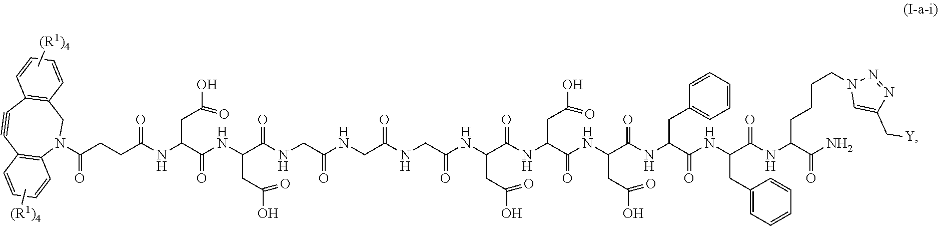

or a salt thereof. In certain embodiments, the compound is of Formula (I-a-ii):

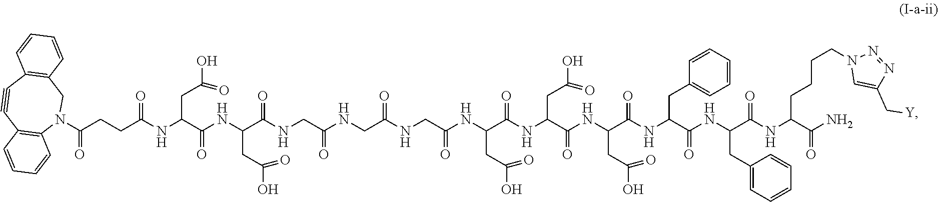

or a salt thereof. In certain embodiments, the compound is of Formula (I-b-i):

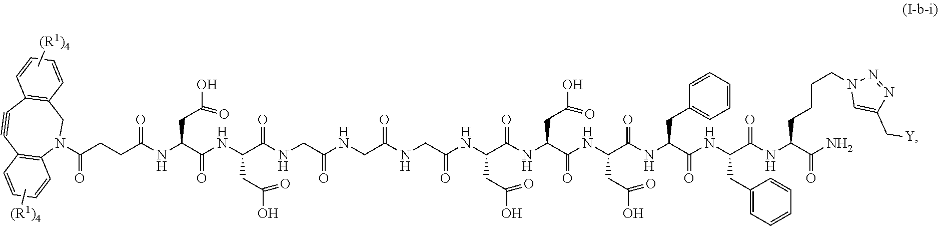

or a salt thereof. In certain embodiments, the compound is of Formula (I-b-ii):

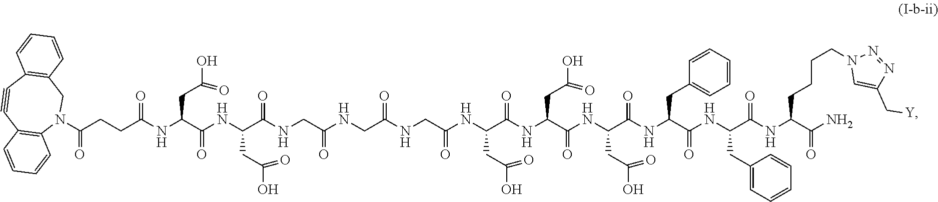

or a salt thereof.

In certain embodiments, the oligonucleotide comprises Q24 (5′-CCACGCGTGGAACCCTTGGGATCCA-3′(SEQ ID NO: 42). In certain embodiments, at least one strand of the oligonucleotide has a sequence that is at least 50%, at least 55%, at least 60%, at least 65%, at least 70%, at least 75%, at least 80%, at least 85%, at least 90%, at least 95%, at least 99%, or 100% identical to 5′-CCACGCGTGGAACCCTTGGGATCCA-3′ (SEQ ID NO: 42). In certain embodiments, at least one strand of the oligonucleotide has a sequence that is at least 50%, at least 55%, at least 60%, at least 65%, at least 70%, at least 75%, at least 80%, at least 85%, at least 90%, at least 95%, at least 99%, or 100% identical to 5′-TGG AGT CAA GGT CCT CTG ATG CCA T-3′ (SEQ ID NO: 70).

In certain embodiments, the oligonucleotide comprises at least about 10 bases. In certain embodiments, the oligonucleotide comprises at least about 15 bases. In certain embodiments, the oligonucleotide comprises at least about 20 bases. In certain embodiments, the oligonucleotide comprises at least about 25 bases. In certain embodiments, the oligonucleotide comprises at least about 30 bases. In certain embodiments, the oligonucleotide comprises at least about 35 bases. In certain embodiments, the oligonucleotide comprises at least about 40 bases. In certain embodiments, the oligonucleotide comprises at least about 45 bases. In certain embodiments, the oligonucleotide comprises at least about 50 bases. In certain embodiments, the oligonucleotide comprises between about 10 and about 50 bases. In certain embodiments, the oligonucleotide comprises between about 15 and about 50 bases. In certain embodiments, the oligonucleotide comprises between about 20 and about 50 bases. In certain embodiments, the oligonucleotide comprises between about 25 and about 50 bases. In certain embodiments, the oligonucleotide comprises between about 25 and about 45 bases. In certain embodiments, the oligonucleotide comprises between about 25 and about 40 bases. In certain embodiments, the oligonucleotide comprises between about 25 and about 35 bases. In certain embodiments, the oligonucleotide comprises between about 25 and about 30 bases. In certain embodiments, the oligonucleotide comprises 10 bases. In certain embodiments, the oligonucleotide comprises 15 bases. In certain embodiments, the oligonucleotide comprises 20 bases. In certain embodiments, the oligonucleotide comprises 25 bases (e.g., the oligonucleotide is a 25-mer). In certain embodiments, the oligonucleotide comprises 30 bases. In certain embodiments, the oligonucleotide comprises 35 bases. In certain embodiments, the oligonucleotide comprises 40 bases. In certain embodiments, the oligonucleotide comprises 45 bases. In certain embodiments, the oligonucleotide comprises 50 bases.

In certain embodiments, the oligonucleotide comprises between about 10 and about 50 bases, and the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), or a salt thereof, wherein Cy is cysteic acid. In certain embodiments, the oligonucleotide comprises between about 25 and about 50 bases, and the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), or a salt thereof, wherein Cy is cysteic acid. In certain embodiments, the oligonucleotide comprises between about 25 and about 45 bases, and the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), or a salt thereof, wherein Cy is cysteic acid. In certain embodiments, the oligonucleotide comprises between about 25 and about 40 bases, and the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), or a salt thereof, wherein Cy is cysteic acid. In certain embodiments, the oligonucleotide comprises between about 25 and about 35 bases, and the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), or a salt thereof, wherein Cy is cysteic acid. In certain embodiments, the oligonucleotide comprises between about 25 and about 30 bases, and the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), or a salt thereof, wherein Cy is cysteic acid. In certain embodiments, the oligonucleotide comprises 25 bases (e.g., the oligonucleotide is a 25-mer), and the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), or a salt thereof, wherein Cy is cysteic acid.

In certain embodiments, the oligonucleotide comprises Q24, and the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), or a salt thereof, wherein Cy is cysteic acid. In certain embodiments, the oligonucleotide comprises Q24, and the polypeptidyl group comprises a sequence GPPPPPPPPG (SEQ ID NO: 61), or a salt thereof. In certain embodiments, the oligonucleotide comprises Q24, and the polypeptidyl group comprises a sequence isoEGWRW (SEQ ID NO: 62), or a salt thereof. In certain embodiments, the oligonucleotide comprises Q24, and the polypeptidyl group comprises a sequence DDGGGDDDFF (SEQ ID NO: 32), or a salt thereof. In certain embodiments, the oligonucleotide comprises Q24, and the polypeptidyl group comprises a sequence GGSSSGSGNDEEFQ (SEQ ID NO: 59), or a salt thereof. In certain embodiments, the oligonucleotide comprises Q24, and the polypeptidyl group comprises a sequence GGGGGDPDPD (SEQ ID NO: 54), or a salt thereof. In certain embodiments, the oligonucleotide comprises Q24, and the polypeptidyl group comprises a sequence GGGGGDPDPDFF (SEQ ID NO: 55), or a salt thereof. In certain embodiments, the oligonucleotide comprises Q24, and the polypeptidyl group comprises a sequence GGGGGGDPDPD (SEQ ID NO: 57), or a salt thereof. In certain embodiments, the oligonucleotide comprises Q24, and the polypeptidyl group comprises a sequence GDGDGDGDGDFF (SEQ ID NO: 53), or a salt thereof. In certain embodiments, the oligonucleotide comprises Q24, and the polypeptidyl group comprises a sequence GDDGDGDGDFF (SEQ ID NO: 51), or a salt thereof. In certain embodiments, the oligonucleotide comprises Q24, and the polypeptidyl group comprises a sequence NNGGGNNNFF (SEQ ID NO: 65), or a salt thereof. In certain embodiments, the oligonucleotide comprises Q24, and the polypeptidyl group comprises a sequence DDGGGCyCyCyFF (SEQ ID NO: 45), or a salt thereof, wherein Cy is cysteic acid. In certain embodiments, the oligonucleotide comprises Q24, and the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), wherein Cy is cysteic acid. In certain embodiments, the oligonucleotide comprises Q24, and the polypeptidyl group comprises a sequence GPPPPPPPPG (SEQ ID NO: 61). In certain embodiments, the oligonucleotide comprises Q24, and the polypeptidyl group comprises a sequence isoEGWRW (SEQ ID NO: 62). In certain embodiments, the oligonucleotide comprises Q24, and the polypeptidyl group comprises a sequence DDGGGDDDFF (SEQ ID NO: 32). In certain embodiments, the oligonucleotide comprises Q24, and the polypeptidyl group comprises a sequence GGSSSGSGNDEEFQ (SEQ ID NO: 59). In certain embodiments, the oligonucleotide comprises Q24, and the polypeptidyl group comprises a sequence GGGGGDPDPD (SEQ ID NO: 54). In certain embodiments, the oligonucleotide comprises Q24, and the polypeptidyl group comprises a sequence GGGGGDPDPDFF (SEQ ID NO: 55). In certain embodiments, the oligonucleotide comprises Q24, and the polypeptidyl group comprises a sequence GGGGGGDPDPD (SEQ ID NO: 57). In certain embodiments, the oligonucleotide comprises Q24, and the polypeptidyl group comprises a sequence GDGDGDGDGDFF (SEQ ID NO: 53). In certain embodiments, the oligonucleotide comprises Q24, and the polypeptidyl group comprises a sequence GDDGDGDGDFF (SEQ ID NO: 51). In certain embodiments, the oligonucleotide comprises Q24, and the polypeptidyl group comprises a sequence NNGGGNNNFF (SEQ ID NO: 65). In certain embodiments, the oligonucleotide comprises Q24, and the polypeptidyl group comprises a sequence DDGGGCyCyCyFF (SEQ ID NO: 45), wherein Cy is cysteic acid.

In certain embodiments, Y further comprises at least one biotin moiety. In certain embodiments, Y further comprises a biotin moiety. In certain embodiments, Y further comprises two or more biotin moieties. In certain embodiments, at least one biotin moiety is a bis-biotin moiety. In certain embodiments, the biotin moiety is a bis-biotin moiety. In some embodiments, Y further comprises a tag sequence. In some embodiments, a tag sequence comprises at least one biotin ligase recognition sequence that permits biotinylation of Y (e.g., incorporation of one or more biotin molecules, including biotin and bis-biotin moieties). In some embodiments, the tag sequence comprises two biotin ligase recognition sequences oriented in tandem. In some embodiments, a biotin ligase recognition sequence refers to an amino acid sequence that is recognized by a biotin ligase, which catalyzes a covalent linkage between the sequence and a biotin molecule. Each biotin ligase recognition sequence of a tag sequence can be covalently linked to a biotin moiety, such that a tag sequence having multiple biotin ligase recognition sequences can be covalently linked to multiple biotin molecules. A region of a tag sequence having one or more biotin ligase recognition sequences can be generally referred to as a biotinylation tag or a biotinylation sequence. In some embodiments, a bis-biotin or bis-biotin moiety can refer to two biotins bound to two biotin ligase recognition sequences oriented in tandem. In some embodiments, Y comprises at least one biotin ligase recognition sequence having the biotin moiety attached thereto. In some embodiments, Y comprises at least one biotin ligase recognition sequence having the bis-biotin moiety attached thereto. In some embodiments, Y comprises at least two biotin ligase recognition sequences having the biotin moiety attached thereto. In some embodiments, Y comprises at least two biotin ligase recognition sequences having the bis-biotin moiety attached thereto. In certain embodiments, the oligonucleotide comprises Q24, and Y further comprises at least one biotin moiety. In certain embodiments, the oligonucleotide comprises Q24, and Y further comprises a biotin moiety. In certain embodiments, the oligonucleotide comprises Q24, and Y further comprises two or more biotin moieties. In certain embodiments, the oligonucleotide comprises Q24, and at least one biotin moiety is a bis-biotin moiety. In certain embodiments, the oligonucleotide comprises Q24, and the biotin moiety is a bis-biotin moiety. In some embodiments, the oligonucleotide comprises Q24, and Y further comprises a tag sequence. In some embodiments, the oligonucleotide comprises Q24, and Y comprises at least one biotin ligase recognition sequence having the biotin moiety attached thereto. In some embodiments, the oligonucleotide comprises Q24, and Y comprises at least one biotin ligase recognition sequence having the bis-biotin moiety attached thereto. In some embodiments, the oligonucleotide comprises Q24, and Y comprises at least two biotin ligase recognition sequences having the biotin moiety attached thereto. In some embodiments, the oligonucleotide comprises Q24, and Y comprises at least two biotin ligase recognition sequences having the bis-biotin moiety attached thereto.

In certain embodiments, Y further comprises an avidin protein. In certain embodiments, the avidin protein is avidin, streptavidin, traptavidin, tamavidin, bradavidin, xenavidin, or a homolog or variant thereof. In certain embodiments, the avidin protein is avidin, streptavidin, traptavidin, tamavidin, bradavidin, or xenavidin. In certain embodiments, the avidin protein is avidin. In certain embodiments, the avidin protein is streptavidin. In certain embodiments, the avidin protein is traptavidin. In certain embodiments, the avidin protein is tamavidin. In certain embodiments, the avidin protein is bradavidin. In certain embodiments, the avidin protein is xenavidin. In certain embodiments, the avidin protein is in a monomeric, dimeric, or tetrameric form. In certain embodiments, the avidin protein is in a monomeric form. In certain embodiments, the avidin protein is in a dimeric form. In certain embodiments, the avidin protein is in a tetrameric form. In some embodiments, the avidin protein is streptavidin in a tetrameric form (e.g., a homotetramer). In certain embodiments, the oligonucleotide comprises Q24, and Y further comprises an avidin protein. In certain embodiments, the oligonucleotide comprises Q24, and the avidin protein is avidin, streptavidin, traptavidin, tamavidin, bradavidin, xenavidin, or a homolog or variant thereof. In certain embodiments, the oligonucleotide comprises Q24, and the avidin protein is streptavidin. In certain embodiments, the oligonucleotide comprises Q24, and the avidin protein is in a monomeric, dimeric, or tetrameric form. In certain embodiments, the oligonucleotide comprises Q24, and the avidin protein is in a monomeric form. In certain embodiments, the oligonucleotide comprises Q24, and the avidin protein is in a dimeric form. In certain embodiments, the oligonucleotide comprises Q24, and the avidin protein is in a tetrameric form. In some embodiments, the oligonucleotide comprises Q24, and the avidin protein is streptavidin in a tetrameric form (e.g., a homotetramer).

In some embodiments, the avidin protein comprises one or more biotin binding sites. In some embodiments, the one or more biotin binding sites of an avidin protein provide attachment sites for Y. In some embodiments, the one or more biotin binding sites of an avidin protein provide attachment sites for Y, wherein Y further comprises at least one biotin moiety. In some embodiments, the at least one biotin moiety binds to the one or more biotin binding sites of an avidin protein. In some embodiments, the at least one biotin moiety is a bis-biotin moiety, and the bis-biotin moiety is bound to two biotin binding sites on the avidin protein.

In certain embodiments, Y is immobilized to a surface. In certain embodiments, the oligonucleotide comprises Q24, and Y is immobilized to a surface. As used herein, in some embodiments, a surface refers to a surface of a substrate or solid support. In some embodiments, a solid support refers to a material, layer, or other structure having a surface, such as a receiving surface, that is capable of supporting a deposited material, such as a compound described herein. In some embodiments, a receiving surface of a substrate may optionally have one or more features, including nanoscale or microscale recessed features such as an array of sample wells. In some embodiments, an array is a planar arrangement of elements such as sensors or sample wells. An array may be one or two dimensional. A one dimensional array is an array having one column or row of elements in the first dimension and a plurality of columns or rows in the second dimension. The number of columns or rows in the first and second dimensions may or may not be the same. In some embodiments, the array may include, for example, 10², 10³, 10⁴, 10⁵, 10⁶, or 10⁷ sample wells. In some embodiments, the surface is functionalized with a complementary functional moiety configured for attachment (e.g., covalent or non-covalent attachment) to Y. In some embodiments, the complementary functional moiety is a biotin moiety. In some embodiments, the complementary functional moiety is a bis-biotin moiety. In some embodiments, Y is immobilized to a bottom surface or a sidewall surface of a sample well. In some embodiments, surface immobilization of Y allows the compound to be confined to a desired region of a surface for real-time monitoring of a reaction involving the compound. In certain embodiments, the compound is immobilized to a surface through Y. In certain embodiments, the compound is immobilized to a surface through Y such that the compound may be monitored without interference from other reaction components in solution. In some embodiments, surface immobilization of Y allows the compound to be confined to a desired region of a surface for real-time monitoring of a reaction involving the compound.

### Methods of Preparation

In another aspect, provided herein is a method of preparing a compound of Formula (II):

Z-L-Y (II),

or a salt thereof, comprising reacting a compound of Formula (I):

L-Y (I),

or a salt thereof, with a compound of formula Z—N₃, or a salt thereof, wherein:
L comprises a polypeptidyl group;
Y is an oligonucleotide; and
Z is a polypeptide.

In certain embodiments, reacting a compound of Formula (I), or a salt thereof, with a compound of formula Z—N₃, or a salt thereof, comprises a click chemistry reaction. In certain embodiments, reacting a compound of Formula (I), or a salt thereof, with a compound of formula Z—N3, or a salt thereof, comprises an azide-alkyne cycloaddition.

In certain embodiments, the method further comprises reacting a compound of formula L-N₃, or a salt thereof, with a compound of formula Y-propargyl, or a salt thereof, to provide the compound of Formula (I):

L-Y (I),

or a salt thereof.

In certain embodiments, the polypeptidyl group comprises at least 10 amino acid residues. In certain embodiments, the polypeptidyl group comprises at least 11 amino acid residues. In certain embodiments, the polypeptidyl group comprises at least 12 amino acid residues. In certain embodiments, the polypeptidyl group comprises at least 13 amino acid residues. In certain embodiments, the polypeptidyl group comprises at least 14 amino acid residues. In certain embodiments, the polypeptidyl group comprises at least 15 amino acid residues. In certain embodiments, the polypeptidyl group comprises between 10 and 15 amino acid residues, inclusive. In certain embodiments, the polypeptidyl group comprises between 10 and 14 amino acid residues, inclusive. In certain embodiments, the polypeptidyl group comprises between 10 and 13 amino acid residues, inclusive. In certain embodiments, the polypeptidyl group comprises between 10 and 12 amino acid residues, inclusive. In certain embodiments, the polypeptidyl group comprises 10 amino acid residues. In certain embodiments, the polypeptidyl group comprises 11 amino acid residues. In certain embodiments, the polypeptidyl group comprises 12 amino acid residues.

In certain embodiments, the polypeptidyl group is at least about 30 Å in length. In certain embodiments, the polypeptidyl group is at least about 33 Å in length. In certain embodiments, the polypeptidyl group is at least about 35 Å in length. In certain embodiments, the polypeptidyl group is between about 25 Å and about 50 Å in length, inclusive. In certain embodiments, the polypeptidyl group is between about 25 Å and about 45 Å in length, inclusive. In certain embodiments, the polypeptidyl group is between about 25 Å and about 40 Å in length, inclusive. In certain embodiments, the polypeptidyl group is between about 25 Å and about 35 Å in length, inclusive. In certain embodiments, the polypeptidyl group is between about 30 Å and about 35 Å in length, inclusive. In certain embodiments, the polypeptidyl group is about 33 Å in length.

In certain embodiments, the polypeptidyl group comprises between 10 and 15 amino acid residues, inclusive, and the polypeptidyl group is between about 25 Å and about 50 Å in length, inclusive. In certain embodiments, the polypeptidyl group comprises between 10 and 14 amino acid residues, inclusive, and the polypeptidyl group is between about 25 Å and about 50 Å in length, inclusive. In certain embodiments, the polypeptidyl group comprises between 10 and 13 amino acid residues, inclusive, and the polypeptidyl group is between about 25 Å and about 50 Å in length, inclusive. In certain embodiments, the polypeptidyl group comprises between 10 and 12 amino acid residues, inclusive, and the polypeptidyl group is between about 25 Å and about 50 Å in length, inclusive. In certain embodiments, the polypeptidyl group comprises 10 amino acid residues, and the polypeptidyl group is between about 25 Å and about 50 Å in length, inclusive. In certain embodiments, the polypeptidyl group comprises 11 amino acid residues, and the polypeptidyl group is between about 25 Å and about 50 Å in length, inclusive. In certain embodiments, the polypeptidyl group comprises 12 amino acid residues, and the polypeptidyl group is between about 25 Å and about 50 Å in length, inclusive. In certain embodiments, the polypeptidyl group comprises between 10 and 15 amino acid residues, inclusive, and the polypeptidyl group is between about 25 Å and about 45 Å in length, inclusive. In certain embodiments, the polypeptidyl group comprises between 10 and 14 amino acid residues, inclusive, and the polypeptidyl group is between about 25 Å and about 45 Å in length, inclusive. In certain embodiments, the polypeptidyl group comprises between 10 and 13 amino acid residues, inclusive, and the polypeptidyl group is between about 25 Å and about 45 Å in length, inclusive. In certain embodiments, the polypeptidyl group comprises between 10 and 12 amino acid residues, inclusive, and the polypeptidyl group is between about 25 Å and about 45 Å in length, inclusive. In certain embodiments, the polypeptidyl group comprises 10 amino acid residues, and the polypeptidyl group is between about 25 Å and about 45 Å in length, inclusive. In certain embodiments, the polypeptidyl group comprises 11 amino acid residues, and the polypeptidyl group is between about 25 Å and about 45 Å in length, inclusive. In certain embodiments, the polypeptidyl group comprises 12 amino acid residues, and the polypeptidyl group is between about 25 Å and about 45 Å in length, inclusive. In certain embodiments, the polypeptidyl group comprises between 10 and 15 amino acid residues, inclusive, and the polypeptidyl group is between about 25 Å and about 40 Å in length, inclusive. In certain embodiments, the polypeptidyl group comprises between 10 and 14 amino acid residues, inclusive, and the polypeptidyl group is between about 25 Å and about 40 Å in length, inclusive. In certain embodiments, the polypeptidyl group comprises between 10 and 13 amino acid residues, inclusive, and the polypeptidyl group is between about 25 Å and about 40 Å in length, inclusive. In certain embodiments, the polypeptidyl group comprises between 10 and 12 amino acid residues, inclusive, and the polypeptidyl group is between about 25 Å and about 40 Å in length, inclusive. In certain embodiments, the polypeptidyl group comprises 10 amino acid residues, and the polypeptidyl group is between about 25 Å and about 40 Å in length, inclusive. In certain embodiments, the polypeptidyl group comprises 11 amino acid residues, and the polypeptidyl group is between about 25 Å and about 40 Å in length, inclusive. In certain embodiments, the polypeptidyl group comprises 12 amino acid residues, and the polypeptidyl group is between about 25 Å and about 40 Å in length, inclusive. In certain embodiments, the polypeptidyl group comprises between 10 and 15 amino acid residues, inclusive, and the polypeptidyl group is between about 25 Å and about 35 Å in length, inclusive. In certain embodiments, the polypeptidyl group comprises between 10 and 14 amino acid residues, inclusive, and the polypeptidyl group is between about 25 Å and about 35 Å in length, inclusive. In certain embodiments, the polypeptidyl group comprises between 10 and 13 amino acid residues, inclusive, and the polypeptidyl group is between about 25 Å and about 35 Å in length, inclusive. In certain embodiments, the polypeptidyl group comprises between 10 and 12 amino acid residues, inclusive, and the polypeptidyl group is between about 25 Å and about 35 Å in length, inclusive. In certain embodiments, the polypeptidyl group comprises 10 amino acid residues, and the polypeptidyl group is between about 25 Å and about 35 Å in length, inclusive. In certain embodiments, the polypeptidyl group comprises 11 amino acid residues, and the polypeptidyl group is between about 25 Å and about 35 Å in length, inclusive. In certain embodiments, the polypeptidyl group comprises 12 amino acid residues, and the polypeptidyl group is between about 25 Å and about 35 Å in length, inclusive. In certain embodiments, the polypeptidyl group comprises between 10 and 15 amino acid residues, inclusive, and the polypeptidyl group is between about 30 Å and about 35 Å in length, inclusive. In certain embodiments, the polypeptidyl group comprises between 10 and 14 amino acid residues, inclusive, and the polypeptidyl group is between about 30 Å and about 35 Å in length, inclusive. In certain embodiments, the polypeptidyl group comprises between 10 and 13 amino acid residues, inclusive, and the polypeptidyl group is between about 30 Å and about 35 Å in length, inclusive. In certain embodiments, the polypeptidyl group comprises between 10 and 12 amino acid residues, inclusive, and the polypeptidyl group is between about 30 Å and about 35 Å in length, inclusive. In certain embodiments, the polypeptidyl group comprises 10 amino acid residues, and the polypeptidyl group is between about 30 Å and about 35 Å in length, inclusive. In certain embodiments, the polypeptidyl group comprises 11 amino acid residues, and the polypeptidyl group is between about 30 Å and about 35 Å in length, inclusive. In certain embodiments, the polypeptidyl group comprises 12 amino acid residues, and the polypeptidyl group is between about 30 Å and about 35 Å in length, inclusive. In certain embodiments, the polypeptidyl group comprises between 10 and 15 amino acid residues, inclusive, and the polypeptidyl group is about 33 Å in length. In certain embodiments, the polypeptidyl group comprises between 10 and 14 amino acid residues, inclusive, and the polypeptidyl group is about 33 Å in length. In certain embodiments, the polypeptidyl group comprises between 10 and 13 amino acid residues, inclusive, and the polypeptidyl group is about 33 Å in length. In certain embodiments, the polypeptidyl group comprises between 10 and 12 amino acid residues, inclusive, and the polypeptidyl group is about 33 Å in length. In certain embodiments, the polypeptidyl group comprises 10 amino acid residues, and the polypeptidyl group is about 33 Å in length. In certain embodiments, the polypeptidyl group comprises 11 amino acid residues, and the polypeptidyl group is about 33 Å in length. In certain embodiments, the polypeptidyl group comprises 12 amino acid residues, and the polypeptidyl group is about 33 Å in length.

In certain embodiments, the polypeptidyl group comprises at least 5 negatively charged moieties at physiological pH. In certain embodiments, the polypeptidyl group comprises at least 6 negatively charged moieties at physiological pH. In certain embodiments, the polypeptidyl group comprises between 3 and 6 negatively charged moieties at physiological pH, inclusive. In certain embodiments, the polypeptidyl group comprises between 4 and 6 negatively charged moieties at physiological pH, inclusive. In certain embodiments, the polypeptidyl group comprises between 5 and 6 negatively charged moieties at physiological pH, inclusive. In certain embodiments, the polypeptidyl group comprises 4 negatively charged moieties at physiological pH. In certain embodiments, the polypeptidyl group comprises 5 negatively charged moieties at physiological pH. In certain embodiments, the polypeptidyl group comprises 6 negatively charged moieties at physiological pH.

In certain embodiments, the polypeptidyl group comprises between 3 and 6 negatively charged moieties at physiological pH, inclusive, and the polypeptidyl group is between about 25 Å and about 50 Å in length, inclusive. In certain embodiments, the polypeptidyl group comprises between 3 and 6 negatively charged moieties at physiological pH, inclusive, and the polypeptidyl group is between about 25 Å and about 45 Å in length, inclusive. In certain embodiments, the polypeptidyl group comprises between 3 and 6 negatively charged moieties at physiological pH, inclusive, and the polypeptidyl group is between about 25 Å and about 40 Å in length, inclusive. In certain embodiments, the polypeptidyl group comprises between 3 and 6 negatively charged moieties at physiological pH, inclusive, and the polypeptidyl group is between about 25 Å and about 35 Å in length, inclusive. In certain embodiments, the polypeptidyl group comprises between 3 and 6 negatively charged moieties at physiological pH, inclusive, and the polypeptidyl group is between about 30 Å and about 35 Å in length, inclusive. In certain embodiments, the polypeptidyl group comprises between 3 and 6 negatively charged moieties at physiological pH, inclusive, and the polypeptidyl group is about 33 Å in length. In certain embodiments, the polypeptidyl group comprises between 4 and 6 negatively charged moieties at physiological pH, inclusive, and the polypeptidyl group is between about 25 Å and about 50 Å in length, inclusive. In certain embodiments, the polypeptidyl group comprises between 4 and 6 negatively charged moieties at physiological pH, inclusive, and the polypeptidyl group is between about 25 Å and about 45 Å in length, inclusive. In certain embodiments, the polypeptidyl group comprises between 4 and 6 negatively charged moieties at physiological pH, inclusive, and the polypeptidyl group is between about 25 Å and about 40 Å in length, inclusive. In certain embodiments, the polypeptidyl group comprises between 4 and 6 negatively charged moieties at physiological pH, inclusive, and the polypeptidyl group is between about 25 Å and about 35 Å in length, inclusive. In certain embodiments, the polypeptidyl group comprises between 4 and 6 negatively charged moieties at physiological pH, inclusive, and the polypeptidyl group is between about 30 Å and about 35 Å in length, inclusive. In certain embodiments, the polypeptidyl group comprises between 4 and 6 negatively charged moieties at physiological pH, inclusive, and the polypeptidyl group is about 33 Å in length. In certain embodiments, the polypeptidyl group comprises between 5 and 6 negatively charged moieties at physiological pH, inclusive, and the polypeptidyl group is between about 25 Å and about 50 Å in length, inclusive. In certain embodiments, the polypeptidyl group comprises between 5 and 6 negatively charged moieties at physiological pH, inclusive, and the polypeptidyl group is between about 25 Å and about 45 Å in length, inclusive. In certain embodiments, the polypeptidyl group comprises between 5 and 6 negatively charged moieties at physiological pH, inclusive, and the polypeptidyl group is between about 25 Å and about 40 Å in length, inclusive. In certain embodiments, the polypeptidyl group comprises between 5 and 6 negatively charged moieties at physiological pH, inclusive, and the polypeptidyl group is between about 25 Å and about 35 Å in length, inclusive. In certain embodiments, the polypeptidyl group comprises between 5 and 6 negatively charged moieties at physiological pH, inclusive, and the polypeptidyl group is between about 30 Å and about 35 Å in length, inclusive. In certain embodiments, the polypeptidyl group comprises between 5 and 6 negatively charged moieties at physiological pH, inclusive, and the polypeptidyl group is about 33 Å in length.

In certain embodiments, the polypeptidyl group comprises between 3 and 7 aspartate residues, inclusive. In certain embodiments, the polypeptidyl group comprises between 4 and 7 aspartate residues, inclusive. In certain embodiments, the polypeptidyl group comprises between 5 and 7 aspartate residues, inclusive. In certain embodiments, the polypeptidyl group comprises between 3 and 6 aspartate residues, inclusive. In certain embodiments, the polypeptidyl group comprises between 4 and 6 aspartate residues, inclusive. In certain embodiments, the polypeptidyl group comprises between 5 and 6 aspartate residues, inclusive. In certain embodiments, the polypeptidyl group comprises 5 aspartate residues. In certain embodiments, the polypeptidyl group comprises 6 aspartate residues.

In certain embodiments, the polypeptidyl group comprises between 1 and 4 phenylalanine residues, inclusive. In certain embodiments, the polypeptidyl group comprises between 1 and 3 phenylalanine residues, inclusive. In certain embodiments, the polypeptidyl group comprises between 1 and 2 phenylalanine residues, inclusive. In certain embodiments, the polypeptidyl group comprises between 2 and 3 phenylalanine residues, inclusive. In certain embodiments, the polypeptidyl group comprises 1 phenylalanine residue. In certain embodiments, the polypeptidyl group comprises 2 phenylalanine residues.

In certain embodiments, the polypeptidyl group comprises between 3 and 7 aspartate residues, inclusive, and the polypeptidyl group comprises between 1 and 4 phenylalanine residues, inclusive. In certain embodiments, the polypeptidyl group comprises between 4 and 7 aspartate residues, inclusive, and the polypeptidyl group comprises between 1 and 4 phenylalanine residues, inclusive. In certain embodiments, the polypeptidyl group comprises between 5 and 7 aspartate residues, inclusive, and the polypeptidyl group comprises between 1 and 4 phenylalanine residues, inclusive. In certain embodiments, the polypeptidyl group comprises between 4 and 6 aspartate residues, inclusive, and the polypeptidyl group comprises between 1 and 4 phenylalanine residues, inclusive. In certain embodiments, the polypeptidyl group comprises between 5 and 6 aspartate residues, inclusive, and the polypeptidyl group comprises between 1 and 4 phenylalanine residues, inclusive. In certain embodiments, the polypeptidyl group comprises 5 aspartate residues, and the polypeptidyl group comprises between 1 and 4 phenylalanine residues, inclusive. In certain embodiments, the polypeptidyl group comprises 6 aspartate residues, and the polypeptidyl group comprises between 1 and 4 phenylalanine residues, inclusive. In certain embodiments, the polypeptidyl group comprises between 3 and 7 aspartate residues, inclusive, and the polypeptidyl group comprises between 1 and 3 phenylalanine residues, inclusive. In certain embodiments, the polypeptidyl group comprises between 4 and 7 aspartate residues, inclusive, and the polypeptidyl group comprises between 1 and 3 phenylalanine residues, inclusive. In certain embodiments, the polypeptidyl group comprises between 5 and 7 aspartate residues, inclusive, and the polypeptidyl group comprises between 1 and 3 phenylalanine residues, inclusive. In certain embodiments, the polypeptidyl group comprises between 4 and 6 aspartate residues, inclusive, and the polypeptidyl group comprises between 1 and 3 phenylalanine residues, inclusive. In certain embodiments, the polypeptidyl group comprises between 5 and 6 aspartate residues, inclusive, and the polypeptidyl group comprises between 1 and 3 phenylalanine residues, inclusive. In certain embodiments, the polypeptidyl group comprises 5 aspartate residues, and the polypeptidyl group comprises between 1 and 3 phenylalanine residues, inclusive. In certain embodiments, the polypeptidyl group comprises 6 aspartate residues, and the polypeptidyl group comprises between 1 and 3 phenylalanine residues, inclusive. In certain embodiments, the polypeptidyl group comprises between 3 and 7 aspartate residues, inclusive, and the polypeptidyl group comprises between 1 and 2 phenylalanine residues, inclusive. In certain embodiments, the polypeptidyl group comprises between 4 and 7 aspartate residues, inclusive, and the polypeptidyl group comprises between 1 and 2 phenylalanine residues, inclusive. In certain embodiments, the polypeptidyl group comprises between 5 and 7 aspartate residues, inclusive, and the polypeptidyl group comprises between 1 and 2 phenylalanine residues, inclusive. In certain embodiments, the polypeptidyl group comprises between 4 and 6 aspartate residues, inclusive, and the polypeptidyl group comprises between 1 and 2 phenylalanine residues, inclusive. In certain embodiments, the polypeptidyl group comprises between 5 and 6 aspartate residues, inclusive, and the polypeptidyl group comprises between 1 and 2 phenylalanine residues, inclusive. In certain embodiments, the polypeptidyl group comprises 5 aspartate residues, and the polypeptidyl group comprises between 1 and 2 phenylalanine residues, inclusive. In certain embodiments, the polypeptidyl group comprises 6 aspartate residues, and the polypeptidyl group comprises between 1 and 2 phenylalanine residues, inclusive. In certain embodiments, the polypeptidyl group comprises between 3 and 7 aspartate residues, inclusive, and the polypeptidyl group comprises 1 phenylalanine residue. In certain embodiments, the polypeptidyl group comprises between 4 and 7 aspartate residues, inclusive, and the polypeptidyl group comprises 1 phenylalanine residue. In certain embodiments, the polypeptidyl group comprises between 5 and 7 aspartate residues, inclusive, and the polypeptidyl group comprises 1 phenylalanine residue. In certain embodiments, the polypeptidyl group comprises between 4 and 6 aspartate residues, inclusive, and the polypeptidyl group comprises 1 phenylalanine residue. In certain embodiments, the polypeptidyl group comprises between 5 and 6 aspartate residues, inclusive, and the polypeptidyl group comprises 1 phenylalanine residue. In certain embodiments, the polypeptidyl group comprises 5 aspartate residues, and the polypeptidyl group comprises 1 phenylalanine residue. In certain embodiments, the polypeptidyl group comprises 6 aspartate residues, and the polypeptidyl group comprises 1 phenylalanine residue. In certain embodiments, the polypeptidyl group comprises between 3 and 7 aspartate residues, inclusive, and the polypeptidyl group comprises 2 phenylalanine residues. In certain embodiments, the polypeptidyl group comprises between 4 and 7 aspartate residues, inclusive, and the polypeptidyl group comprises 2 phenylalanine residues. In certain embodiments, the polypeptidyl group comprises between 5 and 7 aspartate residues, inclusive, and the polypeptidyl group comprises 2 phenylalanine residues. In certain embodiments, the polypeptidyl group comprises between 4 and 6 aspartate residues, inclusive, and the polypeptidyl group comprises 2 phenylalanine residues. In certain embodiments, the polypeptidyl group comprises between 5 and 6 aspartate residues, inclusive, and the polypeptidyl group comprises 2 phenylalanine residues. In certain embodiments, the polypeptidyl group comprises 5 aspartate residues, and the polypeptidyl group comprises 2 phenylalanine residues. In certain embodiments, the polypeptidyl group comprises 6 aspartate residues, and the polypeptidyl group comprises 2 phenylalanine residues.

In certain embodiments, the polypeptidyl group comprises between 1 and 6 glycine residues, inclusive. In certain embodiments, the polypeptidyl group comprises between 1 and 5 glycine residues, inclusive. In certain embodiments, the polypeptidyl group comprises between 1 and 4 glycine residues, inclusive. In certain embodiments, the polypeptidyl group comprises between 1 and 3 glycine residues, inclusive. In certain embodiments, the polypeptidyl group comprises between 1 and 2 glycine residues, inclusive. In certain embodiments, the polypeptidyl group comprises 1 glycine residue. In certain embodiments, the polypeptidyl group comprises 2 glycine residues. In certain embodiments, the polypeptidyl group comprises 3 glycine residues. In certain embodiments, the polypeptidyl group comprises 4 glycine residues. In certain embodiments, the polypeptidyl group comprises 5 glycine residues. In certain embodiments, the polypeptidyl group comprises 6 glycine residues.

In certain embodiments, the polypeptidyl group comprises between 3 and 7 aspartate residues, inclusive, and the polypeptidyl group comprises between 1 and 4 glycine residues, inclusive. In certain embodiments, the polypeptidyl group comprises between 4 and 7 aspartate residues, inclusive, and the polypeptidyl group comprises between 1 and 4 glycine residues, inclusive. In certain embodiments, the polypeptidyl group comprises between 5 and 7 aspartate residues, inclusive, and the polypeptidyl group comprises between 1 and 4 glycine residues, inclusive. In certain embodiments, the polypeptidyl group comprises between 4 and 6 aspartate residues, inclusive, and the polypeptidyl group comprises between 1 and 4 glycine residues, inclusive. In certain embodiments, the polypeptidyl group comprises between 5 and 6 aspartate residues, inclusive, and the polypeptidyl group comprises between 1 and 4 glycine residues, inclusive. In certain embodiments, the polypeptidyl group comprises 5 aspartate residues, and the polypeptidyl group comprises between 1 and 4 glycine residues, inclusive. In certain embodiments, the polypeptidyl group comprises 6 aspartate residues, and the polypeptidyl group comprises between 1 and 4 glycine residues, inclusive. In certain embodiments, the polypeptidyl group comprises between 3 and 7 aspartate residues, inclusive, and the polypeptidyl group comprises between 1 and 3 glycine residues, inclusive. In certain embodiments, the polypeptidyl group comprises between 4 and 7 aspartate residues, inclusive, and the polypeptidyl group comprises between 1 and 3 glycine residues, inclusive. In certain embodiments, the polypeptidyl group comprises between 5 and 7 aspartate residues, inclusive, and the polypeptidyl group comprises between 1 and 3 glycine residues, inclusive. In certain embodiments, the polypeptidyl group comprises between 4 and 6 aspartate residues, inclusive, and the polypeptidyl group comprises between 1 and 3 glycine residues, inclusive. In certain embodiments, the polypeptidyl group comprises between 5 and 6 aspartate residues, inclusive, and the polypeptidyl group comprises between 1 and 3 glycine residues, inclusive. In certain embodiments, the polypeptidyl group comprises 5 aspartate residues, and the polypeptidyl group comprises between 1 and 3 glycine residues, inclusive. In certain embodiments, the polypeptidyl group comprises 6 aspartate residues, and the polypeptidyl group comprises between 1 and 3 glycine residues, inclusive. In certain embodiments, the polypeptidyl group comprises between 3 and 7 aspartate residues, inclusive, and the polypeptidyl group comprises between 2 and 3 glycine residues, inclusive. In certain embodiments, the polypeptidyl group comprises between 4 and 7 aspartate residues, inclusive, and the polypeptidyl group comprises between 2 and 3 glycine residues, inclusive. In certain embodiments, the polypeptidyl group comprises between 5 and 7 aspartate residues, inclusive, and the polypeptidyl group comprises between 2 and 3 glycine residues, inclusive. In certain embodiments, the polypeptidyl group comprises between 4 and 6 aspartate residues, inclusive, and the polypeptidyl group comprises between 2 and 3 glycine residues, inclusive. In certain embodiments, the polypeptidyl group comprises between 5 and 6 aspartate residues, inclusive, and the polypeptidyl group comprises between 2 and 3 glycine residues, inclusive. In certain embodiments, the polypeptidyl group comprises 5 aspartate residues, and the polypeptidyl group comprises between 2 and 3 glycine residues, inclusive. In certain embodiments, the polypeptidyl group comprises 6 aspartate residues, and the polypeptidyl group comprises between 2 and 3 glycine residues, inclusive. In certain embodiments, the polypeptidyl group comprises between 3 and 7 aspartate residues, inclusive, and the polypeptidyl group comprises 2 glycine residues. In certain embodiments, the polypeptidyl group comprises between 4 and 7 aspartate residues, inclusive, and the polypeptidyl group comprises 2 glycine residues. In certain embodiments, the polypeptidyl group comprises between 5 and 7 aspartate residues, inclusive, and the polypeptidyl group comprises 2 glycine residues. In certain embodiments, the polypeptidyl group comprises between 4 and 6 aspartate residues, inclusive, and the polypeptidyl group comprises 2 glycine residues. In certain embodiments, the polypeptidyl group comprises between 5 and 6 aspartate residues, inclusive, and the polypeptidyl group comprises 2 glycine residues. In certain embodiments, the polypeptidyl group comprises 5 aspartate residues, and the polypeptidyl group comprises 2 glycine residues. In certain embodiments, the polypeptidyl group comprises 6 aspartate residues, and the polypeptidyl group comprises 2 glycine residues. In certain embodiments, the polypeptidyl group comprises between 3 and 7 aspartate residues, inclusive, and the polypeptidyl group comprises 3 glycine residues. In certain embodiments, the polypeptidyl group comprises between 4 and 7 aspartate residues, inclusive, and the polypeptidyl group comprises 3 glycine residues. In certain embodiments, the polypeptidyl group comprises between 5 and 7 aspartate residues, inclusive, and the polypeptidyl group comprises 3 glycine residues. In certain embodiments, the polypeptidyl group comprises between 4 and 6 aspartate residues, inclusive, and the polypeptidyl group comprises 3 glycine residues. In certain embodiments, the polypeptidyl group comprises between 5 and 6 aspartate residues, inclusive, and the polypeptidyl group comprises 3 glycine residues. In certain embodiments, the polypeptidyl group comprises 5 aspartate residues, and the polypeptidyl group comprises 3 glycine residues. In certain embodiments, the polypeptidyl group comprises 6 aspartate residues, and the polypeptidyl group comprises 3 glycine residues.

In certain embodiments, the polypeptidyl group comprises between 1 and 4 proline residues, inclusive. In certain embodiments, the polypeptidyl group comprises between 1 and 3 proline residues, inclusive. In certain embodiments, the polypeptidyl group comprises between 1 and 2 proline residues, inclusive. In certain embodiments, the polypeptidyl group comprises between 2 and 10 proline residues, inclusive. In certain embodiments, the polypeptidyl group comprises 1 proline residue. In certain embodiments, the polypeptidyl group comprises 2 proline residues.

In certain embodiments, the polypeptidyl group comprises between 3 and 7 aspartate residues, inclusive, and the polypeptidyl group comprises between 1 and 4 proline residues, inclusive. In certain embodiments, the polypeptidyl group comprises between 4 and 7 aspartate residues, inclusive, and the polypeptidyl group comprises between 1 and 4 proline residues, inclusive. In certain embodiments, the polypeptidyl group comprises between 5 and 7 aspartate residues, inclusive, and the polypeptidyl group comprises between 1 and 4 proline residues, inclusive. In certain embodiments, the polypeptidyl group comprises between 4 and 6 aspartate residues, inclusive, and the polypeptidyl group comprises between 1 and 4 proline residues, inclusive. In certain embodiments, the polypeptidyl group comprises between 5 and 6 aspartate residues, inclusive, and the polypeptidyl group comprises between 1 and 4 proline residues, inclusive. In certain embodiments, the polypeptidyl group comprises 5 aspartate residues, and the polypeptidyl group comprises between 1 and 4 proline residues, inclusive. In certain embodiments, the polypeptidyl group comprises 6 aspartate residues, and the polypeptidyl group comprises between 1 and 4 proline residues, inclusive. In certain embodiments, the polypeptidyl group comprises between 3 and 7 aspartate residues, inclusive, and the polypeptidyl group comprises between 1 and 3 proline residues, inclusive. In certain embodiments, the polypeptidyl group comprises between 4 and 7 aspartate residues, inclusive, and the polypeptidyl group comprises between 1 and 3 proline residues, inclusive. In certain embodiments, the polypeptidyl group comprises between 5 and 7 aspartate residues, inclusive, and the polypeptidyl group comprises between 1 and 3 proline residues, inclusive. In certain embodiments, the polypeptidyl group comprises between 4 and 6 aspartate residues, inclusive, and the polypeptidyl group comprises between 1 and 3 proline residues, inclusive. In certain embodiments, the polypeptidyl group comprises between 5 and 6 aspartate residues, inclusive, and the polypeptidyl group comprises between 1 and 3 proline residues, inclusive. In certain embodiments, the polypeptidyl group comprises 5 aspartate residues, and the polypeptidyl group comprises between 1 and 3 proline residues, inclusive. In certain embodiments, the polypeptidyl group comprises 6 aspartate residues, and the polypeptidyl group comprises between 1 and 3 proline residues, inclusive. In certain embodiments, the polypeptidyl group comprises between 3 and 7 aspartate residues, inclusive, and the polypeptidyl group comprises between 1 and 2 proline residues, inclusive. In certain embodiments, the polypeptidyl group comprises between 4 and 7 aspartate residues, inclusive, and the polypeptidyl group comprises between 1 and 2 proline residues, inclusive. In certain embodiments, the polypeptidyl group comprises between 5 and 7 aspartate residues, inclusive, and the polypeptidyl group comprises between 1 and 2 proline residues, inclusive. In certain embodiments, the polypeptidyl group comprises between 4 and 6 aspartate residues, inclusive, and the polypeptidyl group comprises between 1 and 2 proline residues, inclusive. In certain embodiments, the polypeptidyl group comprises between 5 and 6 aspartate residues, inclusive, and the polypeptidyl group comprises between 1 and 2 proline residues, inclusive. In certain embodiments, the polypeptidyl group comprises 5 aspartate residues, and the polypeptidyl group comprises between 1 and 2 proline residues, inclusive. In certain embodiments, the polypeptidyl group comprises 6 aspartate residues, and the polypeptidyl group comprises between 1 and 2 proline residues, inclusive. In certain embodiments, the polypeptidyl group comprises between 3 and 7 aspartate residues, inclusive, and the polypeptidyl group comprises 1 proline residue. In certain embodiments, the polypeptidyl group comprises between 4 and 7 aspartate residues, inclusive, and the polypeptidyl group comprises 1 proline residue. In certain embodiments, the polypeptidyl group comprises between 5 and 7 aspartate residues, inclusive, and the polypeptidyl group comprises 1 proline residue. In certain embodiments, the polypeptidyl group comprises between 4 and 6 aspartate residues, inclusive, and the polypeptidyl group comprises 1 proline residue. In certain embodiments, the polypeptidyl group comprises between 5 and 6 aspartate residues, inclusive, and the polypeptidyl group comprises 1 proline residue. In certain embodiments, the polypeptidyl group comprises 5 aspartate residues, and the polypeptidyl group comprises 1 proline residue. In certain embodiments, the polypeptidyl group comprises 6 aspartate residues, and the polypeptidyl group comprises 1 proline residue. In certain embodiments, the polypeptidyl group comprises between 3 and 7 aspartate residues, inclusive, and the polypeptidyl group comprises 2 proline residues. In certain embodiments, the polypeptidyl group comprises between 4 and 7 aspartate residues, inclusive, and the polypeptidyl group comprises 2 proline residues. In certain embodiments, the polypeptidyl group comprises between 5 and 7 aspartate residues, inclusive, and the polypeptidyl group comprises 2 proline residues. In certain embodiments, the polypeptidyl group comprises between 4 and 6 aspartate residues, inclusive, and the polypeptidyl group comprises 2 proline residues. In certain embodiments, the polypeptidyl group comprises between 5 and 6 aspartate residues, inclusive, and the polypeptidyl group comprises 2 proline residues. In certain embodiments, the polypeptidyl group comprises 5 aspartate residues, and the polypeptidyl group comprises 2 proline residues. In certain embodiments, the polypeptidyl group comprises 6 aspartate residues, and the polypeptidyl group comprises 2 proline residues.

In certain embodiments, the polypeptidyl group comprises at least 1 GP repeat. In certain embodiments, the polypeptidyl group comprises at least 2 GP repeats. In certain embodiments, the polypeptidyl group comprises at least 3 GP repeats. In certain embodiments, the polypeptidyl group comprises at least 4 GP repeats. In certain embodiments, the polypeptidyl group comprises at least 5 GP repeats. In certain embodiments, the polypeptidyl group comprises between 1 and 5 GP repeats, inclusive. In certain embodiments, the polypeptidyl group comprises between 1 and 4 GP repeats, inclusive. In certain embodiments, the polypeptidyl group comprises between 1 and 3 GP repeats, inclusive. In certain embodiments, the polypeptidyl group comprises between 1 and 2 GP repeats, inclusive. In certain embodiments, the polypeptidyl group comprises 1 GP repeat. In certain embodiments, the polypeptidyl group comprises 2 GP repeats. In certain embodiments, the polypeptidyl group comprises 3 GP repeats. In certain embodiments, the polypeptidyl group comprises 4 GP repeats. In certain embodiments, the polypeptidyl group comprises 5 GP repeats.

In certain embodiments, the polypeptidyl group comprises between 1 and 3 GG repeats, inclusive. In certain embodiments, the polypeptidyl group comprises between 1 and 2 GG repeats, inclusive. In certain embodiments, the polypeptidyl group comprises 1 GG repeat. In certain embodiments, the polypeptidyl group comprises 2 GG repeats. In certain embodiments, the polypeptidyl group comprises 3 GG repeats.

In certain embodiments, the polypeptidyl group comprises between 1 and 3 GGG repeats, inclusive. In certain embodiments, the polypeptidyl group comprises between 1 and 2 GGG repeats, inclusive. In certain embodiments, the polypeptidyl group comprises 1 GGG repeat. In certain embodiments, the polypeptidyl group comprises 2 GGG repeats. In certain embodiments, the polypeptidyl group comprises 3 GGG repeats.

In certain embodiments, the polypeptidyl group comprises between 1 and 3 DD repeats, inclusive. In certain embodiments, the polypeptidyl group comprises between 1 and 2 DD repeats, inclusive. In certain embodiments, the polypeptidyl group comprises 1 DD repeat. In certain embodiments, the polypeptidyl group comprises 2 DD repeats. In certain embodiments, the polypeptidyl group comprises 3 DD repeats.

In certain embodiments, the polypeptidyl group comprises between 1 and 3 GG repeats, inclusive, and the polypeptidyl group comprises between 1 and 3 DD repeats, inclusive. In certain embodiments, the polypeptidyl group comprises between 1 and 2 GG repeats, inclusive, and the polypeptidyl group comprises between 1 and 3 DD repeats, inclusive. In certain embodiments, the polypeptidyl group comprises 1 GG repeat, and the polypeptidyl group comprises between 1 and 3 DD repeats, inclusive. In certain embodiments, the polypeptidyl group comprises 2 GG repeats, and the polypeptidyl group comprises between 1 and 3 DD repeats, inclusive. In certain embodiments, the polypeptidyl group comprises between 1 and 3 GG repeats, inclusive, and the polypeptidyl group comprises between 1 and 2 DD repeats, inclusive. In certain embodiments, the polypeptidyl group comprises between 1 and 2 GG repeats, inclusive, and the polypeptidyl group comprises between 1 and 2 DD repeats, inclusive. In certain embodiments, the polypeptidyl group comprises 1 GG repeat, and the polypeptidyl group comprises between 1 and 2 DD repeats, inclusive. In certain embodiments, the polypeptidyl group comprises 2 GG repeats, and the polypeptidyl group comprises between 1 and 2 DD repeats, inclusive. In certain embodiments, the polypeptidyl group comprises between 1 and 3 GG repeats, inclusive, and the polypeptidyl group comprises 1 DD repeat. In certain embodiments, the polypeptidyl group comprises between 1 and 2 GG repeats, inclusive, and the polypeptidyl group comprises 1 DD repeat. In certain embodiments, the polypeptidyl group comprises 1 GG repeat, and the polypeptidyl group comprises 1 DD repeat. In certain embodiments, the polypeptidyl group comprises 2 GG repeats, and the polypeptidyl group comprises 1 DD repeat. In certain embodiments, the polypeptidyl group comprises between 1 and 3 GG repeats, inclusive, and the polypeptidyl group comprises 2 DD repeats. In certain embodiments, the polypeptidyl group comprises between 1 and 2 GG repeats, inclusive, and the polypeptidyl group comprises 2 DD repeats. In certain embodiments, the polypeptidyl group comprises 1 GG repeat, and the polypeptidyl group comprises 2 DD repeats. In certain embodiments, the polypeptidyl group comprises 2 GG repeats, and the polypeptidyl group comprises 2 DD repeats. In certain embodiments, the polypeptidyl group comprises between 1 and 3 GG repeats, inclusive, and the polypeptidyl group comprises 3 DD repeats. In certain embodiments, the polypeptidyl group comprises between 1 and 2 GG repeats, inclusive, and the polypeptidyl group comprises 3 DD repeats. In certain embodiments, the polypeptidyl group comprises 1 GG repeat, and the polypeptidyl group comprises 3 DD repeats. In certain embodiments, the polypeptidyl group comprises 2 GG repeats, and the polypeptidyl group comprises 3 DD repeats.

In certain embodiments, the polypeptidyl group comprises between 1 and 3 GGG repeats, inclusive, and the polypeptidyl group comprises between 1 and 3 DD repeats, inclusive. In certain embodiments, the polypeptidyl group comprises between 1 and 2 GGG repeats, inclusive, and the polypeptidyl group comprises between 1 and 3 DD repeats, inclusive. In certain embodiments, the polypeptidyl group comprises 1 GGG repeat, and the polypeptidyl group comprises between 1 and 3 DD repeats, inclusive. In certain embodiments, the polypeptidyl group comprises 2 GGG repeats, and the polypeptidyl group comprises between 1 and 3 DD repeats, inclusive. In certain embodiments, the polypeptidyl group comprises between 1 and 3 GGG repeats, inclusive, and the polypeptidyl group comprises between 1 and 2 DD repeats, inclusive. In certain embodiments, the polypeptidyl group comprises between 1 and 2 GGG repeats, inclusive, and the polypeptidyl group comprises between 1 and 2 DD repeats, inclusive. In certain embodiments, the polypeptidyl group comprises 1 GGG repeat, and the polypeptidyl group comprises between 1 and 2 DD repeats, inclusive. In certain embodiments, the polypeptidyl group comprises 2 GGG repeats, and the polypeptidyl group comprises between 1 and 2 DD repeats, inclusive. In certain embodiments, the polypeptidyl group comprises between 1 and 3 GGG repeats, inclusive, and the polypeptidyl group comprises 1 DD repeat. In certain embodiments, the polypeptidyl group comprises between 1 and 2 GGG repeats, inclusive, and the polypeptidyl group comprises 1 DD repeat. In certain embodiments, the polypeptidyl group comprises 1 GGG repeat, and the polypeptidyl group comprises 1 DD repeat. In certain embodiments, the polypeptidyl group comprises 2 GGG repeats, and the polypeptidyl group comprises 1 DD repeat. In certain embodiments, the polypeptidyl group comprises between 1 and 3 GGG repeats, inclusive, and the polypeptidyl group comprises 2 DD repeats. In certain embodiments, the polypeptidyl group comprises between 1 and 2 GGG repeats, inclusive, and the polypeptidyl group comprises 2 DD repeats. In certain embodiments, the polypeptidyl group comprises 1 GGG repeat, and the polypeptidyl group comprises 2 DD repeats. In certain embodiments, the polypeptidyl group comprises 2 GGG repeats, and the polypeptidyl group comprises 2 DD repeats. In certain embodiments, the polypeptidyl group comprises between 1 and 3 GGG repeats, inclusive, and the polypeptidyl group comprises 3 DD repeats. In certain embodiments, the polypeptidyl group comprises between 1 and 2 GGG repeats, inclusive, and the polypeptidyl group comprises 3 DD repeats. In certain embodiments, the polypeptidyl group comprises 1 GGG repeat, and the polypeptidyl group comprises 3 DD repeats. In certain embodiments, the polypeptidyl group comprises 2 GGG repeats, and the polypeptidyl group comprises 3 DD repeats.

In certain embodiments, the polypeptidyl group comprises between 1 and 3 DDD repeats, inclusive. In certain embodiments, the polypeptidyl group comprises between 1 and 2 DDD repeats, inclusive. In certain embodiments, the polypeptidyl group comprises 1 DDD repeat. In certain embodiments, the polypeptidyl group comprises 2 DDD repeats. In certain embodiments, the polypeptidyl group comprises 3 DDD repeats.

In certain embodiments, the polypeptidyl group comprises between 1 and 3 GG repeats, inclusive, and the polypeptidyl group comprises between 1 and 3 DDD repeats, inclusive. In certain embodiments, the polypeptidyl group comprises between 1 and 2 GG repeats, inclusive, and the polypeptidyl group comprises between 1 and 3 DDD repeats, inclusive. In certain embodiments, the polypeptidyl group comprises 1 GG repeat, and the polypeptidyl group comprises between 1 and 3 DDD repeats, inclusive. In certain embodiments, the polypeptidyl group comprises 2 GG repeats, and the polypeptidyl group comprises between 1 and 3 DDD repeats, inclusive. In certain embodiments, the polypeptidyl group comprises between 1 and 3 GG repeats, inclusive, and the polypeptidyl group comprises between 1 and 2 DDD repeats, inclusive. In certain embodiments, the polypeptidyl group comprises between 1 and 2 GG repeats, inclusive, and the polypeptidyl group comprises between 1 and 2 DDD repeats, inclusive. In certain embodiments, the polypeptidyl group comprises 1 GG repeat, and the polypeptidyl group comprises between 1 and 2 DDD repeats, inclusive. In certain embodiments, the polypeptidyl group comprises 2 GG repeats, and the polypeptidyl group comprises between 1 and 2 DDD repeats, inclusive. In certain embodiments, the polypeptidyl group comprises between 1 and 3 GG repeats, inclusive, and the polypeptidyl group comprises 1 DDD repeat. In certain embodiments, the polypeptidyl group comprises between 1 and 2 GG repeats, inclusive, and the polypeptidyl group comprises 1 DDD repeat. In certain embodiments, the polypeptidyl group comprises 1 GG repeat, and the polypeptidyl group comprises 1 DDD repeat. In certain embodiments, the polypeptidyl group comprises 2 GG repeats, and the polypeptidyl group comprises 1 DDD repeat. In certain embodiments, the polypeptidyl group comprises between 1 and 3 GG repeats, inclusive, and the polypeptidyl group comprises 2 DDD repeats. In certain embodiments, the polypeptidyl group comprises between 1 and 2 GG repeats, inclusive, and the polypeptidyl group comprises 2 DDD repeats. In certain embodiments, the polypeptidyl group comprises 1 GG repeat, and the polypeptidyl group comprises 2 DDD repeats. In certain embodiments, the polypeptidyl group comprises 2 GG repeats, and the polypeptidyl group comprises 2 DDD repeats. In certain embodiments, the polypeptidyl group comprises between 1 and 3 GG repeats, inclusive, and the polypeptidyl group comprises 3 DDD repeats. In certain embodiments, the polypeptidyl group comprises between 1 and 2 GG repeats, inclusive, and the polypeptidyl group comprises 3 DDD repeats. In certain embodiments, the polypeptidyl group comprises 1 GG repeat, and the polypeptidyl group comprises 3 DDD repeats. In certain embodiments, the polypeptidyl group comprises 2 GG repeats, and the polypeptidyl group comprises 3 DDD repeats.

In certain embodiments, the polypeptidyl group comprises between 1 and 3 GGG repeats, inclusive, and the polypeptidyl group comprises between 1 and 3 DDD repeats, inclusive. In certain embodiments, the polypeptidyl group comprises between 1 and 2 GGG repeats, inclusive, and the polypeptidyl group comprises between 1 and 3 DDD repeats, inclusive. In certain embodiments, the polypeptidyl group comprises 1 GGG repeat, and the polypeptidyl group comprises between 1 and 3 DDD repeats, inclusive. In certain embodiments, the polypeptidyl group comprises 2 GGG repeats, and the polypeptidyl group comprises between 1 and 3 DDD repeats, inclusive. In certain embodiments, the polypeptidyl group comprises between 1 and 3 GGG repeats, inclusive, and the polypeptidyl group comprises between 1 and 2 DDD repeats, inclusive. In certain embodiments, the polypeptidyl group comprises between 1 and 2 GGG repeats, inclusive, and the polypeptidyl group comprises between 1 and 2 DDD repeats, inclusive. In certain embodiments, the polypeptidyl group comprises 1 GGG repeat, and the polypeptidyl group comprises between 1 and 2 DDD repeats, inclusive. In certain embodiments, the polypeptidyl group comprises 2 GGG repeats, and the polypeptidyl group comprises between 1 and 2 DDD repeats, inclusive. In certain embodiments, the polypeptidyl group comprises between 1 and 3 GGG repeats, inclusive, and the polypeptidyl group comprises 1 DDD repeat. In certain embodiments, the polypeptidyl group comprises between 1 and 2 GGG repeats, inclusive, and the polypeptidyl group comprises 1 DDD repeat. In certain embodiments, the polypeptidyl group comprises 1 GGG repeat, and the polypeptidyl group comprises 1 DDD repeat. In certain embodiments, the polypeptidyl group comprises 2 GGG repeats, and the polypeptidyl group comprises 1 DDD repeat. In certain embodiments, the polypeptidyl group comprises between 1 and 3 GGG repeats, inclusive, and the polypeptidyl group comprises 2 DDD repeats. In certain embodiments, the polypeptidyl group comprises between 1 and 2 GGG repeats, inclusive, and the polypeptidyl group comprises 2 DDD repeats. In certain embodiments, the polypeptidyl group comprises 1 GGG repeat, and the polypeptidyl group comprises 2 DDD repeats. In certain embodiments, the polypeptidyl group comprises 2 GGG repeats, and the polypeptidyl group comprises 2 DDD repeats. In certain embodiments, the polypeptidyl group comprises between 1 and 3 GGG repeats, inclusive, and the polypeptidyl group comprises 3 DDD repeats. In certain embodiments, the polypeptidyl group comprises between 1 and 2 GGG repeats, inclusive, and the polypeptidyl group comprises 3 DDD repeats. In certain embodiments, the polypeptidyl group comprises 1 GGG repeat, and the polypeptidyl group comprises 3 DDD repeats. In certain embodiments, the polypeptidyl group comprises 2 GGG repeats, and the polypeptidyl group comprises 3 DDD repeats.

In certain embodiments, the polypeptidyl group comprises between 1 and 3 FF repeats, inclusive. In certain embodiments, the polypeptidyl group comprises between 1 and 2 FF repeats, inclusive. In certain embodiments, the polypeptidyl group comprises 1 FF repeat. In certain embodiments, the polypeptidyl group comprises 2 FF repeats. In certain embodiments, the polypeptidyl group comprises 3 FF repeats.

In certain embodiments, the polypeptidyl group comprises between 1 and 3 GG repeats, inclusive, and the polypeptidyl group comprises between 1 and 3 FF repeats, inclusive. In certain embodiments, the polypeptidyl group comprises between 1 and 2 GG repeats, inclusive, and the polypeptidyl group comprises between 1 and 3 FF repeats, inclusive. In certain embodiments, the polypeptidyl group comprises 1 GG repeat, and the polypeptidyl group comprises between 1 and 3 FF repeats, inclusive. In certain embodiments, the polypeptidyl group comprises 2 GG repeats, and the polypeptidyl group comprises between 1 and 3 FF repeats, inclusive. In certain embodiments, the polypeptidyl group comprises between 1 and 3 GG repeats, inclusive, and the polypeptidyl group comprises between 1 and 2 FF repeats, inclusive. In certain embodiments, the polypeptidyl group comprises between 1 and 2 GG repeats, inclusive, and the polypeptidyl group comprises between 1 and 2 FF repeats, inclusive. In certain embodiments, the polypeptidyl group comprises 1 GG repeat, and the polypeptidyl group comprises between 1 and 2 FF repeats, inclusive. In certain embodiments, the polypeptidyl group comprises 2 GG repeats, and the polypeptidyl group comprises between 1 and 2 FF repeats, inclusive. In certain embodiments, the polypeptidyl group comprises between 1 and 3 GG repeats, inclusive, and the polypeptidyl group comprises 1 FF repeat. In certain embodiments, the polypeptidyl group comprises between 1 and 2 GG repeats, inclusive, and the polypeptidyl group comprises 1 FF repeat. In certain embodiments, the polypeptidyl group comprises 1 GG repeat, and the polypeptidyl group comprises 1 FF repeat. In certain embodiments, the polypeptidyl group comprises 2 GG repeats, and the polypeptidyl group comprises 1 FF repeat. In certain embodiments, the polypeptidyl group comprises between 1 and 3 GG repeats, inclusive, and the polypeptidyl group comprises 2 FF repeats. In certain embodiments, the polypeptidyl group comprises between 1 and 2 GG repeats, inclusive, and the polypeptidyl group comprises 2 FF repeats. In certain embodiments, the polypeptidyl group comprises 1 GG repeat, and the polypeptidyl group comprises 2 FF repeats. In certain embodiments, the polypeptidyl group comprises 2 GG repeats, and the polypeptidyl group comprises 2 FF repeats.

In certain embodiments, the polypeptidyl group comprises between 1 and 3 GGG repeats, inclusive, and the polypeptidyl group comprises between 1 and 3 FF repeats, inclusive. In certain embodiments, the polypeptidyl group comprises between 1 and 2 GGG repeats, inclusive, and the polypeptidyl group comprises between 1 and 3 FF repeats, inclusive. In certain embodiments, the polypeptidyl group comprises 1 GGG repeat, and the polypeptidyl group comprises between 1 and 3 FF repeats, inclusive. In certain embodiments, the polypeptidyl group comprises 2 GGG repeats, and the polypeptidyl group comprises between 1 and 3 FF repeats, inclusive. In certain embodiments, the polypeptidyl group comprises between 1 and 3 GGG repeats, inclusive, and the polypeptidyl group comprises between 1 and 2 FF repeats, inclusive. In certain embodiments, the polypeptidyl group comprises between 1 and 2 GGG repeats, inclusive, and the polypeptidyl group comprises between 1 and 2 FF repeats, inclusive. In certain embodiments, the polypeptidyl group comprises 1 GGG repeat, and the polypeptidyl group comprises between 1 and 2 FF repeats, inclusive. In certain embodiments, the polypeptidyl group comprises 2 GGG repeats, and the polypeptidyl group comprises between 1 and 2 FF repeats, inclusive. In certain embodiments, the polypeptidyl group comprises between 1 and 3 GGG repeats, inclusive, and the polypeptidyl group comprises 1 FF repeat. In certain embodiments, the polypeptidyl group comprises between 1 and 2 GGG repeats, inclusive, and the polypeptidyl group comprises 1 FF repeat. In certain embodiments, the polypeptidyl group comprises 1 GGG repeat, and the polypeptidyl group comprises 1 FF repeat. In certain embodiments, the polypeptidyl group comprises 2 GGG repeats, and the polypeptidyl group comprises 1 FF repeat. In certain embodiments, the polypeptidyl group comprises between 1 and 3 GGG repeats, inclusive, and the polypeptidyl group comprises 2 FF repeats. In certain embodiments, the polypeptidyl group comprises between 1 and 2 GGG repeats, inclusive, and the polypeptidyl group comprises 2 FF repeats. In certain embodiments, the polypeptidyl group comprises 1 GGG repeat, and the polypeptidyl group comprises 2 FF repeats. In certain embodiments, the polypeptidyl group comprises 2 GGG repeats, and the polypeptidyl group comprises 2 FF repeats.

In certain embodiments, the polypeptidyl group comprises between 1 and 3 FF repeats, inclusive, and the polypeptidyl group comprises between 1 and 3 DD repeats, inclusive. In certain embodiments, the polypeptidyl group comprises between 1 and 2 FF repeats, inclusive, and the polypeptidyl group comprises between 1 and 3 DD repeats, inclusive. In certain embodiments, the polypeptidyl group comprises 1 FF repeat, and the polypeptidyl group comprises between 1 and 3 DD repeats, inclusive. In certain embodiments, the polypeptidyl group comprises 2 FF repeats, and the polypeptidyl group comprises between 1 and 3 DD repeats, inclusive. In certain embodiments, the polypeptidyl group comprises between 1 and 3 FF repeats, inclusive, and the polypeptidyl group comprises between 1 and 2 DD repeats, inclusive. In certain embodiments, the polypeptidyl group comprises between 1 and 2 FF repeats, inclusive, and the polypeptidyl group comprises between 1 and 2 DD repeats, inclusive. In certain embodiments, the polypeptidyl group comprises 1 FF repeat, and the polypeptidyl group comprises between 1 and 2 DD repeats, inclusive. In certain embodiments, the polypeptidyl group comprises 2 FF repeats, and the polypeptidyl group comprises between 1 and 2 DD repeats, inclusive. In certain embodiments, the polypeptidyl group comprises between 1 and 3 FF repeats, inclusive, and the polypeptidyl group comprises 1 DD repeat. In certain embodiments, the polypeptidyl group comprises between 1 and 2 FF repeats, inclusive, and the polypeptidyl group comprises 1 DD repeat. In certain embodiments, the polypeptidyl group comprises 1 FF repeat, and the polypeptidyl group comprises 1 DD repeat. In certain embodiments, the polypeptidyl group comprises 2 FF repeats, and the polypeptidyl group comprises 1 DD repeat. In certain embodiments, the polypeptidyl group comprises between 1 and 3 FF repeats, inclusive, and the polypeptidyl group comprises 2 DD repeats. In certain embodiments, the polypeptidyl group comprises between 1 and 2 FF repeats, inclusive, and the polypeptidyl group comprises 2 DD repeats. In certain embodiments, the polypeptidyl group comprises 1 FF repeat, and the polypeptidyl group comprises 2 DD repeats. In certain embodiments, the polypeptidyl group comprises 2 FF repeats, and the polypeptidyl group comprises 2 DD repeats. In certain embodiments, the polypeptidyl group comprises between 1 and 3 FF repeats, inclusive, and the polypeptidyl group comprises 3 DD repeats. In certain embodiments, the polypeptidyl group comprises between 1 and 2 FF repeats, inclusive, and the polypeptidyl group comprises 3 DD repeats. In certain embodiments, the polypeptidyl group comprises 1 FF repeat, and the polypeptidyl group comprises 3 DD repeats. In certain embodiments, the polypeptidyl group comprises 2 FF repeats, and the polypeptidyl group comprises 3 DD repeats.

In certain embodiments, the polypeptidyl group comprises between 1 and 3 FF repeats, inclusive, and the polypeptidyl group comprises between 1 and 3 DDD repeats, inclusive. In certain embodiments, the polypeptidyl group comprises between 1 and 2 FF repeats, inclusive, and the polypeptidyl group comprises between 1 and 3 DDD repeats, inclusive. In certain embodiments, the polypeptidyl group comprises 1 FF repeat, and the polypeptidyl group comprises between 1 and 3 DDD repeats, inclusive. In certain embodiments, the polypeptidyl group comprises 2 FF repeats, and the polypeptidyl group comprises between 1 and 3 DDD repeats, inclusive. In certain embodiments, the polypeptidyl group comprises between 1 and 3 FF repeats, inclusive, and the polypeptidyl group comprises between 1 and 2 DDD repeats, inclusive. In certain embodiments, the polypeptidyl group comprises between 1 and 2 FF repeats, inclusive, and the polypeptidyl group comprises between 1 and 2 DDD repeats, inclusive. In certain embodiments, the polypeptidyl group comprises 1 FF repeat, and the polypeptidyl group comprises between 1 and 2 DDD repeats, inclusive. In certain embodiments, the polypeptidyl group comprises 2 FF repeats, and the polypeptidyl group comprises between 1 and 2 DDD repeats, inclusive. In certain embodiments, the polypeptidyl group comprises between 1 and 3 FF repeats, inclusive, and the polypeptidyl group comprises 1 DDD repeat. In certain embodiments, the polypeptidyl group comprises between 1 and 2 FF repeats, inclusive, and the polypeptidyl group comprises 1 DDD repeat. In certain embodiments, the polypeptidyl group comprises 1 FF repeat, and the polypeptidyl group comprises 1 DDD repeat. In certain embodiments, the polypeptidyl group comprises 2 FF repeats, and the polypeptidyl group comprises 1 DDD repeat. In certain embodiments, the polypeptidyl group comprises between 1 and 3 FF repeats, inclusive, and the polypeptidyl group comprises 2 DDD repeats. In certain embodiments, the polypeptidyl group comprises between 1 and 2 FF repeats, inclusive, and the polypeptidyl group comprises 2 DDD repeats. In certain embodiments, the polypeptidyl group comprises 1 FF repeat, and the polypeptidyl group comprises 2 DDD repeats. In certain embodiments, the polypeptidyl group comprises 2 FF repeats, and the polypeptidyl group comprises 2 DDD repeats. In certain embodiments, the polypeptidyl group comprises between 1 and 3 FF repeats, inclusive, and the polypeptidyl group comprises 3 DDD repeats. In certain embodiments, the polypeptidyl group comprises between 1 and 2 FF repeats, inclusive, and the polypeptidyl group comprises 3 DDD repeats. In certain embodiments, the polypeptidyl group comprises 1 FF repeat, and the polypeptidyl group comprises 3 DDD repeats. In certain embodiments, the polypeptidyl group comprises 2 FF repeats, and the polypeptidyl group comprises 3 DDD repeats.

In certain embodiments, the oligonucleotide and the polypeptide are separated by at least 30 Å. In certain embodiments, the oligonucleotide and the polypeptide are separated by at least 33 Å. In certain embodiments, the oligonucleotide and the polypeptide are separated by at least 35 Å. In certain embodiments, the oligonucleotide and the polypeptide are separated by between about 25 Å and about 50 Å, inclusive. In certain embodiments, the oligonucleotide and the polypeptide are separated by between about 25 Å and about 45 Å, inclusive. In certain embodiments, the oligonucleotide and the polypeptide are separated by between about 25 Å and about 40 Å, inclusive. In certain embodiments, the oligonucleotide and the polypeptide are separated by between about 25 Å and about 35 Å, inclusive. In certain embodiments, the oligonucleotide and the polypeptide are separated by between about 30 Å and about 35 Å, inclusive. In certain embodiments, the oligonucleotide and the polypeptide are separated by about 33 Å.

In certain embodiments, the polypeptidyl group comprises

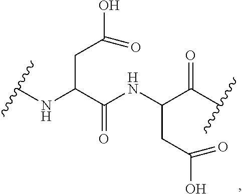 [SMILES: *NC(CC(=O)O)C(=O)NC(CC(=O)O)C(*)=O]

or a salt thereof. In certain embodiments, the polypeptidyl group comprises

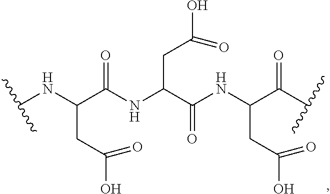 [SMILES: *NC(CC(=O)O)C(=O)NC(CC(=O)O)C(=O)NC(CC(=O)O)C(*)=O]

or a salt thereof. In certain embodiments, the polypeptidyl group comprises

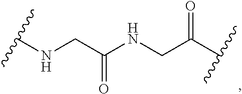 [SMILES: *NCC(=O)NCC(*)=O]

or a salt thereof. In certain embodiments, the polypeptidyl group comprises

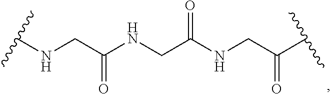 [SMILES: *NCC(=O)NCC(=O)NCC(*)=O]

or a salt thereof. In certain embodiments, the polypeptidyl group comprises

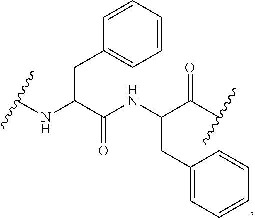 [SMILES: *NC(Cc1ccccc1)C(=O)NC(Cc1ccccc1)C(*)=O]

or a salt thereof. In certain embodiments, the polypeptidyl group comprises

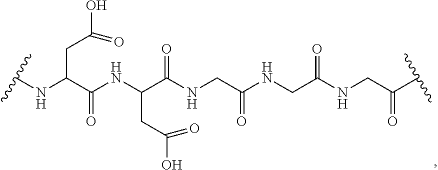 [SMILES: *NC(CC(=O)O)C(=O)NC(CC(=O)O)C(=O)NCC(=O)NCC(=O)NCC(*)=O]

or a salt thereof. In certain embodiments, the polypeptidyl group comprises

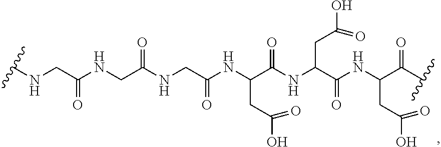 [SMILES: *NCC(=O)NCC(=O)NCC(=O)NC(CC(=O)O)C(=O)NC(CC(=O)O)C(=O)NC(CC(=O)O)C(*)=O]

or a salt thereof. In certain embodiments, the polypeptidyl group comprises

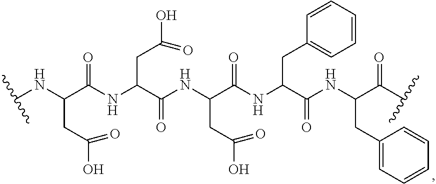 [SMILES: *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof.

In certain embodiments, the polypeptidyl group comprises

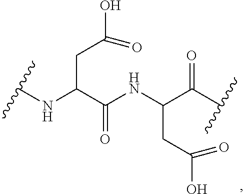 [SMILES: *NC(CC(=O)O)C(=O)NC(CC(=O)O)C(*)=O]

or a salt thereof, and

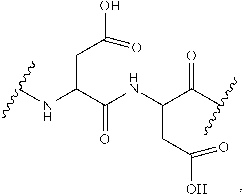 [SMILES: *NC(CC(=O)O)C(=O)NC(CC(=O)O)C(*)=O]

or a salt thereof. In certain embodiments, the polypeptidyl group comprises

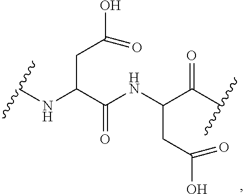 [SMILES: *NC(CC(=O)O)C(=O)NC(CC(=O)O)C(*)=O]

OH or a salt thereof, and

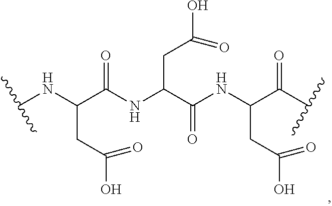 [SMILES: *NC(CC(=O)O)C(=O)NC(CC(=O)O)C(=O)NC(CC(=O)O)C(*)=O]

or a salt thereof. In certain embodiments, the polypeptidyl group comprises

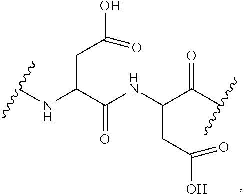 [SMILES: *NC(CC(=O)O)C(=O)NC(CC(=O)O)C(*)=O]

or a salt thereof, and

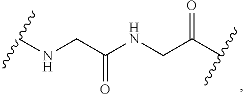 [SMILES: *NCC(=O)NCC(*)=O]

or a salt thereof. In certain embodiments, the polypeptidyl group comprises

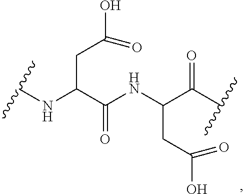 [SMILES: *NC(CC(=O)O)C(=O)NC(CC(=O)O)C(*)=O]

or a salt thereof, and

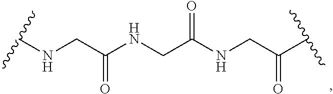 [SMILES: *NCC(=O)NCC(=O)NCC(*)=O]

or a salt thereof. In certain embodiments, the polypeptidyl group comprises

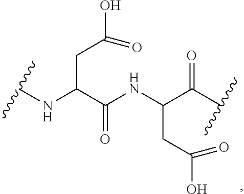 [SMILES: *NC(CC(=O)O)C(=O)NC(CC(=O)O)C(*)=O]

or a salt thereof, and

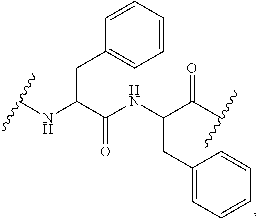 [SMILES: *NC(Cc1ccccc1)C(=O)NC(Cc1ccccc1)C(*)=O]

or a salt thereof. In certain embodiments, the polypeptidyl group comprises

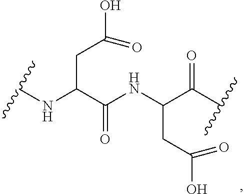 [SMILES: *NC(CC(=O)O)C(=O)NC(CC(=O)O)C(*)=O]

or a salt thereof, and

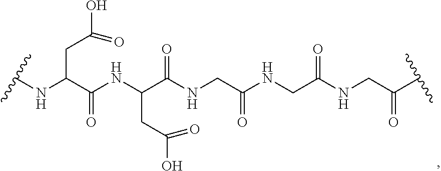 [SMILES: *NC(CC(=O)O)C(=O)NC(CC(=O)O)C(=O)NCC(=O)NCC(=O)NCC(*)=O]

or a salt thereof. In certain embodiments, the polypeptidyl group comprises

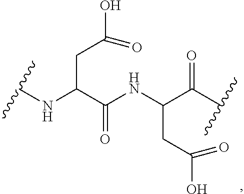 [SMILES: *NC(CC(=O)O)C(=O)NC(CC(=O)O)C(*)=O]

or a salt thereof, and

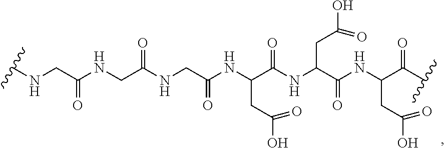 [SMILES: *NCC(=O)NCC(=O)NCC(=O)NC(CC(=O)O)C(=O)NC(CC(=O)O)C(=O)NC(CC(=O)O)C(*)=O]

or a salt thereof. In certain embodiments, the polypeptidyl group comprises

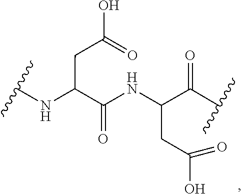 [SMILES: *NC(CC(=O)O)C(=O)NC(CC(=O)O)C(*)=O]

or a salt thereof, and

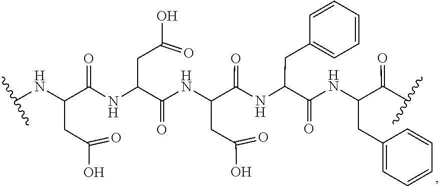 [SMILES: *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof. In certain embodiments, the polypeptidyl group comprises

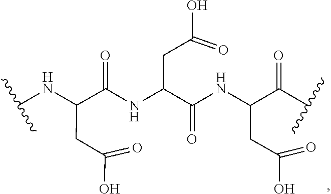 [SMILES: *NC(CC(=O)O)C(=O)NC(CC(=O)O)C(=O)NC(CC(=O)O)C(*)=O]

or a salt thereof, and

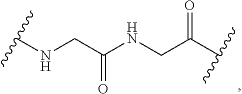 [SMILES: *NCC(=O)NCC(*)=O]

or a salt thereof. In certain embodiments, the polypeptidyl group comprises

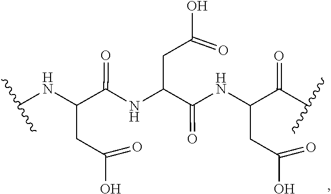 [SMILES: *NC(CC(=O)O)C(=O)NC(CC(=O)O)C(=O)NC(CC(=O)O)C(*)=O]

or a salt thereof, and

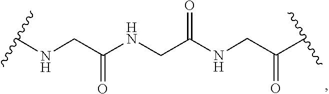 [SMILES: *NCC(=O)NCC(=O)NCC(*)=O]

or a salt thereof. In certain embodiments, the polypeptidyl group comprises

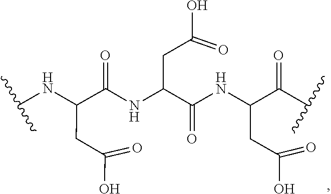 [SMILES: *NC(CC(=O)O)C(=O)NC(CC(=O)O)C(=O)NC(CC(=O)O)C(*)=O]

or a salt thereof, and

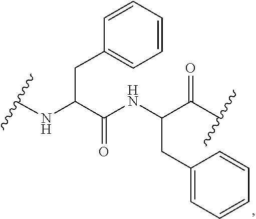 [SMILES: *NC(Cc1ccccc1)C(=O)NC(Cc1ccccc1)C(*)=O]

or a salt thereof. In certain embodiments, the polypeptidyl group comprises

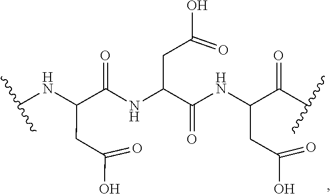 [SMILES: *NC(CC(=O)O)C(=O)NC(CC(=O)O)C(=O)NC(CC(=O)O)C(*)=O]

or a salt thereof, and

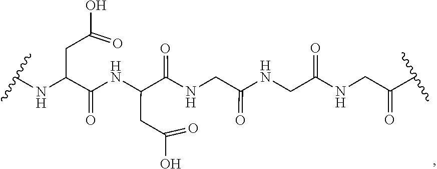 [SMILES: *NC(CC(=O)O)C(=O)NC(CC(=O)O)C(=O)NCC(=O)NCC(=O)NCC(*)=O]

or a salt thereof. In certain embodiments, the polypeptidyl group comprises

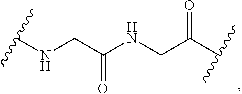 [SMILES: *NCC(=O)NCC(*)=O]

or a salt thereof, and

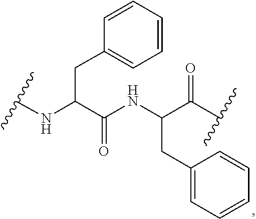 [SMILES: *NC(Cc1ccccc1)C(=O)NC(Cc1ccccc1)C(*)=O]

or a salt thereof. In certain embodiments, the polypeptidyl group comprises

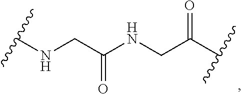 [SMILES: *NCC(=O)NCC(*)=O]

or a salt thereof, and

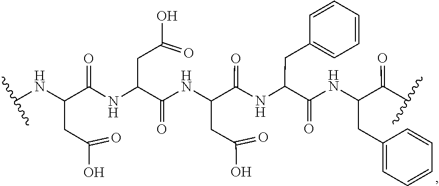 [SMILES: *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof. In certain embodiments, the polypeptidyl group comprises

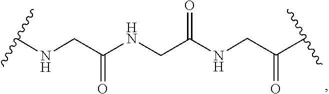 [SMILES: *NCC(=O)NCC(=O)NCC(*)=O]

or a salt thereof, and

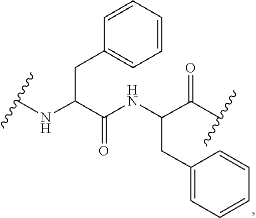 [SMILES: *NC(Cc1ccccc1)C(=O)NC(Cc1ccccc1)C(*)=O]

or a salt thereof. In certain embodiments, the polypeptidyl group comprises

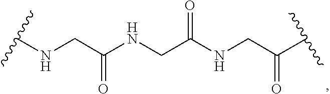 [SMILES: *NCC(=O)NCC(=O)NCC(*)=O]

or a salt thereof, and

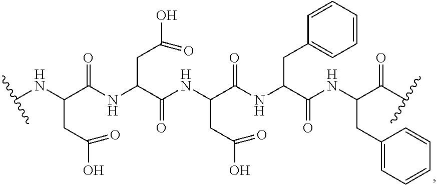 [SMILES: *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof. In certain embodiments, the polypeptidyl group comprises

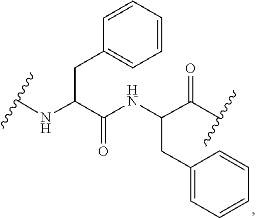 [SMILES: *NC(Cc1ccccc1)C(=O)NC(Cc1ccccc1)C(*)=O]

or a salt thereof, and

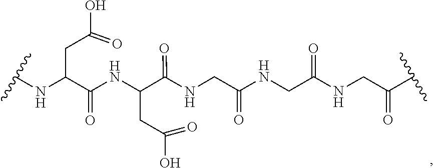 [SMILES: *NC(CC(=O)O)C(=O)NC(CC(=O)O)C(=O)NCC(=O)NCC(=O)NCC(*)=O]

or a salt thereof. In certain embodiments, the polypeptidyl group comprises

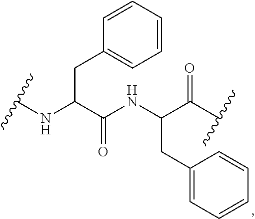 [SMILES: *NC(Cc1ccccc1)C(=O)NC(Cc1ccccc1)C(*)=O]

or a salt thereof, and

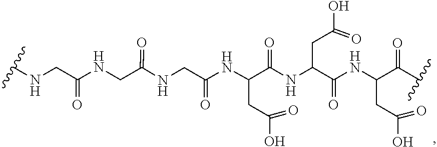 [SMILES: *NCC(=O)NCC(=O)NCC(=O)NC(CC(=O)O)C(=O)NC(CC(=O)O)C(=O)NC(CC(=O)O)C(*)=O]

or a salt thereof. In certain embodiments, the polypeptidyl group comprises

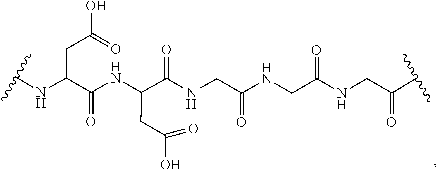 [SMILES: *NC(CC(=O)O)C(=O)NC(CC(=O)O)C(=O)NCC(=O)NCC(=O)NCC(*)=O]

or a salt thereof, and

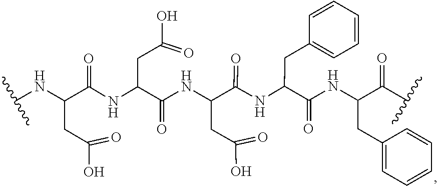 [SMILES: *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof.

In certain embodiments, the polypeptidyl group comprises

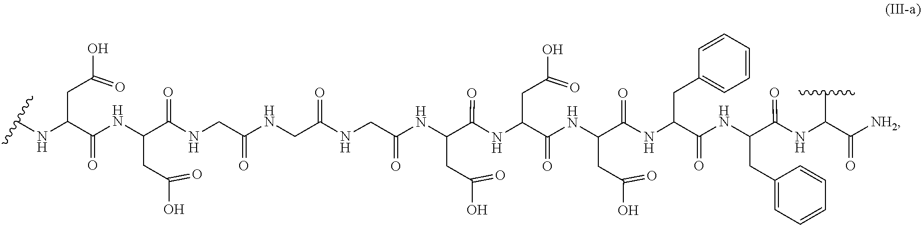 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain-embodiments, the polypeptidyl group comprises

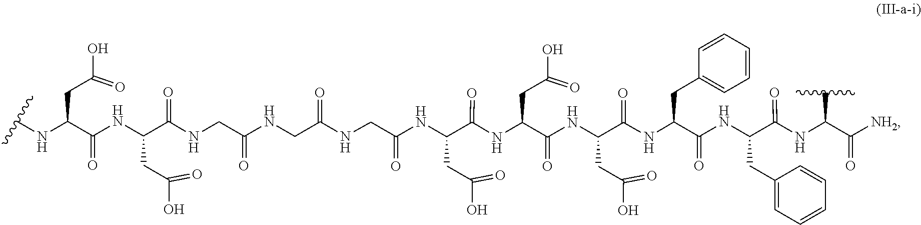 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof. In certain embodiments, the polypeptidyl group comprises

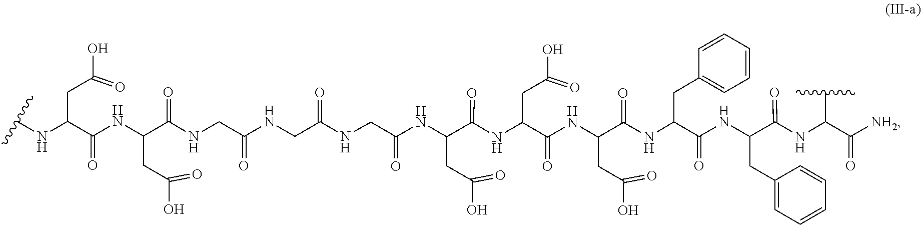 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, the polypeptidyl group comprises

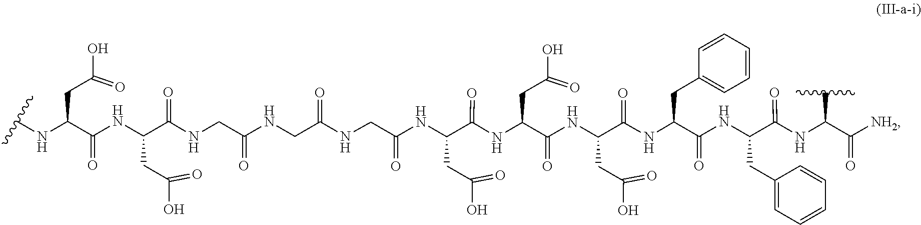 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof.

In certain embodiments, the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), or a salt thereof, wherein Cy is cysteic acid. In certain embodiments, the polypeptidyl group comprises a sequence DDGGGDDDFF (SEQ ID NO: 32), or a salt thereof.

In certain embodiments, L further comprises at least one of optionally substituted alkylene, optionally substituted alkenylene, optionally substituted alkynylene, optionally substituted heteroalkylene, optionally substituted heteroalkenylene, optionally substituted heteroalkynylene, optionally substituted heterocyclylene, optionally substituted carbocyclylene, optionally substituted arylene, optionally substituted heteroarylene, a peptidyl group, a dipeptidyl group, a polypeptidyl group, a click chemistry handle, or a combination thereof.

In certain embodiments, L further comprises optionally substituted C₁₋₆ alkylene. In certain embodiments, L further comprises substituted C₁₋₆ alkylene substituted with two oxo groups. In certain embodiments, L further comprises unsubstituted n-butylene. In certain embodiments, L further comprises substituted n-butylene. In certain embodiments, L further comprises substituted n-butylene substituted with one or more oxo groups. In certain embodiments, L further comprises substituted n-butylene substituted with one oxo group. In certain embodiments, L further comprises substituted n-butylene substituted with two oxo groups. In certain embodiments, L further comprises

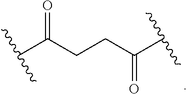 [SMILES: *C(=O)CCC(*)=O]

In certain embodiments, L further comprises optionally substituted 5-14 membered heteroarylene. In certain embodiments, L further comprises optionally substituted triazolylene. In certain embodiments, L further comprises

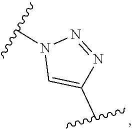 [SMILES: *c1cn(*)nn1]

or a salt thereof. In certain embodiments, L further comprises

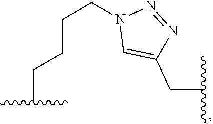 [SMILES: *CCCCn1cc(C*)nn1]

or a salt thereof.

In certain embodiments, L further comprises a click chemistry handle. In certain embodiments, the click chemistry handle comprises an alkyne. In certain embodiments, the click chemistry handle comprises a strained alkyne. In certain embodiments, the click chemistry handle comprises an optionally substituted cyclooctyne. In certain embodiments, the click chemistry handle comprises a substituted cyclooctyne.

In certain embodiments, the click chemistry handle is of Formula (IV) or Formula (V):

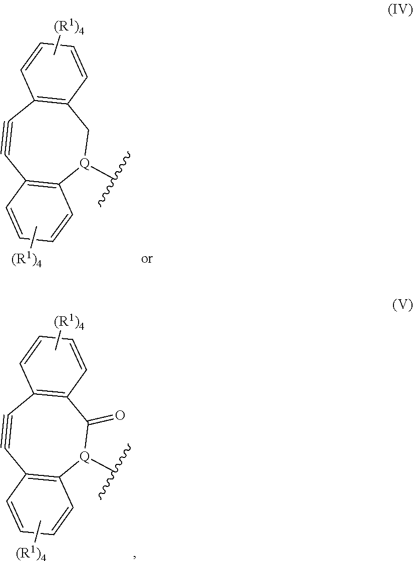

or a salt thereof, wherein:
each instance of R¹ is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
Q is CH or N.

In certain embodiments, at least one instance of R¹ is hydrogen. In certain embodiments, all instances of R¹ are hydrogen. In certain embodiments, Q is CH. In certain embodiments, Q is N. In certain embodiments, at least one instance of R¹ is hydrogen, Q is N. In certain embodiments, all instances of R¹ are hydrogen, and Q is N. In certain embodiments, the click chemistry handle is of formula

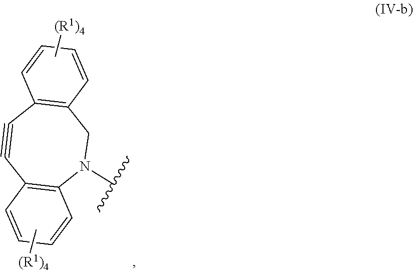

or a salt thereof. In certain embodiments, the click chemistry handle is of formula

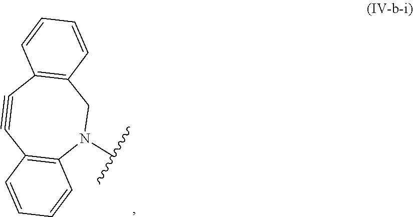 [SMILES: *N1Cc2ccccc2C#Cc2ccccc21]

or a salt thereof.

In certain embodiments, L comprises a click chemistry handle of Formula (IV) or Formula (V) and optionally substituted C₁₋₆ alkylene. In certain embodiments, L comprises a click chemistry handle of Formula (IV) or Formula (V) and substituted C₁₋₆ alkylene substituted with one or more oxo groups. In certain embodiments, L comprises a click chemistry handle of Formula (IV) or Formula (V) and unsubstituted methylene. In certain embodiments, L comprises a click chemistry handle of Formula (IV) or Formula (V) and substituted methylene. In certain embodiments, L comprises a click chemistry handle of Formula (IV) or Formula (V) and unsubstituted n-butylene. In certain embodiments, L comprises a click chemistry handle of Formula (IV) or Formula (V) and substituted n-butylene. In certain embodiments, L comprises a click chemistry handle of Formula (IV) or Formula (V) and substituted n-butylene substituted with one or more oxo groups. In certain embodiments, L comprises a click chemistry handle of Formula (IV) or Formula (V) and substituted n-butylene substituted with one oxo group. In certain embodiments, L comprises a click chemistry handle of Formula (IV) or Formula (V) and substituted n-butylene substituted with two oxo groups. In certain embodiments, L comprises

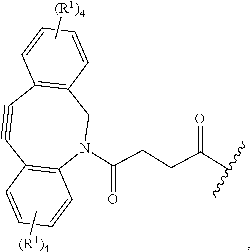

or a salt thereof. In certain embodiments, L comprises

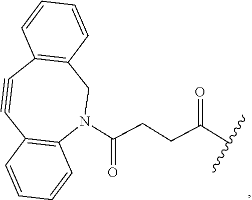 [SMILES: *C(=O)CCC(=O)N1Cc2ccccc2C#Cc2ccccc21]

or a salt thereof. In certain embodiments, L comprises

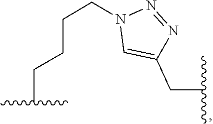 [SMILES: *CCCCn1cc(C*)nn1]

or a salt thereof. In certain embodiments, L comprises

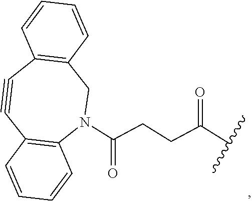 [SMILES: *C(=O)CCC(=O)N1Cc2ccccc2C#Cc2ccccc21]

or a salt thereof, and

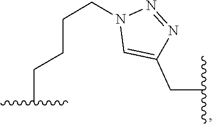 [SMILES: *CCCCn1cc(C*)nn1]

or a salt thereof. In certain embodiments, L comprises

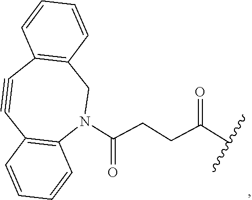 [SMILES: *C(=O)CCC(=O)N1Cc2ccccc2C#Cc2ccccc21]

or a salt thereof, and

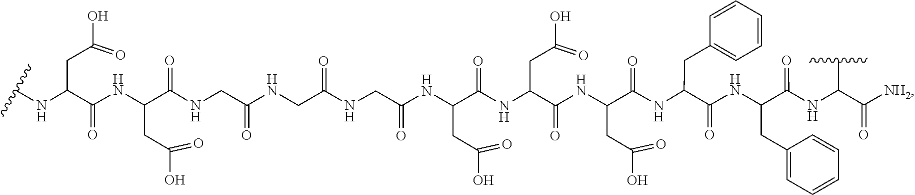 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

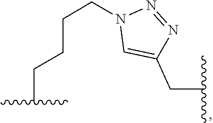 [SMILES: *CCCCn1cc(C*)nn1]

or a salt thereof, and

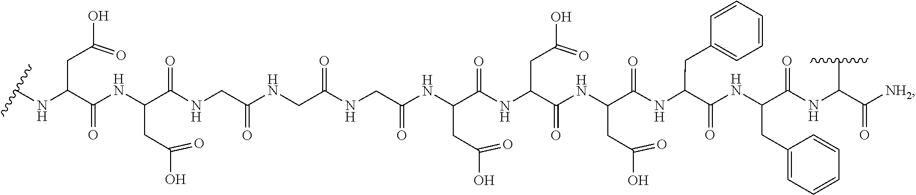 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

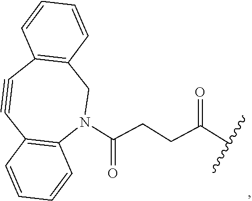 [SMILES: *C(=O)CCC(=O)N1Cc2ccccc2C#Cc2ccccc21]

or a salt thereof,

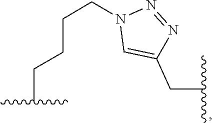 [SMILES: *CCCCn1cc(C*)nn1]

or a salt thereof, and

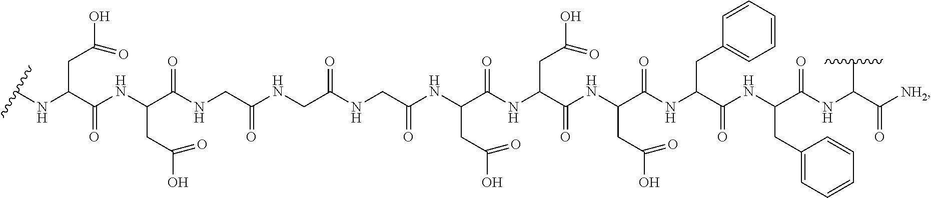 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof.

In certain embodiments, the click chemistry handle is of Formula (VI):

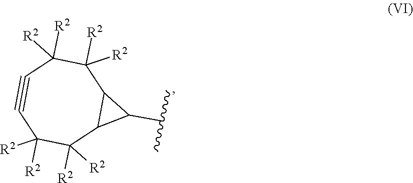

or a salt thereof, wherein:
each instance of R² is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂, or two instances of R² attached to the same carbon atom are taken together to form ═O or ═S;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
Ring A is optionally substituted carbocyclyl, optionally substituted heterocyclyl, optionally substituted aryl, or optionally substituted heteroaryl.

In certain embodiments, at least one instance of R² is hydrogen. In certain embodiments, all instances of R² are hydrogen. In certain embodiments, the click chemistry handle is of formula

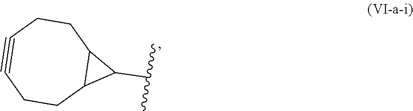 [SMILES: *C1C2CCC#CCCC12]

or a salt thereof. In certain embodiments, L comprises a click chemistry handle of Formula (VI) and optionally substituted C₁₋₆ alkylene. In certain embodiments, L comprises a click chemistry handle of Formula (VI) and substituted C₁₋₆ alkylene substituted with one or more oxo groups. In certain embodiments, L comprises

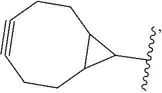 [SMILES: *C1C2CCC#CCCC12]

or a salt thereof. In certain embodiments, L comprises

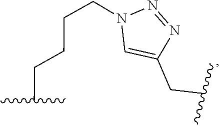 [SMILES: *CCCCn1cc(C*)nn1]

or a salt thereof. In certain embodiments, L comprises

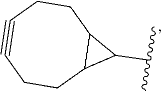 [SMILES: *C1C2CCC#CCCC12]

or a salt thereof, and

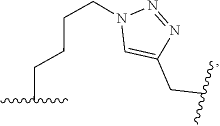 [SMILES: *CCCCn1cc(C*)nn1]

or a salt thereof. In certain embodiments, L comprises

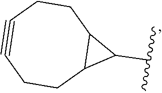 [SMILES: *C1C2CCC#CCCC12]

or a salt thereof, and

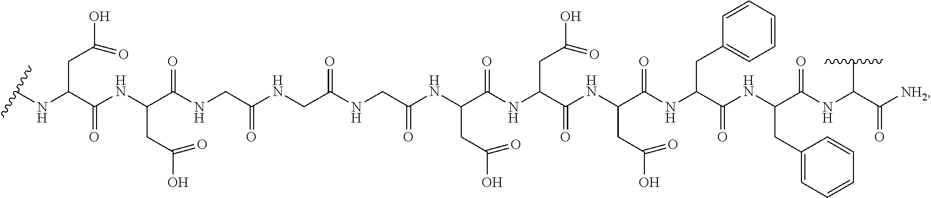 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

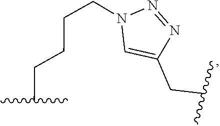 [SMILES: *CCCCn1cc(C*)nn1]

or a salt thereof, and

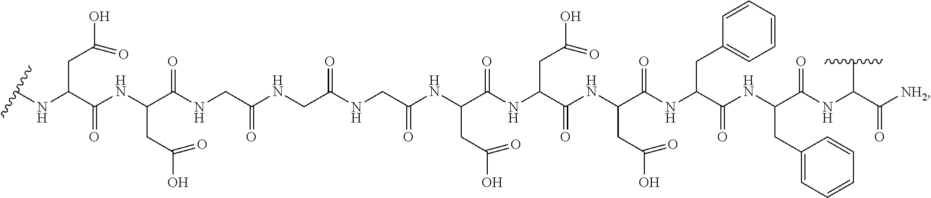 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

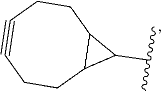 [SMILES: *C1C2CCC#CCCC12]

or a salt thereof,

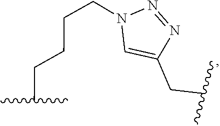 [SMILES: *CCCCn1cc(C*)nn1]

or a salt thereof, and

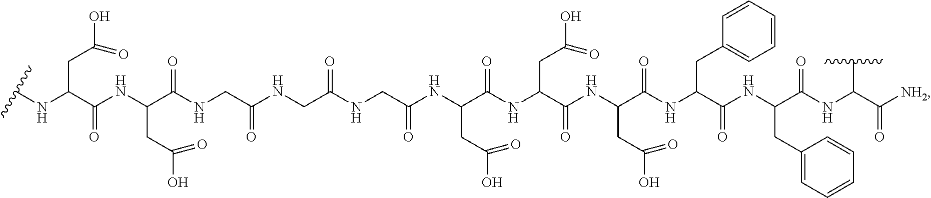 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof.

In certain embodiments, the click chemistry handle is of Formulae (VII-a), (VII-b), (VII-c), or (VII-d):

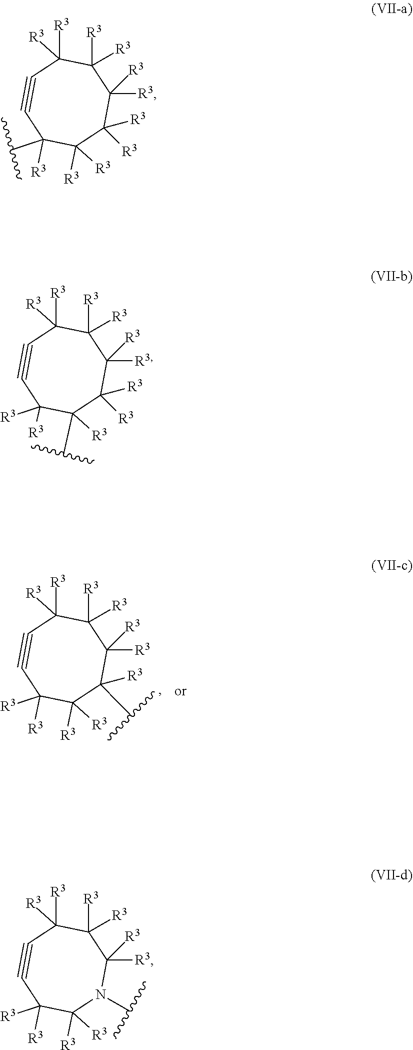

or a salt thereof, wherein:
each instance of R³ is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring.

In certain embodiments, at least one instance of R³ is hydrogen. In certain embodiments, at least one instance of R³ is halogen. In certain embodiments, at least two instances of R³ are halogen. In certain embodiments, at least one instance of R³ is fluorine. In certain embodiments, at least two instances of R³ are fluorine. In certain embodiments, two instances of R³ are halogen, and nine instances of R³ are hydrogen. In certain embodiments, two instances of R³ are fluorine, and nine instances of R³ are hydrogen. In certain embodiments, the click chemistry handle is of formula

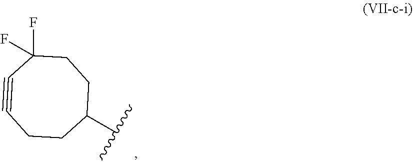 [SMILES: *C1CCC#CC(F)(F)CC1]

or a salt thereof.

In certain embodiments, L comprises a click chemistry handle of Formulae (VII-a), (VII-b), (VII-c), or (VII-d) and optionally substituted C₁₋₆ alkylene. In certain embodiments, L comprises a click chemistry handle of Formulae (VII-a), (VII-b), (VII-c), or (VII-d) and substituted C₁₋₆ alkylene substituted with one or more oxo groups. In certain embodiments, L comprises

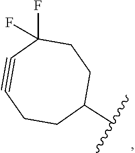 [SMILES: *C1CCC#CC(F)(F)CC1]

or a salt thereof. In certain embodiments, L comprises

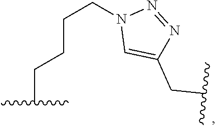 [SMILES: *CCCCn1cc(C*)nn1]

or a salt thereof. In certain embodiments, L comprises

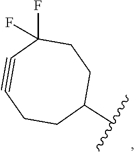 [SMILES: *C1CCC#CC(F)(F)CC1]

or a salt thereof, and

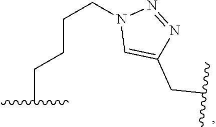 [SMILES: *CCCCn1cc(C*)nn1]

or a salt thereof. In certain embodiments, L comprises

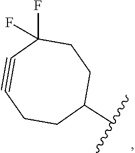 [SMILES: *C1CCC#CC(F)(F)CC1]

or a salt thereof, and

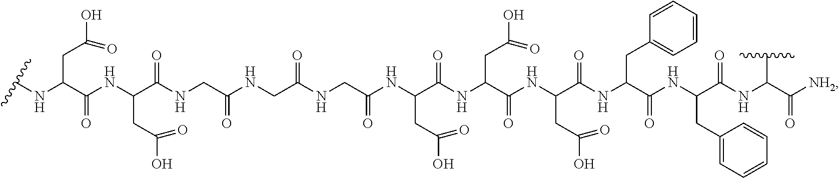 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

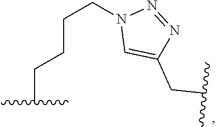 [SMILES: *CCCCn1cc(C*)nn1]

or a salt thereof, and

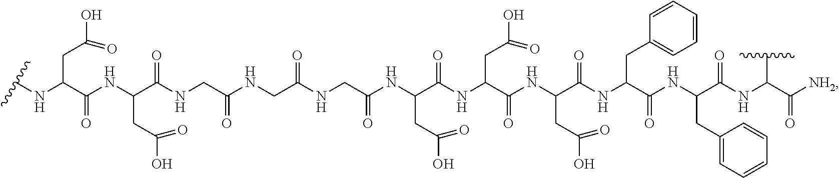 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof. In certain embodiments, L comprises

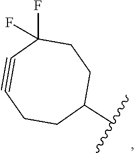 [SMILES: *C1CCC#CC(F)(F)CC1]

or a salt thereof,

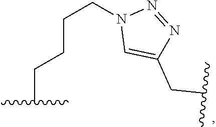 [SMILES: *CCCCn1cc(C*)nn1]

or a salt thereof, and

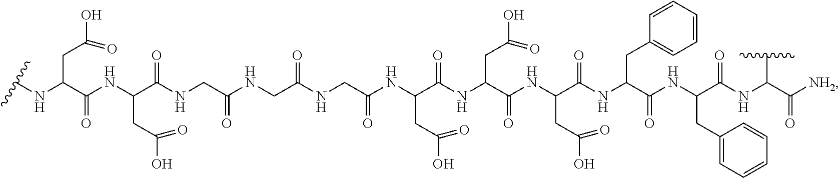 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O]

or a salt thereof.

In certain embodiments, L comprises

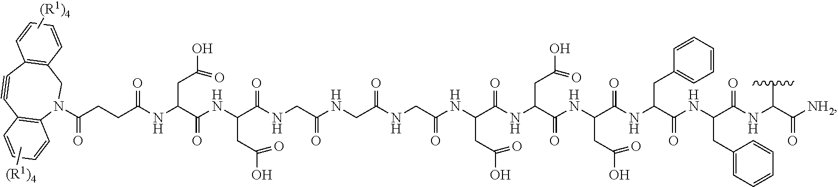

or a salt thereof. In certain embodiments, L comprises

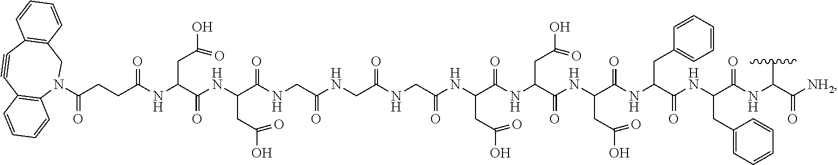 [SMILES: *C(NC(=O)C(Cc1ccccc1)NC(=O)C(Cc1ccccc1)NC(=O)C(CC(=O)O)NC(=O)C(CC(=O)O)NC(=O)C(CC(=O)O)NC(=O)CNC(=O)CNC(=O)CNC(=O)C(CC(=O)O)NC(=O)C(CC(=O)O)NC(=O)CCC(=O)N1Cc2ccccc2C#Cc2ccccc21)C(N)=O]

or a salt thereof. In certain embodiments, L comprises

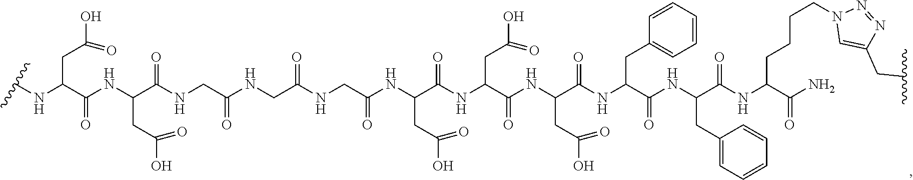 [SMILES: *Cc1cn(CCCCC(NC(=O)C(Cc2ccccc2)NC(=O)C(Cc2ccccc2)NC(=O)C(CC(=O)O)NC(=O)C(CC(=O)O)NC(=O)C(CC(=O)O)NC(=O)CNC(=O)CNC(=O)CNC(=O)C(CC(=O)O)NC(=O)C(CC(=O)O)N*)C(N)=O)nn1]

or a salt thereof. In certain embodiments, L comprises

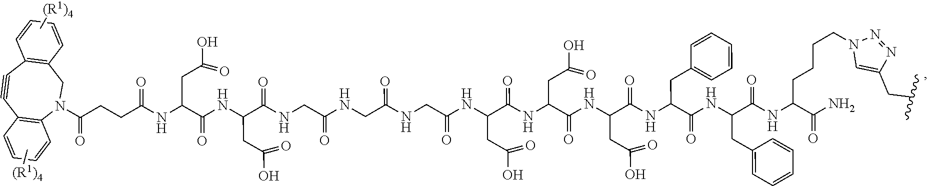

or a salt thereof. In certain embodiments, L comprises

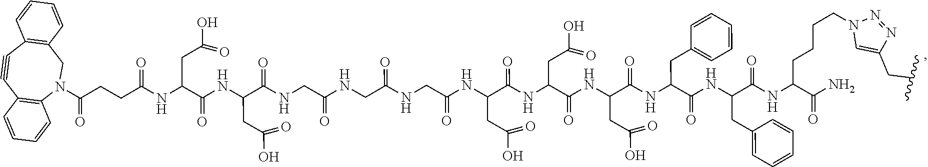 [SMILES: *Cc1cn(CCCCC(NC(=O)C(Cc2ccccc2)NC(=O)C(Cc2ccccc2)NC(=O)C(CC(=O)O)NC(=O)C(CC(=O)O)NC(=O)C(CC(=O)O)NC(=O)CNC(=O)CNC(=O)CNC(=O)C(CC(=O)O)NC(=O)C(CC(=O)O)NC(=O)CCC(=O)N2Cc3ccccc3C#Cc3ccccc32)C(N)=O)nn1]

or a salt thereof.

In certain embodiments, the compound of Formula (I) is of Formula (I-a-i):

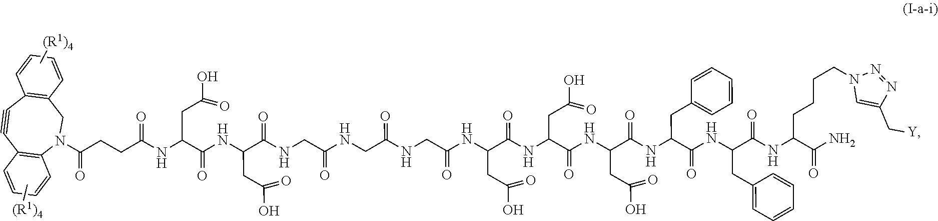

or a salt thereof. In certain embodiments, the compound of Formula (I) is of Formula (I-a-ii):

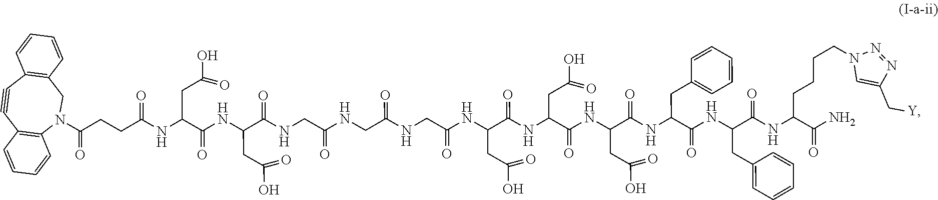

or a salt thereof.

In certain embodiments, the oligonucleotide comprises Q24. In certain embodiments, the oligonucleotide comprises between about 10 and about 50 bases. In certain embodiments, the oligonucleotide comprises between about 25 and about 50 bases. In certain embodiments, the oligonucleotide comprises between about 25 and about 45 bases. In certain embodiments, the oligonucleotide comprises between about 25 and about 40 bases. In certain embodiments, the oligonucleotide comprises between about 25 and about 35 bases. In certain embodiments, the oligonucleotide comprises between about 25 and about 30 bases. In certain embodiments, the oligonucleotide comprises 25 bases (e.g., the oligonucleotide is a 25-mer). In certain embodiments, the oligonucleotide comprises between about 10 and about 50 bases, and the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), or a salt thereof, wherein Cy is cysteic acid. In certain embodiments, the oligonucleotide comprises between about 25 and about 50 bases, and the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), or a salt thereof, wherein Cy is cysteic acid. In certain embodiments, the oligonucleotide comprises 25 bases (e.g., the oligonucleotide is a 25-mer), and the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), or a salt thereof, wherein Cy is cysteic acid.

In certain embodiments, Y further comprises a biotin moiety. In certain embodiments, the biotin moiety is a bis-biotin moiety. In certain embodiments, Y further comprises an avidin protein. In certain embodiments, the avidin protein is streptavidin. In some embodiments, the avidin protein is streptavidin in a tetrameric form (e.g., a homotetramer). In some embodiments, the avidin protein comprises one or more biotin binding sites. In certain embodiments, Y is immobilized to a surface.

In certain embodiments, the compound of formula L-N₃ comprises a moiety selected from:

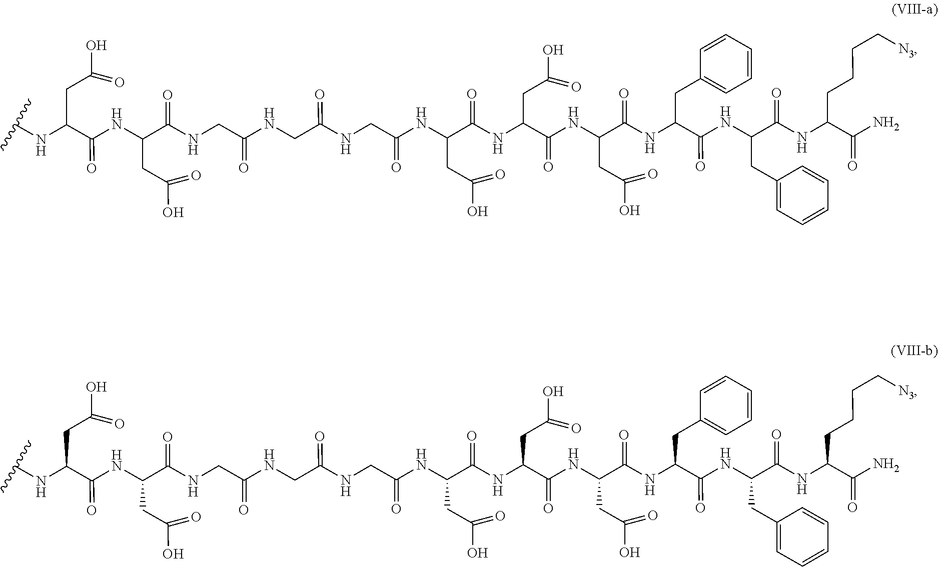 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(CCCCN=[N+]=[N-])C(N)=O ; *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](CCCCN=[N+]=[N-])C(N)=O]

or a salt thereof. In certain embodiments, the compound of formula L-N₃ comprises

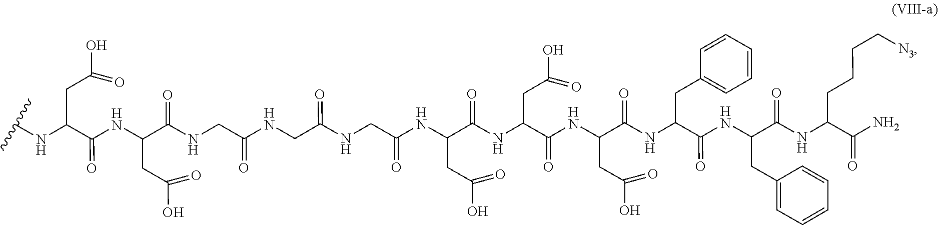 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(CCCCN=[N+]=[N-])C(N)=O]

or a salt thereof. In certain embodiments, the compound of formula L-N₃ comprises

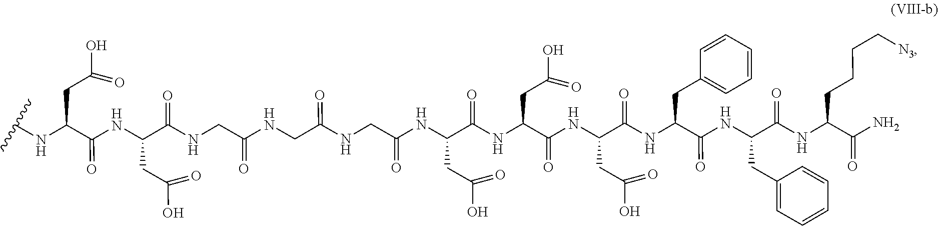 [SMILES: *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](CCCCN=[N+]=[N-])C(N)=O]

or a salt thereof.

In certain embodiments, the compound of formula L-N₃ is of formula:

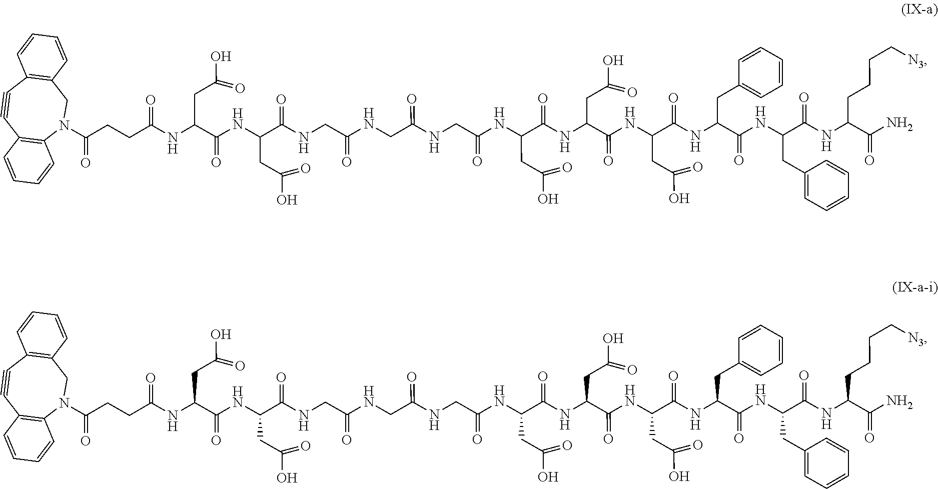 [SMILES: [N-]=[N+]=NCCCCC(NC(=O)C(Cc1ccccc1)NC(=O)C(Cc1ccccc1)NC(=O)C(CC(=O)O)NC(=O)C(CC(=O)O)NC(=O)C(CC(=O)O)NC(=O)CNC(=O)CNC(=O)CNC(=O)C(CC(=O)O)NC(=O)C(CC(=O)O)NC(=O)CCC(=O)N1Cc2ccccc2C#Cc2ccccc21)C(N)=O ; [N-]=[N+]=NCCCC[C@H](NC(=O)[C@H](Cc1ccccc1)NC(=O)[C@H](Cc1ccccc1)NC(=O)[C@H](CC(=O)O)NC(=O)[C@H](CC(=O)O)NC(=O)[C@H](CC(=O)O)NC(=O)CNC(=O)CNC(=O)CNC(=O)[C@H](CC(=O)O)NC(=O)[C@H](CC(=O)O)NC(=O)CCC(=O)N1Cc2ccccc2C#Cc2ccccc21)C(N)=O]

or a salt thereof. In certain embodiments, the compound of formula L-N₃ is of formula

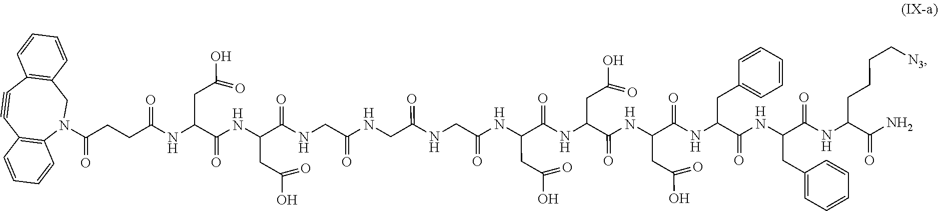 [SMILES: [N-]=[N+]=NCCCCC(NC(=O)C(Cc1ccccc1)NC(=O)C(Cc1ccccc1)NC(=O)C(CC(=O)O)NC(=O)C(CC(=O)O)NC(=O)C(CC(=O)O)NC(=O)CNC(=O)CNC(=O)CNC(=O)C(CC(=O)O)NC(=O)C(CC(=O)O)NC(=O)CCC(=O)N1Cc2ccccc2C#Cc2ccccc21)C(N)=O]

or a salt thereof. In certain embodiments, the compound of formula L-N₃ is of formula

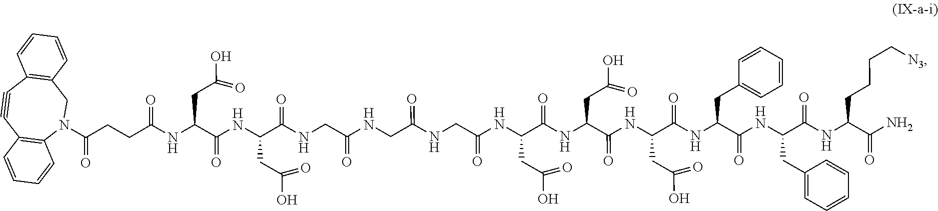 [SMILES: [N-]=[N+]=NCCCC[C@H](NC(=O)[C@H](Cc1ccccc1)NC(=O)[C@H](Cc1ccccc1)NC(=O)[C@H](CC(=O)O)NC(=O)[C@H](CC(=O)O)NC(=O)[C@H](CC(=O)O)NC(=O)CNC(=O)CNC(=O)CNC(=O)[C@H](CC(=O)O)NC(=O)[C@H](CC(=O)O)NC(=O)CCC(=O)N1Cc2ccccc2C#Cc2ccccc21)C(N)=O]

or a salt thereof.

In certain embodiments, the method of preparing a compound of Formula (II) comprises a “click chemistry” reaction (e.g., a Huisgen alkyne-azide cycloaddition).

Various conditions are suitable for the reaction of a compound of Formula (I), or a salt thereof, with a compound of formula Z—N₃, or a salt thereof, to produce a compound of Formula (II), or a salt thereof, and one of ordinary skill in the art will readily understand that such conditions may be substituted and still be compatible using the methods disclosed herein. For example, such a reaction may be performed in the presence of a solvent. Suitable solvents for performing this reaction include, but are not limited to, water, aqueous NaHCO₃(e.g., 0.1 M NaHCO₃), dimethylsulfoxide, dimethylformamide, acetonitrile, and combinations thereof. In some embodiments, the reaction of a compound of Formula (I), or a salt thereof, with a compound of formula Z—N₃, or a salt thereof, is performed in water, aqueous NaHCO₃(e.g., 0.1 M NaHCO₃), or a combination thereof.

The reaction of a compound of Formula (I), or a salt thereof, with a compound of formula Z—N₃, or a salt thereof, to produce a compound of Formula (II), or a salt thereof, may be also performed for varying amounts of time. The reaction may comprise a reaction time of approximately 5 minutes, approximately 10 minutes, approximately 15 minutes, approximately 20 minutes, approximately 25 minutes, approximately 30 minutes, approximately 35 minutes, approximately 40 minutes, approximately 45 minutes, approximately 50 minutes, approximately 55 minutes, approximately 1 hour, approximately 2 hours, approximately 3 hours, approximately 4 hours, or approximately 5 hours. In some embodiments, the reaction of a compound of Formula (I), or a salt thereof, with a compound of formula Z—N₃, or a salt thereof, is performed for a reaction time of approximately 20 minutes. In some embodiments, the reaction of a compound of Formula (I), or a salt thereof, with a compound of formula Z—N₃, or a salt thereof, is performed for a reaction time of approximately 40 minutes.

The reaction of a compound of Formula (I), or a salt thereof, with a compound of formula Z—N₃, or a salt thereof, to produce a compound of Formula (II), or a salt thereof, may be performed at various temperatures. For example, the reaction of a compound of Formula (I), or a salt thereof, with a compound of formula Z—N₃, or a salt thereof, may comprise a reaction temperature of approximately 15° C., approximately 20° C., approximately 25° C., approximately 30° C., approximately 35° C., approximately 37° C., approximately 40° C., approximately 45° C., or approximately 50° C. In certain embodiments, the reaction temperature may be in a range of approximately 15° C. to approximately 50° C., approximately 15° C. to approximately 45° C., approximately 15° C. to approximately 40° C., approximately 15° C. to approximately 35° C., approximately 15° C. to approximately 30° C., approximately 15° C. to approximately 25° C., approximately 15° C. to approximately 20° C., approximately 35° C. to approximately 45° C., or approximately 35° C. to approximately 40° C. In certain embodiments, the reaction temperature is approximately 20° C. In certain embodiments, the reaction temperature is approximately 25° C. In certain embodiments, the reaction temperature is room temperature.

The reaction of a compound of Formula (I), or a salt thereof, with a compound of formula Z—N₃, or a salt thereof, to produce a compound of Formula (II), or a salt thereof, may be performed with a reducing agent. Suitable reducing agents for performing this reaction include, but are not limited to, sodium ascorbate, hydroxylamine, triethylamine, diisopropylethylamine, and combinations thereof. In some embodiments, the reaction of a compound of Formula (I), or a salt thereof, with a compound of formula Z—N₃, or a salt thereof, is performed with sodium ascorbate as the reducing agent. In some embodiments, the reaction of a compound of Formula (I), or a salt thereof, with a compound of formula Z—N₃, or a salt thereof, is performed with sodium ascorbate as the reducing agent, wherein the sodium ascorbate is added in one portion. In some embodiments, the reaction of a compound of Formula (I), or a salt thereof, with a compound of formula Z—N₃, or a salt thereof, is performed with sodium ascorbate as the reducing agent, wherein the sodium ascorbate is added in two or more portions. In some embodiments, the reaction of a compound of Formula (I), or a salt thereof, with a compound of formula Z—N₃, or a salt thereof, is performed with sodium ascorbate as the reducing agent, wherein the sodium ascorbate is added in two portions.

The reaction of a compound of Formula (I), or a salt thereof, with a compound of formula Z—N₃, or a salt thereof, to produce a compound of Formula (II), or a salt thereof, may be performed with a copper (II) compound. Suitable copper (II) compounds for performing this reaction include, but are not limited to, copper (II) tris(3-hydroxypropyltriazolylmethyl)amine (Cu(THPTA)), copper (II) sulfate, copper (II) acetate, and combinations thereof. In some embodiments, the reaction of a compound of Formula (I), or a salt thereof, with a compound of formula Z—N₃, or a salt thereof, is performed with Cu(THPTA) as the copper (II) compound. In some embodiments, the reaction of a compound of Formula (I), or a salt thereof, with a compound of formula Z—N₃, or a salt thereof, to produce a compound of Formula (II), or a salt thereof, may be performed with a copper (II) compound and a ligand. Suitable ligands for performing this reaction include, but are not limited to, tris(3-hydroxypropyltriazolylmethyl)amine, aminoguanidine, tris[(1-benzyl-1H-1,2,3-triazol-4-yl)methyl]amine, and combinations thereof. In some embodiments, the reaction of a compound of Formula (I), or a salt thereof, with a compound of formula Z—N₃, or a salt thereof, is performed with tris(3-hydroxypropyltriazolylmethyl)amine as the ligand. The reaction of a compound of Formula (I), or a salt thereof, with a compound of formula Z—N₃, or a salt thereof, to produce a compound of Formula (II), or a salt thereof, may be performed with a copper (I) compound. Suitable copper (I) compounds include, but are not limited to, copper (I) iodide, copper (I) bromide, copper (I) chloride, copper (I) thiophene-2-carboxylate (CuTC), tetrakis(acetonitrile)copper(I) hexafluorophosphate, tetrakis(acetonitrile)copper(I) tetrafluoroborate, and combinations thereof.

The reaction of a compound of Formula (I), or a salt thereof, with a compound of formula Z—N₃, or a salt thereof, to produce a compound of Formula (II), or a salt thereof, may be performed with various molar ratios of the reagents to one another. For example, the ratio of the compound of Formula (I), or a salt thereof, to the compound of formula Z—N₃, or a salt thereof, may be approximately 1:1, approximately 1:2, approximately 1:3, approximately 1:4, approximately 1:5, approximately 1:6, approximately 1:7, approximately 1:8, approximately 1:9, or approximately 1:10. In certain embodiments, a ratio greater than approximately 1:10 may be used. In certain embodiments, a ratio of the compound of Formula (I), or a salt thereof, to the compound of formula Z—N₃, or a salt thereof, of approximately 1:4 is used. In certain embodiments, a ratio of the compound of Formula (I), or a salt thereof, to the compound of formula Z—N₃, or a salt thereof, of approximately 1:3 is used. In certain embodiments, a ratio of the compound of Formula (I), or a salt thereof, to the compound of formula Z—N₃, or a salt thereof, of approximately 1:3.3 is used. For example, the ratio of the compound of Formula (I), or a salt thereof, to the reducing agent may be approximately 1:1, approximately 1:10, approximately 1:20, approximately 1:30, approximately 1:40, approximately 1:50, approximately 1:60, approximately 1:70, approximately 1:80, approximately 1:90, approximately 1:100, approximately 1:120, or approximately 1:150. In certain embodiments, a ratio of the compound of Formula (I), or a salt thereof, to the reducing agent of approximately 1:40 is used. In certain embodiments, a ratio of the compound of Formula (I), or a salt thereof, to the reducing agent of approximately 1:80 is used. In certain embodiments, a ratio of the compound of Formula (I), or a salt thereof, to the reducing agent of approximately 1:40 is used, wherein the reducing agent is added in two or more portions. In certain embodiments, a ratio of the compound of Formula (I), or a salt thereof, to the reducing agent of approximately 1:80 is used, wherein the reducing agent is added in two or more portions. For example, the ratio of the compound of Formula (I), or a salt thereof, to the copper (I) compound may be approximately 1:1, approximately 1:0.9, approximately 1:0.8, approximately 1:0.7, approximately 1:0.6, approximately 1:0.5, approximately 1:0.4, approximately 1:0.3, approximately 1:0.0, or approximately 1:0.1. In certain embodiments, a ratio of the compound of Formula (I), or a salt thereof, to the copper (I) compound of greater than approximately 1:1 may be used. In certain embodiments, a ratio of the compound of Formula (I), or a salt thereof, to the copper (I) compound of approximately 1:0.8 is used.

Any reaction described herein may further comprise a work up, which can consist of a single step or multiple steps. Various steps are suitable for the work up, and one of ordinary skill in the art will readily understand that such steps may be substituted and still be compatible using the methods disclosed herein. In some embodiments, a reaction may be concentrated under reduced pressure using evaporation or lyophilization. In some embodiments, a reaction may be purified using silica gel chromatography. In some embodiments, a reaction may be subjected to liquid-liquid extraction. In some embodiments, a reaction may be quenched. In some embodiments, a reaction may be quenched with a base (e.g. EDTA).

### Methods of Sequencing a Polypeptide

In another aspect, provided herein is a method of sequencing a polypeptide Z, the method comprising reacting a compound of Formula (II):

Z-L-Y (II),

or a salt thereof, with a peptidase, wherein:
L comprises a polypeptidyl group; and
Y is an oligonucleotide;
reacting the compound of Formula (II), or salt thereof, with a peptidase, in a degradation process;
obtaining data during the degradation process;
analyzing the data to determine portions of the data corresponding to amino acids that are sequentially exposed at a terminus of the polypeptide during the degradation process; and
outputting an amino acid sequence representative of the polypeptide.

In certain embodiments, the methods of sequencing a polypeptide further comprise reacting a compound of Formula (I):

L-Y (I),

or a salt thereof, with a functionalized polypeptide, or salt thereof, to provide the compound of Formula (II):

Z-L-Y (II),

or a salt thereof, wherein the functionalized polypeptide, or salt thereof, comprises a click chemistry handle, and the compound of Formula (I), or salt thereof, comprises a click chemistry handle.

In certain embodiments, L, Y, and Z are as described herein.

In certain embodiments, a functionalized polypeptide is a polypeptide that has been chemically modified to comprise at least one reactive functional group. In certain embodiments, the at least one reactive functional group is a click chemistry handle. In certain embodiments, the at least one reactive functional group is shown in Tables 1 and 2. In certain embodiments, the at least one reactive functional group is an azide. In certain embodiments, the at least one reactive functional group is capable of participating in a coupling reaction (e.g., formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2]cycloaddition)). In certain embodiments, the at least one reactive functional group is capable of participating in a click chemistry reaction (e.g., azide-alkyne Huisgen cycloaddition; Diels-Alder reactions (e.g., tetrazine [4+2] cycloaddition)).

In certain embodiments, the method comprises a coupling reaction (e.g., formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2]cycloaddition)). In certain embodiments, the method comprises a click chemistry reaction (e.g., azide-alkyne Huisgen cycloaddition; Diels-Alder reactions (e.g., tetrazine [4+2] cycloaddition)). In certain embodiments, the method comprises an azide-alkyne cycloaddition.

In certain embodiments, the method comprises iterative detection and cleavage at a terminal end of a polypeptide.

In certain embodiments, the peptidase is an exopeptidase. An exopeptidase generally requires a polypeptide substrate to comprise at least one of a free amino group at its amino-terminus or a free carboxyl group at its carboxy-terminus. In some embodiments, an exopeptidase in accordance with the application hydrolyses a bond at or near a terminus of a polypeptide. In some embodiments, an exopeptidase hydrolyses a bond not more than three residues from a polypeptide terminus. For example, in some embodiments, a single hydrolysis reaction catalyzed by an exopeptidase cleaves a single amino acid, a dipeptide, or a tripeptide from a polypeptide terminal end.

In some embodiments, an exopeptidase in accordance with the application is an aminopeptidase or a carboxypeptidase, which cleaves a single amino acid from an amino- or a carboxy-terminus, respectively. In some embodiments, an exopeptidase in accordance with the application is a dipeptidyl-peptidase or a peptidyl-dipeptidase, which cleave a dipeptide from an amino- or a carboxy-terminus, respectively. In yet other embodiments, an exopeptidase in accordance with the application is a tripeptidyl-peptidase, which cleaves a tripeptide from an amino-terminus. Peptidase classification and activities of each class or subclass thereof is well known and described in the literature (see, e.g., Gurupriya, V. S. & Roy, S. C. Proteases and Protease Inhibitors in Male Reproduction. Proteases in Physiology and Pathology 195-216 (2017); and Brix, K. & Stöcker, W. Proteases: Structure and Function. Chapter 1). In some embodiments, a peptidase in accordance with the application removes more than three amino acids from a polypeptide terminus. Accordingly, in some embodiments, the peptidase is an endopeptidase, e.g., that cleaves preferentially at particular positions (e.g., before or after a particular amino acid). In some embodiments, the size of a polypeptide cleavage product of endopeptidase activity will depend on the distribution of cleavage sites (e.g., amino acids) within the polypeptide being analyzed.

An exopeptidase in accordance with the application may be selected or engineered based on the directionality of a sequencing reaction. For example, in embodiments of sequencing from an amino-terminus to a carboxy-terminus of a polypeptide, an exopeptidase comprises aminopeptidase activity. Conversely, in embodiments of sequencing from a carboxy-terminus to an amino-terminus of a polypeptide, an exopeptidase comprises carboxypeptidase activity. Examples of carboxypeptidases that recognize specific carboxy-terminal amino acids have been described in the literature (see, e.g., Garcia-Guerrero, M. C., et al. (2018) PNAS 115(17)).

In some embodiments, the peptidase is an aminopeptidase that selectively binds one or more types of amino acids. In some embodiments, an aminopeptidase is non-specific such that it cleaves most or all types of amino acids from a terminal end of a polypeptide. In some embodiments, an aminopeptidase is more efficient at cleaving one or more types of amino acids from a terminal end of a polypeptide as compared to other types of amino acids at the terminal end of the polypeptide. For example, an aminopeptidase in accordance with the application specifically cleaves alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and/or valine. In some embodiments, an aminopeptidase is a proline aminopeptidase. In some embodiments, an aminopeptidase is a proline iminopeptidase. In some embodiments, an aminopeptidase is a glutamate/aspartate-specific aminopeptidase. In some embodiments, an aminopeptidase is a methionine-specific aminopeptidase. In some embodiments, an aminopeptidase is a non-specific aminopeptidase. In some embodiments, a non-specific aminopeptidase is a zinc metalloprotease.

In some aspects, the disclosure provides an aminopeptidase having an amino acid sequence selected from Table 3. It should be appreciated that the example sequences in Table 3 and other examples described herein are meant to be non-limiting, and aminopeptidases in accordance with the disclosure can include any homologs, variants, or fragments thereof minimally containing domains or subdomains responsible for amino acid cleavage.

In some embodiments, an aminopeptidase has an amino acid sequence that is at least 80% identical to an amino acid sequence selected from Table 3. In some embodiments, an aminopeptidase has at least 40%, at least 50%, at least 60%, at least 70%, at least 80%, at least 85%, at least 90%, at least 92%, at least 94%, at least 95%, at least 96%, at least 98%, or higher, amino acid sequence identity to an amino acid sequence selected from Table 3. In some embodiments, an aminopeptidase has 25-50%, 50-60%, 60-70%, 70-80%, 80-90%, 90-95%, 92-99%, 94-99%, 95-99%, 40-100%, 50-100%, 60-100%, 70-100%, 80-100%, 90-100%, 92-100%, 94-100%, 95-100%, 96-100%, or 100% amino acid sequence identity to an amino acid sequence selected from Table 3.

In some embodiments, the aminopeptidase is a synthetic or recombinant aminopeptidase. In some embodiments, the aminopeptidase is a monomeric aminopeptidase. In some embodiments, the aminopeptidase is a multimeric aminopeptidase (e.g., a multimeric complex of monomeric subunits, which may be the same or different). In some embodiments, the aminopeptidase is a modified aminopeptidase and includes one or more amino acid mutations relative to a sequence set forth in Table 3.

In some embodiments, the aminopeptidase is an aminopeptidase obtained or derived from a particular source (e.g., organism). As described herein, in some embodiments, an aminopeptidase identified as being from a particular organism does not impart a requirement that the aminopeptidase have an amino acid sequence that is 100% identical to a naturally-occurring aminopeptidase from the organism, although it may in some embodiments.

TABLE 3
Non-limiting examples of aminopeptidases.

| | | SEQ |
|---|---|---|
| | | ID |
| Name | Sequence | NO |
| Pyrococcus | MEVRNMVDYELLKKVVEAPGVSGYEFLGIRDVVIEEIKDYVDEVKVDKLGNVI | 1 |
| horikoshii TET II | AHKKGEGPKVMIAAHMDQIGLMVTHIEKNGFLRVAPIGGVDPKTLIAQRFKVW | |
| Aminopeptidase | IDKGKFIYGVGASVPPHIQKPEDRKKAPDWDQIFIDIGAESKEEAEDMGVKIG | |
| (hTET II) | TVITWDGRLERLGKHRFVSIAFDDRIAVYTILEVAKQLKDAKADVYFVATVQE | |
| | EVGLRGARTSAFGIEPDYGFAIDVTIAADIPGTPEHKQVTHLGKGTAIKIMDR | |
| | SVICHPTIVRWLEELAKKHEIPYQLEILLGGGTDAGAIHLTKAGVPTGALSVP | |
| | ARYIHSNTEVVDERDVDATVELMTKALENIHELKI | |
| AP30 | MEVRNMVDYELLKKVVEAPGVSGYEFLGIRDVVIEEIKDYVDEVKVDKLGNVI | 2 |
| | AHKKGEGPKVMIAAHMDQIGLMVTHIEKNGFLRVAPIGGVDPKTLIAQRFKVW | |
| | IDKGKFIYGVGASVPPHIQKPEDRKKAPDWDQIFIDIGAESKEEAEDMGVKIG | |
| | TVITWDGRLERLGKHRFVSIAFDDRIAVYTILEVAKQLKDAKADVYFVATVQE | |
| | EVGLRGARTSAFGIEPDYGFAIDVTIAADIPGTPEHKQVTHLGKGTAIKIMDR | |
| | SVICHPTIVRWLEELAKKHEIPYQLEILLGGGTDAGAIHLTKAGVPTGALSVP | |
| | ARYIHSNTEVVDERDVDATVELMTKALENIHELKIGGSHHHHHHHHHHGGGSG | |
| | GGSGGGSGLNDFFEAQKIEWHEGGGSGGGSGGGSGLNDFFEAQKIEWHE | |
| Pyrococcus | MDLKGGESMVDWKLMQEIIEAPGVSGYEHLGIRDIVVDVLKEVADEVKVDKLG | 3 |
| horikoshii TET III | NVIAHFKGSSPRIMVAAHMDKIGVMVNHIDKDGYLHIVPIGGVLPETLVAQRI | |
| Aminopeptidase | RFFTEKGERYGVVGVLPPHLRRGQEDKGSKIDWDQIVVDVGASSKEEAEEMGF | |
| (hTET III) | RVGTVGEFAPNFTRLNEHRFATPYLDDRICLYAMIEAARQLGDHEADIYIVGS | |
| | VQEEVGLRGARVASYAINPEVGIAMDVTFAKQPHDKGKIVPELGKGPVMDVGP | |
| | NINPKLRAFADEVAKKYEIPLQVEPSPRPTGTDANMQINREGVATAVLSIPIR | |
| | YMHSQVELADARDVDNTIKLAKALLEELKPMDFTP | |
| AP37 | MDLKGGESMVDWKLMQEIIEAPGVSGYEHLGIRDIVVDVLKEVADEVKVDKLG | 4 |
| | NVIAHFKGSSPRIMVAAHMDKIGVMVNHIDKDGYLHIVPIGGVLPETLVAQRI | |
| | RFFTEKGERYGVVGVLPPHLRRGQEDKGSKIDWDQIVVDVGASSKEEAEEMGF | |
| | RVGTVGEFAPNFTRLNEHRFATPYLDDRICLYAMIEAARQLGDHEADIYIVGS | |
| | VQEEVGLRGARVASYAINPEVGIAMDVTFAKQPHDKGKIVPELGKGPVMDVGP | |
| | NINPKLRAFADEVAKKYEIPLQVEPSPRPTGTDANMQINREGVATAVLSIPIR | |
| | YMHSQVELADARDVDNTIKLAKALLEELKPMDFTPGHHHHHHHHHH | |
| Yersinia pestis | MTQQEYQNRRQALLAKMAPGSAAIIFAAPEATRSADSEYPYRQNSDFSYLTGF | 5 |
| Xaa-Prolyl | NEPEAVLILVKSDETHNHSVLFNRIRDLTAEIWFGRRLGQEAAPTKLAVDRAL | |
| aminopeptidase | PFDEINEQLYLLLNRLDVIYHAQGQYAYADNIVFAALEKLRHGFRKNLRAPAT | |
| (yPIP) | LTDWRPWLHEMRLFKSAEEIAVLRRAGEISALAHTRAMEKCRPGMFEYQLEGE | |
| | ILHEFTRHGARYPAYNTIVGGGENGCILHYTENECELRDGDLVLIDAGCEYRG | |
| | YAGDITRTFPVNGKFTPAQRAVYDIVLAAINKSLTLFRPGTSIREVTEEVVRI | |
| | MVVGLVELGILKGDIEQLIAEQAHRPFFMHGLSHWLGMDVHDVGDYGSSDRGR | |
| | ILEPGMVLTVEPGLYIAPDADVPPQYRGIGIRIEDDIVITATGNENLTASVVK | |
| | DPDDIEALMALNHAGENLYFQLE | |
| yPIP-6x His | MTQQEYQNRRQALLAKMAPGSAAIIFAAPEATRSADSEYPYRQNSDFSYLTGF | 6 |
| | NEPEAVLILVKSDETHNHSVLFNRIRDLTAEIWFGRRLGQEAAPTKLAVDRAL | |
| | PFDEINEQLYLLLNRLDVIYHAQGQYAYADNIVFAALEKLRHGFRKNLRAPAT | |
| | LTDWRPWLHEMRLFKSAEEIAVLRRAGEISALAHTRAMEKCRPGMFEYQLEGE | |
| | ILHEFTRHGARYPAYNTIVGGGENGCILHYTENECELRDGDLVLIDAGCEYRG | |
| | YAGDITRTFPVNGKFTPAQRAVYDIVLAAINKSLTLFRPGTSIREVTEEVVRI | |
| | MVVGLVELGILKGDIEQLIAEQAHRPFFMHGLSHWLGMDVHDVGDYGSSDRGR | |
| | ILEPGMVLTVEPGLYIAPDADVPPQYRGIGIRIEDDIVITATGNENLTASVVK | |
| | DPDDIEALMALNHAGENLYFQLEHHHHHH | |
| yPIP (truncated) | MTQQEYQNRRQALLAKMAPGSAAIIFAAPEATRSADSEYPYRQNSDFSYLTGF | 7 |
| | NEPEAVLILVKSDETHNHSVLFNRIRDLTAEIWFGRRLGQEAAPTKLAVDRAL | |
| | PFDEINEQLYLLLNRLDVIYHAQGQYAYADNIVFAALEKLRHGFRKNLRAPAT | |
| | LTDWRPWLHEMRLFKSAEEIAVLRRAGEISALAHTRAMEKCRPGMFEYQLEGE | |
| | ILHEFTRHGARYPAYNTIVGGGENGCILHYTENECELRDGDLVLIDAGCEYRG | |
| | YAGDITRTFPVNGKFTPAQRAVYDIVLAAINKSLTLFRPGTSIREVTEEVVRI | |
| | MVVGLVELGILKGDIEQLIAEQAHRPFFMHGLSHWLGMDVHDVGDYGSSDRGR | |
| | ILEPGMVLTVEPGLYIAPDADVPPQYRGIGIRIEDDIVITATGNENLTASVVK | |
| | DPDDIEALMALNHAGENLYFQ | |
| AP70 | MTQQEYQNRRQALLAKMAPGSAAIIFAAPEATRSADSEYPYRQNSDFSYLTGF | 8 |
| | NEPEAVLILVKSDETHNHSVLFNRIRDLTAEIWFGRRLGQEAAPTKLAVDRAL | |
| | PFDEINEQLYLLLNRLDVIYHAQGQYAYADNIVFAALEKLRHGFRKNLRAPAT | |
| | LTDWRPWLHEMRLFKSAEEIAVLRRAGEISALAHTRAMEKCRPGMFEYQLEGE | |
| | ILHEFTRHGARYPAYNTIVGGGENGCILHYTENECELRDGDLVLIDAGCEYRG | |
| | YAGDITRTFPVNGKFTPAQRAVYDIVLAAINKSLTLFRPGTSIREVTEEVVRI | |
| | MVVGLVELGILKGDIEQLIAEQAHRPFFMHGLSHWLGMDVHDVGDYGSSDRGR | |
| | ILEPGMVLTVEPGLYIAPDADVPPQYRGIGIRIEDDIVITATGNENLTASVVK | |
| | DPDDIEALMALNHAGENLYFQGGSHHHHHH | |
| L. pneumophila | MMVKQGVFMKTDQSKVKKLSDYKSLDYFVIHVDLQIDLSKKPVESKARLTVVP | 9 |
| M1 | NLNVDSHSNDLVLDGENMTLVSLQMNDNLLKENEYELTKDSLIIKNIPQNTPF | |
| Aminopeptidase | TIEMTSLLGENTDLFGLYETEGVALVKAESEGLRRVFYLPDRPDNLATYKTTI | |
| (Glu/Asp Specific) | IANQEDYPVLLSNGVLIEKKELPLGLHSVTWLDDVPKPSYLFALVAGNLQRSV | |
| | TYYQTKSGRELPIEFYVPPSATSKCDFAKEVLKEAMAWDERTFNLECALRQHM | |
| | VAGVDKYASGASEPTGLNLFNTENLFASPETKTDLGILRVLEVVAHEFFHYWS | |
| | GDRVTIRDWFNLPLKEGLTTFRAAMFREELFGTDLIRLLDGKNLDERAPRQSA | |
| | YTAVRSLYTAAAYEKSADIFRMMMLFIGKEPFIEAVAKFFKDNDGGAVTLEDF | |
| | IESISNSSGKDLRSFLSWFTESGIPELIVTDELNPDTKQYFLKIKTVNGRNRP | |
| | IPILMGLLDSSGAEIVADKLLIVDQEEIEFQFENIQTRPIPSLLRSFSAPVHM | |
| | KYEYSYQDLLLLMQFDTNLYNRCEAAKQLISALINDFCIGKKIELSPQFFAVY | |
| | KALLSDNSLNEWMLAELITLPSLEELIENQDKPDFEKLNEGRQLIQNALANEL | |
| | KTDFYNLLFRIQISGDDDKQKLKGFDLKQAGLRRLKSVCFSYLLNVDFEKTKE | |
| | KLILQFEDALGKNMTETALALSMLCEINCEEADVALEDYYHYWKNDPGAVNNW | |
| | FSIQALAHSPDVIERVKKLMRHGDFDLSNPNKVYALLGSFIKNPFGFHSVTGE | |
| | GYQLVADAIFDLDKINPTLAANLTEKFTYWDKYDVNRQAMMISTLKIIYSNAT | |
| | SSDVRTMAKKGLDKVKEDLPLPIHLTFHGGSTMQDRTAQLIADGNKENAYQLH | |
| E. coli methionine | MGTAISIKTPEDIEKMRVAGRLAAEVLEMIEPYVKPGVSTGELDRICNDYIVN | 10 |
| aminopeptidase | EQHAVSACLGYHGYPKSVCISINEVVCHGIPDDAKLLKDGDIVNIDVTVIKDG | |
| (Met specific) | FHGDTSKMFIVGKPTIMGERLCRITQESLYLALRMVKPGINLREIGAAIQKFV | |
| | EAEGFSVVREYCGHGIGRGFHEEPQVLHYDSRETNVVLKPGMTFTIEPMVNAG | |
| | KKEIRTMKDGWTVKTKDRSLSAQYEHTIVVTDNGCEILTLRKDDTIPAIISHD | |
| M. smegmatis | MGTLEANTNGPGSMLSRMPVSSRTVPFGDHETWVQVTTPENAQPHALPLIVLH | 11 |
| Proline | GGPGMAHNYVANIAALADETGRTVIHYDQVGCGNSTHLPDAPADFWTPQLFVD | |
| iminopeptidase | EFHAVCTALGIERYHVLGQSWGGMLGAEIAVRQPSGLVSLAICNSPASMRLWS | |
| (Pro specific) | EAAGDLRAQLPAETRAALDRHEAAGTITHPDYLQAAAEFYRRHVCRVVPTPQD | |
| | FADSVAQMEAEPTVYHTMNGPNEFHVVGTLGDWSVIDRLPDVTAPVLVIAGEH | |
| | DEATPKTWQPFVDHIPDVRSHVFPGTSHCTHLEKPEEFRAVVAQFLHQHDLAA | |
| | DARV | |
| P. furiosus | MDTEKLMKAGEIAKKVREKAIKLARPGMLLLELAESIEKMIMELGGKPAFPVN | 12 |
| methionine | LSINEIAAHYTPYKGDTTVLKEGDYLKIDVGVHIDGFIADTAVTVRVGMEEDE | |
| aminopeptidase | LMEAAKEALNAAISVARAGVEIKELGKAIENEIRKRGFKPIVNLSGHKIERYK | |
| | LHAGISIPNIYRPHDNYVLKEGDVFAIEPFATIGAGQVIEVPPTLIYMYVRDV | |
| | PVRVAQARFLLAKIKREYGTLPFAYRWLQNDMPEGQLKLALKTLEKAGAIYGY | |
| | PVLKEIRNGIVAQFEHTIIVEKDSVIVTQDMINKSTLE | |
| Aeromonas sobria | HMSSPLHYVLDGIHCEPHFFTVPLDHQQPDDEETITLFGRTLCRKDRLDDELP | 13 |
| Proline | WLLYLQGGPGFGAPRPSANGGWIKRALQEFRVLLLDQRGTGHSTPIHAELLAH | |
| aminopeptidase | LNPRQQADYLSHFRADSIVRDAELIREQLSPDHPWSLLGQSFGGFCSLTYLSL | |
| | FPDSLHEVYLTGGVAPIGRSADEVYRATYQRVADKNRAFFARFPHAQAIANRL | |
| | ATHLQRHDVRLPNGQRLTVEQLQQQGLDLGASGAFEELYYLLEDAFIGEKLNP | |
| | AFLYQVQAMQPFNTNPVFAILHELIYCEGAASHWAAERVRGEFPALAWAQGKD | |
| | FAFTGEMIFPWMFEQFRELIPLKEAAHLLAEKADWGPLYDPVQLARNKVPVAC | |
| | AVYAEDMYVEFDYSRETLKGLSNSRAWITNEYEHNGLRVDGEQILDRLIRLNR | |
| | DCLE | |
| Pyrococcus | MKERLEKLVKFMDENSIDRVFIAKPVNVYYFSGTSPLGGGYIIVDGDEATLYV | 14 |
| furiosus Proline | PELEYEMAKEESKLPVVKFKKFDEIYEILKNTETLGIEGTLSYSMVENFKEKS | |
| Aminopeptidase | NVKEFKKIDDVIKDLRIIKTKEEIEIIEKACEIADKAVMAAIEEITEGKRERE | |
| (X-/-Pro) | VAAKVEYLMKMNGAEKPAFDTIIASGHRSALPHGVASDKRIERGDLVVIDLGA | |
| | LYNHYNSDITRTIVVGSPNEKQREIYEIVLEAQKRAVEAAKPGMTAKELDSIA | |
| | REIIKEYGYGDYFIHSLGHGVGLEIHEWPRISQYDETVLKEGMVITIEPGIYI | |
| | PKLGGVRIEDTVLITENGAKRLTKTERELL | |
| Elizabethkingia | MIPITTPVGNFKVWTKRFGTNPKIKVLLLHGGPAMTHEYMECFETFFQREGFE | 15 |
| meningoseptica | FYEYDQLGSYYSDQPTDEKLWNIDRFVDEVEQVRKAIHADKENFYVLGNSWGG | |
| Proline | ILAMEYALKYQQNLKGLIVANMMASAPEYVKYAEVLSKQMKPEVLAEVRAIEA | |
| aminopeptidase | KKDYANPRYTELLFPNYYAQHICRLKEWPDALNRSLKHVNSTVYTLMQGPSEL | |
| | GMSSDARLAKWDIKNRLHEIATPTLMIGARYDTMDPKAMEEQSKLVQKGRYLY | |
| | CPNGSHLAMWDDQKVFMDGVIKFIKDVDTKSFN | |
| N. gonorrhoeae | MYEIKQPFHSGYLQVSEIHQIYWEESGNPDGVPVIFLHGGPGAGASPECRGFF | 16 |
| Proline | NPDVFRIVIIDQRGCGRSHPYACAEDNTTWDLVADIEKVREMLGIGKWLVFGG | |
| Iminopeptidase | SWGSTLSLAYAQTHPERVKGLVLRGIFLCRPSETAWLNEAGGVSRIYPEQWQK | |
| | FVAPIAENRRNRLIEAYHGLLFHQDEEVCLSAAKAWADWESYLIRFEPEGVDE | |
| | DAYASLAIARLENHYFVNGGWLQGDKAILNNIGKIRHIPTVIVQGRYDLCTPM | |
| | QSAWELSKAFPEAELRVVQAGHCAFDPPLADALVQAVEDILPRLL | |
| E. coli | MTQQPQAKYRHDYRAPDYQITDIDLTFDLDAQKTVVTAVSQAVRHGASDAPLR | 17 |
| Aminopeptidase N | LNGEDLKLVSVHINDEPWTAWKEEEGALVISNLPERFTLKIINEISPAANTAL | |
| (Zinc | EGLYQSGDALCTQCEAEGFRHITYYLDRPDVLARFTTKIIADKIKYPFLLSNG | |
| Metalloprotease) | NRVAQGELENGRHWVQWQDPFPKPCYLFALVAGDFDVLRDTFTTRSGREVALE | |
| | LYVDRGNLDRAPWAMTSLKNSMKWDEERFGLEYDLDIYMIVAVDFFNMGAMEN | |
| | KGLNIFNSKYVLARTDTATDKDYLDIERVIGHEYFHNWTGNRVTCRDWFQLSL | |
| | KEGLTVFRDQEFSSDLGSRAVNRINNVRTMRGLQFAEDASPMAHPIRPDMVIE | |
| | MNNFYTLTVYEKGAEVIRMIHTLLGEENFQKGMQLYFERHDGSAATCDDFVQA | |
| | MEDASNVDLSHFRRWYSQSGTPIVTVKDDYNPETEQYTLTISQRTPATPDQAE | |
| | KQPLHIPFAIELYDNEGKVIPLQKGGHPVNSVLNVTQAEQTFVFDNVYFQPVP | |
| | ALLCEFSAPVKLEYKWSDQQLTFLMRHARNDFSRWDAAQSLLATYIKLNVARH | |
| | QQGQPLSLPVHVADAFRAVLLDEKIDPALAAEILTLPSVNEMAELFDIIDPIA | |
| | IAEVREALTRTLATELADELLAIYNANYQSEYRVEHEDIAKRTLRNACLRFLA | |
| | FGETHLADVLVSKQFHEANNMTDALAALSAAVAAQLPCRDALMQEYDDKWHQN | |
| | GLVMDKWFILQATSPAANVLETVRGLLQHRSFTMSNPNRIRSLIGAFAGSNPA | |
| | AFHAEDGSGYLFLVEMLTDLNSRNPQVASRLIEPLIRLKRYDAKRQEKMRAAL | |
| | EQLKGLENLSGDLYEKITKALA | |
| P. falciparum M1 | PKIHYRKDYKPSGFIINQVTLNINIHDQETIVRSVLDMDISKHNVGEDLVFDG | 18 |
| aminopeptidase | VGLKINEISINNKKLVEGEEYTYDNEFLTIFSKFVPKSKFAFSSEVIIHPETN | |
| | YALTGLYKSKNIIVSQCEATGFRRITFFIDRPDMMAKYDVTVTADKEKYPVLL | |
| | SNGDKVNEFEIPGGRHGARFNDPPLKPCYLFAVVAGDLKHLSATYITKYTKKK | |
| | VELYVFSEEKYVSKLQWALECLKKSMAFDEDYFGLEYDLSRLNLVAVSDFNVG | |
| | AMENKGLNIFNANSLLASKKNSIDFSYARILTVVGHEYFHQYTGNRVTLRDWF | |
| | QLTLKEGLTVHRENLFSEEMTKTVTTRLSHVDLLRSVQFLEDSSPLSHPIRPE | |
| | SYVSMENFYTTTVYDKGSEVMRMYLTILGEEYYKKGFDIYIKKNDGNTATCED | |
| | FNYAMEQAYKMKKADNSANLNQYLLWFSQSGTPHVSFKYNYDAEKKQYSIHVN | |
| | QYTKPDENQKEKKPLFIPISVGLINPENGKEMISQTTLELTKESDTFVFNNIA | |
| | VKPIPSLFRGFSAPVYIEDQLTDEERILLLKYDSDAFVRYNSCTNIYMKQILM | |
| | NYNEFLKAKNEKLESFQLTPVNAQFIDAIKYLLEDPHADAGFKSYIVSLPQDR | |
| | YIINFVSNLDTDVLADTKEYIYKQIGDKLNDVYYKMFKSLEAKADDLTYFNDE | |
| | SHVDFDQMNMRTLRNTLLSLLSKAQYPNILNEIIEHSKSPYPSNWLTSLSVSA | |
| | YFDKYFELYDKTYKLSKDDELLLQEWLKTVSRSDRKDIYEILKKLENEVLKDS | |
| | KNPNDIRAVYLPFTNNLRRFHDISGKGYKLIAEVITKTDKFNPMVATQLCEPF | |
| | KLWNKLDTKRQELMLNEMNTMLQEPQISNNLKEYLLRLTNK | |
| Puromycin- | MWLAAAAPSLARRLLFLGPPPPPLLLLVFSRSSRRRLHSLGLAAMPEKRPFER | 19 |
| sensitive | LPADVSPINYSLCLKPDLLDFTFEGKLEAAAQVRQATNQIVMNCADIDIITAS | |
| aminopeptidase | YAPEGDEEIHATGFNYQNEDEKVTLSFPSTLQTGTGTLKIDFVGELNDKMKGF | |
| (NPEPPS) | YRSKYTTPSGEVRYAAVTQFEATDARRAFPCWDEPAIKATFDISLVVPKDRVA | |
| | LSNMNVIDRKPYPDDENLVEVKFARTPVMSTYLVAFVVGEYDFVETRSKDGVC | |
| | VRVYTPVGKAEQGKFALEVAAKTLPFYKDYFNVPYPLPKIDLIAIADFAAGAM | |
| | ENWGLVTYRETALLIDPKNSCSSSRQWVALVVGHELAHQWFGNLVTMEWWTHL | |
| | WLNEGFASWIEYLCVDHCFPEYDIWTQFVSADYTRAQELDALDNSHPIEVSVG | |
| | HPSEVDEIFDAISYSKGASVIRMLHDYIGDKDFKKGMNMYLTKFQQKNAATED | |
| | LWESLENASGKPIAAVMNTWTKQMGFPLIYVEAEQVEDDRLLRLSQKKFCAGG | |
| | SYVGEDCPQWMVPITISTSEDPNQAKLKILMDKPEMNVVLKNVKPDQWVKLNL | |
| | GTVGFYRTQYSSAMLESLLPGIRDLSLPPVDRLGLQNDLFSLARAGIISTVEV | |
| | LKVMEAFVNEPNYTVWSDLSCNLGILSTLLSHTDFYEEIQEFVKDVFSPIGER | |
| | LGWDPKPGEGHLDALLRGLVLGKLGKAGHKATLEEARRRFKDHVEGKQILSAD | |
| | LRSPVYLTVLKHGDGTTLDIMLKLHKQADMQEEKNRIERVLGATLLPDLIQKV | |
| | LTFALSEEVRPQDTVSVIGGVAGGSKHGRKAAWKFIKDNWEELYNRYQGGFLI | |
| | SRLIKLSVEGFAVDKMAGEVKAFFESHPAPSAERTIQQCCENILLNAAWLKRD | |
| | AESIHQYLLQRKASPPTV | |
| NPEPPS E366V | MWLAAAAPSLARRLLFLGPPPPPLLLLVFSRSSRRRLHSLGLAAMPEKRPFER | 20 |
| | LPADVSPINYSLCLKPDLLDFTFEGKLEAAAQVRQATNQIVMNCADIDIITAS | |
| | YAPEGDEEIHATGFNYQNEDEKVTLSFPSTLQTGTGTLKIDFVGELNDKMKGF | |
| | YRSKYTTPSGEVRYAAVTQFEATDARRAFPCWDEPAIKATFDISLVVPKDRVA | |
| | LSNMNVIDRKPYPDDENLVEVKFARTPVMSTYLVAFVVGEYDFVETRSKDGVC | |
| | VRVYTPVGKAEQGKFALEVAAKTLPFYKDYFNVPYPLPKIDLIAIADFAAGAM | |
| | ENWGLVTYRETALLIDPKNSCSSSRQWVALVVGHVLAHQWFGNLVTMEWWTHL | |
| | WLNEGFASWIEYLCVDHCFPEYDIWTQFVSADYTRAQELDALDNSHPIEVSVG | |
| | HPSEVDEIFDAISYSKGASVIRMLHDYIGDKDFKKGMNMYLTKFQQKNAATED | |
| | LWESLENASGKPIAAVMNTWTKQMGFPLIYVEAEQVEDDRLLRLSQKKFCAGG | |
| | SYVGEDCPQWMVPITISTSEDPNQAKLKILMDKPEMNVVLKNVKPDQWVKLNL | |
| | GTVGFYRTQYSSAMLESLLPGIRDLSLPPVDRLGLQNDLFSLARAGIISTVEV | |
| | LKVMEAFVNEPNYTVWSDLSCNLGILSTLLSHTDFYEEIQEFVKDVFSPIGER | |
| | LGWDPKPGEGHLDALLRGLVLGKLGKAGHKATLEEARRRFKDHVEGKQILSAD | |
| | LRSPVYLTVLKHGDGTTLDIMLKLHKQADMQEEKNRIERVLGATLLPDLIQKV | |
| | LTFALSEEVRPQDTVSVIGGVAGGSKHGRKAAWKFIKDNWEELYNRYQGGFLI | |
| | SRLIKLSVEGFAVDKMAGEVKAFFESHPAPSAERTIQQCCENILLNAAWLKRD | |
| | AESIHQYLLQRKASPPTV | |
| Francisella | MIYEFVMTDPKIKYLKDYKPSNYLIDETHLIFELDESKTRVTANLYIVANREN | 21 |
| tularensis | RENNTLVLDGVELKLLSIKLNNKHLSPAEFAVNENQLIINNVPEKFVLQTVVE | |
| Aminopeptidase N | INPSANTSLEGLYKSGDVFSTQCEATGFRKITYYLDRPDVMAAFTVKIIADKK | |
| | KYPIILSNGDKIDSGDISDNQHFAVWKDPFKKPCYLFALVAGDLASIKDTYIT | |
| | KSQRKVSLEIYAFKQDIDKCHYAMQAVKDSMKWDEDRFGLEYDLDTFMIVAVP | |
| | DFNAGAMENKGLNIFNTKYIMASNKTATDKDFELVQSVVGHEYFHNWTGDRVT | |
| | CRDWFQLSLKEGLTVFRDQEFTSDLNSRDVKRIDDVRIIRSAQFAEDASPMSH | |
| | PIRPESYIEMNNFYTVTVYNKGAEIIRMIHTLLGEEGFQKGMKLYFERHDGQA | |
| | VTCDDFVNAMADANNRDFSLFKRWYAQSGTPNIKVSENYDASSQTYSLTLEQT | |
| | TLPTADQKEKQALHIPVKMGLINPEGKNIAEQVIELKEQKQTYTFENIAAKPV | |
| | ASLFRDFSAPVKVEHKRSEKDLLHIVKYDNNAFNRWDSLQQIATNIILNNADL | |
| | NDEFLNAFKSILHDKDLDKALISNALLIPIESTIAEAMRVIMVDDIVLSRKNV | |
| | VNQLADKLKDDWLAVYQQCNDNKPYSLSAEQIAKRKLKGVCLSYLMNASDQKV | |
| | GTDLAQQLFDNADNMTDQQTAFTELLKSNDKQVRDNAINEFYNRWRHEDLVVN | |
| | KWLLSQAQISHESALDIVKGLVNHPAYNPKNPNKVYSLIGGFGANFLQYHCKD | |
| | GLGYAFMADTVLALDKFNHQVAARMARNLMSWKRYDSDRQAMMKNALEKIKAS | |
| | NPSKNVFEIVSKSLES | |
| T. aquaticus | MDAFTENLNKLAELAIRVGLNLEEGQEIVATAPIEAVDFVRLLAEKAYENGAS | 22 |
| Aminopeptidase T | LFTVLYGDNLIARKRLALVPEAHLDRAPAWLYEGMAKAFHEGAARLAVSGNDP | |
| | KALEGLPPERVGRAQQAQSRAYRPTLSAITEFVTNWTIVPFAHPGWAKAVFPG | |
| | LPEEEAVQRLWQAIFQATRVDQEDPVAAWEAHNRVLHAKVAFLNEKRFHALHF | |
| | QGPGTDLTVGLAEGHLWOGGATPTKKGRLCNPNLPTEEVFTAPHRERVEGVVR | |
| | ASRPLALSGQLVEGLWARFEGGVAVEVGAEKGEEVLKKLLDTDEGARRLGEVA | |
| | LVPADNPIAKTGLVFFDTLFDENAASHIAFGQAYAENLEGRPSGEEFRRRGGN | |
| | ESMVHVDWMIGSEEVDVDGLLEDGTRVPLMRRGRWVI | |
| Bacillus | MAKLDETLTMLKALTDAKGVPGNEREARDVMKTYIAPYADEVTTDGLGSLIAK | 23 |
| stearothermophilus | KEGKSGGPKVMIAGHLDEVGFMVTQIDDKGFIRFQTLGGWWSQVMLAQRVTIV | |
| Peptidase M28 | TKKGDITGVIGSKPPHILPSEARKKPVEIKDMFIDIGATSREEAMEWGVRPGD | |
| | MIVPYFEFTVLNNEKMLLAKAWDNRIGCAVAIDVLKQLKGVDHPNTVYGVGTV | |
| | QEEVGLRGARTAAQFIQPDIAFAVDVGIAGDTPGVSEKEAMGKLGAGPHIVLY | |
| | DATMVSHRGLREFVIEVAEELNIPHHFDAMPGVGTDAGAIHLTGIGVPSLTIA | |
| | IPTRYIHSHAAILHRDDYENTVKLLVEVIKRLDADKVKQLTFDE | |
| Vibrio cholera | MEDKVWISMGADAVGSLNPALSESLLPHSFASGSQVWIGEVAIDELAELSHTM | 24 |
| Aminopeptidase | HEQHNRCGGYMVHTSAQGAMAALMMPESIANFTIPAPSQQDLVNAWLPQVSAD | |
| | QITNTIRALSSFNNRFYTTTSGAQASDWLANEWRSLISSLPGSRIEQIKHSGY | |
| | NQKSVVLTIQGSEKPDEWVIVGGHLDSTLGSHTNEQSIAPGADDDASGIASLS | |
| | EIIRVLRDNNFRPKRSVALMAYAAEEVGLRGSQDLANQYKAQGKKVVSVLQLD | |
| | MTNYRGSAEDIVFITDYTDSNLTQFLTTLIDEYLPELTYGYDRCGYACSDHAS | |
| | WHKAGFSAAMPFESKFKDYNPKIHTSQDTLANSDPTGNHAVKFTKLGLAYVIE | |
| | MANAGSSQVPDDSVLQDGTAKINLSGARGTQKRFTFELSQSKPLTIQTYGGSG | |
| | DVDLYVKYGSAPSKSNWDCRPYQNGNRETCSFNNAQPGIYHVMLDGYTNYNDV | |
| | ALKASTQ | |
| Photobacterium | MEDKVWISIGSDASQTVKSVMQSNARSLLPESLASNGPVWVGQVDYSQLAELS | 25 |
| halotolerans | HHMHEDHQRCGGYMVHSSPESAIAASNMPQSLVAFSIPEISQQDTVNAWLPQV | |
| Aminopeptidase | NSQAITGTITSLTSFINRFYTTTSGAQASDWLANEWRSLSASLPNASVRQVSH | |
| | FGYNQKSVVLTITGSEKPDEWIVLGGHLDSTIGSHTNEQSVAPGADDDASGIA | |
| | SVTEIIRVLSENNFQPKRSIAFMAYAAEEVGLRGSQDLANQYKAEGKQVISAL | |
| | QLDMTNYKGSVEDIVFITDYTDSNLTTFLSQLVDEYLPSLTYGFDTCGYACSD | |
| | HASWHKAGFSAAMPFEAKFNDYNPMIHTPNDTLQNSDPTASHAVKFTKLGLAY | |
| | AIEMASTTGGTPPPTGNVLKDGVPVNGLSGATGSQVHYSFELPAQKNLQISTA | |
| | GGSGDVDLYVSFGSEATKQNWDCRPYRNGNNEVCTFAGATPGTYSIMLDGYRQ | |
| | FSGVTLKASTQ | |
| Yersinia pestis | MTQQPQAKYRHDYRAPDYTITDIDLDFALDAQKTTVTAVSKVKRQGTDVTPLI | 26 |
| Aminopeptidase N | LNGEDLTLISVSVDGQAWPHYRQQDNTLVIEQLPADFTLTIVNDIHPATNSAL | |
| | EGLYLSGEALCTQCEAEGFRHITYYLDRPDVLARFTTRIVADKSRYPYLLSNG | |
| | NRVGQGELDDGRHWVKWEDPFPKPSYLFALVAGDFDVLQDKFITRSGREVALE | |
| | IFVDRGNLDRADWAMTSLKNSMKWDETRFGLEYDLDIYMIVAVDFFNMGAMEN | |
| | KGLNVFNSKYVLAKAETATDKDYLNIEAVIGHEYFHNWTGNRVTCRDWFQLSL | |
| | KEGLTVFRDQEFSSDLGSRSVNRIENVRVMRAAQFAEDASPMAHAIRPDKVIE | |
| | MNNFYTLTVYEKGSEVIRMMHTLLGEQQFQAGMRLYFERHDGSAATCDDFVQA | |
| | MEDVSNVDLSLFRRWYSQSGTPLLTVHDDYDVEKQQYHLFVSQKTLPTADQPE | |
| | KLPLHIPLDIELYDSKGNVIPLQHNGLPVHHVLNVTEAEQTFTFDNVAQKPIP | |
| | SLLREFSAPVKLDYPYSDQQLTFLMQHARNEFSRWDAAQSLLATYIKLNVAKY | |
| | QQQQPLSLPAHVADAFRAILLDEHLDPALAAQILTLPSENEMAELFTTIDPQA | |
| | ISTVHEAITRCLAQELSDELLAVYVANMTPVYRIEHGDIAKRALRNTCLNYLA | |
| | FGDEEFANKLVSLQYHQADNMTDSLAALAAAVAAQLPCRDELLAAFDVRWNHD | |
| | GLVMDKWFALQATSPAANVLVQVRTLLKHPAFSLSNPNRTRSLIGSFASGNPA | |
| | AFHAADGSGYQFLVEILSDLNTRNPQVAARLIEPLIRLKRYDAGRQALMRKAL | |
| | EQLKTLDNLSGDLYEKITKALAA | |
| Vibrio | MEEKVWISIGGDATQTALRSGAQSLLPENLINQTSVWVGQVPVSELATLSHEM | 27 |
| anguillarum | HENHQRCGGYMVHPSAQSAMSVSAMPLNLNAFSAPEITQQTTVNAWLPSVSAQ | |
| Aminopeptidase | QITSTITTLTQFKNRFYTTSTGAQASNWIADHWRSLSASLPASKVEQITHSGY | |
| | NQKSVMLTITGSEKPDEWVVIGGHLDSTLGSRTNESSIAPGADDDASGIAGVT | |
| | EIIRLLSEQNFRPKRSIAFMAYAAEEVGLRGSQDLANRFKAEGKKVMSVMQLD | |
| | MTNYQGSREDIVFITDYTDSNFTQYLTQLLDEYLPSLTYGFDTCGYACSDHAS | |
| | WHAVGYPAAMPFESKFNDYNPNIHSPQDTLQNSDPTGFHAVKFTKLGLAYVVE | |
| | MGNASTPPTPSNQLKNGVPVNGLSASRNSKTWYQFELQEAGNLSIVLSGGSGD | |
| | ADLYVKYQTDADLQQYDCRPYRSGNNETCQFSNAQPGRYSILLHGYNNYSNAS | |
| | LVANAQ | |
| Salinivibrio | MEDKKVWISIGADAQQTALSSGAQPLLAQSVAHNGQAWIGEVSESELAALSHE | 28 |
| spYCSC6 | MHENHHRCGGYIVHSSAQSAMAASNMPLSRASFIAPAISQQALVTPWISQIDS | |
| Aminopeptidase | ALIVNTIDRLTDFPNRFYTTTSGAQASDWIKQRWQSLSAGLAGASVTQISHSG | |
| | YNQASVMLTIEGSESPDEWVVVGGHLDSTIGSRTNEQSIAPGADDDASGIAAV | |
| | TEVIRVLAQNNFQPKRSIAFVAYAAEEVGLRGSQDVANQFKQAGKDVRGVLQL | |
| | DMTNYQGSAEDIVFITDYTDNQLTQYLTQLLDEYLPTLNYGFDTCGYACSDHA | |
| | SWHQVGYPAAMPFEAKFNDYNPNIHTPQDTLANSDSEGAHAAKFTKLGLAYTV | |
| | ELANADSSPNPGNELKLGEPINGLSGARGNEKYFNYRLDQSGELVIRTYGGSG | |
| | DVDLYVKANGDVSTGNWDCRPYRSGNDEVCRFDNATPGNYAVMLRGYRTYDNV | |
| | SLIVE | |
| Vibrio | MPPITQQATVTAWLPQVDASQITGTISSLESFTNRFYTTTSGAQASDWIASEW | 29 |
| proteolyticus | QALSASLPNASVKQVSHSGYNQKSVVMTITGSEAPDEWIVIGGHLDSTIGSHT | |
| Aminopeptidase I | NEQSVAPGADDDASGIAAVTEVIRVLSENNFQPKRSIAFMAYAAEEVGLRGSQ | |
| | DLANQYKSEGKNVVSALQLDMTNYKGSAQDVVFITDYTDSNFTQYLTQLMDEY | |
| | LPSLTYGFDTCGYACSDHASWHNAGYPAAMPFESKFNDYNPRIHTTQDTLANS | |
| | DPTGSHAKKFTQLGLAYAIEMGSATGDTPTPGNQLE | |
| Vibrio | MPPITQQATVTAWLPQVDASQITGTISSLESFTNRFYTTTSGAQASDWIASEW | 30 |
| proteolyticus | QFLSASLPNASVKQVSHSGYNQKSVVMTITGSEAPDEWIVIGGHLDSTIGSHT | |
| Aminopeptidase I | NEQSVAPGADDDASGIAAVTEVIRVLSENNFQPKRSIAFMAYAAEEVGLRGSQ | |
| (A55F) | DLANQYKSEGKNVVSALQLDMTNYKGSAQDVVFITDYTDSNFTQYLTQLMDEY | |
| | LPSLTYGFDTCGYACSDHASWHNAGYPAAMPFESKFNDYNPRIHTTQDTLANS | |
| | DPTGSHAKKFTQLGLAYAIEMGSATGDTPTPGNQLE | |
| P. furiosus | MVDWELMKKIIESPGVSGYEHLGIRDLVVDILKDVADEVKIDKLGNVIAHFKG | 31 |
| Aminopeptidase I | SAPKVMVAAHMDKIGLMVNHIDKDGYLRVVPIGGVLPETLIAQKIRFFTEKGE | |
| | RYGVVGVLPPHLRREAKDQGGKIDWDSIIVDVGASSREEAEEMGFRIGTIGEF | |
| | APNFTRLSEHRFATPYLDDRICLYAMIEAARQLGEHEADIYIVASVQEEIGLR | |
| | GARVASFAIDPEVGIAMDVTFAKQPNDKGKIVPELGKGPVMDVGPNINPKLRQ | |
| | FADEVAKKYEIPLQVEPSPRPTGTDANVMQINREGVATAVLSIPIRYMHSQVE | |
| | LADARDVDNTIKLAKALLEELKPMDFTPLE | |

In certain embodiments, the peptidase is an exopeptidase. In certain embodiments, the peptidase is an aminopeptidase. In certain embodiments, the peptidase is proline aminopeptidase, a proline iminopeptidase, a glutamate/aspartate-specific aminopeptidase, a methionine-specific aminopeptidase, or a zinc metalloprotease. In certain embodiments, the peptidase is a TET aminopeptidase. In certain embodiments, the TET aminopeptidase is hTet. In certain embodiments, the TET aminopeptidase is pfuTet.

In some embodiments, reacting the compound of Formula (II), or salt thereof, with a peptidase, in a degradation process comprises one or more amino acid recognizers (e.g., one or more amino acid binding proteins not having peptide cleavage activity). In some embodiments, an amino acid recognizer comprises an amino acid binding protein, such as a ClpS protein (e.g., Planctomycetia bacterium ClpS protein), a UBR protein (e.g., Kluyveromyces marxianus UBR protein), an Ntaq1 protein (e.g., Scleropages formosus Ntaq1 protein), or a variant or homolog thereof. In some embodiments, an amino acid recognizer comprises a label (e.g., a detectable label, such as a luminescent label). Examples of amino acid recognizers (e.g., recognition molecules) are described in detail in PCT International Publication No. WO2020/102741A1, filed Nov. 15, 2019, PCT International Publication No. WO2021/236983A2, filed May 20, 2021, and co-pending U.S. Ser. No. 63/395,328, filed Aug. 4, 2022, the relevant content of each of which is incorporated by reference in its entirety.

In some embodiments, reacting the compound of Formula (II), or salt thereof, with a peptidase, in a degradation process (e.g., a reaction mixture) can be configured to achieve a time interval that allows for sufficient association events which provide a desired confidence level with a characteristic pattern. This can be achieved, for example, by configuring the reaction conditions based on various properties, including: linker identity, reagent concentration, molar ratio of one reagent to another (e.g., ratio of amino acid recognizer to cleaving reagent, ratio of one recognizer to another, ratio of one cleaving reagent to another), number of different reagent types (e.g., the number of different types of recognizers and/or cleaving reagents, the number of recognizer types relative to the number of cleaving reagent types), cleavage activity (e.g., aminopeptidase activity), binding properties (e.g., kinetic and/or thermodynamic binding parameters for recognition molecule binding), reagent modification (e.g., polyol and other recognizer modifications which can alter interaction dynamics), reaction mixture components (e.g., one or more components, such as pH, buffering agent, salt, divalent cation, surfactant, and other reaction mixture components described herein), temperature of the reaction, and various other parameters apparent to those skilled in the art, and combinations thereof. The reaction conditions can be configured based on one or more aspects described herein, including, for example, signal pulse information (e.g., pulse duration, interpulse duration, change in magnitude), labeling strategies (e.g., number and/or type of fluorophore, linkers with or without shielding element), surface modification (e.g., modification of sample well surface, including polypeptide immobilization), sample preparation (e.g., polypeptide fragment size, polypeptide modification for immobilization), and other aspects described herein.

In some embodiments, reacting the compound of Formula (II), or salt thereof, with a peptidase, in a degradation process is performed under conditions in which recognition and cleavage of amino acids can occur simultaneously in a single reaction mixture. For example, in some embodiments, a polypeptide sequencing reaction is performed in a reaction mixture having a pH at which association events and cleavage events can occur. Accordingly, in some embodiments, a reaction mixture has a pH of between about 6.5 and about 9.0. In some embodiments, a reaction mixture has a pH of between about 7.0 and about 8.5 (e.g., between about 7.0 and about 8.0, between about 7.5 and about 8.5, between about 7.5 and about 8.0, or between about 8.0 and about 8.5).

In some embodiments, reacting the compound of Formula (II), or salt thereof, with a peptidase, in a degradation process is performed in a reaction mixture comprising one or more buffering agents. In some embodiments, a reaction mixture comprises a buffering agent in a concentration of at least 10 mM (e.g., at least 20 mM and up to 250 mM, at least 50 mM, 10-250 mM, 10-100 mM, 20-100 mM, 50-100 mM, or 100-200 mM). In some embodiments, a reaction mixture comprises a buffering agent in a concentration of between about 10 mM and about 50 mM (e.g., between about 10 mM and about 25 mM, between about 25 mM and about 50 mM, or between about 20 mM and about 40 mM). Examples of buffering agents include, without limitation, HEPES (4-(2-hydroxyethyl)-1-piperazineethanesulfonic acid), Tris (tris(hydroxymethyl)aminomethane), and MOPS (3-(N-morpholino)propanesulfonic acid).

In some embodiments, reacting the compound of Formula (II), or salt thereof, with a peptidase, in a degradation process is performed in a reaction mixture comprising salt in a concentration of at least 10 mM. In some embodiments, a reaction mixture comprises salt in a concentration of at least 10 mM (e.g., at least 20 mM, at least 50 mM, at least 100 mM, or more). In some embodiments, a reaction mixture comprises salt in a concentration of between about 10 mM and about 250 mM (e.g., between about 20 mM and about 200 mM, between about 50 mM and about 150 mM, between about 10 mM and about 50 mM, or between about 10 mM and about 100 mM). Examples of salts include, without limitation, sodium salts, potassium salts, and acetates, such as sodium chloride (NaCl), sodium acetate (NaOAc), and potassium acetate (KOAc).

Additional examples of components for use in reacting the compound of Formula (II), or salt thereof, with a peptidase, in a degradation process (i.e., a reaction mixture) include divalent cations (e.g., Mg²⁺, Co²⁺) and surfactants (e.g., polysorbate 20). In some embodiments, a reaction mixture comprises a divalent cation in a concentration of between about 0.1 mM and about 50 mM (e.g., between about 10 mM and about 50 mM, between about 0.1 mM and about 10 mM, or between about 1 mM and about 20 mM). In some embodiments, a reaction mixture comprises a surfactant in a concentration of at least 0.01% (e.g., between about 0.01% and about 0.10%). In some embodiments, a reaction mixture comprises one or more components useful in single-molecule analysis, such as an oxygen-scavenging system (e.g., a PCA/PCD system or a Pyranose oxidase/Catalase/glucose system) and/or one or more triplet state quenchers (e.g., trolox, COT, and NBA).

In some embodiments, reacting the compound of Formula (II), or salt thereof, with a peptidase, in a degradation process is performed at a temperature at which association events and cleavage events can occur. In some embodiments, a polypeptide sequencing reaction is performed at a temperature of at least 10° C. In some embodiments, a polypeptide sequencing reaction is performed at a temperature of between about 10° C. and about 50° C. (e.g., 15-45° C., 20-40° C., at or around 25° C., at or around 30° C., at or around 35° C., at or around 37° C.). In some embodiments, a polypeptide sequencing reaction is performed at or around room temperature.

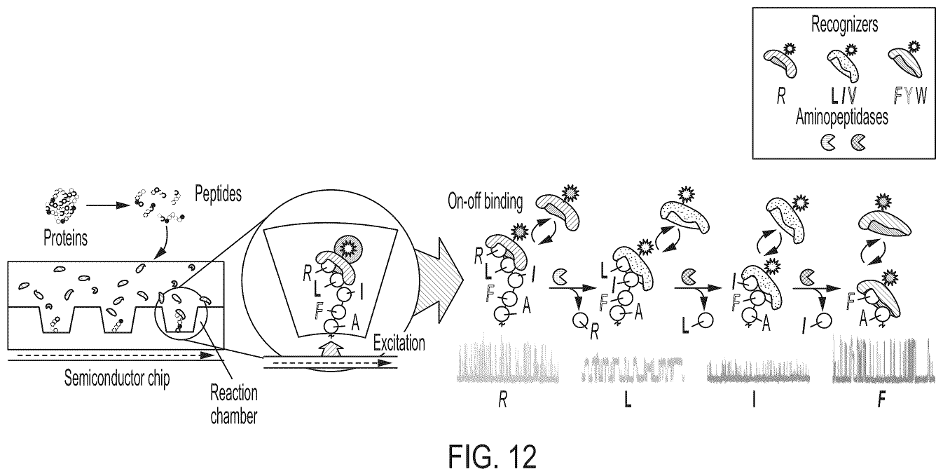

As detailed above, a real-time sequencing process as illustrated by FIG. 12 can generally involve cycles of amino acid recognition and terminal amino acid cleavage. In some embodiments, the relative occurrence of recognition and cleavage can be controlled by a concentration differential between one or more amino acid recognizers and at least one cleaving reagent. In some embodiments, the concentration differential can be optimized such that the number of signal pulses detected during recognition of an individual amino acid provides a desired confidence interval for identification. For example, if an initial sequencing reaction provides signal data with too few signal pulses between cleavage events to permit determination of characteristic patterns with a desired confidence interval, the sequencing reaction can be repeated using a decreased concentration of non-specific exopeptidase relative to recognition molecule.

In some embodiments, reacting the compound of Formula (II), or salt thereof, with a peptidase, in a degradation process may be carried out by contacting a polypeptide with a reaction mixture comprising one or more amino acid recognizers and one or more cleaving reagents (e.g., peptidases). In some embodiments, a reaction mixture comprises an amino acid recognizer at a concentration of between about 10 nM and about 10 μM. In some embodiments, a reaction mixture comprises a cleaving reagent at a concentration of between about 500 nM and about 500 μM.

In some embodiments, reacting the compound of Formula (II), or salt thereof, with a peptidase, in a degradation process (i.e., a reaction mixture) comprises an amino acid recognizer at a concentration of between about 100 nM and about 10 μM, between about 250 nM and about 10 μM, between about 100 nM and about 1 μM, between about 250 nM and about 1 μM, between about 250 nM and about 750 nM, or between about 500 nM and about 1 μM. In some embodiments, a reaction mixture comprises an amino acid recognizer at a concentration of about 100 nM, about 250 nM, about 500 nM, about 750 nM, or about 1 μM. In some embodiments, a reaction mixture comprises a cleaving reagent at a concentration of between about 500 nM and about 250 μM, between about 500 nM and about 100 μM, between about 1 μM and about 100 μM, between about 500 nM and about 50 M, between about 1 μM and about 100 μM, between about 10 μM and about 200 μM, or between about 10 μM and about 100 μM. In some embodiments, a reaction mixture comprises a cleaving reagent at a concentration of about 1 μM, about 5 μM, about 10 μM, about 30 μM, about 50 μM, about 70 μM, or about 100 μM.

In some embodiments, reacting the compound of Formula (II), or salt thereof, with a peptidase, in a degradation process (i.e., a reaction mixture) comprises an amino acid recognizer at a concentration of between about 10 nM and about 10 μM, and a cleaving reagent at a concentration of between about 500 nM and about 500 μM. In some embodiments, a reaction mixture comprises an amino acid recognizer at a concentration of between about 100 nM and about 1 μM, and a cleaving reagent at a concentration of between about 1 μM and about 100 μM. In some embodiments, a reaction mixture comprises an amino acid recognizer at a concentration of between about 250 nM and about 1 μM, and a cleaving reagent at a concentration of between about 10 μM and about 100 μM. In some embodiments, a reaction mixture comprises an amino acid recognizer at a concentration of about 500 nM, and a cleaving reagent at a concentration of between about 25 μM and about 75 μM. In some embodiments, the concentration of an amino acid recognizer and/or the concentration of a cleaving reagent in a reaction mixture is as described elsewhere herein.

In some embodiments, reacting the compound of Formula (II), or salt thereof, with a peptidase, in a degradation process (i.e., a reaction mixture) comprises an amino acid recognizer and a cleaving reagent in a molar ratio of about 500:1, about 400:1, about 300:1, about 200:1, about 100:1, about 75:1, about 50:1, about 25:1, about 10:1, about 5:1, about 2:1, or about 1:1. In some embodiments, a reaction mixture comprises an amino acid recognizer and a cleaving reagent in a molar ratio of between about 10:1 and about 200:1. In some embodiments, a reaction mixture comprises an amino acid recognizer and a cleaving reagent in a molar ratio of between about 50:1 and about 150:1. In some embodiments, the molar ratio of an amino acid recognizer to a cleaving reagent in a reaction mixture is between about 1:1,000 and about 1:1 or between about 1:1 and about 100:1 (e.g., 1:1,000, about 1:500, about 1:200, about 1:100, about 1:10, about 1:5, about 1:2, about 1:1, about 5:1, about 10:1, about 50:1, about 100:1). In some embodiments, the molar ratio of an amino acid recognizer to a cleaving reagent in a reaction mixture is between about 1:100 and about 1:1 or between about 1:1 and about 10:1. In some embodiments, the molar ratio of an amino acid recognizer to a cleaving reagent in a reaction mixture is as described elsewhere herein.

In some embodiments, a reaction mixture comprises one or more amino acid recognizers and one or more cleaving reagents described herein. In some embodiments, a reaction mixture comprises at least three amino acid recognizers and at least one cleaving reagent. In some embodiments, the reaction mixture comprises two or more cleaving reagents. In some embodiments, the reaction mixture comprises at least one and up to ten cleaving reagents (e.g., 1-3 cleaving reagents, 2-10 cleaving reagents, 1-5 cleaving reagents, 3-10 cleaving reagents). In some embodiments, the reaction mixture comprises at least three and up to thirty amino acid recognizers (e.g., between 3 and 25, between 3 and 20, between 3 and 10, between 3 and 5, between 5 and 30, between 5 and 20, between 5 and 10, or between 10 and 20, amino acid recognizers).

In some embodiments, reacting the compound of Formula (II), or salt thereof, with a peptidase, in a degradation process (i.e., a reaction mixture) comprises more than one amino acid recognizer and/or more than one cleaving reagent. In some embodiments, a reaction mixture described as comprising more than one amino acid recognizer or cleaving reagent refers to the mixture as having more than one type of amino acid recognizer or cleaving reagent. For example, in some embodiments, a reaction mixture comprises two or more cleaving reagents, where the two or more cleaving reagents refer to two or more types of aminopeptidases. In some embodiments, one type of aminopeptidase has an amino acid sequence that is different from another type of aminopeptidase in the reaction mixture. In some embodiments, one type of cleaving reagent cleaves an amino acid or subset of amino acids that is different from an amino acid or subset of amino acids cleaved by another type of cleaving reagent in the reaction mixture.

In some aspects, the application provides methods comprising obtaining data during a degradation process of a polypeptide. In some embodiments, the methods comprise analyzing the data to determine portions of the data corresponding to amino acids that are sequentially exposed at a terminus of the polypeptide during the degradation process. In some embodiments, the methods comprise outputting an amino acid sequence representative of the polypeptide. In some embodiments, the data is indicative of amino acid identity at the terminus of the polypeptide during the degradation process. In some embodiments, the data is indicative of a luminescent signal generated during the degradation process. In some embodiments, the data is indicative of an electrical signal generated during the degradation process.

In some embodiments, analyzing the data further comprises detecting a series of cleavage events and determining the portions of the data between successive cleavage events. In some embodiments, analyzing the data further comprises determining a type of amino acid for each of the individual portions. In some embodiments, each of the individual portions comprises a pulse pattern (e.g., a characteristic pattern), and analyzing the data further comprises determining a type of amino acid for one or more of the portions based on its respective pulse pattern. In some embodiments, determining the type of amino acid further comprises identifying an amount of time within a portion when the data is above a threshold value and comparing the amount of time to a duration of time for the portion. In some embodiments, determining the type of amino acid further comprises identifying at least one pulse duration for each of the one or more portions. In some embodiments, the pulse pattern comprises a mean pulse duration of between about 1 millisecond and about 10 seconds. In some embodiments, determining the type of amino acid further comprises identifying at least one interpulse duration for each of the one or more portions. In some embodiments, the amino acid sequence includes a series of amino acids corresponding to the portions. In some embodiments, the pulse pattern is produced by an amino acid recognizer associated with one or more reagents of a sequencing reaction. In some embodiments, the pulse pattern is produced by association and dissociation of an amino acid recognizer with one or more reagents of a sequencing reaction.

A non-limiting example of polypeptide structure analysis by detecting single molecule binding interactions during a polypeptide degradation process is illustrated in FIG. 12. An example signal trace is shown depicting different association (e.g., binding) events at times corresponding to changes in the signal. As shown, an association event between an amino acid recognizer and a terminal end of a polypeptide produces a change in magnitude of the signal that persists for a duration of time. Different association events are illustrated for different amino acids exposed at the terminal end of the polypeptide. As described herein, an amino acid that is “exposed” at the terminus of a polypeptide is an amino acid that is still attached to the polypeptide and that becomes the terminal amino acid upon removal of the prior terminal amino acid during degradation (e.g., either alone or along with one or more additional amino acids).

As generically depicted, the association events between amino acid recognizers and different types of amino acids at the terminal end of the polypeptide produce distinctive changes in the signal, referred to herein as a characteristic pattern, which may be used to determine chemical characteristics of the polypeptide. In some embodiments, a characteristic pattern corresponding to one type of terminal amino acid can be used to determine structural information for the terminal amino acid and one or more amino acids contiguous to the terminal amino acid. Accordingly, in some embodiments, a characteristic pattern corresponding to one type of terminal amino acid can be used to determine structural information for at least two (e.g., at least three, at least four, at least five, two, three, four, or between two and five) amino acids of a polypeptide.

In some embodiments, a transition from one characteristic pattern to another is indicative of amino acid cleavage. As used herein, in some embodiments, amino acid cleavage refers to the removal of at least one amino acid from a terminus of a polypeptide (e.g., the removal of at least one terminal amino acid from the polypeptide). In some embodiments, amino acid cleavage is determined by inference based on a time duration between characteristic patterns. In some embodiments, amino acid cleavage is determined by detecting a change in signal produced by association of a labeled cleaving reagent with an amino acid at the terminus of the polypeptide. As amino acids are sequentially cleaved from the terminus of the polypeptide during degradation, a series of changes in magnitude, or a series of signal pulses, is detected.

In some embodiments, signal data can be analyzed to extract signal pulse information by applying threshold levels to one or more parameters of the signal data. For example, in some embodiments, a threshold magnitude level may be applied to the signal data of a signal trace. In some embodiments, the threshold magnitude level is a minimum difference between a signal detected at a point in time and a baseline determined for a given set of data. In some embodiments, a signal pulse is assigned to each portion of the data that is indicative of a change in magnitude exceeding the threshold magnitude level and persisting for a duration of time. In some embodiments, a threshold time duration may be applied to a portion of the data that satisfies the threshold magnitude level to determine whether a signal pulse is assigned to that portion. For example, experimental artifacts may give rise to a change in magnitude exceeding the threshold magnitude level but that does not persist for a duration of time sufficient to assign a signal pulse with a desired confidence (e.g., transient association events which could be non-discriminatory for amino acid type, non-specific detection events such as diffusion into an observation region or reagent sticking within an observation region). Accordingly, in some embodiments, a signal pulse is extracted from signal data based on a threshold magnitude level and a threshold time duration.

In some embodiments, a peak in magnitude of a signal pulse is determined by averaging the magnitude detected over a duration of time that persists above the threshold magnitude level. It should be appreciated that, in some embodiments, a “signal pulse” as used herein can refer to a change in signal data that persists for a duration of time above a baseline (e.g., raw signal data), or to signal pulse information extracted therefrom (e.g., processed signal data).

In some embodiments, signal pulse information can be analyzed to identify different types of amino acids in a polypeptide based on different characteristic patterns in a series of signal pulses. For example, as shown in FIG. 12, the signal pulse information is indicative of different types of amino acids at a terminal end of a polypeptide (e.g., arginine, leucine, isoleucine, phenylalanine). By way of example, the signal pulses detected at the earliest time points provide information indicative of (at least) arginine at the terminus of the polypeptide based on a first characteristic pattern, and the signal pulses detected at the latest time points provide information indicative of at least phenylalanine at the terminus of the polypeptide based on a second characteristic pattern.

In some embodiments, each signal pulse of a characteristic pattern comprises a pulse duration corresponding to an association event between an amino acid recognizer and an amino acid ligand. In some embodiments, the pulse duration is characteristic of a dissociation rate of binding. In some embodiments, each signal pulse of a characteristic pattern is separated from another signal pulse of the characteristic pattern by an interpulse duration. In some embodiments, the interpulse duration is characteristic of an association rate of binding. In some embodiments, a change in magnitude in a signal can be determined for a signal pulse based on a difference between baseline and the peak of a signal pulse. In some embodiments, a characteristic pattern is determined based on pulse duration. In some embodiments, a characteristic pattern is determined based on pulse duration and interpulse duration. In some embodiments, a characteristic pattern is determined based on any one or more of pulse duration, interpulse duration, and change in magnitude.

Accordingly, as illustrated by FIG. 12, in some embodiments, polypeptide analysis is performed by detecting a series of signal pulses indicative of association of one or more amino acid recognizers with successive amino acids exposed at the terminus of a polypeptide in an ongoing degradation reaction. The series of signal pulses can be analyzed to determine characteristic patterns in the series of signal pulses, and the time course of characteristic patterns can be used to determine chemical characteristics throughout an amino acid sequence of the polypeptide.

As described herein, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, the mean pulse duration is between about 50 milliseconds and about 2 seconds, between about 50 milliseconds and about 500 milliseconds, or between about 500 milliseconds and about 2 seconds.

In some embodiments, different characteristic patterns corresponding to different types of amino acids in a single polypeptide may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). In some embodiments, the difference in mean pulse duration is at least 50 ms, at least 100 ms, at least 250 ms, at least 500 ms, or more. In some embodiments, the difference in mean pulse duration is between about 50 ms and about 1 s, between about 50 ms and about 500 ms, between about 50 ms and about 250 ms, between about 100 ms and about 500 ms, between about 250 ms and about 500 ms, or between about 500 ms and about 1 s. In some embodiments, the mean pulse duration of one characteristic pattern is different from the mean pulse duration of another characteristic pattern by about 10-25%, 25-50%, 50-75%, 75-100%, or more than 100%, for example by about 2-fold, 3-fold, 4-fold, 5-fold, or more. It should be appreciated that, in some embodiments, smaller differences in mean pulse duration between different characteristic patterns may require a greater number of pulse durations within each characteristic pattern to distinguish one from another with statistical confidence.

In some embodiments, a characteristic pattern generally refers to a plurality of association events between an amino acid of a polypeptide and a means for binding the amino acid (e.g., an amino acid recognition molecule). In some embodiments, a characteristic pattern comprises at least 10 association events (e.g., at least 25, at least 50, at least 75, at least 100, at least 250, at least 500, at least 1,000, or more, association events). In some embodiments, a characteristic pattern comprises between about 10 and about 1,000 association events (e.g., between about 10 and about 500 association events, between about 10 and about 250 association events, between about 10 and about 100 association events, or between about 50 and about 500 association events). In some embodiments, the plurality of association events is detected as a plurality of signal pulses.

In some embodiments, a characteristic pattern refers to a plurality of signal pulses which may be characterized by a summary statistic as described herein. In some embodiments, a characteristic pattern comprises at least 10 signal pulses (e.g., at least 25, at least 50, at least 75, at least 100, at least 250, at least 500, at least 1,000, or more, signal pulses). In some embodiments, a characteristic pattern comprises between about 10 and about 1,000 signal pulses (e.g., between about 10 and about 500 signal pulses, between about 10 and about 250 signal pulses, between about 10 and about 100 signal pulses, or between about 50 and about 500 signal pulses).

In some embodiments, a characteristic pattern refers to a plurality of association events between an amino acid recognition molecule and an amino acid of a polypeptide occurring over a time interval prior to removal of the amino acid (e.g., a cleavage event). In some embodiments, a characteristic pattern refers to a plurality of association events occurring over a time interval between two cleavage events (e.g., prior to removal of the amino acid and after removal of an amino acid previously exposed at the terminus). In some embodiments, the time interval of a characteristic pattern is between about 1 minute and about 30 minutes (e.g., between about 1 minute and about 20 minutes, between about 1 minute and 10 minutes, between about 5 minutes and about 20 minutes, between about 5 minutes and about 15 minutes, or between about 5 minutes and about 10 minutes).

In some embodiments, the series of signal pulses comprises a series of changes in magnitude of an optical signal over time. In some embodiments, the series of changes in the optical signal comprises a series of changes in luminescence produced during association events. In some embodiments, luminescence is produced by a detectable label associated with one or more reagents of a sequencing reaction. For example, in some embodiments, each of the one or more amino acid recognizers comprises a luminescent label. In some embodiments, a cleaving reagent comprises a luminescent label. Examples of luminescent labels and their use in accordance with the application are provided herein.

In some embodiments, the series of signal pulses comprises a series of changes in magnitude of an electrical signal over time. In some embodiments, the series of changes in the electrical signal comprises a series of changes in conductance produced during association events. In some embodiments, conductivity is produced by a detectable label associated with one or more reagents of a sequencing reaction. For example, in some embodiments, each of the one or more amino acid recognizers comprises a conductivity label. Examples of conductivity labels and their use in accordance with the application are provided elsewhere herein. Methods for identifying single molecules using conductivity labels have been described (see, e.g., U.S. Patent Publication No. 2017/0037462).

In some embodiments, the series of changes in conductance comprises a series of changes in conductance through a nanopore. For example, methods of evaluating receptor-ligand interactions using nanopores have been described (see, e.g., Thakur, A. K. & Movileanu, L. (2019) Nature Biotechnology 37(1)). The inventors have recognized and appreciated that such nanopores may be used to monitor polypeptide sequencing reactions in accordance with the application. Accordingly, in some embodiments, the disclosure provides methods of polypeptide analysis comprising contacting a single polypeptide molecule with one or more amino acid recognizers described herein, where the single polypeptide molecule is immobilized to a nanopore. In some embodiments, the methods further comprise detecting a series of changes in conductance through the nanopore indicative of association of the one or more amino acid recognizers with successive amino acids exposed at a terminus of the single polypeptide while the single polypeptide is being degraded.

As described herein, in some embodiments, amino acid recognizers of the disclosure may be used to determine at least one chemical characteristic of a polypeptide. In some embodiments, determining at least one chemical characteristic comprises determining the type of amino acid that is present at a terminal end of a polypeptide and/or the types of amino acids that are present at one or more positions contiguous to the amino acid at the terminal end. In some embodiments, determining the type of amino acid comprises determining the actual amino acid identity, for example by determining which of the naturally-occurring 20 amino acids is present. In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

In some embodiments, determining at least one chemical characteristic of a polypeptide comprises determining a subset of potential amino acids that can be present in the polypeptide. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be in the polypeptide (e.g., using a recognizer that binds to a specified subset of two or more amino acids).

In some embodiments, determining at least one chemical characteristic of a polypeptide comprises determining that an amino acid comprises a post-translational modification. Non-limiting examples of post-translational modifications include acetylation (e.g., acetylated lysine), ADP-ribosylation, caspase cleavage, citrullination, formylation, N-linked glycosylation (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation, sumoylation (e.g., sumoylated lysine), and ubiquitination (e.g., ubiquitinated lysine).

In some embodiments, determining at least one chemical characteristic of a polypeptide comprises determining that an amino acid comprises an arginine post-translational modification. For example, as described herein, amino acid recognizers of the disclosure are capable of distinguishing between different arginine modifications, including symmetric dimethylarginine (SDMA), asymmetric dimethylarginine (ADMA), and citrullinated arginine.

In some embodiments, determining at least one chemical characteristic of a polypeptide comprises determining that an amino acid comprises a phosphorylated side chain. For example, in some embodiments, determining at least one chemical characteristic of a polypeptide comprises determining that an amino acid comprises phosphorylated threonine (e.g., phospho-threonine). In some embodiments, determining at least one chemical characteristic of a polypeptide comprises determining that an amino acid comprises phosphorylated tyrosine (e.g., phospho-tyrosine). In some embodiments, determining at least one chemical characteristic of a polypeptide comprises determining that an amino acid comprises phosphorylated serine (e.g., phospho-serine).

In some embodiments, determining at least one chemical characteristic of a polypeptide comprises determining that an amino acid comprises a chemically modified variant, an unnatural amino acid, or a proteinogenic amino acid such as selenocysteine and pyrrolysine. Examples of unnatural amino acids include, without limitation, 2-naphthyl-alanine, statine, homoalanine, a-amino acid, β2-amino acid, β3-amino acid, γ-amino acid, 3-pyridyl-alanine, 4-fluorophenyl-alanine, cyclohexyl-alanine, N-alkyl amino acid, peptoid amino acid, homo-cysteine, penicillamine, 3-nitrotyrosine, homo-phenyl-alanine, t-leucine, hydroxy-proline, 3-Abz, 5-F-tryptophan, and azabicyclo-[2.2.1]heptane.

In some embodiments, determining at least one chemical characteristic of a polypeptide comprises determining that an amino acid comprises an oxidative modification. For example, as described herein, amino acid recognizers of the disclosure are capable of distinguishing between oxidized methionine and its unmodified variant. In some embodiments, the oxidative modification comprises an oxidatively-damaged side chain of an amino acid. In some embodiments, the oxidatively-damaged side chain comprises a cysteine-derived product (e.g., disulfide, sulfinic acid, sulfonic acid, sulfenic acid, S-nitrosocysteine), a tyrosine-derived product (e.g., di-tyrosine, 3,4-dihydroxyphenylalanine, 3-chlorotyrosine, 3-nitrotyrosine), a histidine-derived product (e.g., 2-oxohistidine, 4-hydroxy-2-oxohistidine, di-histidine, asparagine, aspartic acid, urea), a methionine-derived product (e.g., sulfoxide, sulfone), a tryptophan-derived product (e.g., di-tryptophan, N-formylkynurenine, kynurenine, 2-oxo-tryptophan oxindolylalanine, 6-nitrotryptophan, hydroxytryptophan), a phenylalanine-derived product (e.g., meta-tyrosine, ortho-tyrosine), or a generic side-chain product (e.g., alcohol, hydroperoxide, aldehyde/ketone carbonyl). Examples of oxidatively damaged amino acids are known in the art, see, e.g., Hawkins, C. L., Davies, M. J. Detection, identification, and quantification of oxidative protein modifications. J Biol Chem. 2019 Dec. 20; 294(51):19683-19708.

In some embodiments, determining at least one chemical characteristic of a polypeptide comprises determining that an amino acid comprises a side chain characterized by one or more biochemical properties. For example, an amino acid may comprise a nonpolar aliphatic side chain, a positively charged side chain, a negatively charged side chain, a nonpolar aromatic side chain, or a polar uncharged side chain. Non-limiting examples of an amino acid comprising a nonpolar aliphatic side chain include alanine, glycine, valine, leucine, methionine, and isoleucine. Non-limiting examples of an amino acid comprising a positively charged side chain includes lysine, arginine, and histidine. Non-limiting examples of an amino acid comprising a negatively charged side chain include aspartate and glutamate. Non-limiting examples of an amino acid comprising a nonpolar, aromatic side chain include phenylalanine, tyrosine, and tryptophan. Non-limiting examples of an amino acid comprising a polar uncharged side chain include serine, threonine, cysteine, proline, asparagine, and glutamine.

In some embodiments, a protein or polypeptide can be digested into a plurality of smaller polypeptides and chemical characteristics can be determined for one or more of these smaller polypeptides. In some embodiments, a first terminus (e.g., N or C terminus) of a polypeptide is immobilized and the other terminus (e.g., the C or N terminus) is analyzed as described herein.

As used herein, sequencing a polypeptide refers to determining sequence information for a polypeptide. In some embodiments, this can involve determining the identity of each sequential amino acid for a portion (or all) of the polypeptide. However, in some embodiments, this can involve assessing the identity of a subset of amino acids within the polypeptide (e.g., and determining the relative position of one or more amino acid types without determining the identity of each amino acid in the polypeptide). However, in some embodiments, amino acid content information can be obtained from a polypeptide without directly determining the relative position of different types of amino acids in the polypeptide. The amino acid content alone may be used to infer the identity of the polypeptide that is present (e.g., by comparing the amino acid content to a database of polypeptide information and determining which polypeptide(s) have the same amino acid content).

In some embodiments, sequence information for a plurality of polypeptide products obtained from a longer polypeptide or protein (e.g., via enzymatic and/or chemical cleavage) can be analyzed to reconstruct or infer the sequence of the longer polypeptide or protein.

In some aspects, the polypeptide analysis described herein generates data indicating how a polypeptide interacts with a binding means while the polypeptide is being degraded by a cleaving means. As discussed above, the data can include a series of characteristic patterns corresponding to association events at a terminus of a polypeptide in between cleavage events at the terminus. In some embodiments, methods of polypeptide analysis described herein comprise contacting a single polypeptide molecule with a binding means and a cleaving means, where the binding means and the cleaving means are configured to achieve at least 10 association events prior to a cleavage event. In some embodiments, the means are configured to achieve the at least 10 association events between two cleavage events.

In some embodiments, a plurality of single-molecule sequencing reactions are performed in parallel in an array of sample wells. In some embodiments, an array comprises between about 10,000 and about 1,000,000 sample wells. The volume of a sample well may be between about 10⁻²¹ liters and about 10⁻¹⁵ liters, in some implementations. Because the sample well has a small volume, detection of single-molecule events may be possible as only about one polypeptide may be within a sample well at any given time. Statistically, some sample wells may not contain a single-molecule sequencing reaction and some may contain more than one single polypeptide molecule. However, an appreciable number of sample wells may each contain a single-molecule reaction (e.g., at least 30% in some embodiments), so that single-molecule analysis can be carried out in parallel for a large number of sample wells. In some embodiments, the binding means and the cleaving means are configured to achieve at least 10 association events prior to a cleavage event in at least 10% (e.g., 10-50%, more than 50%, 25-75%, at least 80%, or more) of the sample wells in which a single-molecule reaction is occurring. In some embodiments, the binding means and the cleaving means are configured to achieve at least 10 association events prior to a cleavage event for at least 50% (e.g., more than 50%, 50-75%, at least 80%, or more) of the amino acids of a polypeptide in a single-molecule reaction.

In some embodiments, a luminescent label refers to a fluorophore or a dye. Typically, a luminescent label comprises an aromatic or heteroaromatic compound and can be a pyrene, anthracene, naphthalene, naphthylamine, acridine, stilbene, indole, benzindole, oxazole, carbazole, thiazole, benzothiazole, benzoxazole, phenanthridine, phenoxazine, porphyrin, quinoline, ethidium, benzamide, cyanine, carbocyanine, salicylate, anthranilate, coumarin, fluoroscein, rhodamine, xanthene, or other like compound.

In some embodiments, a luminescent label comprises a dye selected from one or more of the following: 5/6-Carboxyrhodamine 6G, 5-Carboxyrhodamine 6G, 6-Carboxyrhodamine 6G, 6-TAMRA, Abberior® STAR 440SXP, Abberior® STAR 470SXP, Abberior® STAR 488, Abberior® STAR 512, Abberior® STAR 520SXP, Abberior® STAR 580, Abberior® STAR 600, Abberior® STAR 635, Abberior® STAR 635P, Abberior® STAR RED, Alexa Fluor® 350, Alexa Fluor® 405, Alexa Fluor® 430, Alexa Fluor® 480, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610-X, Alexa Fluor® 633, Alexa Fluor® 647, Alexa Fluor® 660, Alexa Fluor® 680, Alexa Fluor® 700, Alexa Fluor® 750, Alexa Fluor® 790, AMCA, ATTO 390, ATTO 425, ATTO 465, ATTO 488, ATTO 495, ATTO 514, ATTO 520, ATTO 532, ATTO 542, ATTO 550, ATTO 565, ATTO 590, ATTO 610, ATTO 620, ATTO 633, ATTO 647, ATTO 647N, ATTO 655, ATTO 665, ATTO 680, ATTO 700, ATTO 725, ATTO 740, ATTO Oxa12, ATTO Rho101, ATTO Rho11, ATTO Rho12, ATTO Rho13, ATTO Rho14, ATTO Rho3B, ATTO Rho6G, ATTO Thio12, BD Horizon™ V450, BODIPY® 493/501, BODIPY® 530/550, BODIPY® 558/568, BODIPY® 564/570, BODIPY® 576/589, BODIPY® 581/591, BODIPY® 630/650, BODIPY® 650/665, BODIPY® FL, BODIPY® FL-X, BODIPY® R6G, BODIPY® TMR, BODIPY® TR, CAL Fluor® Gold 540, CAL Fluor® Green 510, CAL Fluor® Orange 560, CAL Fluor® Red 590, CAL Fluor® Red 610, CAL Fluor® Red 615, CAL Fluor® Red 635, Cascade® Blue, CF™350, CF™405M, CF™405S, CF™488A, CF™514, CF™532, CF™543, CF™546, CF™555, CF™568, CF™594, CF™620R, CF™633, CF™633-V1, CF™640R, CF™640R-V1, CF™640R-V2, CF™660C, CF™660R, CF™680, CF™680R, CF™680R-V1, CF™750, CF™770, CF™790, Chromeo™ 642, Chromis 425N, Chromis 500N, Chromis 515N, Chromis 530N, Chromis 550A, Chromis 550C, Chromis 550Z, Chromis 560N, Chromis 570N, Chromis 577N, Chromis 600N, Chromis 630N, Chromis 645A, Chromis 645C, Chromis 645Z, Chromis 678A, Chromis 678C, Chromis 678Z, Chromis 770A, Chromis 770C, Chromis 800A, Chromis 800C, Chromis 830A, Chromis 830C, Cy®3, Cy®3.5, Cy®3B, Cy®5, Cy®5.5, Cy®7, DyLight® 350, DyLight® 405, DyLight® 415-Co1, DyLight® 425Q, DyLight® 485-LS, DyLight® 488, DyLight® 504Q, DyLight® 510-LS, DyLight® 515-LS, DyLight® 521-LS, DyLight® 530-R2, DyLight® 543Q, DyLight® 550, DyLight® 554-R0, DyLight® 554-R1, DyLight® 590-R2, DyLight® 594, DyLight® 610-B1, DyLight® 615-B2, DyLight® 633, DyLight® 633-B1, DyLight® 633-B2, DyLight® 650, DyLight® 655-B1, DyLight® 655-B2, DyLight® 655-B3, DyLight® 655-B4, DyLight® 662Q, DyLight® 675-B1, DyLight® 675-B2, DyLight® 675-B3, DyLight® 675-B4, DyLight® 679-C5, DyLight® 680, DyLight® 683Q, DyLight® 690-B1, DyLight® 690-B2, DyLight® 696Q, DyLight® 700-B1, DyLight® 700-B1, DyLight® 730-B1, DyLight® 730-B2, DyLight® 730-B3, DyLight® 730-B4, DyLight® 747, DyLight® 747-B1, DyLight® 747-B2, DyLight® 747-B3, DyLight® 747-B4, DyLight® 755, DyLight® 766Q, DyLight® 775-B2, DyLight® 775-B3, DyLight® 775-B4, DyLight® 780-B1, DyLight® 780-B2, DyLight® 780-B3, DyLight® 800, DyLight® 830-B2, Dyomics-350, Dyomics-350XL, Dyomics-360XL, Dyomics-370XL, Dyomics-375XL, Dyomics-380XL, Dyomics-390XL, Dyomics-405, Dyomics-415, Dyomics-430, Dyomics-431, Dyomics-478, Dyomics-480XL, Dyomics-481XL, Dyomics-485XL, Dyomics-490, Dyomics-495, Dyomics-505, Dyomics-510XL, Dyomics-511XL, Dyomics-520XL, Dyomics-521XL, Dyomics-530, Dyomics-547, Dyomics-547P1, Dyomics-548, Dyomics-549, Dyomics-549P1, Dyomics-550, Dyomics-554, Dyomics-555, Dyomics-556, Dyomics-560, Dyomics-590, Dyomics-591, Dyomics-594, Dyomics-601XL, Dyomics-605, Dyomics-610, Dyomics-615, Dyomics-630, Dyomics-631, Dyomics-632, Dyomics-633, Dyomics-634, Dyomics-635, Dyomics-636, Dyomics-647, Dyomics-647P1, Dyomics-648, Dyomics-648P1, Dyomics-649, Dyomics-649P1, Dyomics-650, Dyomics-651, Dyomics-652, Dyomics-654, Dyomics-675, Dyomics-676, Dyomics-677, Dyomics-678, Dyomics-679P1, Dyomics-680, Dyomics-681, Dyomics-682, Dyomics-700, Dyomics-701, Dyomics-703, Dyomics-704, Dyomics-730, Dyomics-731, Dyomics-732, Dyomics-734, Dyomics-749, Dyomics-749P1, Dyomics-750, Dyomics-751, Dyomics-752, Dyomics-754, Dyomics-776, Dyomics-777, Dyomics-778, Dyomics-780, Dyomics-781, Dyomics-782, Dyomics-800, Dyomics-831, eFluor® 450, Eosin, FITC, Fluorescein, HiLyte™ Fluor 405, HiLyte™ Fluor 488, HiLyte™ Fluor 532, HiLyte™ Fluor 555, HiLyte™ Fluor 594, HiLyte™ Fluor 647, HiLyte™ Fluor 680, HiLyte™ Fluor 750, IRDye® 680LT, IRDye® 750, IRDye® 800CW, JOE, LightCycler® 640R, LightCycler® Red 610, LightCycler® Red 640, LightCycler® Red 670, LightCycler® Red 705, Lissamine Rhodamine B, Napthofluorescein, Oregon Green® 488, Oregon Green® 514, Pacific Blue™, Pacific Green™ Pacific Orange™, PET, PF350, PF405, PF415, PF488, PF505, PF532, PF546, PF555P, PF568, PF594, PF610, PF633P, PF647P, Quasar® 570, Quasar® 670, Quasar® 705, Rhodamine 123, Rhodamine 6G, Rhodamine B, Rhodamine Green, Rhodamine Green-X, Rhodamine Red, ROX, Seta™ 375, Seta™ 470, Seta™ 555, Seta™ 632, Seta™ 633, Seta™ 650, Seta™ 660, Seta™ 670, Seta™ 680, Seta™ 700, Seta™ 750, Seta™ 780, Seta™ APC-780, Seta™ PerCP-680, Seta™ R-PE-670, Seta™ 646, SeTau 380, SeTau 425, SeTau 647, SeTau 405, Square 635, Square 650, Square 660, Square 672, Square 680, Sulforhodamine 101, TAMRA, TET, Texas Red®, TMR, TRITC, Yakima Yellow™, Zenon®, Zy3, Zy5, Zy5.5, and Zy7.

In certain embodiments, the cut depth of the compound of Formula (II) is improved compared to the cut depth of a compound of Formula Z-L¹-Y (X), wherein Y and Z are as defined herein, and L¹ is

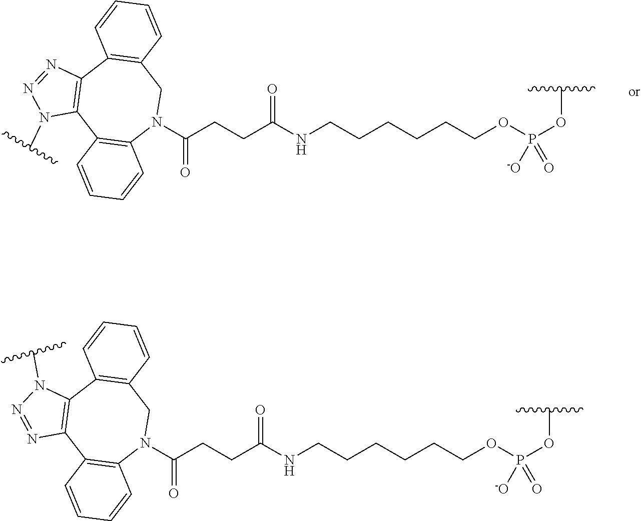

(C6 linker). In certain embodiments, the cut depth of the compound of Formula (II) is improved by at least about 10% compared to the cut depth of the compound of Formula (X). In certain embodiments, the cut depth of the compound of Formula (II) is improved by at least about 15% compared to the cut depth of the compound of Formula (X). In certain embodiments, the cut depth of the compound of Formula (II) is improved by at least about 20% compared to the cut depth of the compound of Formula (X). In certain embodiments, the cut depth of the compound of Formula (II) is improved by at least about 25% compared to the cut depth of the compound of Formula (X). In certain embodiments, the cut depth of the compound of Formula (II) is improved by at least about 30% compared to the cut depth of the compound of Formula (X). In certain embodiments, the cut depth of the compound of Formula (II) is improved by at least about 35% compared to the cut depth of the compound of Formula (X). In certain embodiments, the cut depth of the compound of Formula (II) is improved by at least about 40% compared to the cut depth of the compound of Formula (X). In certain embodiments, the cut depth of the compound of Formula (II) is improved by at least about 45% compared to the cut depth of the compound of Formula (X). In certain embodiments, the cut depth of the compound of Formula (II) is improved by at least about 50% compared to the cut depth of the compound of Formula (X). In certain embodiments, the cut depth of the compound of Formula (II) is improved by at least about 55% compared to the cut depth of the compound of Formula (X). In certain embodiments, the cut depth of the compound of Formula (II) is improved by at least about 60% compared to the cut depth of the compound of Formula (X). In certain embodiments, the cut depth of the compound of Formula (II) is improved by at least about 65% compared to the cut depth of the compound of Formula (X). In certain embodiments, the cut depth of the compound of Formula (II) is improved by at least about 70% compared to the cut depth of the compound of Formula (X). In certain embodiments, the cut depth of the compound of Formula (II) is improved by at least about 75% compared to the cut depth of the compound of Formula (X). In certain embodiments, the cut depth of the compound of Formula (II) is improved by at least about 80% compared to the cut depth of the compound of Formula (X). In certain embodiments, the cut depth of the compound of Formula (II) is improved by at least about 85% compared to the cut depth of the compound of Formula (X). In certain embodiments, the cut depth of the compound of Formula (II) is improved by at least about 90% compared to the cut depth of the compound of Formula (X). In certain embodiments, the cut depth of the compound of Formula (II) is improved by at least about 95% compared to the cut depth of the compound of Formula (X). In certain embodiments, the cut depth of the compound of Formula (II) is improved by at least about 100% compared to the cut depth of the compound of Formula (X). In certain embodiments, the cut depth of the compound of Formula (II) is improved by between about 10% and about 100% compared to the cut depth of the compound of Formula (X). In certain embodiments, the cut depth of the compound of Formula (II) is improved by between about 10% and about 90% compared to the cut depth of the compound of Formula (X). In certain embodiments, the cut depth of the compound of Formula (II) is improved by between about 10% and about 80% compared to the cut depth of the compound of Formula (X). In certain embodiments, the cut depth of the compound of Formula (II) is improved by between about 10% and about 70% compared to the cut depth of the compound of Formula (X). In certain embodiments, the cut depth of the compound of Formula (II) is improved by between about 10% and about 60% compared to the cut depth of the compound of Formula (X). In certain embodiments, the cut depth of the compound of Formula (II) is improved by between about 10% and about 50% compared to the cut depth of the compound of Formula (X). In certain embodiments, the cut depth of the compound of Formula (II) is improved by between about 10% and about 40% compared to the cut depth of the compound of Formula (X). In certain embodiments, the cut depth of the compound of Formula (II) is improved by between about 10% and about 30% compared to the cut depth of the compound of Formula (X). In certain embodiments, the cut depth of the compound of Formula (II) is improved by between about 10% and about 20% compared to the cut depth of the compound of Formula (X). In certain embodiments, the cut depth of the compound of Formula (II) is improved by between about 20% and about 100% compared to the cut depth of the compound of Formula (X). In certain embodiments, the cut depth of the compound of Formula (II) is improved by between about 30% and about 100% compared to the cut depth of the compound of Formula (X). In certain embodiments, the cut depth of the compound of Formula (II) is improved by between about 40% and about 100% compared to the cut depth of the compound of Formula (X). In certain embodiments, the cut depth of the compound of Formula (II) is improved by between about 40% and about 90% compared to the cut depth of the compound of Formula (X). In certain embodiments, the cut depth of the compound of Formula (II) is improved by between about 40% and about 80% compared to the cut depth of the compound of Formula (X). In certain embodiments, the cut depth of the compound of Formula (II) is improved by between about 50% and about 100% compared to the cut depth of the compound of Formula (X). In certain embodiments, the cut depth of the compound of Formula (II) is improved by between about 50% and about 90% compared to the cut depth of the compound of Formula (X). In certain embodiments, the cut depth of the compound of Formula (II) is improved by between about 50% and about 80% compared to the cut depth of the compound of Formula (X). In certain embodiments, the cut depth of the compound of Formula (II) is improved by between about 60% and about 100% compared to the cut depth of the compound of Formula (X). In certain embodiments, the cut depth of the compound of Formula (II) is improved by between about 60% and about 90% compared to the cut depth of the compound of Formula (X). In certain embodiments, the cut depth of the compound of Formula (II) is improved by between about 60% and about 80% compared to the cut depth of the compound of Formula (X). In certain embodiments, the cut depth of the compound of Formula (II) is improved by between about 70% and about 100% compared to the cut depth of the compound of Formula (X). In certain embodiments, the cut depth of the compound of Formula (II) is improved by between about 70% and about 90% compared to the cut depth of the compound of Formula (X). In certain embodiments, the cut depth of the compound of Formula (II) is improved by between about 70% and about 80% compared to the cut depth of the compound of Formula (X). In certain embodiments, the cut depth of the compound of Formula (II) is improved by between about 80% and about 100% compared to the cut depth of the compound of Formula (X). In certain embodiments, the cut depth of the compound of Formula (II) is improved by between about 90% and about 100% compared to the cut depth of the compound of Formula (X). In certain embodiments, the cut depth of the compound of Formula (II) is improved by about 10% compared to the cut depth of the compound of Formula (X). In certain embodiments, the cut depth of the compound of Formula (II) is improved by about 15% compared to the cut depth of the compound of Formula (X). In certain embodiments, the cut depth of the compound of Formula (II) is improved by about 20% compared to the cut depth of the compound of Formula (X). In certain embodiments, the cut depth of the compound of Formula (II) is improved by about 25% compared to the cut depth of the compound of Formula (X). In certain embodiments, the cut depth of the compound of Formula (II) is improved by about 30% compared to the cut depth of the compound of Formula (X). In certain embodiments, the cut depth of the compound of Formula (II) is improved by about 35% compared to the cut depth of the compound of Formula (X). In certain embodiments, the cut depth of the compound of Formula (II) is improved by about 40% compared to the cut depth of the compound of Formula (X). In certain embodiments, the cut depth of the compound of Formula (II) is improved by about 45% compared to the cut depth of the compound of Formula (X). In certain embodiments, the cut depth of the compound of Formula (II) is improved by about 50% compared to the cut depth of the compound of Formula (X). In certain embodiments, the cut depth of the compound of Formula (II) is improved by about 55% compared to the cut depth of the compound of Formula (X). In certain embodiments, the cut depth of the compound of Formula (II) is improved by about 60% compared to the cut depth of the compound of Formula (X). In certain embodiments, the cut depth of the compound of Formula (II) is improved by about 65% compared to the cut depth of the compound of Formula (X). In certain embodiments, the cut depth of the compound of Formula (II) is improved by about 70% compared to the cut depth of the compound of Formula (X). In certain embodiments, the cut depth of the compound of Formula (II) is improved by about 75% compared to the cut depth of the compound of Formula (X). In certain embodiments, the cut depth of the compound of Formula (II) is improved by about 80% compared to the cut depth of the compound of Formula (X). In certain embodiments, the cut depth of the compound of Formula (II) is improved by about 85% compared to the cut depth of the compound of Formula (X). In certain embodiments, the cut depth of the compound of Formula (II) is improved by about 90% compared to the cut depth of the compound of Formula (X). In certain embodiments, the cut depth of the compound of Formula (II) is improved by about 95% compared to the cut depth of the compound of Formula (X). In certain embodiments, the cut depth of the compound of Formula (II) is improved by about 100% compared to the cut depth of the compound of Formula (X). In certain embodiments, the cut depth of the compound of Formula (II) is improved by about 76% compared to the cut depth of the compound of Formula (X).

In certain embodiments, the percentage of reads that terminate at a specific residue of the compound of Formula (II) is improved compared to the percentage of reads that terminate at a specific residue of a compound of Formula Z-L¹-Y (X), wherein Y and Z are as defined herein, and L¹ is

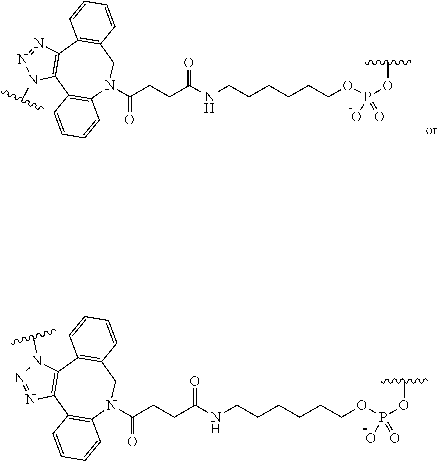 [SMILES: *OP(=O)(O)OCCCCCCNC(=O)CCC(=O)N1Cc2ccccc2-c2nnn(*)c2-c2ccccc21 ; *OP(=O)(O)OCCCCCCNC(=O)CCC(=O)N1Cc2ccccc2-c2c(nnn2*)-c2ccccc21]

(C6 linker). In certain embodiments, the percentage of reads that terminate at a specific residue of the compound of Formula (II) is improved by at least about 100% compared to the percentage of reads that terminate at a specific residue of the compound of Formula (X). In certain embodiments, the percentage of reads that terminate at a specific residue of the compound of Formula (II) is improved by at least about 200% compared to the percentage of reads that terminate at a specific residue of the compound of Formula (X). In certain embodiments, the percentage of reads that terminate at a specific residue of the compound of Formula (II) is improved by at least about 300% compared to the percentage of reads that terminate at a specific residue of the compound of Formula (X). In certain embodiments, the percentage of reads that terminate at a specific residue of the compound of Formula (II) is improved by at least about 400% compared to the percentage of reads that terminate at a specific residue of the compound of Formula (X). In certain embodiments, the percentage of reads that terminate at a specific residue of the compound of Formula (II) is improved by at least about 500% compared to the percentage of reads that terminate at a specific residue of the compound of Formula (X). In certain embodiments, the percentage of reads that terminate at a specific residue of the compound of Formula (II) is improved by at least about 600% compared to the percentage of reads that terminate at a specific residue of the compound of Formula (X). In certain embodiments, the percentage of reads that terminate at a specific residue of the compound of Formula (II) is improved by at least about 700% compared to the percentage of reads that terminate at a specific residue of the compound of Formula (X). In certain embodiments, the percentage of reads that terminate at a specific residue of the compound of Formula (II) is improved by at least about 800% compared to the percentage of reads that terminate at a specific residue of the compound of Formula (X). In certain embodiments, the percentage of reads that terminate at a specific residue of the compound of Formula (II) is improved by at least about 900% compared to the percentage of reads that terminate at a specific residue of the compound of Formula (X). In certain embodiments, the percentage of reads that terminate at a specific residue of the compound of Formula (II) is improved by at least about 1000% compared to the percentage of reads that terminate at a specific residue of the compound of Formula (X).

In certain embodiments, the percentage of reads that terminate at a specific residue of the compound of Formula (II) is improved by between about 100% and about 1000% compared to the percentage of reads that terminate at a specific residue of the compound of Formula (X), inclusive. In certain embodiments, the percentage of reads that terminate at a specific residue of the compound of Formula (II) is improved by between about 200% and about 900% compared to the percentage of reads that terminate at a specific residue of the compound of Formula (X), inclusive. In certain embodiments, the percentage of reads that terminate at a specific residue of the compound of Formula (II) is improved by between about 300% and about 800% compared to the percentage of reads that terminate at a specific residue of the compound of Formula (X), inclusive. In certain embodiments, the percentage of reads that terminate at a specific residue of the compound of Formula (II) is improved by between about 400% and about 700% compared to the percentage of reads that terminate at a specific residue of the compound of Formula (X), inclusive. In certain embodiments, the percentage of reads that terminate at a specific residue of the compound of Formula (II) is improved by between about 500% and about 600% compared to the percentage of reads that terminate at a specific residue of the compound of Formula (X), inclusive. In certain embodiments, the percentage of reads that terminate at a specific residue of the compound of Formula (II) is improved by between about 400% and about 600% compared to the percentage of reads that terminate at a specific residue of the compound of Formula (X), inclusive. In certain embodiments, the percentage of reads that terminate at a specific residue of the compound of Formula (II) is improved by between about 400% and about 800% compared to the percentage of reads that terminate at a specific residue of the compound of Formula (X), inclusive. In certain embodiments, the percentage of reads that terminate at a specific residue of the compound of Formula (II) is improved by between about 400% and about 900% compared to the percentage of reads that terminate at a specific residue of the compound of Formula (X), inclusive. In certain embodiments, the percentage of reads that terminate at a specific residue of the compound of Formula (II) is improved by between about 400% and about 1000% compared to the percentage of reads that terminate at a specific residue of the compound of Formula (X), inclusive.

In certain embodiments, the cutting rate of the compound of Formula (II) is improved compared to the cutting rate of a compound of Formula Z-L¹-Y (X), wherein Y and Z are as defined herein, and L¹ is

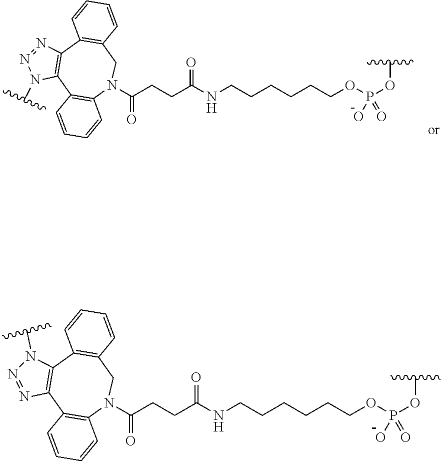 [SMILES: *OP(=O)(O)OCCCCCCNC(=O)CCC(=O)N1Cc2ccccc2-c2nnn(*)c2-c2ccccc21 ; *OP(=O)(O)OCCCCCCNC(=O)CCC(=O)N1Cc2ccccc2-c2c(nnn2*)-c2ccccc21]

(C6 linker). In certain embodiments, the cutting rate of the compound of Formula (II) is at least doubled compared to the cutting rate of the compound of Formula (X). In certain embodiments, the cutting rate of the compound of Formula (II) is at least tripled compared to the cutting rate of the compound of Formula (X). In certain embodiments, the cutting rate of the compound of Formula (II) is at least quadrupled compared to the cutting rate of the compound of Formula (X).

### EXAMPLES

### Example 1: Click Reaction Between Peptide DDGGGDDDFFK(N₃)—NH₂ (SEQ ID NO: 44) and Q24

Q24 has the structure 5′-Bisbiotin-CCACGCGTGGAACCCTTGGGATCC-[02′-propargylA]-3′ (SEQ ID NO: 41). Into a 25 μL solution of 3 mM Q24, 10 mM DDGGGDDDFFK(N₃)—NH₂ (SEQ ID NO: 44) in 0.1 M NaHCO₃, was added 1.5 μL of 40 mM Cu(THPTA), immediately followed by addition of 3 μL 1M sodium ascorbate. Let the solution sit at rt for 20 minutes. Add a second portion of sodium ascorbate (3 μL, 1M). Wait for 20 minutes before quenching the reaction with 10 mM EDTA (final concentration). The mixture was injected to C18-HPLC to obtain the polypeptidyl-oligonucleotide conjugate (Q24D).

### Example 2: Conjugation of Q24D with DBCO

A solution of the polypeptidyl-oligonucleotide conjugate (Q24D) (20 nmol) in 25 μL H₂O was mixed with a solution of DBCO-NHS (1 mg) in 75 μL DMSO. Add 10 μL of 1M NaHCO₃ to the reaction mixture. The mixture was vortexed and placed on a shaker for 2 h. The reaction was diluted with 190 uL water and passed through two Zeba spin desalting columns (7k MWCO) at 3.2×10³ rpm for 1 min. The filtrate was purified by reverse-phase HPLC to provide the DBCO conjugation product (DBCO-Q24D).

### Example 3: Conjugation of Streptavidin to DBCO-Q24D

A solution of DBCO-Q24D (5 mL, 10 uM in water) was added to a fast-stirring solution of streptavidin (1×PBS, 10 mg/mL, 7 mL) through a syringe pump over 30 minutes. The mixture was allowed to stir at room temperature before it was injected to a preparative SEC HPLC (isocratic 1×PBS) to isolate the DBCO-Q24D-streptavidin complex.

### Example 4: Click Reaction Between DBCO-Q24D-Streptavidin Conjugate and Functionalized

### Peptide

Dilute 3.4 μL of 29 uM DBCO-Q24D-streptavidin complex into 16.1 uL 1×PBS. Add 0.5 uL of 2 mM functionalized peptide (e.g., azide-functionalized peptide). Let the mixture sit at room temperature overnight. The reaction was filtered through a Zeba spin column that is pre-equilibrated with 60 mM KOAc, 50 mM MOPS (pH 8.0). The concentration of the filtrate is quantified by UV-vis measurement at the Cy3B absorption channel.

### Example 5: Studies of Novel Linkers

With synthetic peptides, addition of DDD or similar sequences to the C-terminus improves cutting efficiency. Increasing the length and/or amount of negative charge on the synthetic peptide also improves cutting efficiency. With a library of naturally occurring proteins, the cutting efficiency can be modulated by the addition of a linker with the desired properties (e.g., DDD or similar sequences, increased length, and/or increased negative charge), such as through addition of a linker by a click chemistry reaction. In particular, multiple experiments with synthetic and naturally occurring protein libraries show a large improvement in cutting efficiency, cut depth, and information content of reads using the Q24D linker.

Table 4 shows linkers tested, and the resulting changes to cutting efficiency. These linkers contain a click chemistry handle (e.g., a strained alkyne (e.g., DBCO)) for polypeptide attachment, a polypeptidyl sequence, and an oligonucleotide (e.g., Q24) for attachment to an avidin protein (e.g., streptavidin).

Table 5 shows the sequences of several synthetic polypeptides used in sequencing for assessment of the linkers in Table 4. Table 6 shows the change in metrics between the C6 linker and the Q24D linker. For almost all synthetic polypeptides studied, use of the Q24D linker instead of the C6 linker decreased the region of interest (ROI) duration, indicating that the rate at which amino acids are sequentially exposed at the terminus of the polypeptide during sequencing (i.e., the cut rate) increased accordingly. Similarly, when a trend was observable, use of the Q24D linker instead of the C6 linker increased the cut depth up to 29%, indicating that up to 29% more of the polypeptide was sequenced.

TABLE 4
Polypeptidyl Linkers.

| Linker Structure | Observations | Design Rationale |
|---|---|---|
| C6-PEG13-DBCO | Slowed down the conjugation | Polarity/length. |
| | rate. | |
| DBCO-PEG3-sulfo-C6 | Slowed down the conjugation | Polarity/length. |
| | rate. | |
| BCN-C6 | No significant change in | Size. |
| | cutting rate on QP425. May | |
| | be faster on shorter peptides. | |
| tau-DIBO-C6 | No significant change cutting | Side chain charge. |
| | rate on QP425. | |
| sulfo-ODIBO-C6 | No significant changes in | Reaction rate. |
| | cutting on QP425. Cutting | |
| | was slower on QP433. | |
| DBCO-polyurea dendrimer-C6 | Challenging in synthesis. | Side chain barrier. |
| DBCO-GPPPPPPPPG-C2′-Am | Cutting was slower on QP423 | Rigidity. |
| (SEQ ID NO: 61) | compared to C6. | |
| DBCO-isoEGWRW-C2′Am | Challenging in synthesis. | Rigidity. |
| (SEQ ID NO: 62) | | |
| DBCO-DDGGGDDDFFK(N3)- | Improved cutting on all | Charge/length. |
| (SEQ ID NO: 44) (also referred | peptides tested except QP425 | |
| to as the “peptide-oligo spacer”, | compared to C6. | |
| the “D peptide,” or “linker D,” | | |
| or as “DBCO-Q24D”) | | |
| DBCO- | Challenging in synthesis. | Length/best substrate fitting |
| GGSSSGSGNDEEFQK(N3) | | |
| DBCO- | Improved cutting on leucine | Charge/length/rigidity/substrate |
| GGGGGGDPDPDK(N3) | but not on arginine and | fitting. |
| (Q24GDP) | isoleucine on QP423 | |
| | compared to C6. | |
| DBCO-NNGGGNNNFFK(N3) | Faster than C6 but slower | Length |
| (SEQ ID NO: 66)-(linker N) | than D on QP423 | |
| DBCO- | Faster than C6 but slower | Charge/length/rigidity/substrate |
| GGGGGDPDPDFFK(N3) SEQ | than D on QP423 | fitting. |
| ID NO: 56)-(linker GDPF) | | |
| DBCO- | Cutting rates faster on leucine | Charge/length/substrate fitting. |
| GDDGDGDGDFFK(N3)- | but slower on arginine and | |
| (linker GDFF)(SEQ ID NO: | phenylalanine than D on | |
| 52) | QP423 | |
| DBCO- | Cutting rates slightly faster | Charge softness |
| DDGGGCyCyCyFFK(N3) | than D on QP423 | |
| (SEQ ID NO: 46)- | | |
| (linker Cy) | | |

TABLE 5
Synthetic Polypeptide Sequences.

| Name | Sequence | SEQ ID NO |
|---|---|---|
| QP425 | LASSIAEANRFADIADYP1 | 33 |
| QP433 | RLIFAYPDDD1 | 34 |
| QP423 | RLIFAYPG1 | 35 |
| QP633 | LAQFASIAAYAS1 | 36 |
| QP542 | RRLIFSK | 37 |
| QP544 | LQYRSRLQYMK | 38 |
| QP547 | FRLNLYELK | 39 |
| UBB | DQQRLIFAGK | 40 |

TABLE 6
Change in Metrics
Between C6 Linker and Q24D Linker.

| | | | | | Cut | Important |
|---|---|---|---|---|---|---|
| Name | ROI1 | ROI2 | ROI3 | ROI4 | Depth | Reads |
| QP423 | −277% | −107% | −313% | −158% | +19.7% | +29.3% |
| QP425 | NT | NT | NT | NT | +8.4% or | +10.9% or |
| | | | | | +12.7% | +44.3% |
| QP633 | −70.3% | −55.9% | −160% | NA | NT | NT |
| QP542 | −90% | −273% | −130% | −469% | +8% | +68% |
| QP544 | +23% | +11% | −38% | −89% | +29% | +392% |
| QP547 | −120% | −296% | −232% | NA | NT | NT |
| UBB | −76% | −44% | −235% | NA | NT | +61% |

NT indicates no clear trend, either due to low number of reads to make solid conclusion or different chips showed different trend.

### Example 6: Sequencing Comparison of C6 Linker and Q24D Linker

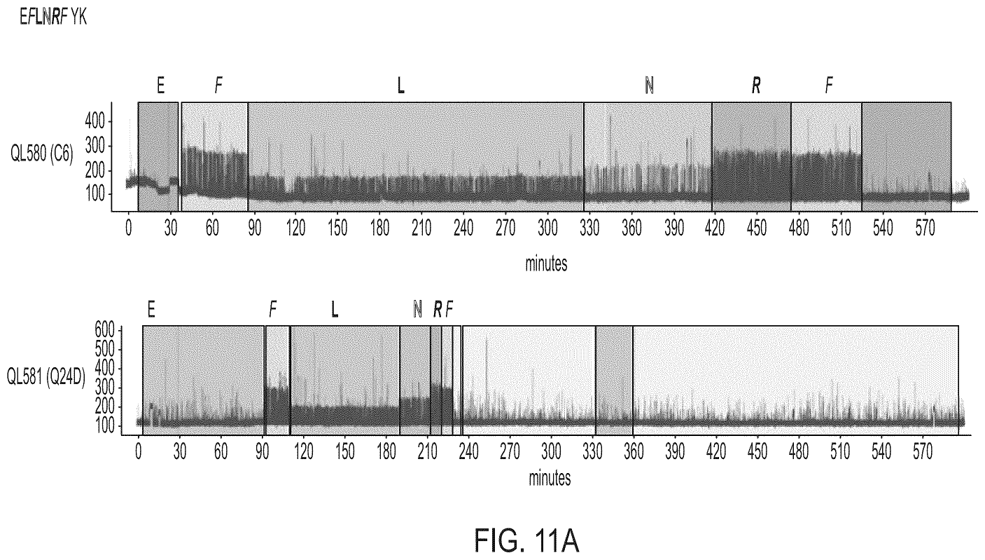

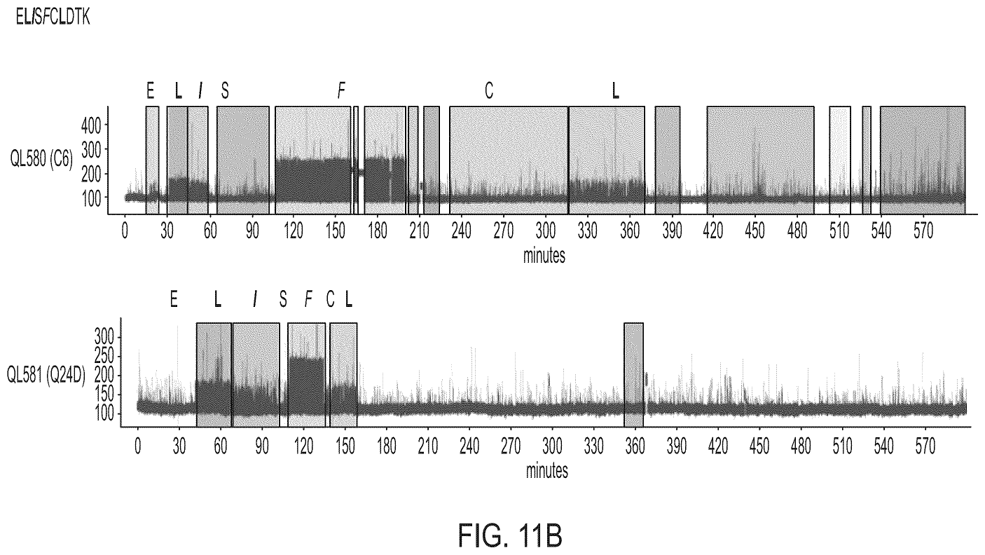

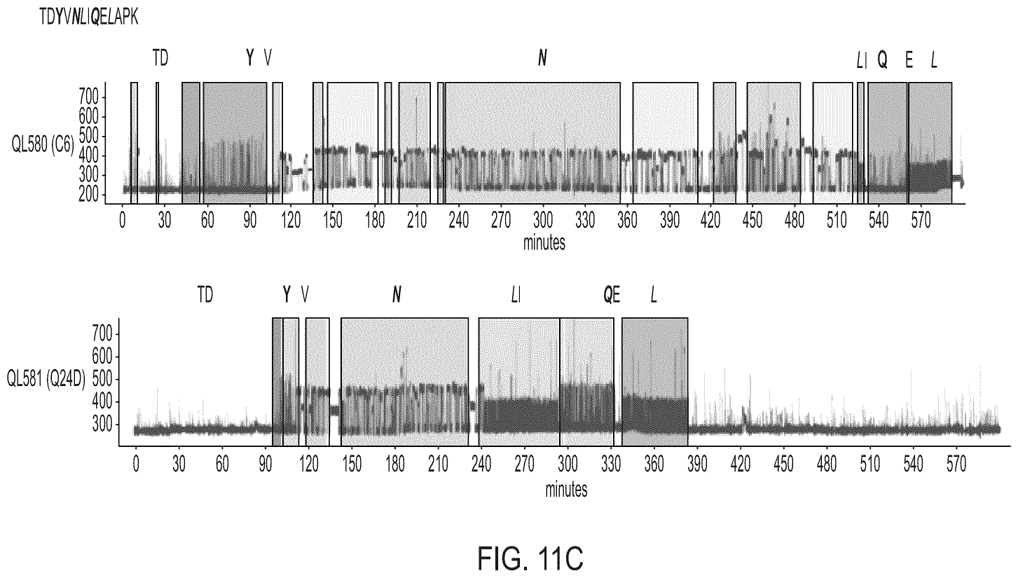

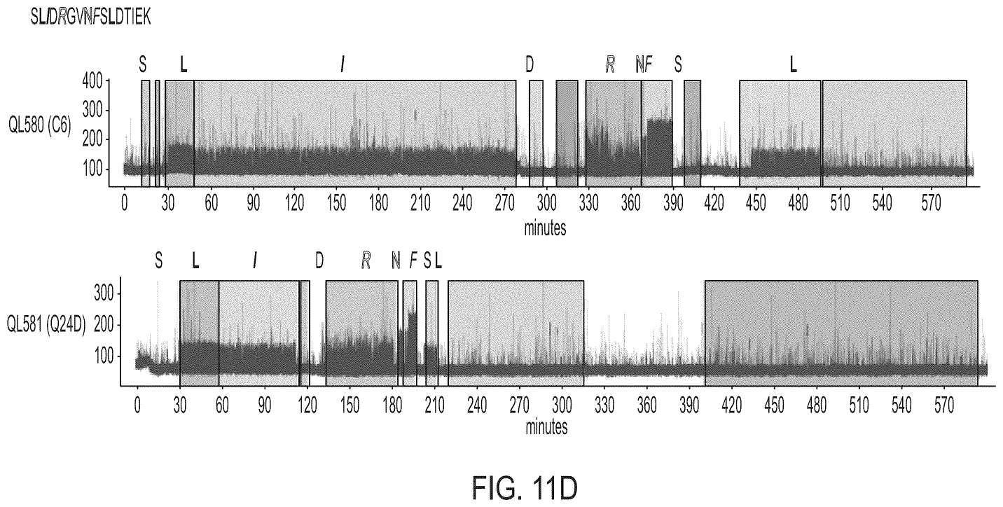

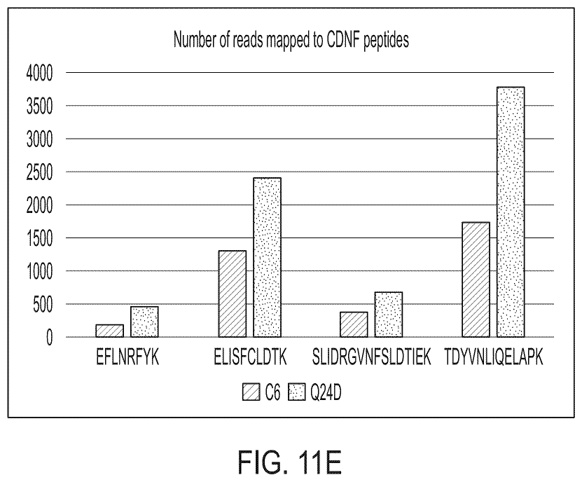

Recombinant human protein CDNF (Cerebral dopamine neurotrophic factor, 161 amino acids) was digested with LysC into peptide fragments and two libraries were prepared by ligation to QL580 (C6 linker attached to Q24 oligonucleotide) or QL581 (linker D attached to Q24 oligonucleotide). QL580 and QL581 libraries were loaded on Quantum-Si chips and sequenced separately. Sequencing was performed with Tet aminopeptidases AP30 and AP37 at 4 μM and 40 μM, respecitively for QL580, and at 2.5 μM and 25 μM, respectively, for QL581. Sequencing data was analyzed to identify traces corresponding to four CDNF peptides: EFLNRFYK (SEQ ID NO: 47), ELISFCLDTK (SEQ ID NO: 49), TDYVNLIQELAPK (SEQ ID NO: 69), and SLIDRGVNFSLDTIEK (SEQ ID NO: 68) (FIGS. 11A-11D). Reads for each peptide displayed faster cleavage rates and longer cut depth on average for QL581 compared to QL580. Representative traces shown for each peptide demonstrate the faster cleavage observed with QL581. Due to improved sequencing performance with longer cut depth and more amino acids recognized in traces on average, software analysis successfully identified substantially more reads corresponding to each peptide with QL581 compared to QL580 (FIG. 11E).

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

### EMBODIMENTS

Embodiments of the present disclosure include:

Embodiment 1. A compound of Formula (I):

L-Y (I),

or a salt thereof, wherein:
L comprises a polypeptidyl group; and
Y comprises an oligonucleotide.
   Embodiment 2. A compound of Formula (II):

Z-L-Y (II),

or a salt thereof, wherein:
L comprises a polypeptidyl group;
Y comprises an oligonucleotide; and
Z is a polypeptide.
   Embodiment 3. The compound of any one of embodiments 1 and 2, wherein the polypeptidyl group comprises between 5 and 20 amino acid residues, inclusive.
   Embodiment 4. The compound of any one of embodiments 1-3, wherein the polypeptidyl group is between about 20 Å and about 75 Å in length, inclusive.
   Embodiment 5. The compound of any one of embodiments 1-4, wherein the polypeptidyl group comprises between 1 and 10 negatively charged moieties at physiological pH, inclusive.
   Embodiment 6. The compound of any one of embodiments 1-5, wherein the polypeptidyl group comprises between 1 and 15 aspartate residues, inclusive.
   Embodiment 7. The compound of any one of embodiments 1-6, wherein the polypeptidyl group comprises between 1 and 10 phenylalanine residues, inclusive.
   Embodiment 8. The compound of any one of embodiments 1-7, wherein the polypeptidyl group comprises between 1 and 10 glycine residues, inclusive.
   Embodiment 9. The compound of any one of embodiments 1-8, wherein the polypeptidyl group comprises between 1 and 5 proline residues, inclusive.
   Embodiment 10. The compound of any one of embodiments 1-9, wherein the polypeptidyl group comprises between 1 and 5 GP repeats, inclusive.
   Embodiment 11. The compound of any one of embodiments 1-10, wherein the polypeptidyl group comprises a moiety selected from:

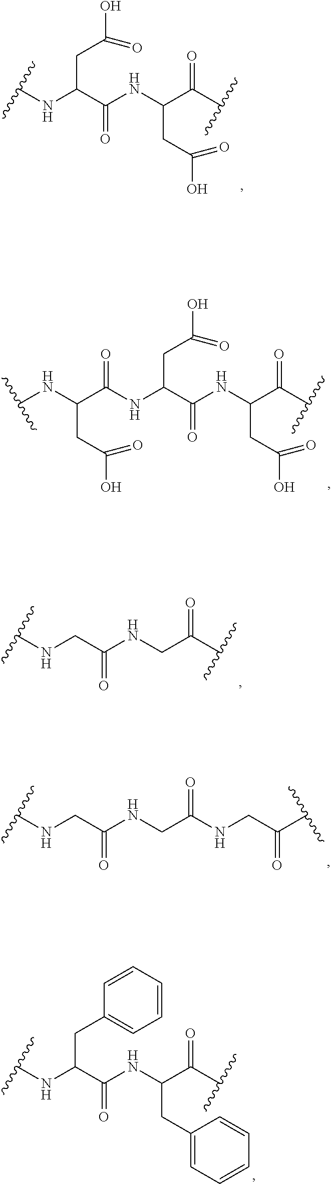 [SMILES: *NC(CC(=O)O)C(=O)NC(CC(=O)O)C(*)=O ; *NC(CC(=O)O)C(=O)NC(CC(=O)O)C(=O)NC(CC(=O)O)C(*)=O ; *NCC(=O)NCC(*)=O ; *NCC(=O)NCC(=O)NCC(*)=O ; *NC(Cc1ccccc1)C(=O)NC(Cc1ccccc1)C(*)=O]

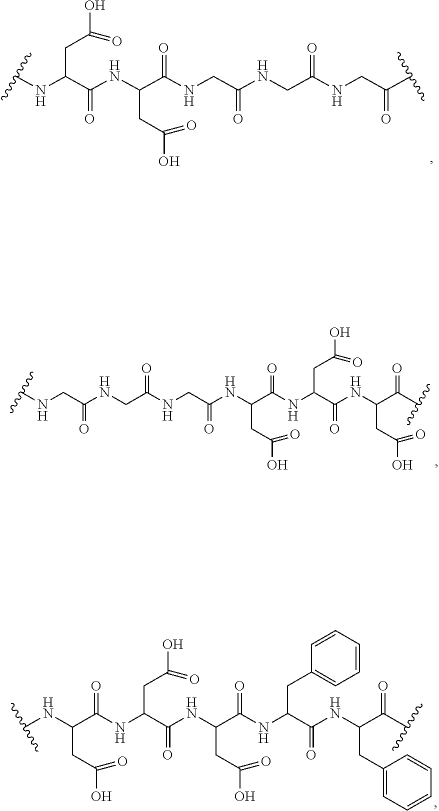 [SMILES: *NC(CC(=O)O)C(=O)NC(CC(=O)O)C(=O)NCC(=O)NCC(=O)NCC(*)=O ; *NCC(=O)NCC(=O)NCC(=O)NC(CC(=O)O)C(=O)NC(CC(=O)O)C(=O)NC(CC(=O)O)C(*)=O ; *NC(CC(=O)O)C(=O)NC(CC(=O)O)C(=O)NC(CC(=O)O)C(=O)NC(Cc1ccccc1)C(=O)NC(Cc1ccccc1)C(*)=O]

or a salt thereof.
Embodiment 12. The compound of any one of embodiments 1-11, wherein the polypeptidyl group comprises a moiety selected from:

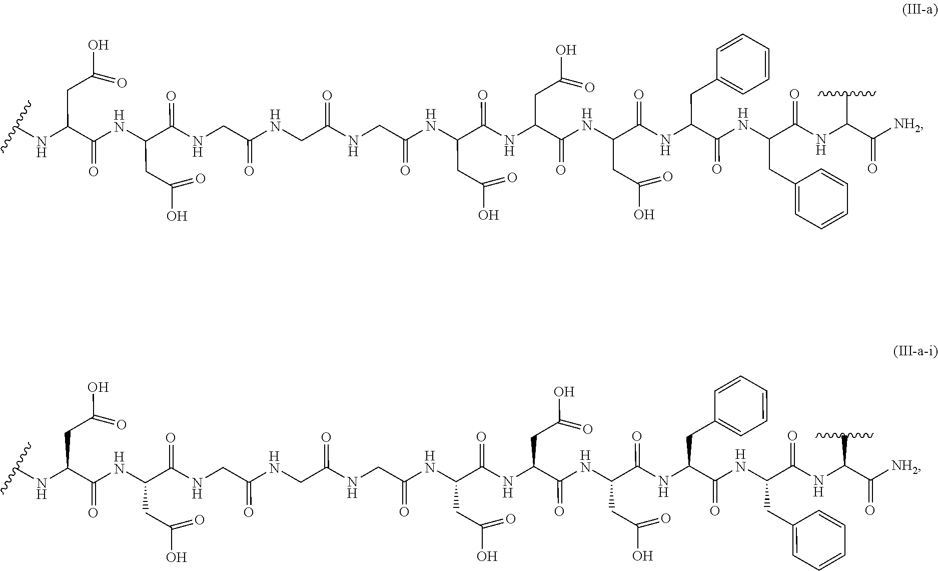 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O ; *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]

or a salt thereof.
Embodiment 13. The compound of any one of embodiments 1-12, wherein the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), or a salt thereof, wherein Cy is cysteic acid.
Embodiment 14. The compound of any one of embodiments 1-13, wherein the polypeptidyl group comprises a sequence DDGGGDDDFF (SEQ ID NO: 32), or a salt thereof.
Embodiment 15. The compound of any one of embodiments 1-14, wherein L further comprises at least one of optionally substituted alkylene, optionally substituted alkenylene, optionally substituted alkynylene, optionally substituted heteroalkylene, optionally substituted heteroalkenylene, optionally substituted heteroalkynylene, optionally substituted heterocyclylene, optionally substituted carbocyclylene, optionally substituted arylene, optionally substituted heteroarylene, a peptidyl group, a dipeptidyl group, a polypeptidyl group, a click chemistry handle, or a combination thereof.
Embodiment 16. The compound of any one of embodiments 1-15, wherein L further comprises a click chemistry handle.
Embodiment 17. The compound of embodiment 16, wherein the click chemistry handle comprises an alkyne.
Embodiment 18. The compound of any one of embodiments 16 and 17, wherein the click chemistry handle comprises a strained alkyne.
Embodiment 19. The compound of any one of embodiments 16-18, wherein the click chemistry handle comprises a cyclooctyne.
Embodiment 20. The compound of any one of embodiments 16-19, wherein the click chemistry handle is of formula (IV):

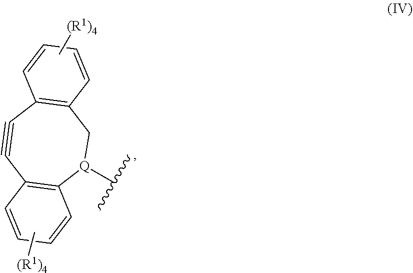

or a salt thereof, wherein:
each instance of R¹ is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂;
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring; and
Q is CH or N.
   Embodiment 21. The compound of any one of embodiments 16-20, wherein the click chemistry handle is of formula (IV-a):

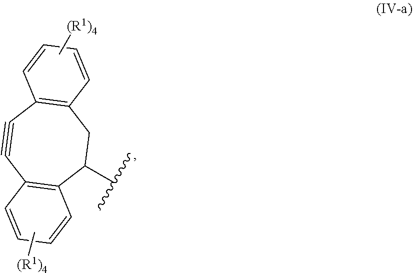

or a salt thereof.
Embodiment 22. The compound of any one of embodiments 16-20, wherein the click chemistry handle is of formula (IV-b):

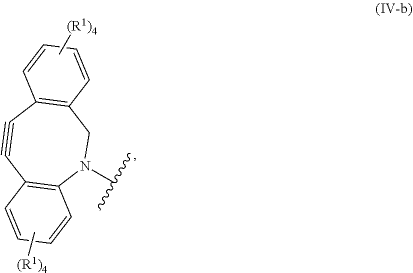

or a salt thereof.
Embodiment 23. The compound of any one of embodiments 16-22, wherein at least one instance of R¹ is hydrogen.
Embodiment 24. The compound of any one of embodiments 16-23, wherein all instances of R¹ are hydrogen.
Embodiment 25. The compound of any one of embodiments 16-20 and 22-24, wherein the click chemistry handle is of formula (IV-b-i):

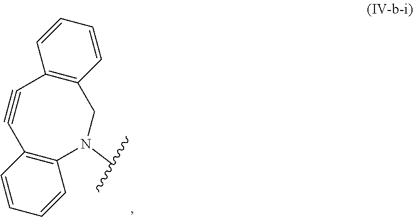 [SMILES: *N1Cc2ccccc2C#Cc2ccccc21]

or a salt thereof.
Embodiment 26. The compound of any one of embodiments 1-25, wherein L further comprises optionally substituted alkylene.
Embodiment 27. The compound of any one of embodiments 1-26, wherein L further comprises optionally substituted C₁₋₁₀ alkylene.
Embodiment 28. The compound of any one of embodiments 1-27, wherein L further comprises optionally substituted C₁₋₆ alkylene.
Embodiment 29. The compound of any one of embodiments 1-28, wherein L further comprises substituted C₁₋₆ alkylene.
Embodiment 30. The compound of any one of embodiments 1-29, wherein L further comprises:

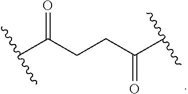 [SMILES: *C(=O)CCC(*)=O]

Embodiment 31. The compound of any one of embodiments 1-20 and 22-30, wherein L comprises:

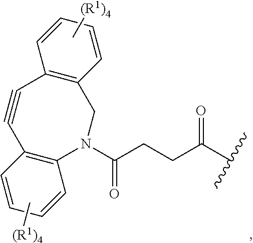

or a salt thereof.
Embodiment 32. The compound of any one of embodiments 1-20 and 22-31, wherein L comprises:

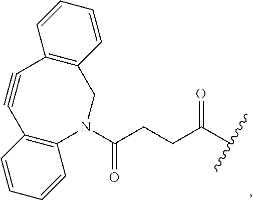 [SMILES: *C(=O)CCC(=O)N1Cc2ccccc2C#Cc2ccccc21]

or a salt thereof.
Embodiment 33. The compound of any one of embodiments 1-20 and 22-32, wherein L comprises a moiety selected from:

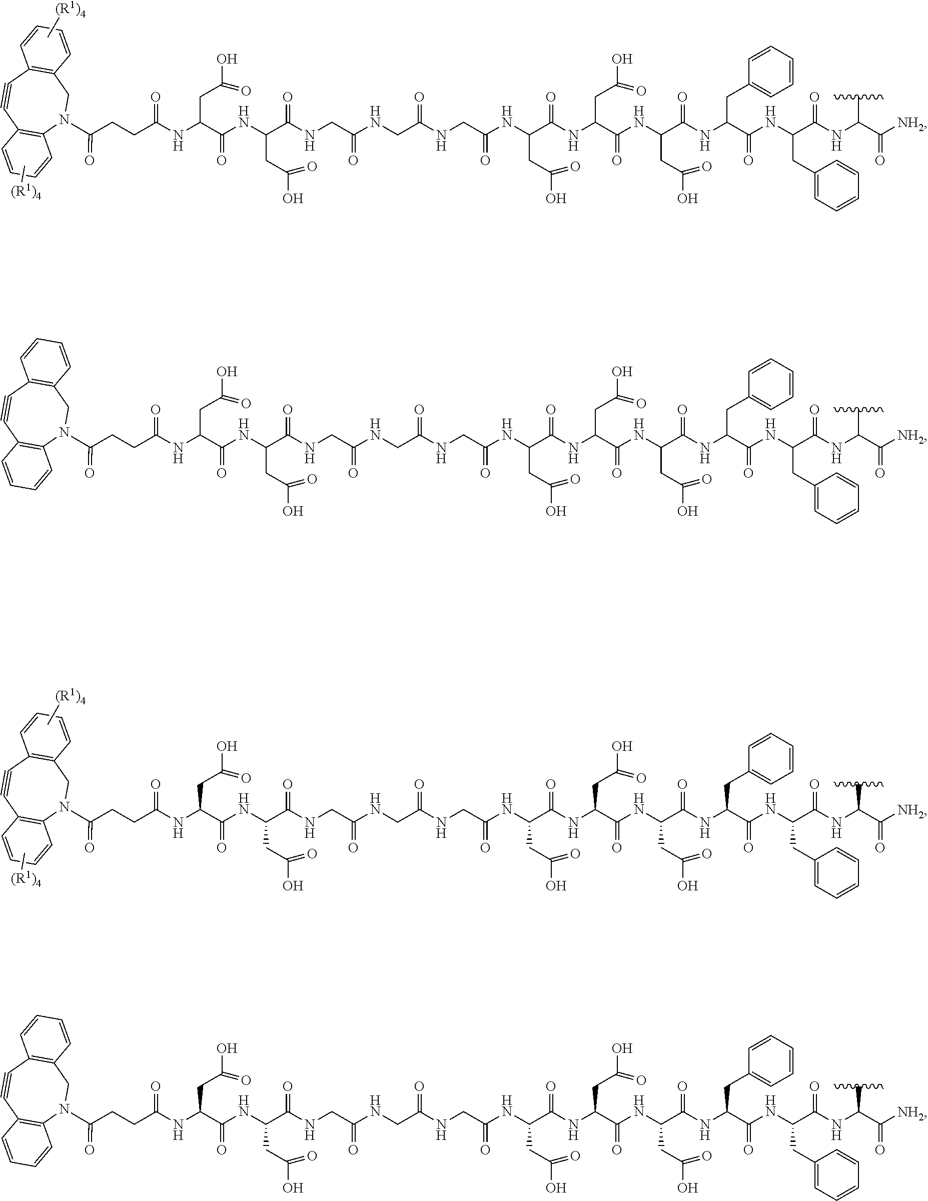

or a salt thereof.
Embodiment 34. The compound of any one of embodiments 1-33, wherein L further comprises optionally substituted heterocyclylene.
Embodiment 35. The compound of any one of embodiments 1-34, wherein L comprises:

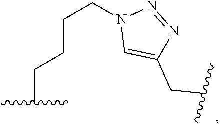 [SMILES: *CCCCn1cc(C*)nn1]

or a salt thereof.
Embodiment 36. The compound of any one of embodiments 1-35, wherein L comprises:

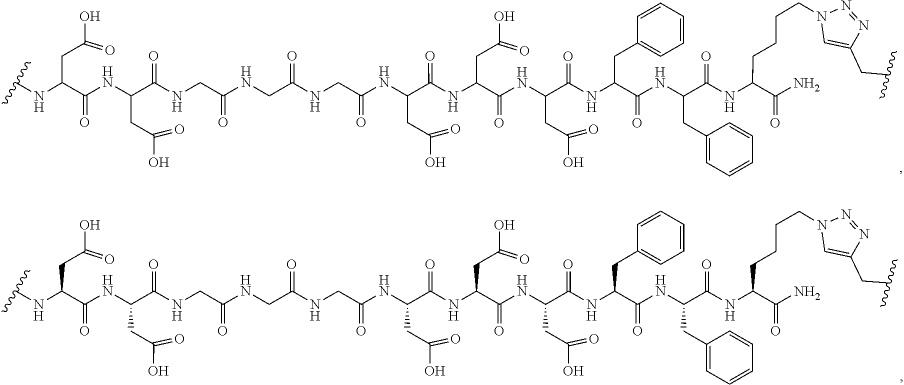 [SMILES: *Cc1cn(CCCCC(NC(=O)C(Cc2ccccc2)NC(=O)C(Cc2ccccc2)NC(=O)C(CC(=O)O)NC(=O)C(CC(=O)O)NC(=O)C(CC(=O)O)NC(=O)CNC(=O)CNC(=O)CNC(=O)C(CC(=O)O)NC(=O)C(CC(=O)O)N*)C(N)=O)nn1 ; *Cc1cn(CCCC[C@H](NC(=O)[C@H](Cc2ccccc2)NC(=O)[C@H](Cc2ccccc2)NC(=O)[C@H](CC(=O)O)NC(=O)[C@H](CC(=O)O)NC(=O)[C@H](CC(=O)O)NC(=O)CNC(=O)CNC(=O)CNC(=O)[C@H](CC(=O)O)NC(=O)[C@H](CC(=O)O)N*)C(N)=O)nn1]

or a salt thereof.
Embodiment 37. The compound of any one of embodiments 1-20 and 22-36, wherein the compound is of formula:

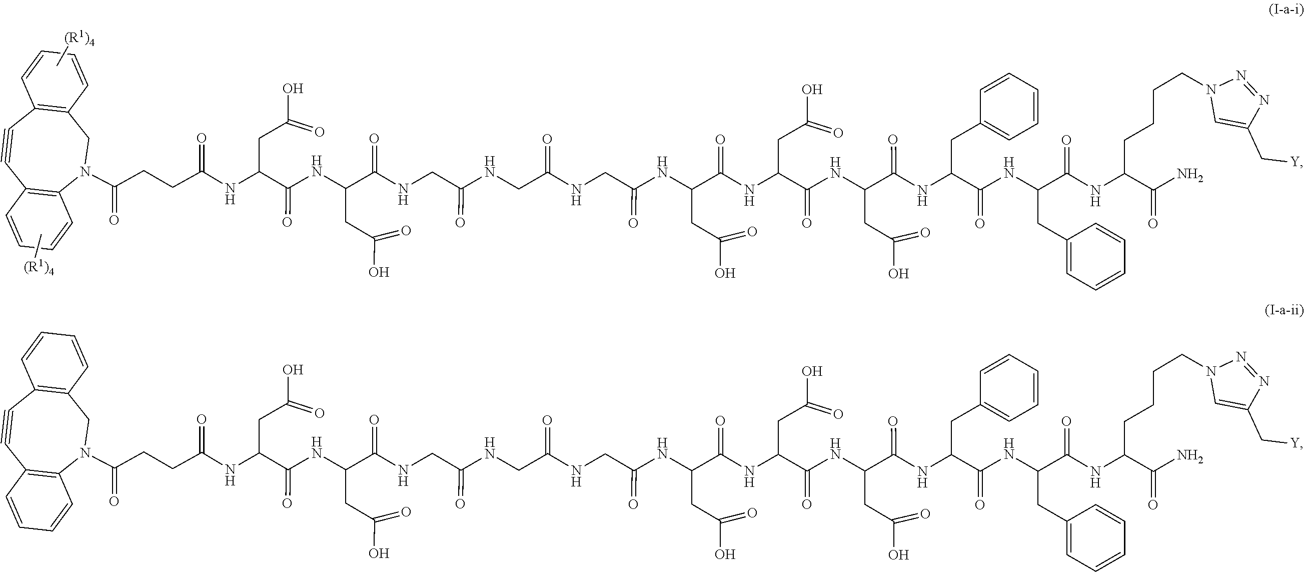

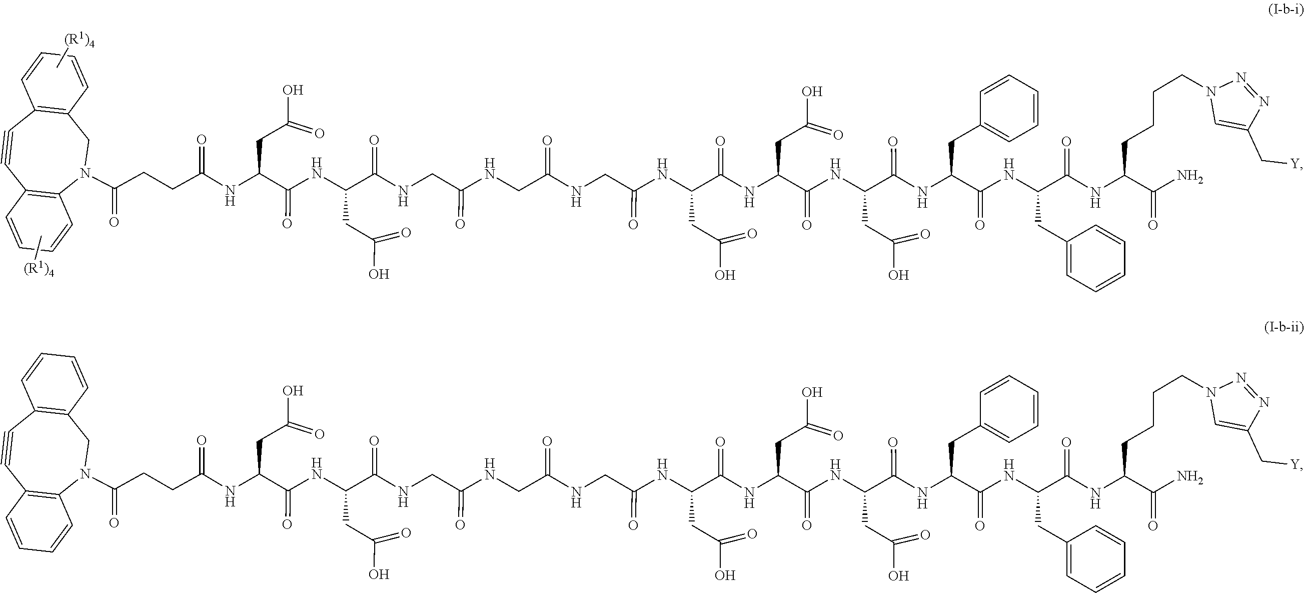

or a salt thereof.
Embodiment 38. The compound of any one of embodiments 1-37, wherein the oligonucleotide comprises Q24.
Embodiment 39. The compound of any one of embodiments 1-38, wherein Y further comprises a biotin moiety.
Embodiment 40. The compound of embodiment 39, wherein the biotin moiety is a bis-biotin moiety.
Embodiment 41. The compound of any one of embodiments 1-40, wherein Y further comprises an avidin protein.
Embodiment 42. The compound of embodiment 41, wherein the avidin protein is streptavidin.
Embodiment 43. The compound of any one of embodiments 1-42, wherein Y is immobilized to a surface.
Embodiment 44. The compound of any one of embodiments 1-43, wherein the oligonucleotide and the polypeptide are separated by between about 25 Å and about 75 Å, inclusive.
Embodiment 45. A method of preparing a compound of Formula (II):

Z-L-Y (II),

or a salt thereof, comprising reacting a compound of Formula (I):

L-Y (I),

or a salt thereof, with a compound of formula Z—N₃, or a salt thereof, wherein:
L comprises a polypeptidyl group;
Y is an oligonucleotide; and
Z is a polypeptide.
   Embodiment 46. The method of embodiment 45, wherein reacting a compound of Formula (I), or a salt thereof, with a compound of formula Z—N₃, or a salt thereof, comprises a click chemistry reaction.
   Embodiment 47. The method of any one of embodiments 45 and 46, wherein reacting a compound of Formula (I), or a salt thereof, with a compound of formula Z—N₃, or a salt thereof, comprises an azide-alkyne cycloaddition.
   Embodiment 48. The method of any one of embodiments 45-47, wherein L further comprises a click chemistry handle.
   Embodiment 49. The method of embodiment 48, wherein the click chemistry handle is of formula (IV-b-i):

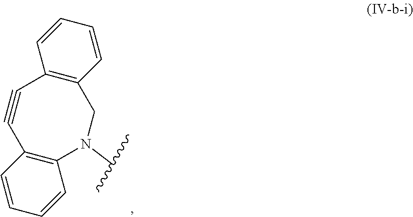 [SMILES: *N1Cc2ccccc2C#Cc2ccccc21]

or a salt thereof.
Embodiment 50. The method of any one of embodiments 45-49, wherein L comprises a moiety selected from:

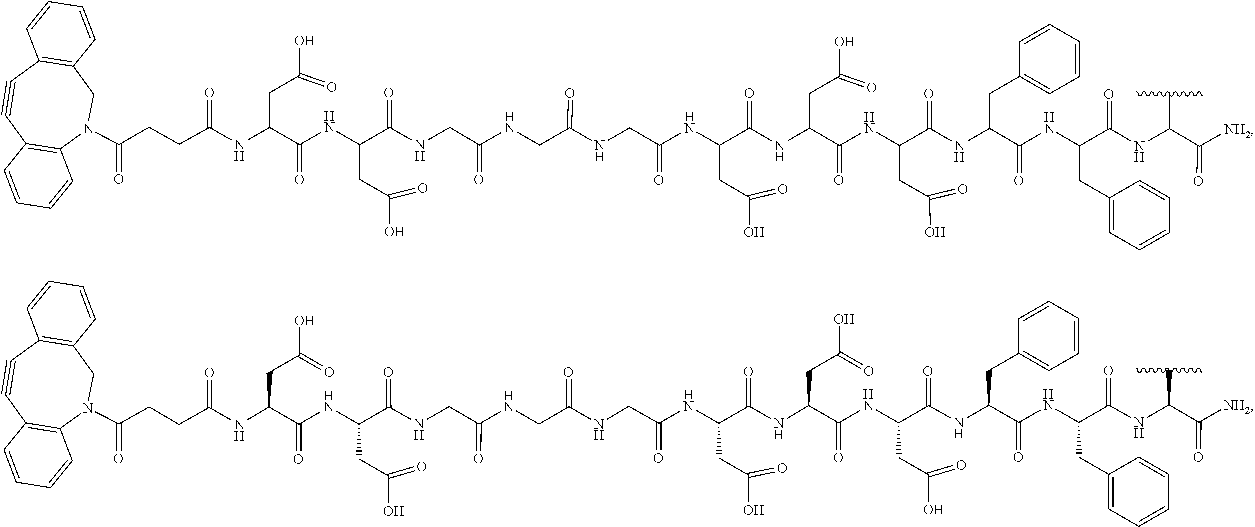 [SMILES: *C(NC(=O)C(Cc1ccccc1)NC(=O)C(Cc1ccccc1)NC(=O)C(CC(=O)O)NC(=O)C(CC(=O)O)NC(=O)C(CC(=O)O)NC(=O)CNC(=O)CNC(=O)CNC(=O)C(CC(=O)O)NC(=O)C(CC(=O)O)NC(=O)CCC(=O)N1Cc2ccccc2C#Cc2ccccc21)C(N)=O ; *[C@H](NC(=O)[C@H](Cc1ccccc1)NC(=O)[C@H](Cc1ccccc1)NC(=O)[C@H](CC(=O)O)NC(=O)[C@H](CC(=O)O)NC(=O)[C@H](CC(=O)O)NC(=O)CNC(=O)CNC(=O)CNC(=O)[C@H](CC(=O)O)NC(=O)[C@H](CC(=O)O)NC(=O)CCC(=O)N1Cc2ccccc2C#Cc2ccccc21)C(N)=O]

or a salt thereof.
Embodiment 51. The method of any one of embodiments 45-50, further comprising reacting a compound of formula L-N₃, or a salt thereof, with a compound of formula Y-propargyl, or a salt thereof, to provide the compound of Formula (I):

L-Y (I),

or a salt thereof.
Embodiment 52. The method of any one of embodiments 45-51, wherein the compound of formula L-N₃ comprises a moiety selected from:

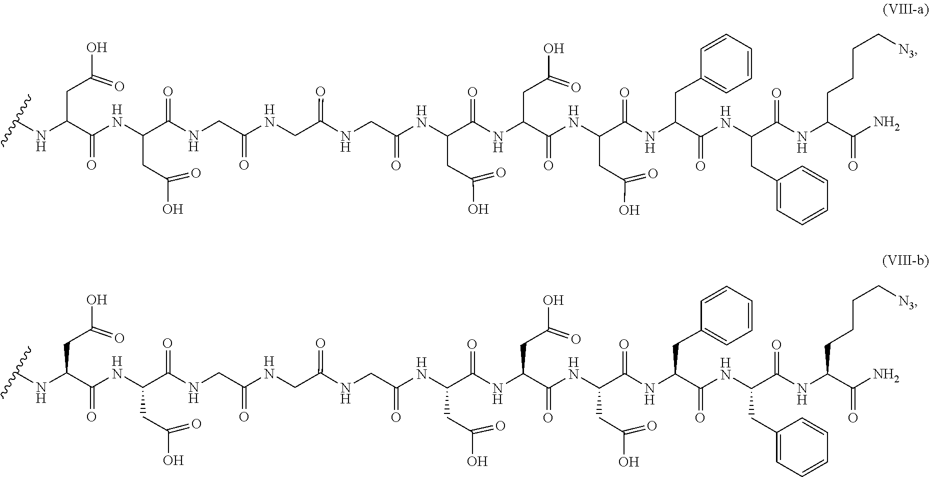 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(CCCCN=[N+]=[N-])C(N)=O ; *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](CCCCN=[N+]=[N-])C(N)=O]

or a salt thereof.
Embodiment 53. The method of any one of embodiments 45-52, wherein the compound of formula L-N₃ is of formula:

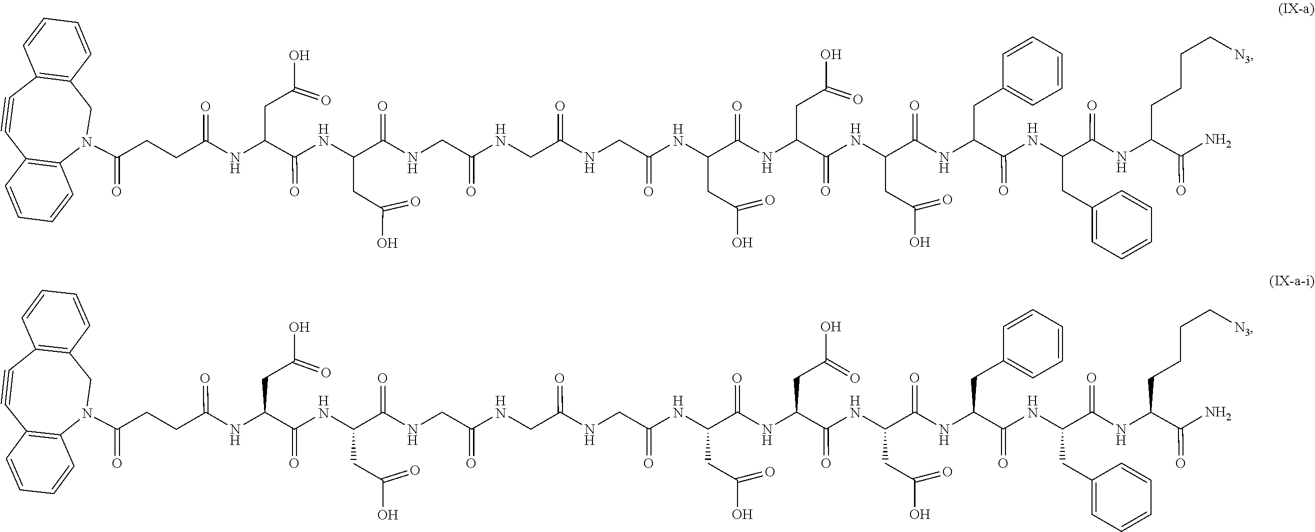 [SMILES: [N-]=[N+]=NCCCCC(NC(=O)C(Cc1ccccc1)NC(=O)C(Cc1ccccc1)NC(=O)C(CC(=O)O)NC(=O)C(CC(=O)O)NC(=O)C(CC(=O)O)NC(=O)CNC(=O)CNC(=O)CNC(=O)C(CC(=O)O)NC(=O)C(CC(=O)O)NC(=O)CCC(=O)N1Cc2ccccc2C#Cc2ccccc21)C(N)=O ; [N-]=[N+]=NCCCC[C@H](NC(=O)[C@H](Cc1ccccc1)NC(=O)[C@H](Cc1ccccc1)NC(=O)[C@H](CC(=O)O)NC(=O)[C@H](CC(=O)O)NC(=O)[C@H](CC(=O)O)NC(=O)CNC(=O)CNC(=O)CNC(=O)[C@H](CC(=O)O)NC(=O)[C@H](CC(=O)O)NC(=O)CCC(=O)N1Cc2ccccc2C#Cc2ccccc21)C(N)=O]

or a salt thereof.
Embodiment 54. The method of any one of embodiments 45-53, wherein the compound of Formula (I) is of formula:

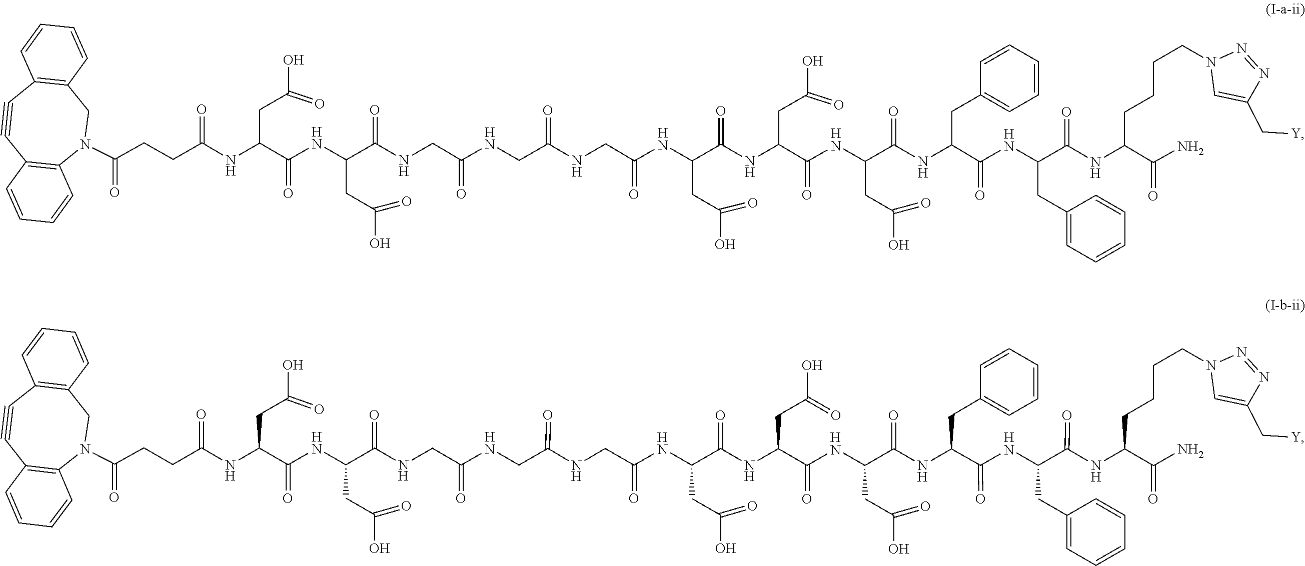

or a salt thereof.
Embodiment 55. A method of sequencing a polypeptide Z, the method comprising reacting a compound of Formula (II):

Z-L-Y (II),

or a salt thereof, with a peptidase, wherein:
L comprises a polypeptidyl group; and
Y is an oligonucleotide;
reacting the compound of Formula (II), or salt thereof, with a peptidase, in a degradation process;
obtaining data during the degradation process;
analyzing the data to determine portions of the data corresponding to amino acids that are sequentially exposed at a terminus of the polypeptide during the degradation process; and
outputting an amino acid sequence representative of the polypeptide.
   Embodiment 56. The method of embodiment 55, further comprising reacting a compound of Formula (I):

L-Y (I),

or a salt thereof, with a functionalized polypeptide, or salt thereof, to provide the compound of Formula (II):

Z-L-Y (II),

or a salt thereof, wherein the functionalized polypeptide, or salt thereof, comprises a click chemistry handle, and the compound of Formula (I), or salt thereof, comprises a click chemistry handle.
Embodiment 57. The method of any one of embodiments 55 and 56, wherein the peptidase is an exopeptidase.
Embodiment 58. The method of any one of embodiments 55-57, wherein the peptidase is an aminopeptidase.
Embodiment 59. The method any one of embodiments 55-58, wherein the peptidase is proline aminopeptidase, a proline aminopeptidase, a glutamate/aspartate-specific aminopeptidase, a methionine-specific aminopeptidase, or a zinc metalloprotease.
Embodiment 60. The method of any one of embodiments 55-59, wherein the peptidase is a TET aminopeptidase.
Embodiment 61. The method of any one of embodiments 55-60, wherein a cut depth of the compound of Formula (II) is improved compared to a cut depth of a compound of Formula (X):

Z-L¹-Y (X),

wherein L¹ is:

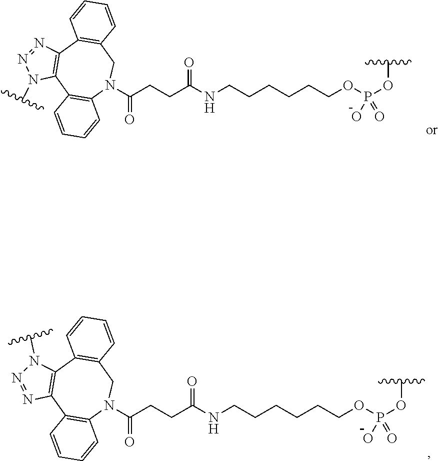 [SMILES: *OP(=O)(O)OCCCCCCNC(=O)CCC(=O)N1Cc2ccccc2-c2nnn(*)c2-c2ccccc21 ; *OP(=O)(O)OCCCCCCNC(=O)CCC(=O)N1Cc2ccccc2-c2c(nnn2*)-c2ccccc21]

or a salt thereof.
Embodiment 62. The method of embodiment 61, wherein the cut depth of the compound of Formula (II) is improved by between about 10% and about 100% compared to the cut depth of the compound of Formula (X).
Embodiment 63. The method of any one of embodiments 55-62, wherein a percentage of reads that terminate at a specific residue of the compound of Formula (II) is improved compared to a percentage of reads that terminate at a specific residue of a compound of Formula (X):

Z-L¹-Y (X),

wherein L¹ is:

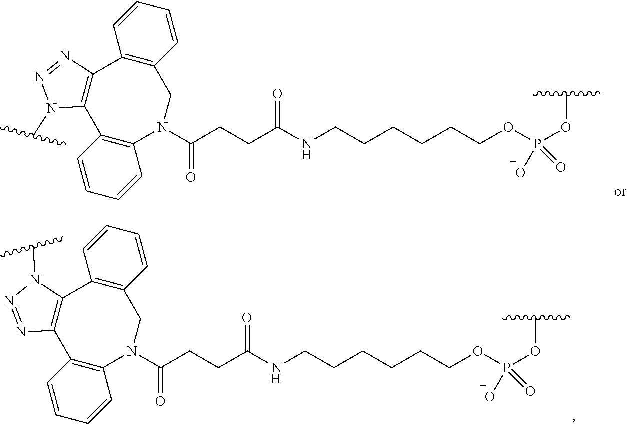

or a salt thereof.
Embodiment 64. The method of embodiment 63, wherein the percentage of reads that terminate at a specific residue of the compound of Formula (II) is improved by between about 100% and about 1000% compared to the percentage of reads that terminate at a specific residue of the compound of Formula (X).
Embodiment 65. The method of any one of embodiments 55-64, wherein a cutting rate of the compound of Formula (II) is improved compared to a cutting rate of a compound of Formula (X):

Z-L¹-Y (X),

wherein L¹ is:

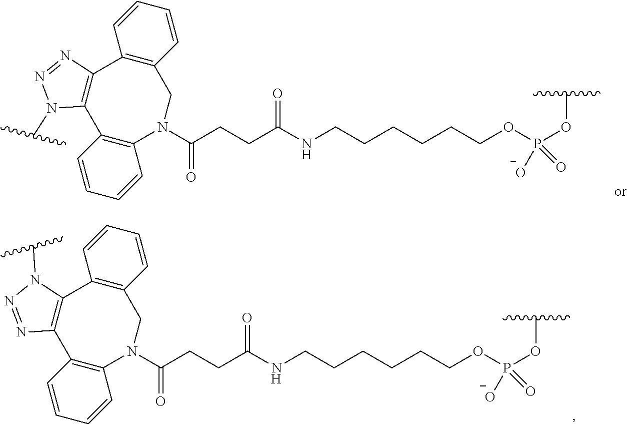

or a salt thereof.
Embodiment 66. The method of embodiment 65, wherein the cutting rate of the compound of Formula (II) is at least doubled, at least tripled, or at least quadrupled compared to the cutting rate of the compound of Formula (X).

1. A compound of Formula (I):
L-Y (I),
or a salt thereof, wherein:
L comprises a polypeptidyl group; and
Y comprises an oligonucleotide.

2. A compound of Formula (II):
Z-L-Y (II),
or a salt thereof, wherein:
L comprises a polypeptidyl group;
Y comprises an oligonucleotide; and
Z is a polypeptide.

3. The compound of claim 1, wherein the polypeptidyl group comprises between 5 and 20 amino acid residues, inclusive.

4. The compound of claim 1, wherein the polypeptidyl group is between about 20 Å and about 75 Å in length, inclusive.

5. The compound of claim 1, wherein the polypeptidyl group comprises between 1 and 10 negatively charged moieties at physiological pH, inclusive.

6. The compound of claim 1, wherein the polypeptidyl group comprises between 1 and 15 aspartate residues, inclusive.

7. The compound of claim 1, wherein the polypeptidyl group comprises between 1 and 10 phenylalanine residues, inclusive.

8. The compound of claim 1, wherein the polypeptidyl group comprises between 1 and 10 glycine residues, inclusive.

9. The compound of claim 1, wherein the polypeptidyl group comprises between 1 and 5 proline residues, inclusive.

10. The compound of claim 1, wherein the polypeptidyl group comprises between 1 and 5 GP repeats, inclusive.

11. (canceled)

12. The compound of claim 1, wherein the polypeptidyl group comprises a moiety selected from: 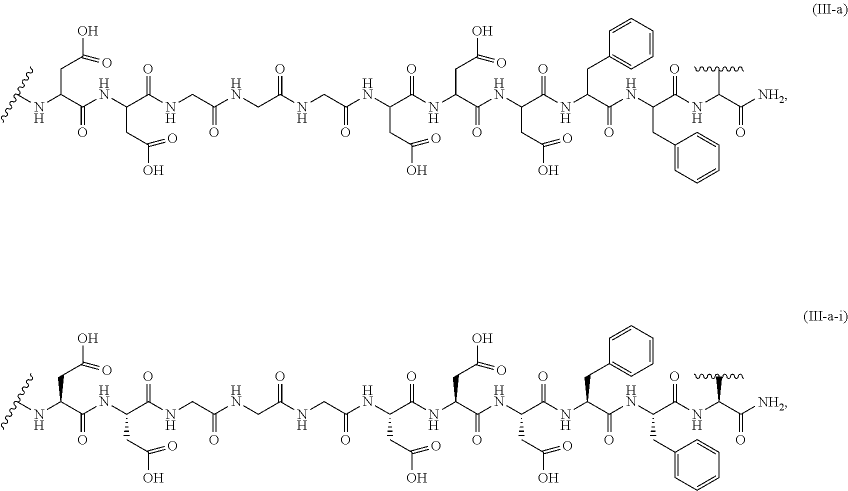 [SMILES: *NC(CC(=O)O)C(=O)NC(CC(=O)O)C(=O)NCC(=O)NCC(=O)NCC(=O)NC(CC(=O)O)C(=O)NC(CC(=O)O)C(=O)NC(CC(=O)O)C(=O)NC(Cc1ccccc1)C(=O)NC(Cc1ccccc1)C(=O)NC(*)C(N)=O ; *N[C@@H](CC(=O)O)C(=O)N[C@@H](CC(=O)O)C(=O)NCC(=O)NCC(=O)NCC(=O)N[C@@H](CC(=O)O)C(=O)N[C@@H](CC(=O)O)C(=O)N[C@@H](CC(=O)O)C(=O)N[C@@H](Cc1ccccc1)C(=O)N[C@@H](Cc1ccccc1)C(=O)N[C@@H](*)C(N)=O]
or a salt thereof.

13. The compound of claim 1, wherein the polypeptidyl group comprises a sequence selected from GPPPPPPPPG (SEQ ID NO: 61), isoEGWRW (SEQ ID NO: 62), DDGGGDDDFF (SEQ ID NO: 32), GGSSSGSGNDEEFQ (SEQ ID NO: 59), GGGGGDPDPD (SEQ ID NO: 54), GGGGGDPDPDFF (SEQ ID NO: 55), GGGGGGDPDPD (SEQ ID NO: 57), GDGDGDGDGDFF (SEQ ID NO: 53), GDDGDGDGDFF (SEQ ID NO: 51), NNGGGNNNFF (SEQ ID NO: 65), or DDGGGCyCyCyFF (SEQ ID NO: 45), or a salt thereof, wherein Cy is cysteic acid.

14-15. (canceled)

16. The compound of claim 1, wherein L further comprises a click chemistry handle.

17-36. (canceled)

37. The compound of claim 1, wherein the compound is of formula: 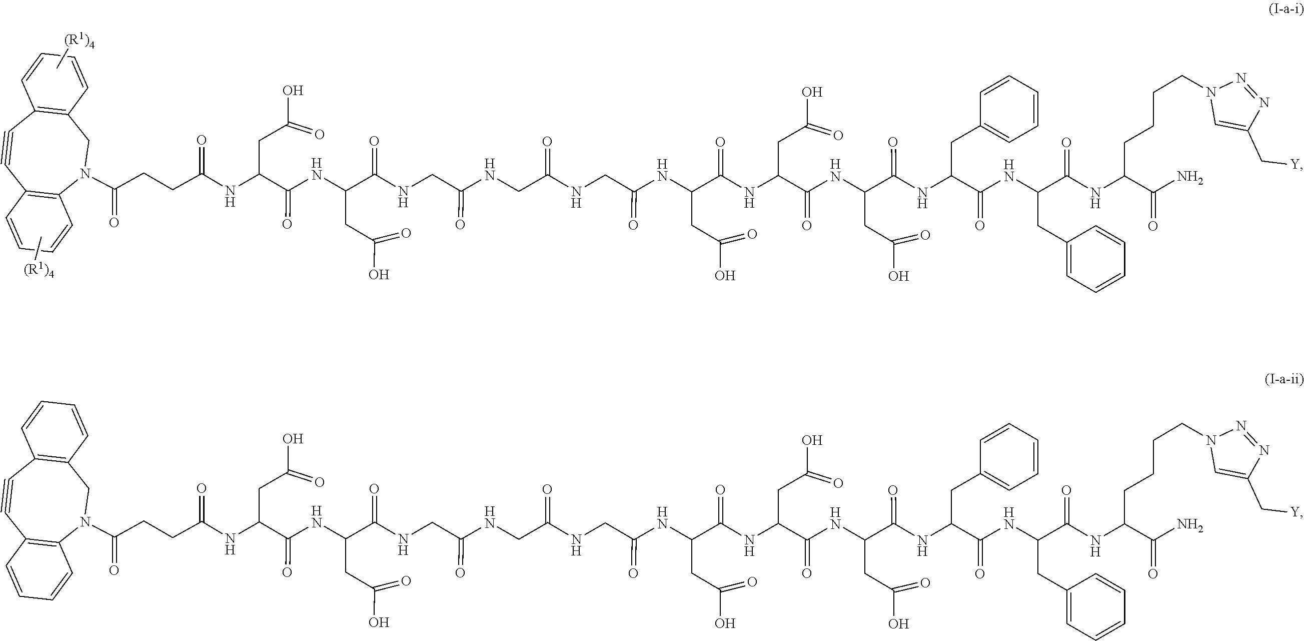 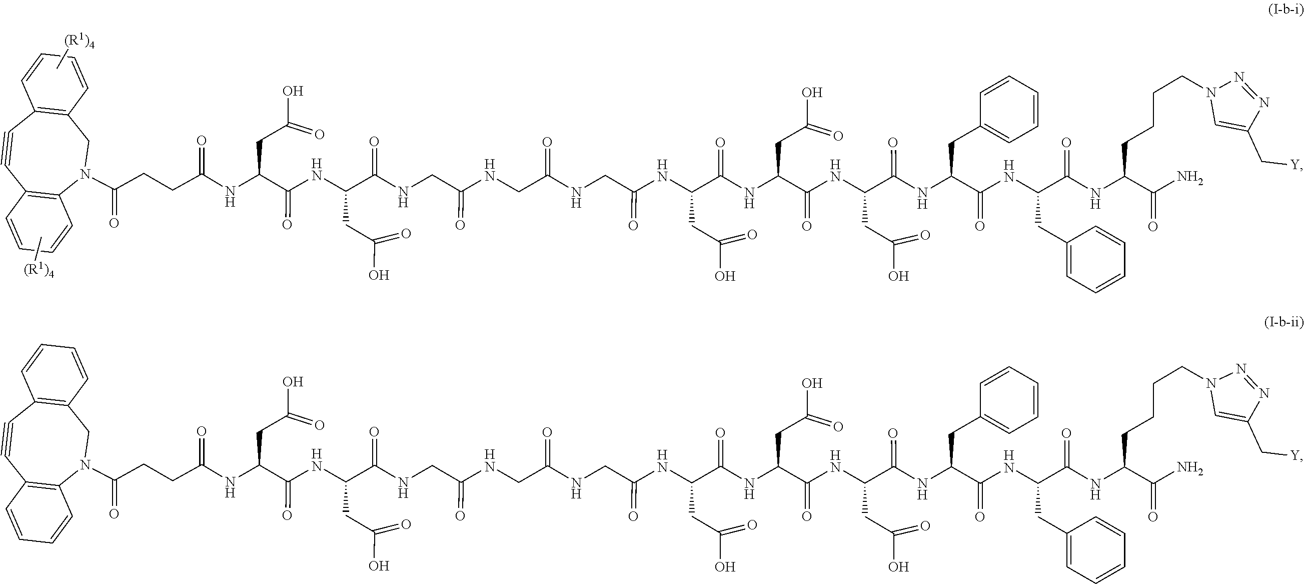
or a salt thereof.

38. The compound of claim 1, wherein the oligonucleotide comprises Q24.

39. The compound of claim 1, wherein Y further comprises a biotin moiety.

40. (canceled)

41. The compound of claim 1, wherein Y further comprises an avidin protein.

42. (canceled)

43. The compound of claim 1, wherein Y is immobilized to a surface.

44. (canceled)

45. A method of preparing a compound of Formula (II):
Z-L-Y (II),
or a salt thereof, comprising reacting a compound of Formula (I):
L-Y (I),
or a salt thereof, with a compound of formula Z—N₃, or a salt thereof, wherein:
L comprises a polypeptidyl group;
Y is an oligonucleotide; and
Z is a polypeptide.

46-54. (canceled)

55. A method of sequencing a polypeptide Z, the method comprising reacting a compound of Formula (II):
Z-L-Y (II),
or a salt thereof, with a peptidase, in a degradation process, wherein:
L comprises a polypeptidyl group; and
Y is an oligonucleotide;
obtaining data during the degradation process;
analyzing the data to determine portions of the data corresponding to amino acids that are sequentially exposed at a terminus of the polypeptide during the degradation process; and,
optionally, outputting an amino acid sequence representative of the polypeptide.

56-66. (canceled)
